(19) United States
(12) Patent Application Publication
(10) Pub. No.: US 2025/0073256 A1
(43) Pub. Date: Mar. 6, 2025

(54) PREKALLIKREIN-MODULATING COMPOSITIONS AND METHODS OF USE THEREOF

(71) Applicant: ADARx Pharmaceuticals, Inc., San Diego, CA (US)

(72) Inventors: Zhen Li, San Diego, CA (US); Rui Zhu, San Diego, CA (US); Zhiqing (Joel) Zhou, San Diego, CA (US); Kimberly Fultz, San Diego, CA (US); Sean Studer, Del Mar, CA (US)

(73) Assignee: ADARx Pharmaceuticals, Inc., San Diego, CA (US)

(21) Appl. No.: 18/673,525

(22) Filed: May 24, 2024

Related U.S. Application Data
(63) Continuation of application No. 17/937,344, filed on Sep. 30, 2022, now Pat. No. 12,042,509.
(60) Provisional application No. 63/287,969, filed on Dec. 9, 2021.
(60) Provisional application No. 63/283,175, filed on Nov. 24, 2021.
(60) Provisional application No. 63/270,504, filed on Oct. 21, 2021.
(60) Provisional application No. 63/252,554, filed on Oct. 5, 2021.
(60) Provisional application No. 63/251,571, filed on Oct. 1, 2021.

(51) Int. Cl. A61K 31/712; A61K 31/7125; A61P 9/10; C12N 15/113

(52) U.S. Cl. CPC A61K 31/712; A61K 31/7125; A61P 9/10; C12N 15/1137; C12N 2310/14; C12N 2310/315; C12N 2310/321; C12N 2310/322; C12N 2310/351

(57) ABSTRACT

Aspects of the disclosure provide compounds, compositions, and methods for modulating the expression or activity of plasma prekallikrein (PKK). In some aspects, the compounds, compositions, and methods of the disclosure can be used to reduce the expression of PKK mRNA in a cell or animal. In some aspects, the compounds, compositions, and methods of the disclosure can be used to reduce the expression of PKK protein in a cell or animal.

### CROSS-REFERENCE TO RELATED APPLICATIONS

This application claims the benefit of U.S. Provisional Application No. 63/251,571, filed Oct. 1, 2021; U.S. Provisional Application No. 63/252,554, filed Oct. 5, 2021; U.S. Provisional Application No. 63/270,504, filed Oct. 21, 2021; U.S. Provisional Application No. 63/283,175, filed Nov. 24, 2021; and U.S. Provisional Application No. 63/287,969, filed Dec. 9, 2021. The disclosure of each of the prior applications is considered part of and is incorporated by reference in its entirety in the disclosure of this application.

### REFERENCE TO AN ELECTRONIC SEQUENCE LISTING

The contents of the electronic sequence listing (A127870009US05-SEQ-JIB.xml; Size: 638,286 bytes; and Date of Creation: Sep. 30, 2022) is herein incorporated by reference in its entirety.

### BACKGROUND

Plasma prekallikrein (PKK) is a glycoprotein that participates in the surface-dependent activation of blood coagulation, fibrinolysis, kinin generation, and inflammation. PKK, which is encoded by the KLKB1 gene, is the precursor of plasma kallikrein (PK). PKK is present in plasma as a contact factor that forms non-covalent complexes with high molecular weight kininogen. PKK is converted to PK by Factor XIIa through the cleavage of an internal Arg-Ile peptide bond. PK is a member of the kinin-kallikrein pathway, which consists of several proteins that play a role in inflammation, blood pressure control, coagulation, and pain. PK liberates kinins from kininogens and also generates plasmin from plasminogen. For example, plasma kallikrein cleaves high molecular weight kininogen (HMWK) to generate bradykinin. The kinins, especially bradykinin, go on to induce downstream effects including vasodilation and edema (See, e.g., Schmaier. J. Thromb. Haemost 14: 28-39, 2016).

Certain mutations in PKK cause PKK deficiency, also known as Fletcher Factor deficiency, a rare coagulation deficiency characterized by a prolonged activated partial thromboplastin time (PTT). PKK deficiency has been linked to inflammatory and thrombotic disorders.

Mutations in PKK prevent the release of plasmin and kinins (e.g., bradykinin), and/or reduce fibrinolysis. This results in reduced vasodilation and increased blood clot formation, which in turn increase the likelihood of contracting inflammatory or thrombotic diseases. People with PKK deficiency are often asymptomatic, but still present a prolonged activated PTT, and are at risk of developing such diseases.

An inflammatory disorder occurs when the immune system mistakenly attacks the body's own cells or tissues. This causes abnormal inflammation that can result in chronic pain, redness, swelling, stiffness and damage to otherwise healthy body tissues. Inflammatory diseases include a vast array of disorders and conditions that are characterized by inflammation. Examples include rheumatoid arthritis, allergy, asthma, autoimmune diseases, coeliac disease, glomerulonephritis, hepatitis, inflammatory bowel disease, pre-perfusion injury and transplant rejection. It is estimated that over 1.36 million adults in the US suffer from rheumatoid arthritis and 3 million from inflammatory bowel disease.

Thrombosis is the formation of a blood clot inside a blood vessel, obstructing the flow of blood through the circulatory system. When a blood vessel is injured, the body uses platelets and fibrin to form a blood clot to prevent blood loss. Even when a blood vessel is not injured, blood clots may form in the body under certain conditions. In healthy people, homeostatic balance exists between procoagulant (clotting) forces and anticoagulant and fibrinolytic forces. Numerous genetic, acquired, and environmental factors can tip the balance in favor of coagulation, leading to the pathologic formation of thrombi in veins (e.g., deep venous thrombosis), arteries (e.g., myocardial infarction, ischemic stroke), or cardiac chambers. Thrombi can obstruct blood flow at the site of formation or detach and embolize to block a distant blood vessel (e.g., pulmonary embolism, embolic stroke). In the US alone, about 900,000 people are affected by blood clots each year, and about 100,000 of those people will die from blood clot-related complications.

Hereditary angioedema (HAE) is a rare inflammatory disease characterized by recurrent episodes of swelling around the head and extremities (Zuraw, B. L. N. Engl. J. Med. 359: 1027-36, 2008). Angioedema attacks occur with unpredictable frequency and are typically focused on the skin, and gastric, oropharyngeal, and laryngeal mucosas. Asphyxiation due to laryngeal swelling can result in mortality. HAE is caused by deficiency or malfunction of the serine protease inhibitor C1-INH (Kaplan, A. P. et al. J. Allergy Clin. Immunol. 109: 195-209, 2002). C1-INH is the primary inhibitor of coagulation factors 12 and 11 (Factor 11) of the intrinsic coagulation pathway as well as plasma kallikrein (Gigli, I. et al. J. Immunol. 104:574-581, 1970). C1-INH mediated inhibition of plasma kallikrein and Factor 12 results in inactivation of the kallikrein pathway and decreased levels of bradykinin (BK). C1-INH deficiency or dysfunction results in overproduction of BK, which is the mechanism by which HAE attacks are believed to occur. Type III HAE has been linked with mutations in the Factor 12 gene, which encodes coagulation protein Factor 12 (Cichon, S. et al. Am. J. Hum. Genet. 79: 1098-1104, 2006).

There is currently no cure for certain inflammatory conditions, such as HAE, or thrombotic conditions associated with dysregulation of PKK or other members of the kallikrein pathway. Accordingly, there is a need to find effective treatments for PKK related diseases.

### SUMMARY

The present disclosure provides compounds, compositions, and methods for modulating the expression or activity of PKK. In certain embodiments, the compounds, compositions, and methods can be used to reduce the expression of PKK mRNA in a cell or animal. In certain embodiments, the compounds, compositions, and methods can be used to reduce the amount of PKK protein in a cell or animal.

In certain embodiments, the animal has an inflammatory or thrombotic disease, disorder or condition or a symptom thereof. In certain embodiments, the disease is hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. Certain compounds, compositions and methods provided herein are directed to reducing an inflammatory or thrombotic disease, disorder or condition or a symptom thereof or hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in an animal. In certain embodiments, the compounds and compositions provided herein are potent and tolerable and inhibit PKK expression, which can be used to treat, prevent, ameliorate, or slow progression of an inflammatory or thrombotic disease, disorder or condition or a symptom thereof or hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct.

In certain embodiments, the compounds and compositions comprise one or more features that are effective for increasing potency. In certain embodiments, the compounds and compositions comprise one or more features that are effective for increasing tolerability. In certain embodiments, compounds and compositions comprise one or more features that are effective for targeting the compound or composition to a cell or tissue. In certain embodiments, the compounds and compositions are more potent or have greater therapeutic value than compounds publicly disclosed.

### DETAILED DESCRIPTION

It is to be understood that both the foregoing summary and the following detailed description are exemplary and explanatory only and are not restrictive of the embodiments, as claimed. The section headings used herein are for organizational purposes only and are not to be construed as limiting the subject matter described.

All documents, or portions of documents, cited in this application, including, but not limited to, patents, patent applications, articles, books, treatises, and GenBank, NCBI and other sequence reference records are hereby expressly incorporated by reference for the portions of the document discussed herein, as well as in their entirety as of the date of filing this application.

It is understood that the sequence set forth in each SEQ ID NO contained herein is independent of any modification to a sugar moiety, an internucleoside linkage, or a nucleobase even if shown in context with a modified compound. As such, compounds defined by a SEQ ID NO may comprise, independently, one or more modifications to a sugar moiety, an internucleoside linkage, or a nucleobase. Oligomeric compounds referenced by Compound Number or Ref ID NO indicate a combination of nucleobase sequence, chemical modification, and motif.

Herein, the use of the singular includes the plural unless specifically stated otherwise. For example, the articles “a” and “an” are used herein to refer to one or to more than one (i.e., to at least one) of the grammatical object of the article. By way of example, “an element” means one element or more than one element, e.g., a plurality of elements. As used herein, the use of “or” means “and/or” unless stated otherwise. Furthermore, the use of the term “including” as well as other forms, such as “includes” and “included”, is not limiting and is used interchangeably with, the phrase “including but not limited to”.

### Definitions

Unless otherwise indicated, the following terms have the following meanings:

“Plasma prekallikrein” or “kallikrein B1,” used interchangeably with the term “PKK,” refers to any nucleic acid or protein of PKK. Exemplary nucleotide and amino acid sequences of PKK can be found, for example, at GenBank Accession No. NM_000892.5 (incorporated herein as SEQ ID NO: 1), NG_012095.2 truncated from 23529 . . . 54493 (incorporated herein as SEQ ID NO: 2), XM_017008181.1 (incorporated herein as SEQ ID NO: 3), NC_000004.12 truncated from 186215714 to 186258477 (incorporated herein as SEQ ID NO: 4), NM_001318394.2 (incorporated herein as SEQ ID NO: 5) and NM_001318396.2 (incorporated herein as SEQ ID NO: 6). Additional examples of PKK sequences are readily available through publicly available databases, e.g., GenBank, UniProt, and OMIM. Further information on PKK can be found, for example, at ncbi.nlm.nih.gov/gene/?term=PKK. PKK, as used herein, also refers to variations of the PKK gene including variants provided in the SNP database. Numerous sequence variations within the PKK gene have been identified and may be found at, for example, NCBI dbSNP and UniProt (see, e.g., ncbi.nlm.nih.gov/snp/?term=PKK). “PKK mRNA” means an mRNA encoding a PKK protein. PKK may be referred to in either upper or lower case.

“PKK specific inhibitor” refers to any agent capable of specifically inhibiting PKK RNA and/or PKK protein expression or activity at the molecular level. For example, PKK specific inhibitors include nucleic acids (including oligonucleotide compounds), peptides, antibodies, small molecules, and other agents capable of inhibiting the expression of PKK RNA and/or PKK protein.

“2′-O-methoxyethyl” or “2′-MOE” means a 2′-O(CH₂)₂—OCH₃ modification. A 2′-O-methoxyethyl modified sugar is a modified sugar with 2′-O(CH₂)₂—OCH₃ in the place of the 2′-OH group of a ribosyl ring.

“5′ start site” means the nucleotide of the target nucleic acid or region which is aligned to the 3′-most nucleoside of an antisense oligonucleotide.

“3′ stop site” means the nucleotide of the target nucleic acid or region which is aligned to the 5′-most nucleoside of an antisense oligonucleotide.

“About” means within ±10% of a value. For example, if it is stated, “a compound achieved about 70% inhibition of PKK”, it is implied that PKK levels are inhibited within a range of 60% and 80%. When about is present before a series of numbers or a range, it is understood that “about” can modify each of the numbers in the series or range.

“Administer” or “administering” refers to routes of introducing a compound or composition provided herein to an individual to perform its intended function. An example, routes of administration that can be used include, but are not limited to, parenteral administration, such as subcutaneous, intravenous, or intramuscular injection or infusion.

“Ameliorate” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense oligonucleotide” or “antisense strand” means an oligonucleotide which includes a region that is complementary to a target nucleic acid, e.g., a PKK RNA or a region thereof.

“Complementarity” in reference to an oligonucleotide means the nucleobase sequence of such oligonucleotide or one or more regions thereof that is complementary to the nucleobase sequence of another oligonucleotide or nucleic acid or one or more regions thereof when the two nucleobase sequences are aligned in opposing directions. Complementary nucleobases, as described herein, are limited to the following pairs: adenine (A) and thymine (T), adenine (A) and uracil (U), and cytosine (C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Composition” or “pharmaceutical composition” means a mixture of substances suitable for administering to an individual. For example, a composition may comprise one or more compounds or salt thereof and a sterile aqueous solution.

“Co-administration” means administration of two or more compounds in any manner in which the pharmacological effects of both are manifest in the patient. Co-administration does not require both compounds to be administered in a single pharmaceutical composition, in the same dosage form, by the same route of administration, or at the same time. The effects of both compounds need not manifest themselves at the same time. The effects need only be overlapping for a period of time and need not be coextensive. Co-administration includes parallel or sequential administration of the one or more compounds.

“Conjugate group” means a group of atoms that is attached to an oligonucleotide. A conjugate group is optionally attached to an oligonucleotide through a conjugate linker. A conjugate group may, for example, alter the distribution, targeting, or half-life of a compound into which it is incorporated. Conjugate groups include targeting moieties.

“Conjugate linker” means a group of atoms comprising at least one bond that connects a linked moiety to an oligonucleotide.

“Identity” in reference to an oligonucleotide means the nucleobase sequence of such oligonucleotide or one or more regions thereof that matches the nucleobase sequence of another oligonucleotide or nucleic acid or one or more regions thereof. Identity of an oligonucleotide to another oligonucleotide or nucleic acid need not require each nucleobase to match and may include one or more different nucleobases. By contrast, “fully identical” or “100% identity” in reference to oligonucleotides means that such oligonucleotides have the same nucleobase at each relative position over its length as the other oligonucleotide or nucleic acid.

“Individual” means a human or non-human animal selected for treatment or therapy.

“Inhibiting the expression or activity” with reference to a target nucleic acid or protein means to reduce or block the expression or activity of such target relative to the expression or activity in an untreated or control sample and does not necessarily indicate a total elimination of expression or activity.

As used herein, the term “internucleoside linkage” is the covalent linkage between adjacent nucleosides in an oligonucleotide. As used herein, “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations as further described below. Unless otherwise indicated, chiral internucleoside linkages of modified oligonucleotides described herein can be stereorandom or in a particular stereochemical configuration.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (²H), tritium (3H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent contains unnatural proportions of one or more isotopes, including, but not limited to, hydrogen (H), deuterium (²H), tritium (3H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I).

It will be understood that, in a therapeutic agent (e.g., a compound and/or modified oligonucleotide disclosed herein), any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

“Mismatch” or “non-complementary” means a nucleobase of a first oligonucleotide or nucleic acid that is not complementary to the corresponding nucleobase of a second oligonucleotide or nucleic acid when the first oligonucleotide/nucleic acid and second oligonucleotide/nucleic acid are aligned in an antiparallel orientation. For example, nucleobases including, but not limited to, a universal nucleobase, inosine, and hypoxanthine, are capable of hybridizing with at least one nucleobase but are still mismatched or non-complementary with respect to the nucleobase to which they are hybridized. As another example, a nucleobase of a first oligonucleotide/nucleic acid that is not capable of hybridizing to the corresponding nucleobase of a second oligonucleotide/nucleic acid when the first and second oligonucleotides are aligned in an antiparallel orientation is a mismatch or non-complementary nucleobase.

“Modified oligonucleotide” means an oligonucleotide, wherein at least one sugar, nucleobase, or internucleoside linkage is modified.

“Modulating” refers to changing or adjusting a feature in a cell, tissue, organ or organism. For example, modulating PKK RNA can mean to increase or decrease the level of PKK RNA and/or PKK protein in a cell, tissue, organ or organism. A “modulator” effects the change in the cell, tissue, organ or organism. For example, a PKK compound can be a modulator that decreases the amount of PKK RNA and/or PKK protein in a cell, tissue, organ or organism.

“Motif” means the pattern of unmodified and modified sugar moieties, nucleobases, and/or internucleoside linkages, in an oligonucleotide.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric Compound” means a compound comprising one or more oligonucleotides and optionally one or more additional features, such as a conjugate group or terminal group. Examples of oligomeric compounds include single-stranded and double-stranded compounds, such as, oligonucleotides, antisense oligonucleotides, interfering RNA compounds (RNAi compounds), microRNA targeting oligonucleotides, occupancy-based compounds (e.g., mRNA processing or translation blocking compounds and splicing compounds). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA) and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

“Oligonucleotide” means a polymer of linked nucleosides, each of which can be modified or unmodified, independent from one another.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Parenteral administration” means administration through injection or infusion. Parenteral administration includes subcutaneous administration, intravenous administration, intramuscular administration, intraarterial administration, intraperitoneal administration, or intracranial administration, e.g., intrathecal or intracerebroventricular administration.

“Pharmaceutically acceptable carrier or diluent” means any substance suitable for use in administering to an individual. In certain embodiments, a pharmaceutically acceptable carrier or diluent aids the administration of a compound to and absorption by an individual and can be included in the compositions of the present disclosure without causing a significant adverse toxicological effect on the patient. Non-limiting examples of pharmaceutically acceptable excipients include water, NaCl, normal saline solutions, and the like. For example, a pharmaceutically acceptable carrier can be a sterile aqueous solution, such as PBS or water-for-injection. One of skill in the art will recognize that other pharmaceutical excipients are useful in the present disclosure.

“Pharmaceutically acceptable salts” means or refers to physiologically and pharmaceutically acceptable salts of compounds, such as oligomeric compounds or oligonucleotides, i.e., salts that retain the desired biological activity of the parent compound and do not impart undesired toxicological effects thereto.

As used herein, a pharmaceutically acceptable salt is any salt of a compound provided herein which retains its biological properties and which is not toxic or otherwise undesirable for pharmaceutical use. The pharmaceutically acceptable salts of the therapeutic agents disclosed herein include salts that are prepared with relatively nontoxic acids or bases, depending on the particular substituents found on the compounds or modified oligonucleotides described herein.

When compounds of the present disclosure contain relatively acidic functionalities, base addition salts can be obtained by contacting the neutral form of such compounds with a sufficient amount of the desired base, either neat or in a suitable inert solvent.

When compounds of the present disclosure contain relatively basic functionalities, acid addition salts can be obtained by contacting the neutral form of such compounds with a sufficient amount of the desired acid, either neat or in a suitable inert solvent.

Thus, the compounds of the present disclosure may exist as salts, such as with pharmaceutically acceptable acids. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia, or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like (see, for example, Berge et al., “Pharmaceutical Salts,” Journal of Pharmaceutical Science, 1977, 66, 1-19).

Pharmaceutically acceptable salts further include, by way of example only and without limitation, sodium, potassium, calcium, magnesium, ammonium, tetraalkylammonium, and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like. In some embodiments, the pharmaceutically acceptable salt of the compounds and modified oligonucleotides disclosed herein is a sodium or a potassium salt. In some embodiments, the pharmaceutically acceptable salt of the compounds and modified oligonucleotides disclosed herein is a sodium salt.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

“Pharmaceutical agent” means a compound that provides a therapeutic benefit when administered to an individual.

“Phosphorothioate linkage” means a modified phosphate linkage in which one of the non-bridging oxygen atoms is replaced with a sulfur atom.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligonucleotide.

“Prevent” refers to delaying or forestalling the onset, development or progression of a disease, disorder, or condition for a period of time.

“RNA interference compound” or “RNAi compound” means a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to double-stranded siRNA, single-stranded siRNA, and microRNA, including microRNA mimics.

“Sense oligonucleotide” or “sense strand” means the strand of a double-stranded compound that includes a region that is substantially complementary to a region of the antisense strand of the compound.

“Specifically inhibit” with reference to a target nucleic acid or protein means to reduce or block expression or activity of the target nucleic acid or protein while minimizing or eliminating effects on non-target nucleic acids or proteins.

“Subunit” with reference to an oligonucleotide means a nucleotide, nucleoside, nucleobase or sugar or a modified nucleotide, nucleoside, nucleobase or sugar as provided herein.

“Target nucleic acid,” “target RNA,” and “nucleic acid target” all mean a nucleic acid capable of being targeted by compounds described herein.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ or region of the body, as, e.g., compared to a compound absent such a moiety.

“Terminal group” means a chemical group or group of atoms that is covalently linked to a terminus of an oligonucleotide.

“Therapeutically effective amount” or “effective amount” means an amount of a compound, pharmaceutical agent, or composition that provides a therapeutic benefit to an individual. A “therapeutically effective amount” or “effective amount” is an amount sufficient for a compound to accomplish a stated purpose relative to the absence of the compound (e.g., achieve the effect for which it is administered, treat, prevent or ameliorate a disease or reduce one or more symptoms of a disease or condition). An example of a “therapeutically effective amount” or “effective amount” is an amount sufficient to contribute to the treatment, prevention, amelioration, or reduction of a symptom or symptoms of a disease. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The term “therapeutically effective amount,” as used herein, refers to that amount of the therapeutic agent sufficient to provide a therapeutic benefit to an individual, such as treating, preventing or ameliorating the disease or disorder or symptom thereof, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. In some instances, chronic administration may be required. For example, the compositions are administered to the subject in an amount and for a duration sufficient to treat the patient.

“Treat” refers to administering a compound or pharmaceutical composition to an animal in order to effect an alteration or improvement of a disease, disorder, or condition in the animal.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure (i.e., the R and S configurations for each asymmetric center). Therefore, single stereochemical isomers as well as enantiomeric and diastereomeric mixtures of the present compounds are within the scope of the disclosure.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

### Certain Embodiments

In certain aspects, the disclosure relates to methods, compounds and compositions for inhibiting PKK. In certain embodiments, PKK is specifically inhibited. In certain embodiments, PKK is specifically degraded. In certain embodiments, PKK expression is inhibited. In certain embodiments, PKK translation is inhibited. In certain embodiments, PKK activity is inhibited. In certain embodiments, PKK expression, translation, or activity is reduced by at least 10% relative to the expression, translation, or activity in an untreated or control sample. For example, in certain embodiments, PKK expression, translation, or activity is reduced by at least 10%, at least 20%, at least 30%, at least 40%, at least 50%, at least 60%, at least 70%, at least 80%, at least 90%, at least 95%, 10-50%, 25-50%, 25-75%, 50-75%, 50-99%, or 75-99% relative to the expression, translation, or activity in an untreated or control sample. In certain embodiments, PKK expression, translation, or activity is reduced as measured by any suitable assay, including but not limited to, an immunoassay, a hybridization-based assay, or a sequencing-based assay (e.g., RNA-Seq).

In certain aspects, the disclosure relates to compounds targeted to a PKK nucleic acid. In certain embodiments, the PKK nucleic acid has the sequence set forth in GENBANK Accession No. NM_000892.5 (incorporated herein as SEQ ID NO: 1), NG_012095.2 truncated from 23529 . . . 54493 (incorporated herein as SEQ ID NO: 2), XM_017008181.1 (incorporated herein as SEQ ID NO: 3), NC_000004.12 truncated from 186215714 to 186258477 (incorporated herein as SEQ ID NO: 4), NM_001318394.2 (incorporated herein as SEQ ID NO: 5) and NM_001318396.2 (incorporated herein as SEQ ID NO: 6).

In certain embodiments, the compound is an oligomeric compound. In certain embodiments, the compound is single-stranded. In certain embodiments, the compound is double-stranded.

Certain embodiments provide a compound comprising a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-313, 626, 627, and 628.

Certain embodiments provide a compound comprising a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628.

Certain embodiments provide a compound comprising a modified oligonucleotide having a nucleobase sequence selected from any one of SEQ ID NOs: 10-313, 626, 627, and 628.

In certain embodiments, the modified oligonucleotide has a nucleobase sequence that is at least 80%, at least 85%, at least 90%, or at least 95% complementary to SEQ ID NO: 1, 3, 5 or 6. In certain embodiments, the modified oligonucleotide comprises at least one modification selected from a modified internucleoside linkage, a modified sugar, and a modified nucleobase. In certain embodiments, the compound is double-stranded.

Certain embodiments provide a compound comprising a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide.

In certain embodiments, the compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence provided in Tables 2-4, 6, and 8, and a second modified oligonucleotide (e.g., of 14 to 30, for example, 14 to 23, linked nucleosides in length) having a region of complementarity to the first modified oligonucleotide.

Certain embodiments provide a compound comprising a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide.

Certain embodiments provide a compound comprising a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide.

In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequences of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequence of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629.

In certain embodiments, the modified oligonucleotide or first modified oligonucleotide of any preceding compound has at least 80%, at least 85%, at least 90%, or at least 95% complementarity or identity to SEQ ID NO: 1, 3, 5 or 6 over its length. In certain embodiments, the modified oligonucleotide or first modified oligonucleotide has at least 1, at least 2, at least 3 mismatches to a region of SEQ ID NO: 1, 3, 5 or 6. In certain embodiments, the region of complementarity between the first modified oligonucleotide or first strand and the second modified oligonucleotide or second strand is 14 to 30 linked nucleosides in length. In certain embodiments, the region of complementarity between the first modified oligonucleotide or first strand and the second modified oligonucleotide or second strand is 14 to 23 linked nucleosides in length. In certain embodiments, the region of complementarity between the first modified oligonucleotide or first strand and the second modified oligonucleotide or second strand is 19 to 23 linked nucleosides in length. In certain embodiments, the region of complementarity between the first modified oligonucleotide or first strand and the second modified oligonucleotide or second strand is 21 to 23 linked nucleosides in length. In certain embodiments, the first modified oligonucleotide is fully complementary to the second modified oligonucleotide.

In certain embodiments, the modified oligonucleotide or first modified oligonucleotide of any preceding compound comprises at least one modification selected from a modified internucleoside linkage, a modified sugar, and a modified nucleobase. In certain embodiments, the second modified oligonucleotide of any preceding compound comprises at least one modification selected from the group consisting of a modified internucleoside linkage, a modified sugar, and a modified nucleobase. In certain embodiments, the modified internucleoside linkage is a phosphorothioate internucleoside linkage or a methylphosphonate internucleoside linkage. In certain embodiments, the phosphorothioate internucleoside linkage or methylphosphonate internucleoside linkage is at the 3′ terminus of the first or second modified oligonucleotide or at the 5′ terminus of the first modified oligonucleotide. In certain embodiments, the modified sugar comprises a modification selected from the group consisting of a halogen, an alkoxy group and a bicyclic sugar. In certain embodiments, the modified sugar comprises a 2′-F modification. In certain embodiments, the modified sugar comprises a 2′-OMe modification. In certain embodiments, each nucleoside of the first modified oligonucleotide comprises a modified sugar. In certain embodiments, each nucleoside of the second modified oligonucleotide comprises a modified sugar. In certain embodiments, the modified sugar comprises a modification selected from the group consisting of a halogen, an alkoxy group and a bicyclic sugar or a combination thereof. In certain embodiments, the modified sugar comprises a modification selected from the group consisting of 2′-MOE, 2′-F, and 2′-OMe or a combination thereof. In certain embodiments, the first modified oligonucleotide comprises no more than ten 2′-F sugar modifications. In certain embodiments, the second modified oligonucleotide comprises no more than five 2′-F sugar modifications.

In certain embodiments, the compound of any preceding embodiment comprises a conjugate group. In certain embodiments, the conjugate group is attached to the 5′ end of the modified oligonucleotide. In certain embodiments, the conjugate group is a targeting moiety. In certain embodiments, the targeting moiety comprises one or more GalNAc. In certain embodiments, the modified oligonucleotide is the second modified oligonucleotide or sense oligonucleotide. In certain embodiments, the one or more GalNAc is attached to the 2′ or 3′ position of the ribosyl ring. In certain embodiments, the one or more GalNAc is attached to the 5′ nucleoside of the modified oligonucleotide. In certain embodiments, the 5′ nucleoside of a modified oligonucleotide is selected from the following Formulae or a salt, solvate, or hydrate thereof, wherein R is the portion of the modified oligonucleotide other than the 5′ nucleoside:

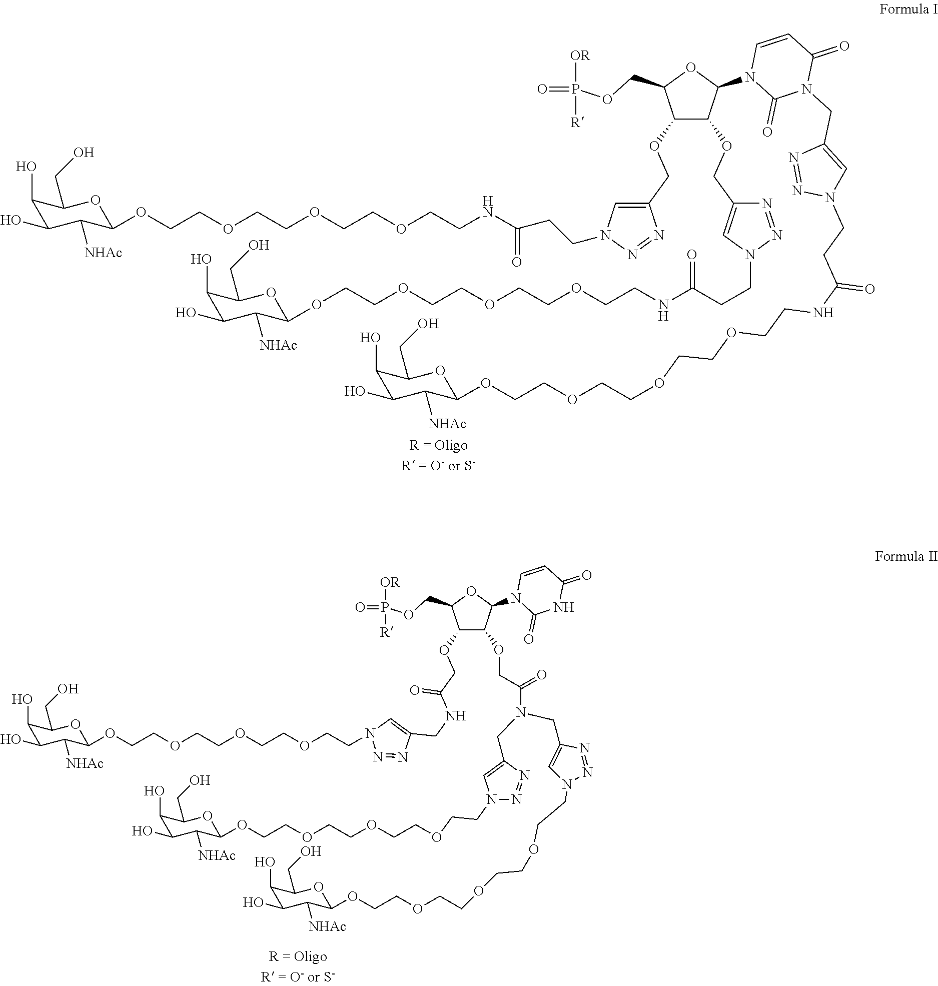

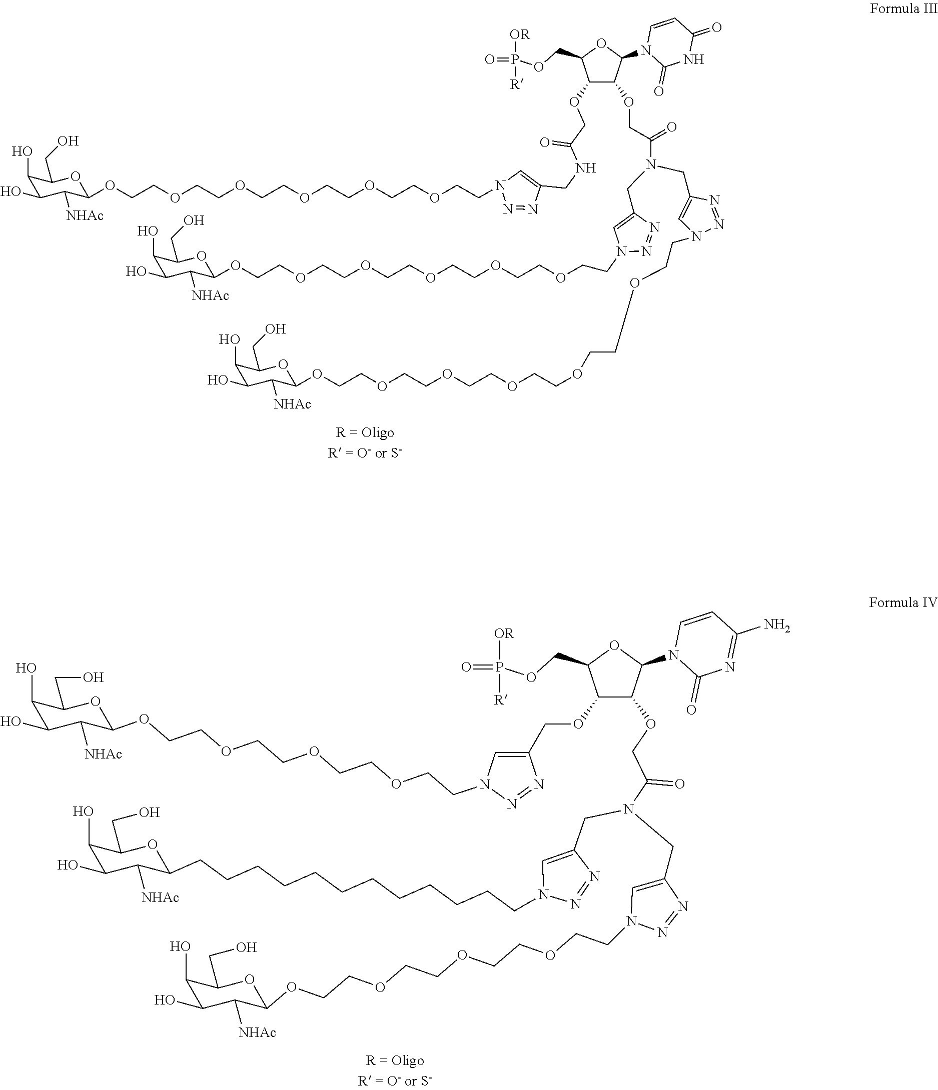

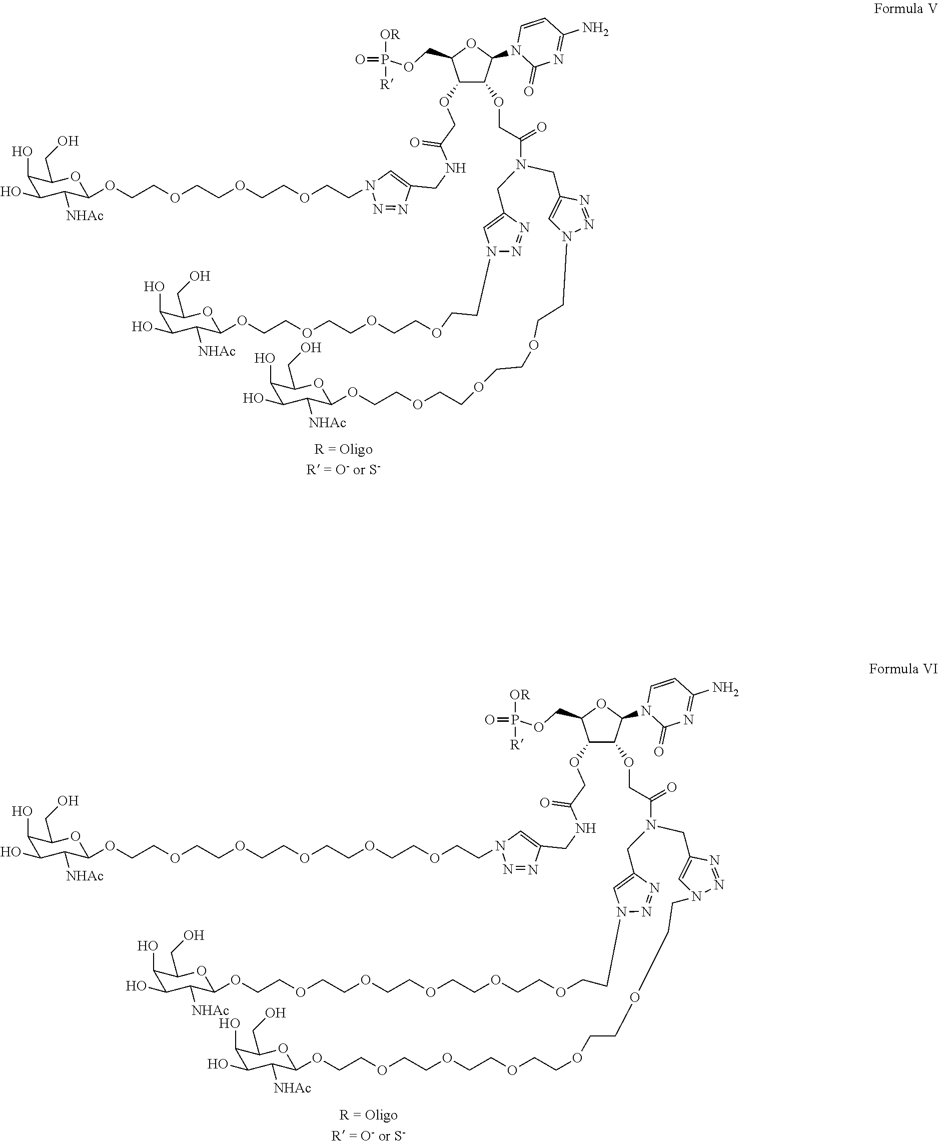

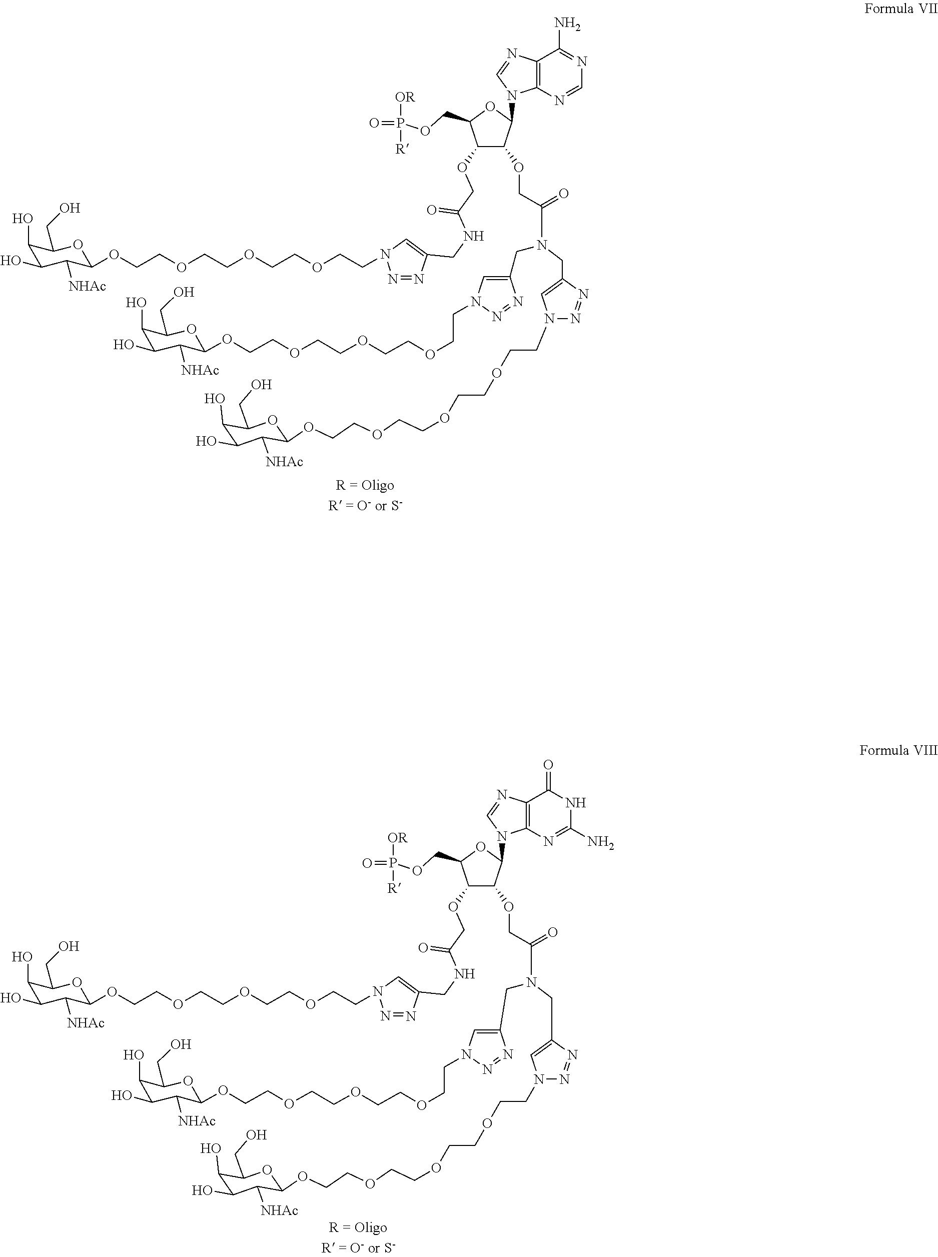

In certain embodiments, R′ is O. In certain embodiments, R′ is S. In certain embodiments, the 5′ nucleoside of the modified oligonucleotide is Formula I and R′ is O. In certain embodiments, the 5′ nucleoside of the modified oligonucleotide is Formula I and R′ is S. In certain embodiments, the 5′ nucleoside of the modified oligonucleotide is Formula II and R′ is O. In certain embodiments, the 5′ nucleoside of the modified oligonucleotide is Formula II and R′ is S. In certain embodiments, the 5′ nucleoside of the modified oligonucleotide is Formula III and R′ is O. In certain embodiments, the 5′ nucleoside of the modified oligonucleotide is Formula III and R′ is S. In certain embodiments, the 5′ nucleoside of the modified oligonucleotide is Formula IV and R′ is O. In certain embodiments, the 5′ nucleoside of the modified oligonucleotide is Formula IV and R′ is S. In certain embodiments, the 5′ nucleoside of the modified oligonucleotide is Formula V and R′ is O. In certain embodiments, the 5′ nucleoside of the modified oligonucleotide is Formula V and R′ is S. In certain embodiments, the 5′ nucleoside of the modified oligonucleotide is Formula VI and R′ is O. In certain embodiments, the 5′ nucleoside of the modified oligonucleotide is Formula VI and R′ is S. In certain embodiments, the 5′ nucleoside of the modified oligonucleotide is Formula VII and R′ is O. In certain embodiments, the 5′ nucleoside of the modified oligonucleotide is Formula VII and R′ is S. In certain embodiments, the 5′ nucleoside of the modified oligonucleotide is Formula VIII and R′ is O. In certain embodiments, the 5′ nucleoside of the modified oligonucleotide is Formula VIII and R′ is S.

Certain embodiments provide a compound comprising a first modified oligonucleotide selected from any one of Ref ID NOs: IA0812-821 and a second modified oligonucleotide 14 to 21 linked nucleosides in length fully complementary to the first modified oligonucleotide.

Certain embodiments provide a compound comprising a first modified oligonucleotide consisting of IA0813 and a second modified oligonucleotide consisting of IS1002.

Certain embodiments provide a compound comprising a first modified oligonucleotide consisting of IA0818 and a second modified oligonucleotide consisting of IS1007.

Certain embodiments provide a compound comprising a first modified oligonucleotide consisting of IA0818 and a second modified oligonucleotide consisting of IS1068.

Certain embodiments provide a compound comprising a first modified oligonucleotide selected from any one of Ref ID NOs: IA0864-866 and a second modified oligonucleotide 14 to 21 linked nucleosides in length fully complementary to the first modified oligonucleotide.

Certain embodiments provide a compound comprising a first modified oligonucleotide selected from Ref ID NOs: IA0818 and IA0864 and a second modified oligonucleotide selected from Ref ID NOs: IS1058 and IS1059.

Certain embodiments provide a compound comprising a first modified oligonucleotide consisting of IA0864 and a second modified oligonucleotide consisting of IS1059. Certain embodiments provide a compound comprising a first modified oligonucleotide consisting of IA0818 and a second modified oligonucleotide consisting of IS1058.

In certain embodiments, the compound comprises a first modified oligonucleotide consisting of IA0864 and a second modified oligonucleotide consisting of IS1059. In certain embodiments, the compound comprises a first modified oligonucleotide consisting of IA0818 and a second modified oligonucleotide consisting of IS1058.

In certain embodiments, the compound of any foregoing embodiment is in a pharmaceutically acceptable salt form. In certain embodiments, the pharmaceutically acceptable salt is a sodium salt. In certain embodiments, the pharmaceutically acceptable salt is a potassium salt.

In an aspect provided herein, is a modified oligonucleotide according to the following chemical structure:

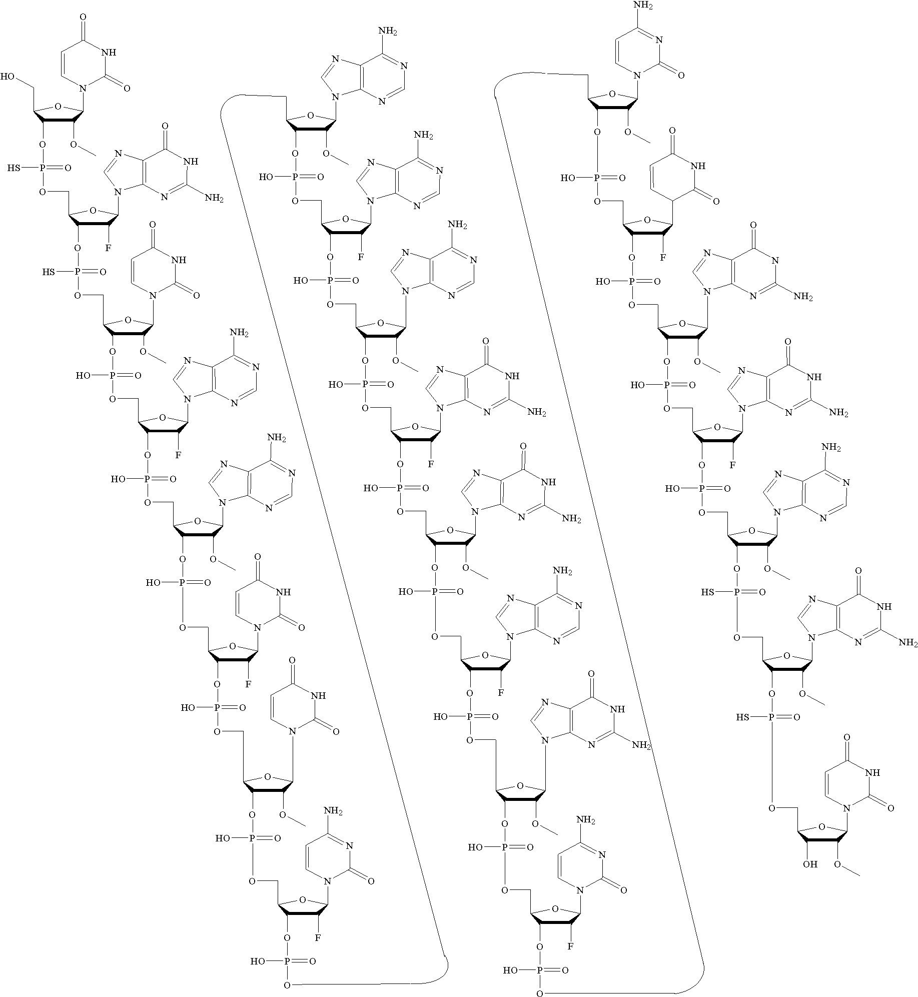

or a pharmaceutically acceptable salt or stereoisomer thereof. In certain embodiments, Ref ID NO: IA0818 is a modified oligonucleotide, or a pharmaceutically acceptable salt or stereoisomer hereof, according to the preceding chemical structure.

In an aspect provided herein, is a modified oligonucleotide according to the following chemical structure:

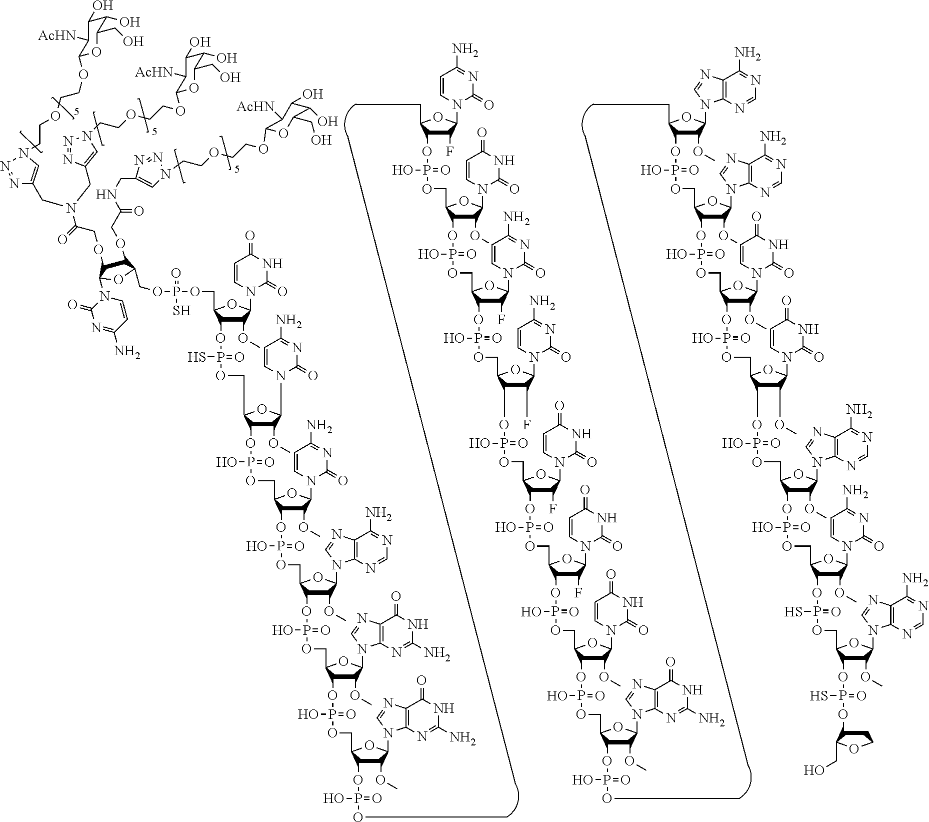

or a pharmaceutically acceptable salt or stereoisomer thereof. In certain embodiments, Ref ID NO: IS1058 is a modified oligonucleotide, or a pharmaceutically acceptable salt or stereoisomer thereof, according to the preceding chemical structure.

In an aspect provided herein, is a modified oligonucleotide according to the following chemical structure:

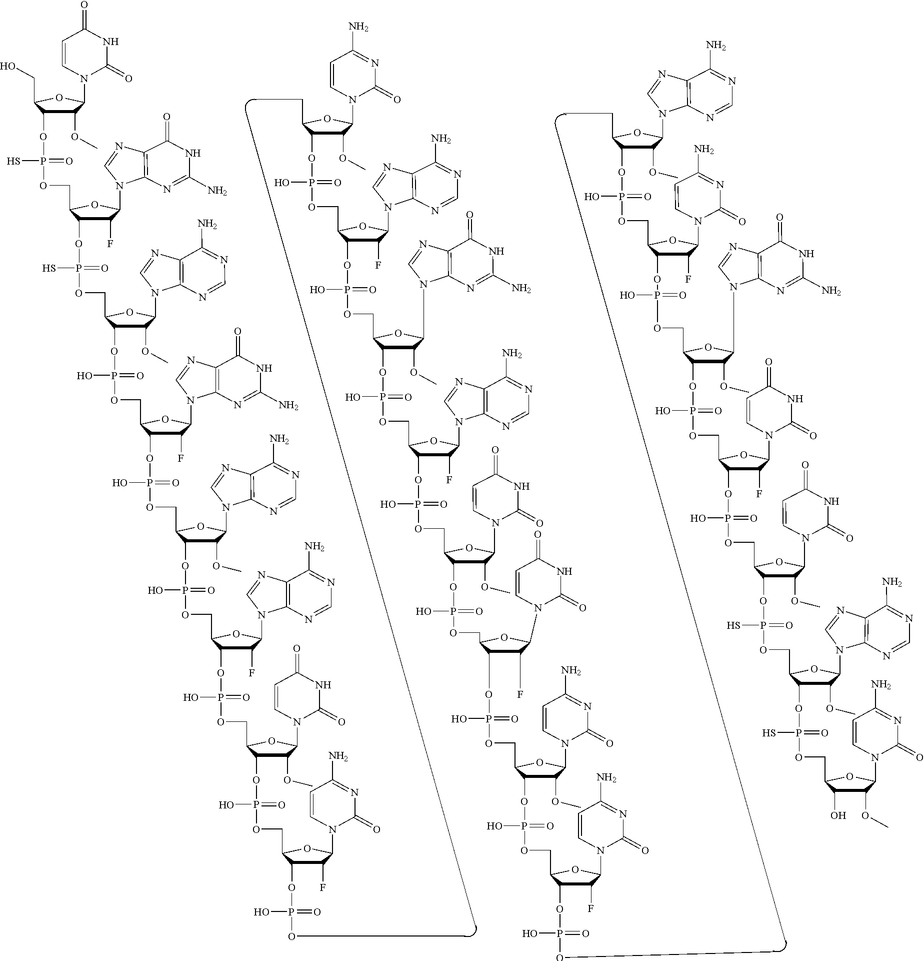

or a pharmaceutically acceptable salt or stereoisomer thereof. In certain embodiments, Ref ID NO: IA0864 is a modified oligonucleotide, or a pharmaceutically acceptable salt or stereoisomer thereof, according to the preceding chemical structure.

In an aspect provided herein, is a modified oligonucleotide according to the following chemical structure:

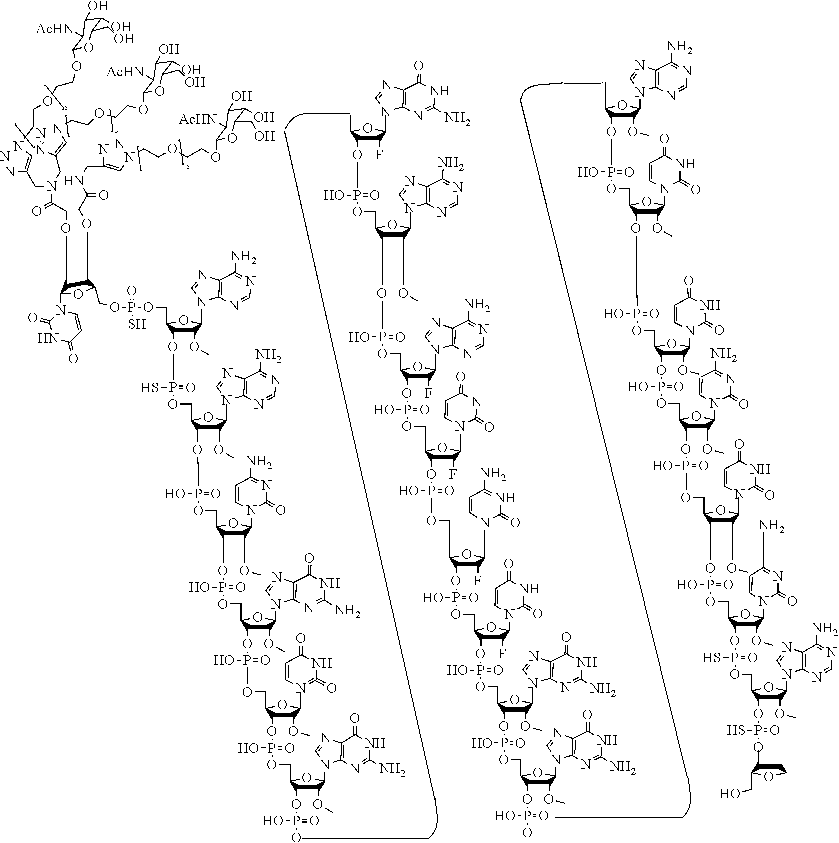

or a pharmaceutically acceptable salt or stereoisomer thereof. In certain embodiments, Ref ID NO: IS1059 is a modified oligonucleotide, or a pharmaceutically acceptable salt or stereoisomer thereof, according to the preceding chemical structure.

In certain embodiments, the pharmaceutically acceptable salt of the modified oligonucleotides provided herein is a sodium salt or a potassium salt.

In an aspect provided herein, is a sodium salt of a modified oligonucleotide according to the following chemical structure:

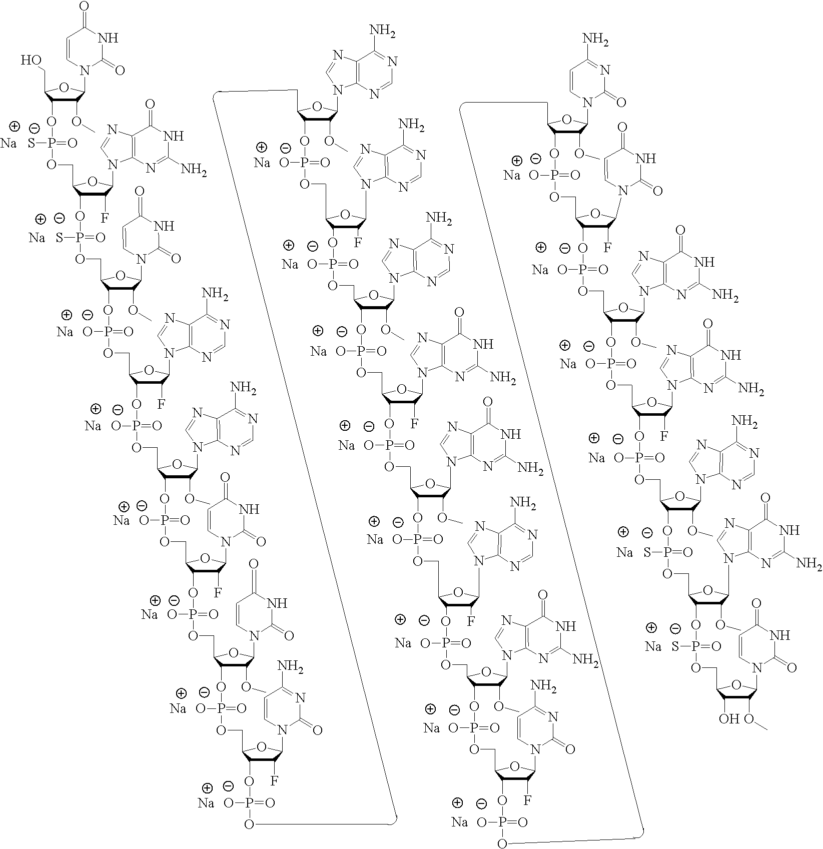

or a stereoisomer thereof. In certain embodiments, Ref ID NO: IA0818 is a modified oligonucleotide or a stereoisomer thereof, according to the preceding chemical structure.

In an aspect provided herein, is a sodium salt of a modified oligonucleotide according to the following chemical structure:

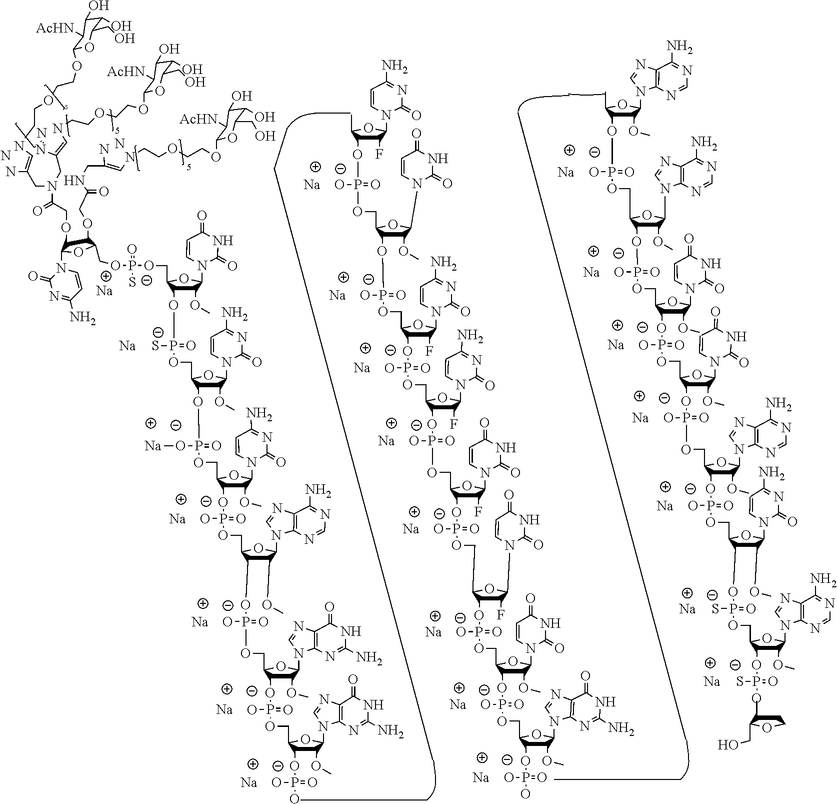

or a stereoisomer thereof. In certain embodiments, Ref ID NO: IS1058 is a modified oligonucleotide or a stereoisomer thereof, according to the preceding chemical structure.

In an aspect provided herein, is a sodium salt of a modified oligonucleotide according to the following chemical structure:

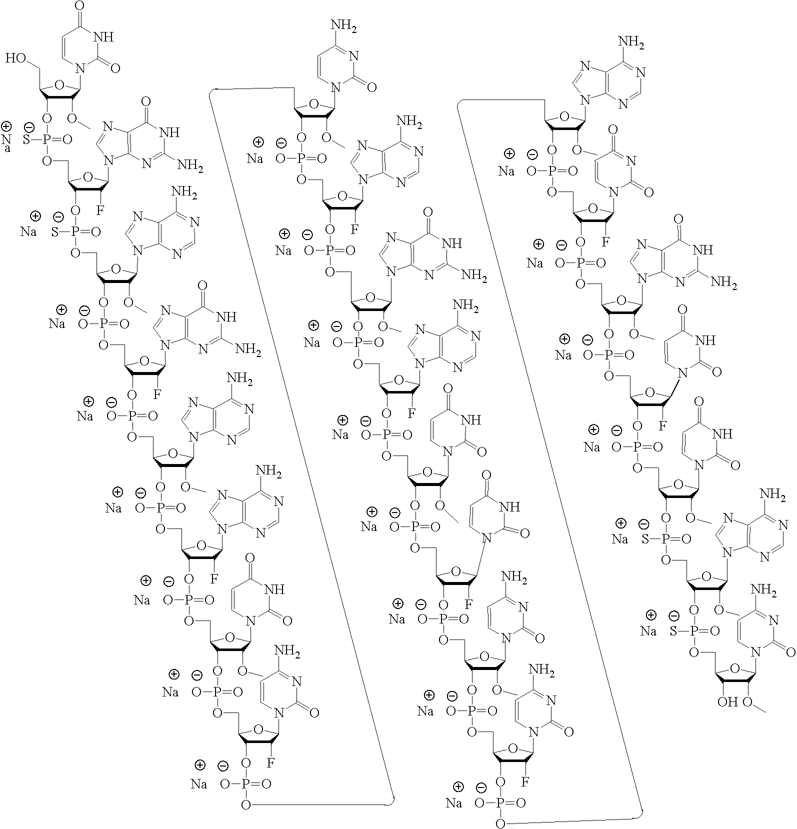

or a stereoisomer thereof. In certain embodiments, Ref ID NO: IA0864 is a modified oligonucleotide or a stereoisomer thereof, according to the preceding chemical structure.

In an aspect provided herein, is a sodium salt of a modified oligonucleotide according to the following chemical structure:

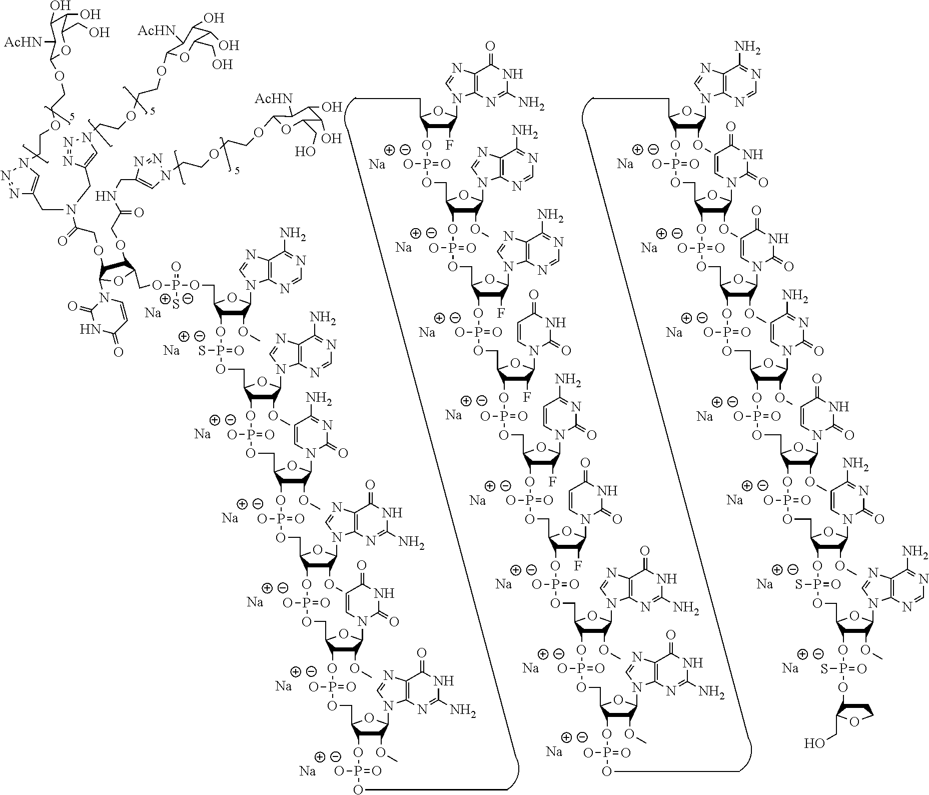

or a stereoisomer thereof. In certain embodiments, Ref ID NO: IS1059 is a modified oligonucleotide or a stereoisomer thereof, according to the preceding chemical structure.

In an aspect provided herein, is a compound according to the following chemical structure:

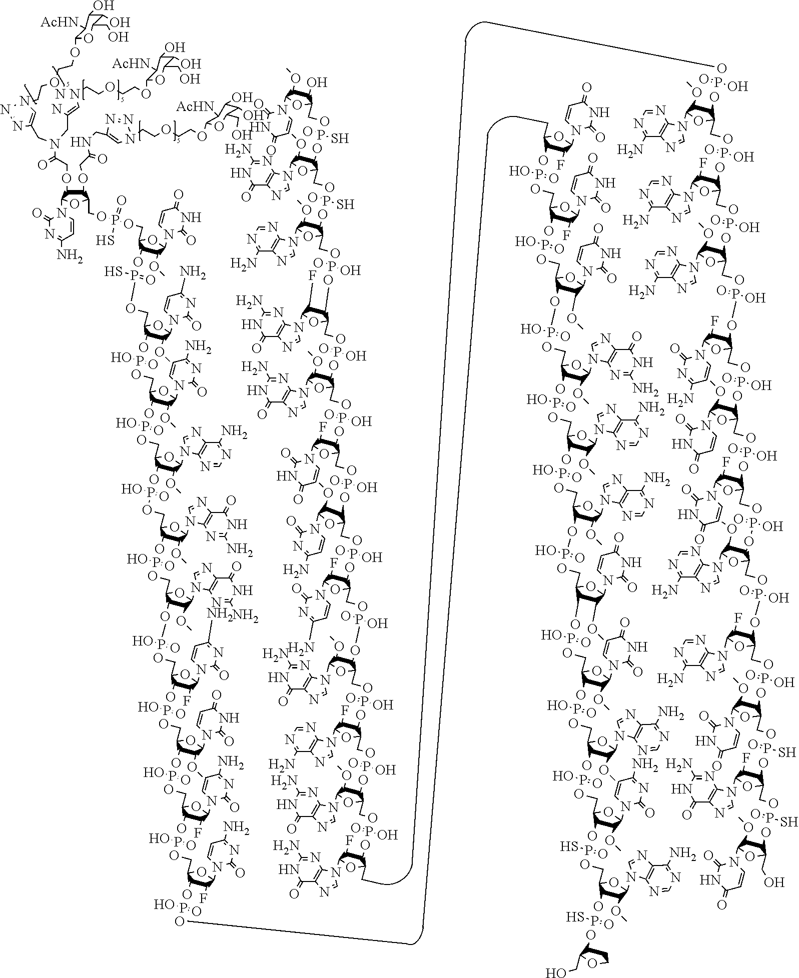

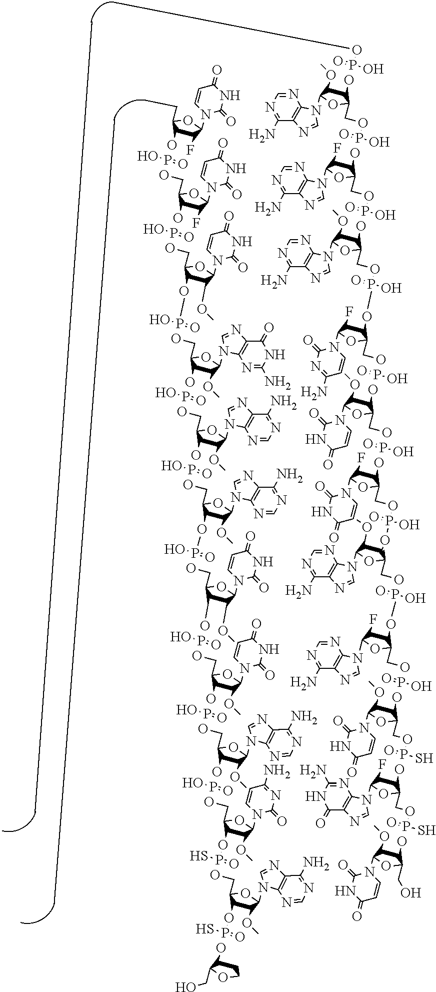

or a pharmaceutically acceptable salt or stereoisomer thereof. In certain embodiments, Compound Number RD2423 is a compound, or a pharmaceutically acceptable salt or stereoisomer thereof, according to the preceding chemical structure.

In an aspect provided herein, is a compound according to the following chemical structure:

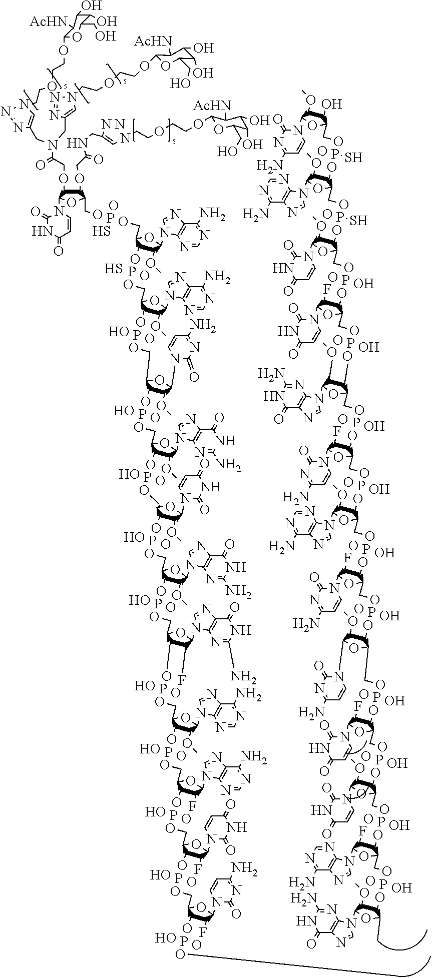

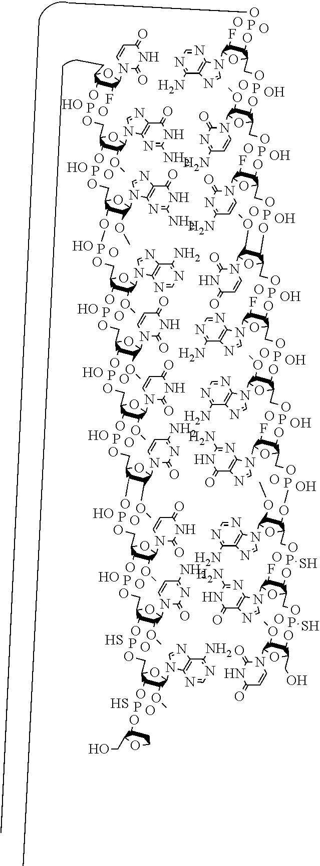

or a pharmaceutically acceptable salt or stereoisomer thereof. In certain embodiments, Compound Number RD2424 is a compound, or a pharmaceutically acceptable salt or stereoisomer thereof, according to the preceding chemical structure.

In certain embodiments, the pharmaceutically acceptable salt of the compounds provided herein is a sodium salt or a potassium salt.

In an aspect provided herein, is a sodium salt of a compound according to the following chemical structure:

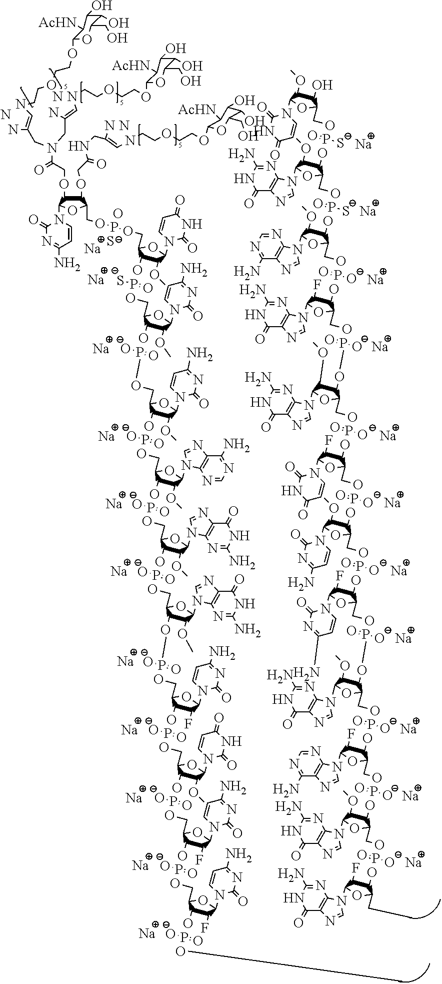

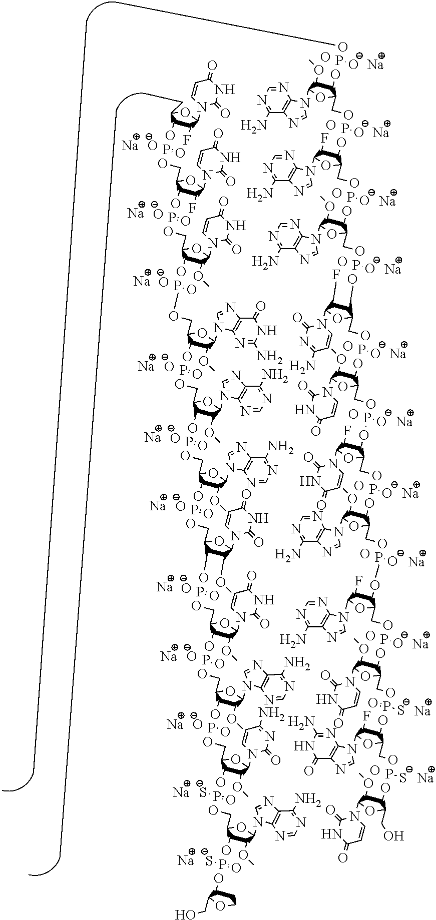

or a stereoisomer thereof. In certain embodiments, Compound Number RD2423 is a compound, or a stereoisomer thereof, according to the preceding chemical structure.

In an aspect provided herein, is a sodium salt of a compound according to the following chemical structure:

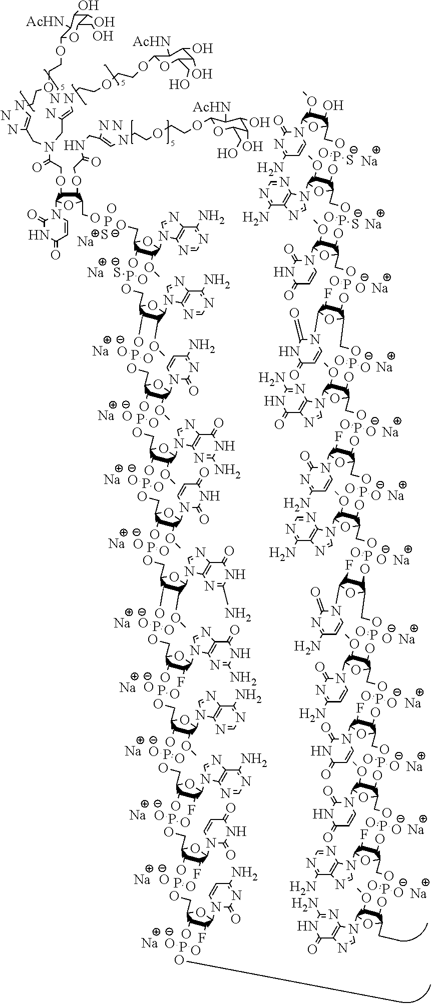

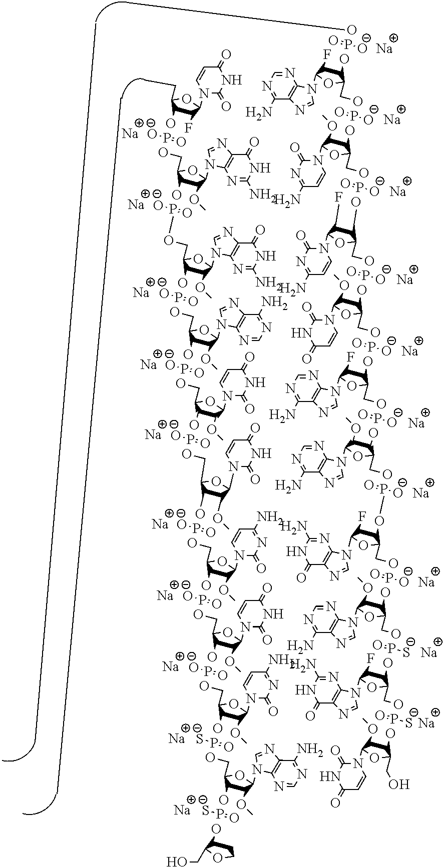

or a stereoisomer thereof. In certain embodiments, Compound Number RD2424 is a compound, or a stereoisomer thereof, according to the preceding chemical structure.

In certain embodiments, provided herein is a population of modified oligonucleotides, wherein all of the phosphorothioate internucleoside linkages of the modified oligonucleotide are stereorandom. In certain embodiments, provided herein is a population of compounds, wherein all of the phosphorothioate internucleoside linkages of the modified oligonucleotide are stereorandom.

Certain embodiments provide a composition comprising the compound of any one of the foregoing embodiments and a pharmaceutically acceptable carrier.

Certain embodiments provide a composition comprising a compound of any preceding embodiment, for use in therapy.

Certain embodiments provide a method of treating, preventing, or ameliorating a disease, disorder or condition associated with PKK and/or a dysregulated kallikrein pathway in an individual comprising administering to the individual a compound targeted to PKK, thereby treating, preventing, or ameliorating the disease, disorder or condition. In certain embodiments, the compound or composition of any foregoing embodiment is administered to an individual. In certain embodiments, the disease, disorder, or condition is an inflammatory or thrombotic disease, disorder or condition or a symptom thereof. In certain embodiments, the disease, disorder, or condition is hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, administering the compound inhibits or reduces or improves an inflammatory or thrombotic disease, disorder or condition or a symptom thereof. In certain embodiments, administering the compound inhibits or reduces or improves hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct, or a symptom thereof.

In certain embodiments, a compound or composition comprising a compound of any preceding embodiment is administered to an individual in a therapeutically effective amount. In certain embodiments, a composition comprising a compound of any preceding embodiment is administered to an individual at a dosage level sufficient to deliver about 1 to 100 mg/kg of body weight of the individual. In certain embodiments, a compound or composition comprising a compound of any preceding embodiment is administered to an individual at a fixed dose of about 25 mg to about 1,000 mg. In certain embodiments, the compound or composition is administered to the individual one or more times in a day up to the dosage level or fixed dose.

In certain embodiments, a composition comprising a compound of any preceding embodiment is administered to an individual daily, weekly, monthly, quarterly or yearly. In certain embodiments, a compound or composition comprising a compound of any preceding embodiment is administered to an individual about once per quarter (i.e., once every three months) to about once per year. In certain embodiments, a compound or composition comprising a compound of any preceding embodiment is administered to an individual about once per quarter, about once every six months or about once per year.

Certain embodiments provide a method of inhibiting expression of PKK in a cell comprising contacting the cell with a compound targeted to PKK, thereby inhibiting expression of PKK in the cell. In certain embodiments, the cell is in the liver of an individual. In certain embodiments, the individual has, or is at risk of having, an inflammatory or thrombotic disease, disorder or condition or a symptom thereof. In certain embodiments, the individual has, or is at risk of having, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct.

Certain embodiments provide a method of reducing or inhibiting an inflammatory or thrombotic disease, disorder or condition or a symptom thereof in an individual, comprising administering a compound targeted to PKK to the individual, thereby reducing or inhibiting an inflammatory or thrombotic disease, disorder or condition or a symptom thereof in the individual. In certain embodiments, the individual has, or is at risk of having, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound is a compound targeted to PKK. In certain embodiments, the compound is any of the foregoing compounds. In certain embodiments, the compound or composition is administered parenterally.

Certain embodiments provide use of a compound targeted to PKK for treating, preventing, or ameliorating a disease, disorder or condition associated with PKK. In certain embodiments, the disease, disorder or condition is an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound is a compound targeted to PKK. In certain embodiments, the compound is any of the foregoing compounds.

Certain embodiments provide use of a compound targeted to PKK in the manufacture of a medicament for treating, preventing, or ameliorating a disease, disorder or condition associated with PKK. In certain embodiments, the disease, disorder or condition is an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound is a compound targeted to PKK. In certain embodiments, the compound is any of the foregoing compounds.

### Certain Indications

In certain aspects, the disclosure relates to methods of inhibiting PKK expression, which can be useful for treating, preventing, or ameliorating a disease, disorder or condition associated with PKK in an individual, by administration of a compound that targets PKK. In certain embodiments, the compound can be a PKK specific inhibitor. In certain embodiments, the compound can be an antisense oligonucleotide, an oligomeric compound, or an oligonucleotide targeted to PKK.

In certain aspects, the disclosure relates to treating, preventing, or ameliorating a disease, disorder or condition associated with PKK. In certain embodiments, diseases, disorders or conditions associated with PKK treatable, preventable, and/or ameliorable with the methods provided herein include an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. Certain compounds provided herein are directed to compounds and compositions that reduce an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in an animal.

In certain embodiments, a method of treating, preventing, or ameliorating a disease, disorder or condition associated with PKK in an individual comprises administering to the individual a compound comprising a PKK specific inhibitor, thereby treating, preventing, or ameliorating the disease, disorder or condition. In certain embodiments, the individual is identified as having, or at risk of having, a disease, disorder or condition associated with PKK. In certain embodiments, the disease, disorder or condition is a an inflammatory disease or a thrombotic disease. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides) in length having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide having a nucleobase sequence selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence consisting of any one of SEQ ID NOs: 307, 312 and 626.

In any of the foregoing embodiments, the compound can be single-stranded or double-stranded. In certain embodiments, a single-stranded compound can be 14 to 30, 14 to 23, 14 to 20, 16 to 20, or 14 to 16, linked nucleosides in length. In certain embodiments, a single-stranded compound can be 14, 15, 16, 17, 18, 19, 20, 21, 22, 23, 24, 25, 26, 27, 28, 29, or 30, linked nucleosides in length. In certain embodiments, a double-stranded compound can comprise two oligonucleotides of the same or different lengths, as described elsewhere herein. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequence of SEQ ID NO: 307, SEQ ID NO: 312 and SEQ ID NO: 626. In certain embodiments, a compound comprises a modified oligonucleotide having a nucleobase sequence selected from the nucleobase sequence of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629.

In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628, and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide. In certain embodiments, the compound is administered to the individual parenterally. In certain embodiments, administering the compound improves, preserves, or prevents an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in an animal.

In certain embodiments, a method of treating, preventing, or ameliorating an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in an animal comprises administering to the individual a compound comprising a PKK specific inhibitor, thereby treating, preventing, or ameliorating an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide having a nucleobase sequence selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequence of SEQ ID NO: 307, SEQ ID NO: 312 and SEQ ID NO: 626. In certain embodiments, a compound comprises a modified oligonucleotide having a nucleobase sequence selected from the nucleobase sequence of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide. In certain embodiments, administering the compound improves, preserves, or prevents an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in an animal. In certain embodiments, the individual is identified as having, or at risk of having, a disease associated with PKK.

In certain embodiments, a method of inhibiting expression of PKK in an individual having, or at risk of having, a disease, disorder or condition associated with PKK comprises administering to the individual a compound comprising a PKK specific inhibitor, thereby inhibiting expression of PKK in the individual. In certain embodiments, administering the compound inhibits expression of PKK in the liver. In certain embodiments, the disease, disorder or condition is an inflammatory disease or a thrombotic disease. In certain embodiments, the individual has, or is at risk of having, an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide having a nucleobase sequence selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence selected from any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide. In certain embodiments, the compound is administered to the individual parenterally. In certain embodiments, administering the compound improves, preserves, or prevents an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct.

In certain embodiments, a method of inhibiting expression of PKK in a cell comprises contacting the cell with a compound comprising a PKK specific inhibitor, thereby inhibiting expression of PKK in the cell. In certain embodiments, the cell is a hepatocyte. In certain embodiments, the cell is in the liver. In certain embodiments, the cell is in the liver of an individual who has, or is at risk of having, an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide having a nucleobase sequence selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629.

In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide.

In certain embodiments, a method of reducing or inhibiting an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in an individual having, or at risk of having, a disease associated with PKK comprises administering to the individual a compound comprising a PKK specific inhibitor, thereby reducing or inhibiting an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct in the individual. In certain embodiments, the individual has, or is at risk of having, an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide. In certain embodiments, the compound is administered to the individual parenterally. In certain embodiments, the individual is identified as having, or at risk of having, a disease, disorder or condition associated with PKK.

Certain embodiments are drawn to a compound comprising a PKK specific inhibitor for use in treating a disease, disorder or condition associated with PKK. In certain embodiments, the disease, disorder or condition is an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide. In certain embodiments, the compound is administered to the individual parenterally.

Certain embodiments are drawn to a compound comprising a PKK specific inhibitor for use in reducing or inhibiting an inflammatory disease, a thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide.

Certain embodiments are drawn to the use of a compound comprising a PKK specific inhibitor for the manufacture or preparation of a medicament for treating a disease, disorder or condition associated with PKK. Certain embodiments are drawn to the use of a compound comprising a PKK specific inhibitor for the preparation of a medicament for treating a disease, disorder or condition associated with PKK. In certain embodiments, the disease is an inflammatory or thrombotic disease. In certain embodiments, the disease is hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide having a nucleobase sequence selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide.

Certain embodiments are drawn to the use of a compound comprising a PKK specific inhibitor for the manufacture or preparation of a medicament for reducing or inhibiting an inflammatory disease, a thrombotic disease in an individual having, or at risk of having, an inflammatory disease or a thrombotic disease associated with PKK. In certain embodiments, the inflammatory disease or thrombotic disease is hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. Certain embodiments are drawn to use of a compound comprising a PKK specific inhibitor for the preparation of a medicament for treating a disease associated with PKK. In certain embodiments, the disease is an inflammatory disease, thrombotic disease, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct. In certain embodiments, the compound comprises an antisense oligonucleotide targeted to PKK. In certain embodiments, the compound comprises an oligonucleotide targeted to PKK. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequences of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising the nucleobase sequence of any one of SEQ ID NOs: 10-313, 626, 627, and 628. In certain embodiments, a compound comprises a modified oligonucleotide selected from the nucleobase sequence of any one of SEQ ID NOs: 10-313, 626, 627, and 628. In certain embodiments, a compound comprises a modified oligonucleotide (e.g., of 14 to 30, for example, 14 to 23, linked nucleosides in length) having a nucleobase sequence comprising any one of SEQ ID NOs: 307, 312 and 626. In certain embodiments, a compound comprises a modified oligonucleotide having a nucleobase sequence selected from any one of SEQ ID NOs: 307, 312 and 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 307, 312 or 626. In certain embodiments, a compound comprises a modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of the nucleobase sequence of SEQ ID NO: 468, 611, 616, 619 or 629. In certain embodiments, a compound comprises a modified oligonucleotide having a nucleobase sequence selected from the nucleobase sequences of SEQ ID NO: 307, SEQ ID NO: 312 and SEQ ID NO: 626. In certain embodiments, a compound comprises a modified oligonucleotide having a nucleobase sequence selected from the nucleobase sequences of SEQ ID NO: 468, SEQ ID NO: 611, SEQ ID NO: 616, SEQ ID NO: 619 and SEQ ID NO: 629. In certain embodiments, the compound comprises a first modified oligonucleotide having a nucleobase sequence of SEQ ID NO: 312 and a second modified oligonucleotide having a nucleobase sequence of SEQ ID NO: 619. In certain embodiments, the compound comprises a first modified oligonucleotide having a nucleobase sequence of SEQ ID NO: 626 and a second modified oligonucleotide having a nucleobase sequence of SEQ ID NO: 629. In any of the foregoing embodiments, the compound can be single-stranded or double-stranded. In any of the foregoing embodiments, the compound can be an antisense oligonucleotide or oligomeric compound. In certain embodiments, a compound comprises a first modified oligonucleotide (e.g., of 14 to 30, for example, 14 to 23, linked nucleosides in length) having a nucleobase sequence comprising at least 14, at least 15, at least 16, at least 17, at least 18, at least 19, at least 20, at least 21, at least 22, at least 23 contiguous nucleobases of any of the nucleobase sequence of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide (e.g., of 14 to 30, for example, 14 to 23, linked nucleosides in length) having a nucleobase sequence comprising the nucleobase sequence of any one of SEQ ID NOs: 10-631 and a second modified oligonucleotide (e.g., of 14 to 30, for example, 14 to 23, linked nucleosides in length) having a region of complementarity to the first modified oligonucleotide. In certain embodiments, a compound comprises a first modified oligonucleotide having a nucleobase sequence selected from any one of SEQ ID NOs: 10-631 and a second modified oligonucleotide 19 to 23 linked nucleosides in length having a region of complementarity to the first modified oligonucleotide.

In any of the foregoing methods or uses, the compound can be an oligomeric compound. In any of the foregoing methods or uses, the compound can be single-stranded or double-stranded. In any of the foregoing methods or uses, the compound can be targeted to PKK. In certain embodiments, the compound comprises or consists of a modified oligonucleotide. In certain embodiments, the compound comprises one or more modified oligonucleotides. In certain embodiments, the compound comprises a first modified oligonucleotide and a second modified oligonucleotide. In certain embodiments, a modified oligonucleotide is 8 to 80 linked nucleosides in length, 10 to 30 linked nucleosides in length, 14 to 30 linked nucleosides in length, 14 to 23 linked nucleosides in length, or 19 to 23 linked nucleosides in length. In certain embodiments, a modified oligonucleotide is at least 80%, at least 85%, at least 90%, at least 95% or 100% complementary to any of the nucleobase sequences recited in SEQ ID NOs: 1, 3, 5 or 6 over its length. In certain embodiments, a modified oligonucleotide comprises at least one modified internucleoside linkage, at least one modified sugar and/or at least one modified nucleobase. In certain embodiments, the modified internucleoside linkage is a phosphorothioate internucleoside linkage. In certain embodiments, the modified sugar is a bicyclic sugar, 2′-MOE, 2′-F, or 2′-OMe. In certain embodiments, the modified nucleobase is a 5-methylcytosine. In any of the foregoing embodiments, each modified oligonucleotide is independently 12 to 30, 14 to 30, 14 to 25, 14 to 24, 14 to 23, 16 to 23, 17 to 23, 18 to 23, 19 to 23, 19 to 22, or 19 to 20 linked nucleosides in length. In certain embodiments, a modified oligonucleotide has at least 1, at least 2, at least 3 mismatches to a region of SEQ ID NOs: 1, 3, 5 or 6.

In any of the forgoing methods or uses, the compound comprises a first and second modified oligonucleotide, wherein there is a region of complementarity between a first modified oligonucleotide and a second modified oligonucleotide. In certain embodiments, the region of complementarity between the first oligonucleotide and the second oligonucleotide is 14 to 23, 19 to 23, or 21 to 23 linked nucleosides in length. In certain embodiments, the first modified oligonucleotide is fully complementary to the second modified oligonucleotide. In certain embodiments, the first modified oligonucleotide comprises at least one modification selected from a modified internucleoside linkage, a modified sugar, and a modified nucleobase. In certain embodiments, the second modified oligonucleotide comprises at least one modification selected from the group consisting of a modified internucleoside linkage, a modified sugar, and a modified nucleobase. In certain embodiments, the modified internucleoside linkage is a phosphorothioate internucleoside linkage or a methylphosphonate internucleoside linkage. In certain embodiments, the modified internucleoside linkage is at the 3′ terminus of the first or second modified oligonucleotide or at the 5′ terminus of the first or second modified oligonucleotide. In certain embodiments, the first or second modified oligonucleotide comprises one or more modified sugars. In certain embodiments, each nucleoside of the first or second modified oligonucleotide comprises a modified sugar. In certain embodiments, the modified sugar comprises a modification selected from the group consisting of a halogen, an alkoxy group and a bicyclic sugar. In certain embodiments, the modified sugar comprises a modification selected from group consisting of 2′-MOE, 2′-F, and 2′-OMe or a combination thereof. In certain embodiments, the first or second modified oligonucleotide comprises no more than ten 2′-F sugar modifications. In certain embodiments, the first or second modified oligonucleotide comprises no more than five 2′-F sugar modifications.

In any of the forgoing methods or uses, a compound comprises a conjugate group. In certain embodiments, the conjugate group is attached to the 5′ end of a modified oligonucleotide. In certain embodiments, the conjugate group is a targeting moiety. In certain embodiments, the targeting moiety comprises one or more GalNAc. In certain embodiments, the one or more GalNAc is attached to the 2′ or 3′ position of the ribosyl ring. In certain embodiments, the one or more GalNAc is attached to the 5′ nucleoside of the modified oligonucleotide. In certain embodiments, the 5′ nucleoside of a modified oligonucleotide is selected from Formulae I-VIII, or a salt, solvate, or hydrate thereof, wherein R is the modified oligonucleotide other than the 5′ nucleoside. In certain embodiments, the 5′ nucleoside of the modified oligonucleotide is Formula I and R′ is O. In certain embodiments, the 5′ nucleoside of the modified oligonucleotide is Formula I and R′ is S. In certain embodiments, the 5′ nucleoside of the modified oligonucleotide is Formula II and R′ is O. In certain embodiments, the 5′ nucleoside of the modified oligonucleotide is Formula II and R′ is S. In certain embodiments, the 5′ nucleoside of the modified oligonucleotide is Formula III and R′ is O. In certain embodiments, the 5′ nucleoside of the modified oligonucleotide is Formula III and R′ is S. In certain embodiments, the 5′ nucleoside of the modified oligonucleotide is Formula IV and R′ is O. In certain embodiments, the 5′ nucleoside of the modified oligonucleotide is Formula IV and R′ is S. In certain embodiments, the 5′ nucleoside of the modified oligonucleotide is Formula V and R′ is O. In certain embodiments, the 5′ nucleoside of the modified oligonucleotide is Formula V and R′ is S. In certain embodiments, the 5′ nucleoside of the modified oligonucleotide is Formula VI and R′ is O. In certain embodiments, the 5′ nucleoside of the modified oligonucleotide is Formula VI and R′ is S. In certain embodiments, the 5′ nucleoside of the modified oligonucleotide is Formula VII and R′ is O. In certain embodiments, the 5′ nucleoside of the modified oligonucleotide is Formula VII and R′ is S. In certain embodiments, the 5′ nucleoside of the modified oligonucleotide is Formula VIII and R′ is O. In certain embodiments, the 5′ nucleoside of the modified oligonucleotide is Formula VIII and R′ is S.

In any of the foregoing methods or uses, the compound comprises a first modified oligonucleotide selected from any one of Ref ID NOs: IA0812-821 and a second modified oligonucleotide 14 to 21 linked nucleosides in length fully complementary to the first modified oligonucleotide. In certain embodiments, the compound comprises a first modified oligonucleotide consisting of IA0813 and a second modified oligonucleotide consisting of IS1002. In certain embodiments, the compound comprises a first modified oligonucleotide consisting of IA0818 and a second modified oligonucleotide consisting of IS1007. In certain embodiments, the compound comprises a first modified oligonucleotide consisting of IA0818 and a second modified oligonucleotide consisting of IS1068.

In any of the foregoing methods or uses, the compound comprises a first modified oligonucleotide selected from any one of Ref ID NOs: IA0864-866 and a second modified oligonucleotide 14 to 21 linked nucleosides in length fully complementary to the first modified oligonucleotide. In certain embodiments, the compound comprises a first modified oligonucleotide consisting of IA0864 and a second modified oligonucleotide consisting of IS1059. Certain embodiments provide a compound comprising a first modified oligonucleotide selected from Ref ID NOs: IA0818 and IA0864 and a second modified oligonucleotide selected from Ref ID NOs: IS1058 and IS1059. Certain embodiments provide a compound comprising a first modified oligonucleotide consisting of IA0864 and a second modified oligonucleotide consisting of IS1059. Certain embodiments provide a compound comprising a first modified oligonucleotide consisting of IA0818 and a second modified oligonucleotide consisting of IS1058.

In certain embodiments, the compound comprises a first modified oligonucleotide selected from Ref ID NOs: IA0818 and IA0864 and a second modified oligonucleotide selected from Ref ID NOs: IS1058 and IS1059. In certain embodiments, the compound comprises a first modified oligonucleotide consisting of IA0864 and a second modified oligonucleotide consisting of IS1059. In certain embodiments, the compound comprises a first modified oligonucleotide consisting of IA0818 and a second modified oligonucleotide consisting of IS1058.

In certain embodiments, the compound is in a pharmaceutically acceptable salt form. In certain embodiments, the pharmaceutically acceptable salt is a sodium salt. In certain embodiments, the pharmaceutically acceptable salt is a potassium salt. In certain embodiments, a composition comprises the compound of any one of the foregoing embodiments and a pharmaceutically acceptable carrier.

In any of the foregoing methods or uses, a compound or composition comprising a compound of any preceding embodiment is administered to an individual in a therapeutically effective amount. In certain embodiments, a compound or composition comprising a compound of any preceding embodiment is administered to an individual at a dosage level sufficient to deliver about 1 to 100 mg/kg of body weight of the individual. In certain embodiments, a compound or composition comprising a compound of any preceding embodiment is administered to an individual at a fixed dose of about 25 mg to about 1,000 mg. In certain embodiments, the composition is administered to the individual one or more times in a day up to the dosage level or fixed dose.

In any of the foregoing methods or uses, a compound or composition comprising a compound of any preceding embodiment is administered to an individual daily, weekly, monthly, quarterly or yearly. In certain embodiments, a compound or composition comprising a compound of any preceding embodiment is administered to an individual about once per quarter (i.e., once every three months) to about once per year. In certain embodiments, a compound or composition comprising a compound of any preceding embodiment is administered to an individual about once per quarter, about once every six months or about once per year.

### Certain Compounds

In certain aspects, the disclosure relates to a compound that comprises or consists of an oligomeric compound. In certain embodiments, the oligomeric compound comprises a nucleobase sequence complementary to that of a target nucleic acid.

In certain aspects, the disclosure relates to a compound that comprises or consists of a modified oligonucleotide. In certain embodiments, the modified oligonucleotide has a nucleobase sequence complementary to that of a target nucleic acid.

In certain aspects, the disclosure relates to a compound that comprises or consists of an antisense oligonucleotide. In certain embodiments, the antisense oligonucleotide has a nucleobase sequence complementary to that of a target nucleic acid.

In certain aspects, the disclosure relates to a compound that is a single-stranded compound. In certain embodiments, the single-stranded compound comprises or consists of an oligomeric compound. In certain embodiments, such an oligomeric compound comprises or consists of an oligonucleotide and optionally a conjugate group. In certain embodiments, the oligonucleotide is a modified oligonucleotide. In certain embodiments, the oligonucleotide is an antisense oligonucleotide. In certain embodiments, the oligonucleotide or modified oligonucleotide of a single-stranded compound comprises a self-complementary nucleobase sequence.

In certain aspects, the disclosure relates to a compound that is a double-stranded compound. In certain embodiments, the double-stranded compound comprises or consists of an oligomeric compound. In certain embodiments, the double-stranded compound comprises a first oligonucleotide and a second oligonucleotide. In certain embodiments, the first oligonucleotide has a region complementarity to a target nucleic acid and the second oligonucleotide has a region complementarity to the first modified oligonucleotide. In certain embodiments, the double-stranded compound comprises a modified oligonucleotide. In certain embodiments, the modified oligonucleotide has a region complementarity to a target nucleic acid. In certain embodiments, the double-stranded compound comprises a first modified oligonucleotide and a second modified oligonucleotide. In certain embodiments, the first modified oligonucleotide has a region complementarity to a target nucleic acid and the second modified oligonucleotide has a region complementarity to the first modified oligonucleotide. In certain embodiments, an oligonucleotide or modified oligonucleotide of a double-stranded compound is an RNA oligonucleotide. In such embodiments, the thymine nucleobase in the modified oligonucleotide is replaced by a uracil nucleobase.

In certain embodiments, a compound described herein comprises a conjugate group. In certain embodiments, the first oligonucleotide or first modified oligonucleotide of a double-stranded compound comprises a conjugate group. In certain embodiments, the second oligonucleotide or second modified oligonucleotide of a double-stranded compound comprises a conjugate group. In certain embodiments, a first oligonucleotide or first modified oligonucleotide and a second oligonucleotide or second modified oligonucleotide of a double-stranded compound each comprises a conjugate group.

In certain embodiments, a compound is 14-30 linked nucleosides in length. In certain embodiments, the first oligonucleotide or first modified oligonucleotide of a double-stranded compound is 14-30 linked nucleosides in length. In certain embodiments, the second oligonucleotide or second modified oligonucleotide is 14-30 linked nucleosides in length. In certain embodiments, the oligonucleotides or modified oligonucleotides of a double-stranded compound are blunt ended at one or both ends of the compound. In certain embodiments, the oligonucleotides or modified oligonucleotides of a double-stranded compound include non-complementary overhanging nucleosides at one or both ends of the compound.

In certain embodiments, a compound has a nucleobase sequence comprising at least 14 contiguous nucleobases of any of SEQ ID NOs: 10-313, 626, 627, and 628. In certain embodiments, one of the oligonucleotides or modified oligonucleotides of a double-stranded compound has a nucleobase sequence comprising at least 14 contiguous nucleobases of any of SEQ ID NOs: 10-313, 626, 627, and 628.

Examples of single-stranded and double-stranded compounds include, but are not limited to, oligonucleotides, antisense oligonucleotides, siRNAs, microRNA targeting oligonucleotides, occupancy-based compounds (e.g., mRNA processing or translation blocking compounds and splicing compounds), and single-stranded RNAi compounds (e.g. small hairpin RNAs (shRNAs), single stranded siRNAs (ssRNAs) and microRNA mimics).

In certain embodiments, a compound described herein has a nucleobase sequence that, when written in the 5′ to 3′ direction, comprises the reverse complement of the target region of a target nucleic acid to which it is targeted.

In certain embodiments, a compound described herein comprises an oligonucleotide 12 to 30 linked subunits in length. In certain embodiments, a compound described herein comprises an oligonucleotide 12 to 23 linked subunits in length. In certain embodiments, compound described herein comprises an oligonucleotide 14 to 30 linked subunits in length. In certain embodiments, compound described herein comprises an oligonucleotide 14 to 23 linked subunits in length. In certain embodiments, a compound described herein comprises an oligonucleotide 15 to 30 linked subunits in length. In certain embodiments, a compound described herein comprises an oligonucleotide 15 to 23 linked subunits in length. In certain embodiments, a compound described herein comprises an oligonucleotide 16 to 30 linked subunits in length. In certain embodiments, a compound described herein comprises an oligonucleotide 16 to 23 linked subunits in length. In certain embodiments, a compound described herein comprises an oligonucleotide 17 to 30 linked subunits in length. In certain embodiments, a compound described herein comprises an oligonucleotide 17 to 23 linked subunits in length. In certain embodiments, a compound described herein comprises an oligonucleotide 18 to 30 linked subunits in length. In certain embodiments, a compound described herein comprises an oligonucleotide 18 to 23 linked subunits in length. In certain embodiments, a compound described herein comprises an oligonucleotide 19 to 30 linked subunits in length. In certain embodiments, a compound described herein comprises an oligonucleotide 19 to 23 linked subunits in length. In other words, such oligonucleotides are 12 to 30 linked subunits, 12 to 23 linked subunits, 14 to 30 linked subunits, 14 to 23 linked subunits, 15 to 30 linked subunits, 15 to 23 linked subunits, 16 to 30 linked subunits, 16 to 23 linked subunits, 17 to 30 linked subunits, 17 to 23 linked subunits, 18 to 30 linked subunits, 18 to 23 linked subunits, 19 to 30 linked subunits or 19 to 23 linked subunits, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In certain embodiments, a compound described herein comprises an oligonucleotide 21 linked subunits in length. In certain embodiments, a compound described herein comprises an oligonucleotide 22 linked subunits in length. In certain embodiments, a compound described herein comprises an oligonucleotide 23 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 23, 18 to 24, 18 to 25, 18 to 50, 19 to 23, 19 to 30, 19 to 50, 20 to 23 or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, or 30 linked subunits in length, or a range defined by any two of the above values.

In certain embodiments, the compound may further comprise an additional moiety, such as a conjugate group or delivery moiety. In certain embodiments, such compounds are oligomeric compounds, and the additional moiety is attached to an oligonucleotide. In certain embodiments, a conjugate group is attached to a nucleoside of an oligonucleotide.

In certain embodiments, compounds may be shortened or truncated. For example, one or more subunits may be deleted from the 5′ end (5′ truncation), or alternatively from the 3′ end (3′ truncation) of an oligonucleotide.

In certain embodiments, compounds may be lengthened. For example, one or more subunits may be attached to the 3′ end or 5′ end of an oligonucleotide. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunits) is attached to the 5′ end of an oligonucleotide. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunits) is attached to the 3′ end of an oligonucleotide. In certain embodiments, at least one or more subunits may be attached to the 3′ end or 5′ end of an oligonucleotide of a double-stranded compound creating a 3′ and/or 5′ end overhang. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunits) is attached to the 5′ end of both oligonucleotides of a double-stranded compound. In certain embodiments, at least one subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more subunit) is attached to the 3′ end of both oligonucleotides of a double-stranded compound. In certain embodiments, subunits are attached to both oligonucleotides of a double-stranded compound at the same end (e.g., that subunits are attached to the 3′ end of one of the oligonucleotides and subunits are attached to the 5′ end of the other oligonucleotide). In certain embodiments, when subunits are attached to both oligonucleotides of a double-stranded compound at the same end, the number of subunits attached to each oligonucleotide may be the same or may be different. In certain embodiments, when subunits are attached to both oligonucleotides of a double-stranded compound at the same end, the number of subunits attached to each oligonucleotide is the same. In certain embodiments, when subunits are attached to both oligonucleotides of a double-stranded compound at the same end, the number of subunits attached to each oligonucleotide is different. This scenario, where subunits are attached to both oligonucleotides of a double-stranded compound at the same end, may occur at one or both ends of a double-stranded compound. In certain embodiments, the subunits attached to the 3′ and/or 5′ end are modified.

In certain embodiments, compounds described herein are oligonucleotides. In certain embodiments, compounds described herein are modified oligonucleotides. In certain embodiments, compounds described herein are antisense oligonucleotides. In certain embodiments, compounds described herein are oligomeric compounds. In certain embodiments, compounds described herein are RNAi compounds. In certain embodiments, compounds described herein are siRNA compounds.

In certain embodiments, a compound described herein can comprise any of the oligonucleotide sequences targeted to PKK described herein. In certain embodiments, the compound can be double-stranded.

In certain embodiments, the compound comprises an oligonucleotide comprising at least an 8, 9, 10, 11, 12, 13, 14, 15, 16, 17, 18, 19, 20, 21, 22 or 23 contiguous nucleobase portion of any one of SEQ ID NOs: 10-313, 626, 627, and 628. In certain embodiments, the compound comprises an oligonucleotide comprising at least an 8, 9, 10, 11, 12, 13, 14, 15, 16, 17, 18, 19, 20, 21, 22 or 23 contiguous nucleobase portion of SEQ ID NO: 307, 312 or 626. In certain embodiments, the compound comprises a second oligonucleotide. In certain embodiments, the compound comprises an oligonucleotide comprising at least an 8, 9, 10, 11, 12, 13, 14, 15, 16, 17, 18, 19, 20, 21, 22 or 23 contiguous nucleobase portion of SEQ ID NO: 468, 611, 616, 619 or 629. In certain embodiments, the compound comprises a first oligonucleotide comprising at least an 8, 9, 10, 11, 12, 13, 14, 15, 16, 17, 18, 19, 20, 21, 22 or 23 contiguous nucleobase portion of SEQ ID NO: 312 and a second oligonucleotide comprising at least an 8, 9, 10, 11, 12, 13, 14, 15, 16, 17, 18, 19, 20, 21, 22 or 23 contiguous nucleobase portion of SEQ ID NO: 619. In certain embodiments, the compound comprises a first oligonucleotide comprising at least an 8, 9, 10, 11, 12, 13, 14, 15, 16, 17, 18, 19, 20, 21, 22 or 23 contiguous nucleobase portion of SEQ ID NO: 626 and a second oligonucleotide comprising at least an 8, 9, 10, 11, 12, 13, 14, 15, 16, 17, 18, 19, 20, 21, 22 or 23 contiguous nucleobase portion of SEQ ID NO: 629.

In certain embodiments, the compound comprises ribonucleotides in which the oligonucleotide has uracil (U) in place of thymine (T) for any of the sequences provided here. In certain embodiments, the compound comprises deoxyribonucleotides in which the oligonucleotide has thymine (T) in place of uracil (U) for any of the sequences provided here.

### Certain Mechanisms

In certain embodiments, compounds described herein comprise or consist of modified oligonucleotides. In certain embodiments, compounds described herein comprise or consist of antisense oligonucleotides. In certain embodiments, compounds comprise or consist of oligomeric compounds. In certain embodiments, compounds described herein are capable of hybridizing to a target nucleic acid. In certain embodiments, compounds described herein selectively affect one or more target nucleic acid. Such compounds comprise a nucleobase sequence that hybridizes to one or more target nucleic acid, resulting in one or more desired activity and does not hybridize to one or more non-target nucleic acid or does not hybridize to one or more non-target nucleic acid in such a way that results in a significant undesired activity.

In certain embodiments, hybridization of a compound described herein to a target nucleic acid results in recruitment of one or more proteins that cause the cleavage of the target nucleic acid. For example, certain compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in the alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to the target nucleic acid results in the alteration of RNA processing. In certain such embodiments, hybridization of the compound to a target nucleic acid results in alteration of translation of the target nucleic acid.

Activities resulting from the hybridization of a compound to a target nucleic acid may be observed directly or indirectly. In certain embodiments, observation or detection of an activity involves observation or detection of a change in an amount of a target nucleic acid or protein encoded by such target nucleic acid, a change in the ratio of splice variants of a nucleic acid or protein, and/or a phenotypic change in a cell or animal.

### Certain Modifications

In certain aspects, the disclosure relates to compounds that comprise or consist of oligonucleotides. Oligonucleotides consist of linked nucleosides. In certain embodiments, oligonucleotides may be unmodified RNA or DNA or may be modified. In certain embodiments, the oligonucleotides are modified oligonucleotides. In certain embodiments, the modified oligonucleotides comprise at least one modified sugar, modified nucleobase or modified internucleoside linkage relative to an unmodified RNA or DNA. In certain embodiments, an oligonucleotide has a modified nucleoside. A modified nucleoside may comprise a modified sugar, a modified nucleobase or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′,3′,4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, O—(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂)ₙO]ₘCH₃, O(CH₂)ₙOCH₃, O(CH₂)ₙCH₃, O(CH₂)ₙONH₂, O(CH₂)ₙNH₂, O(CH₂)ₙSCH₃, and O(CH₂)ₙON[(CH₂)ₙCH₃)]₂, where n and m are independently from 1 to about 10. In certain embodiments, substituent groups at the 2′ position include, but are not limited to, OCH₂CH₂OCH₃ (“MOE”), O(CH₂)₂N(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128. In certain embodiments, substituent groups at the 5′ position suitable for non-bicyclic modified sugars include, but are not limited to, methyl (“Me”) (R or S), vinyl, and methoxy. In certain embodiments, substituents described herein for the 2′,4′ and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugars substituents include, but are not limited to, 5′-Me-2′-F, 5′-Me-2′-OMe (including both R and S isomers). In certain embodiments, modified sugar substituents include those described in Migawa et al., WO 2008/101157 and Rajeev et al., US2013/0203836.

In certain embodiments, a modified sugar is a bicyclic sugar. A bicyclic sugar is a modified sugar comprising two rings, wherein the second ring is formed via a bridge connecting two of the atoms in the first ring thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′-CH₂-2′, 4′-(CH₂)₂—2′, 4′-(CH₂)₃—2′, 4′-CH₂—O-2′ (“LNA”), 4′-CH₂—S—2′, 4′-(CH₂)₂—O-2′ (“ENA”), 4′-CH(CH₃)—O-2′ (“constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (e.g., U.S. Pat. No. 7,399,845), 4′-C(CH₃)(CH₃)—O-2′ and analogs thereof (e.g., U.S. Pat. No. 8,278,283), 4′-CH₂—N(OCH₃)—2′ and analogs thereof (e.g., U.S. Pat. No. 8,278,425), 4′-CH₂—O—N(CH₃)—2′ (e.g., U.S. Patent Publication No. 2004/0171570), 4′-CH₂—N(R)—O-2′, wherein R is H, C₁-C₁₂ alkyl, or a protecting group (e.g., U.S. Pat. No. 7,427,672), 4′-CH₂—C(H)(CH₃)—2′ (e.g., Chattopadhyaya el al., J. Org. Chem., 2009, 74, 118-134), and 4′-CH₂—C(═CH₂)-2′ and analogs thereof (e.g., U.S. Pat. No. 8,278,426). The entire contents of each of the foregoing are hereby incorporated herein by reference. Additional representative U.S. patents and U.S. Patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including for example α-L-ribofuranose and β-D-ribofuranose (see e.g., WO 99/14226). Specified bicyclic nucleosides herein are in the β-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative US patents that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378, the entire contents of which is hereby incorporated herein by reference. Many other bicyclic and tricyclic sugar and sugar surrogate ring systems are known in the art that can be used in modified nucleosides.

In certain aspects, the disclosure relates to compounds comprising at least one oligonucleotide wherein the nucleosides of such oligonucleotide comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif. A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars and the gap comprises a majority of unmodified sugars. In certain embodiments, the nucleosides of the gap are deoxynucleosides. Compounds with a gapmer sugar motif are described in, for example U.S. Pat. No. 8,790,919, the entire contents of which is hereby incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the entire contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications wherein one or more consecutive nucleosides comprising a first sugar modification alternates with one or more consecutive nucleosides comprising a second sugar modification and one or more consecutive nucleosides comprising a third sugar modification, etc. For example, if A, B and C each represent one type of modification to the nucleoside, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′-OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif, is fully modified in that each nucleoside other than the nucleosides comprising the sugar motif comprises a sugar modification.

### Nucleobase Modifications and Motifs

In certain embodiments, compounds described herein comprise modified oligonucleotides. In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S. T., Ed., CRC Press, 2008, 163-166 and 442-443 (Chapters 6 and 15), each of which are hereby incorporated herein by reference.

Publications that teach the preparation of certain of the above noted modified nucleobases, as well as other modified nucleobases include without limitation, US Applications 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096; 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

### Internucleoside Linkage Modifications and Motifs

A 3′ to 5′ phosphodiester linkage is the naturally occurring internucleoside linkage of RNA and DNA. In certain embodiments, compounds described herein have one or more modified, i.e., non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g. methylphosphonates), phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′-CH₂—N(CH₃)—O-5′), amide-3 (3′-CH₂—C(═O)—N(H)—5′), amide-4 (3′-CH₂—N(H)—C(═O)—5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, compounds provided herein comprise at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′-end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

### Conjugate Groups

In certain embodiments, the compounds described herein comprise or consist of one or more oligonucleotides and, optionally, one or more conjugate groups. Conjugate groups may be attached to either or both ends of an oligonucleotide and/or at any internal position. In certain embodiments, a conjugate group is attached at the 3′ end of an oligonucleotide. In certain embodiments, a conjugate group is attached at the 5′ end of an oligonucleotide. In certain embodiments, oligonucleotides are covalently attached to one or more conjugate groups.

In certain embodiments, conjugate groups are terminal groups attached to either or both ends of an oligonucleotide. In certain such embodiments, terminal groups are attached at the 3′ end of an oligonucleotide. In certain such embodiments, terminal groups are attached at the 5′ end of an oligonucleotide. In certain embodiments, terminal groups include, but are not limited to, capping groups, phosphate moieties, protecting groups, modified or unmodified nucleosides, and two or more nucleosides that are independently modified or unmodified, such as an overhang.

In certain embodiments, conjugate groups modify one or more properties of the attached oligonucleotide, including, but not limited to, pharmacodynamics, pharmacokinetics, stability, activity, half-life, binding, absorption, tissue distribution, cellular distribution, cellular uptake, charge and clearance. In certain embodiments, conjugate groups enhance the affinity of a compound for a selected target, e.g., molecule, cell or cell type, compartment, e.g., a cellular or organ compartment, tissue, organ or region of the body, as, e.g., compared to a compound absent such a conjugate group. In certain embodiments, conjugate groups impart a new property on the attached oligonucleotide, e.g., fluorophores or reporter groups that enable detection of the oligonucleotide.

In certain embodiments, conjugate groups include, but are not limited to, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, conjugate groups include an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

In certain embodiments, conjugate groups are targeting moieties. In certain embodiments, a targeting moiety includes, but is not limited to, a lectin, glycoprotein, lipid, protein, peptide, peptide mimetic, receptor ligand, antibody, thyrotropin, melanotropin, surfactant protein A, carbohydrate, carbohydrate derivative, modified carbohydrate, carbohydrate cluster, polysaccharide, modified polysaccharide, or polysaccharide derivative, mucin carbohydrate, multivalent lactose, multivalent galactose, N-acetyl-galactosamine (GalNAc), N-acetylglucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

In certain embodiments, conjugate groups may include, but are not limited to, the conjugate groups described in the following references such as cholesterol (e.g., Letsinger et al., Proc. Natl. Acid. Sci. USA, 1989, 86: 6553-6556), cholic acid (e.g., Manoharan et al., Biorg. Med. Chem. Let., 1994, 4:1053-1060), thioether, e.g., hexyl-S-tritylthiol (e.g., Manoharan et al., Ann. NY. Acad. Sci., 1992, 660:306-309; Manoharan et al., Biorg. Med. Chem. Let., 1993, 3:2765-2770), thiocholesterol (e.g., Oberhauser et al., Nucl. Acids Res., 1992, 20:533-538), aliphatic chains, e.g., do-decan-diol or undecyl residues (e.g., Saison-Behmoaras et al., EMBO J, 1991, 10:1111-1118; Kabanov et al., FEBS Lett., 1990, 259:327-330; Svinarchuk et al., Biochimie, 1993, 75:49-54), phospholipids, e.g., di-hexadecyl-rac-glycerol or triethyl-ammonium 1,2-di-O-hexadecyl-rac-glycero-3-H-phosphonate (e.g., Manoharan et al., Tetrahedron Lett., 1995, 36:3651-3654; Shea et al., Nucl. Acids Res., 1990, 18:3777-3783), polyamines or a polyethylene glycol chains (e.g., Manoharan et al., Nucleosides & Nucleotides, 1995, 14:969-973), adamantane acetic acid (e.g., Manoharan et al., Tetrahedron Lett., 1995, 36:3651-3654), palmityl (e.g., Mishra et al., Biochim. Biophys. Acta, 1995, 1264:229-237), octadecylamine or hexylamino-carbonyloxychole sterol moiety (e.g., Crooke et al. J. Pharmacol. Exp. Ther., 1996, 277:923-937), tocopherol (e.g., Nishina et al., Molecular Therapy Nucleic Acids, 2015, 4, e220 and Nishina et al., Molecular Therapy, 2008, 16:734-740), GalNAc and other carbohydrates (e.g., Maier et al., Bioconjugate Chemistry, 2003, 14, 18-29; Rensen et al., J. Med. Chem. 2004, 47, 5798-5808; WO2009/073809 and U.S. Pat. Nos. 8,106,022; 8,450,467 and 8,828,957; and WO2014/179445; WO2014/179620 and U.S. Pat. Nos. 9,127,276; 9,181,549 and 10,844,379) each of which is incorporated herein by reference in its entirety.

Conjugate groups may be attached to oligonucleotides through conjugate linkers. In certain embodiments, a conjugate linker comprises a chain structure, such as a hydrocarbyl chain, or an oligomer of repeating units or combination of such repeating units. In certain embodiments, a conjugate linker comprises one or more groups selected from alkyl, amino, oxo, amide, disulfide, polyethylene glycol, ether, thioether, and hydroxylamino. In certain embodiments, a conjugate linker comprises at least one phosphorus group. In certain embodiments, a conjugate linker comprises at least one phosphate group. In certain embodiments, a conjugate linker includes at least one neutral linking group. In certain embodiments, conjugate linkers include, but are not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, conjugate linkers comprise 1-10 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, conjugate groups and conjugate linkers as well as other modifications include, without limitation, those described in the following references: U.S. Pat. Nos. 5,994,517; 6,300,319; 6,660,720; 6,906,182; 7,262,177; 7,491,805; 8,106,022; 7,723,509; 9,127,276; US 2006/0148740; US 2011/0123520; WO2013/033230; WO2012/037254, Biessen et al., J. Med. Chem. 1995, 38, 1846-1852; Lee et al., Bioorganic & Medicinal Chemistry 2011,19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carhohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycohiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromh Vase Biol, 2006, 26, 169-175; van Rossenberg et al., Gene Ther, 2004, 11, 457-464; Sato et al., JAm Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2003/0119724; US2011/0207799; US2012/0035115; US2012/0230938; US2005/0164235; US2006/0183886; US2012/0136042; US2012/0095075; US2013/0109817; US2006/0148740; US2008/0206869; US2012/0165393; US2012/0101148; US2013/0121954; US2011/0123520; US2003/0077829; US2008/0108801; and US2009/0203132; each of which is incorporated herein by reference in its entirety.

### Certain Targeting Moieties

In certain embodiments, a compound provided herein comprises a conjugate group. In certain embodiments, an oligonucleotide provided herein comprises a conjugate group. In certain embodiments, the conjugate group is a targeting moiety. In certain embodiments, the targeting moiety comprises one or more GalNAc. In certain embodiments, the one or more GalNAc are attached to one or more positions on a furanose ring. In certain embodiments, the one or more GalNAc are attached to the 2′ or 3′ position on a furanose ring. In certain embodiments, the furanose ring is a subunit of the oligonucleotide. In certain embodiments, the furanose ring is the 5′ nucleoside sugar of an oligonucleotide. In certain embodiments, the furanose ring is the 5′ nucleoside sugar of a sense oligonucleotide. In certain embodiments, a compound or oligonucleotide comprises one or more subunits with the following formula or a salt, solvate, or hydrate thereof:

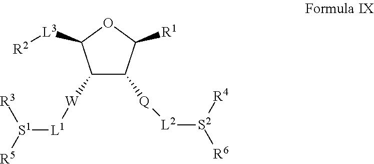

wherein:
R¹ is H, adenine, guanine, thymine, cytosine, uracil, carbocyclyl, heterocyclyl, aryl, heteroaryl, or a nucleobase isostere;
R² is the oligonucleotide sequence;
L¹ is alkyl, or alkyl-C(═O)—NH-alkyl;
L² is alkyl, or alkyl-C(═O)—NH-alkyl;
L³ is a bond, a phosphodiester bond, a phosphorothioate bond, a triazole, a tetrazole, an amide, a reverse-amide, a carbamate, a carbonate, urea, 0, S, S(═O), S(═O)₂, NH, substituted N group, alkyl, alkenyl, dienyl, alkynyl, heteroalkyl, phosphate;
R³ is H, —C═(O)—NH—(CH₂CH₂O)ⱼ-GalNAc, or —C(═O)—NH-alkyl-NH—C(═O)-alkyl-O—GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
W and Q are each independently O, NH, CH₂, or CH₂O;
S¹ and S² are each independently C(R⁷) or N, wherein each instance of R⁷ is independently H, alkyl, heteroalkyl, or halogen;
j is an integer 1-10, inclusive;
k is an integer 1-10, inclusive;
m is an integer 1-10, inclusive; and n is an integer 1-10, inclusive.

In certain embodiments, R³, R⁴, R⁵, and R⁶ are the same. In certain embodiments, R³, R⁵, and R⁶ are the same. In certain embodiments, R³ or R⁴ is H.

In certain embodiments, L¹ and L² are the same.

In certain embodiments, L¹ and L² are each independently alkyl; R³ is H, —C═(O)—NH—(CH₂CH₂O)ⱼ-GalNAc, or —C(═O)—NH-alkyl-NH—C(═O)-alkyl-O-GalNAc; R⁴ is H, —C═(O)—NH—(CH₂CH₂O)ₖ-GalNAc, or —C(═O)—NH-alkyl-NH—C(═O)-alkyl-O-GalNAc; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, L¹ and L² are each independently alkyl-C(═O)—NH-alkyl; R³ is H, —C═(O)—NH—(CH₂CH₂O)ⱼ-GalNAc, or —C(═O)—NH-alkyl-NH—C(═O)-alkyl-O-GalNAc; R⁴ is H, —C═(O)—NH—(CH₂CH₂O)ₖ-GalNAc, or —C(═O)—NH-alkyl-NH—C(═O)-alkyl-O-GalNAc; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, R⁴ is H.

In certain embodiments, L¹ and L² are each independently alkyl; R³ is —C═(O)—NH—(CH₂CH₂O)ⱼ-GalNAc, or —C(═O)—NH-alkyl-NH—C(═O)-alkyl-O-GalNAc; R⁴ is H; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, L¹ and L² are each independently alkyl-C(═O)—NH-alkyl; R³ is —C═(O)—NH—(CH₂CH₂O)ⱼ-GalNAc, or —C(═O)—NH-alkyl-NH—C(═O)-alkyl-O-GalNAc; R⁴ is H; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, R³ is —C═(O)—NH—(CH₂CH₂O)ⱼ-GalNAc; R⁴ is H; R⁵ is —C═(O)—NH—(CH₂CH₂O)ₘ-GalNAc; and R⁶ is —C═(O)—NH—(CH₂CH₂O)ₙ-GalNAc.

In certain embodiments, R³ is —C(═O)—NH-alkyl-NH—C(═O)-alkyl-O-GalNAc; R⁴ is H; R⁵ is —C(═O)—NH-alkyl-NH—C(═O)-alkyl-O-GalNAc; and R⁶ is —C(═O)—NH-alkyl-NH—C(═O)-alkyl-O-GalNAc.

In certain embodiments, a compound or oligonucleotide comprises one or more subunits with the following formula or a salt, solvate, or hydrate thereof:

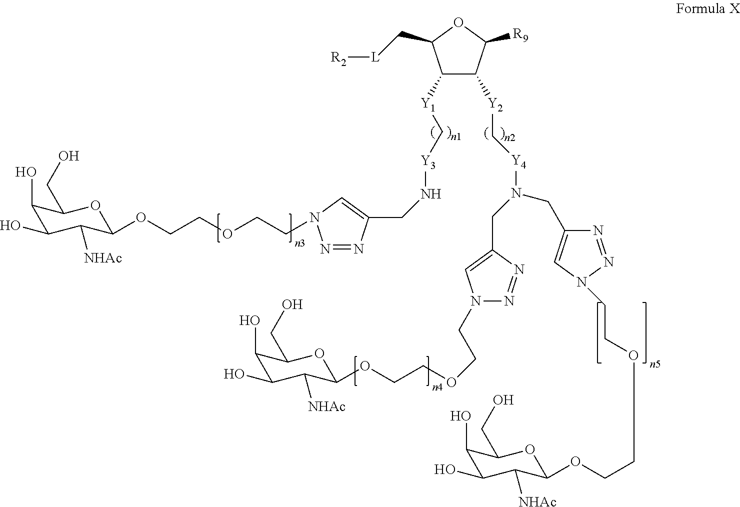

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is the oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each n₁, n₃, n₄ and n₅ is independently 0, 1, 2, 3, 4, 5, 6, 7, 8, 9, or 10.

In certain embodiments, a compound or oligonucleotide comprises one or more subunits with the following formula or a salt, solvate, or hydrate thereof:

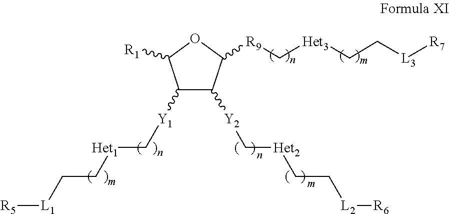

wherein:
each n is independently 1, 2, 3, 4, or 5;
each m is independently 0, 1, 2, 3, 4, 5, or 6;
each o is independently 0, 1, 2, 3, 4, 5, or 6;
each of L₁, L₂, and L₃ is independently absent, C(═O), or C(═O)NH;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each of Het₁, Het₂, and Het₃ is independently optionally substituted heteroaryl or optionally substituted heterocyclyl;
R¹ is the oligonucleotide sequence linked by a bond, a phosphodiester bond, a phosphorothioate bond, a triazole, a tetrazole, an amide, a reverse-amide, a carbamate, a carbonate, urea, alkyl, or heteroalkyl;
each R₅, R₆, and R₇ is independently

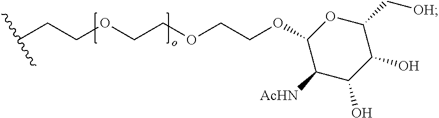

R₉ is optionally substituted heterocyclyl;
each R^{a} is independently H, alkyl, halo, OR^{c}, or SR^{c};
each R^{b} is independently H, alkyl, halo, OR^{c}, or SR^{c}; and
each R^{C} is independently H or alkyl.

In certain embodiments, the subunit is selected from Formulae I through VIII or a salt, solvate, or hydrate thereof, wherein R is the modified oligonucleotide other than the 5′ nucleoside. In certain embodiments, the 5′ nucleoside of the modified oligonucleotide is Formula I and R′ is O. In certain embodiments, the 5′ nucleoside of the modified oligonucleotide is Formula I and R′ is S. In certain embodiments, the 5′ nucleoside of the modified oligonucleotide is Formula II and R′ is O. In certain embodiments, the 5′ nucleoside of the modified oligonucleotide is Formula II and R′ is S. In certain embodiments, the 5′ nucleoside of the modified oligonucleotide is Formula III and R′ is O. In certain embodiments, the 5′ nucleoside of the modified oligonucleotide is Formula III and R′ is S. In certain embodiments, the 5′ nucleoside of the modified oligonucleotide is Formula IV and R′ is O. In certain embodiments, the 5′ nucleoside of the modified oligonucleotide is Formula IV and R′ is S. In certain embodiments, the 5′ nucleoside of the modified oligonucleotide is Formula V and R′ is O. In certain embodiments, the 5′ nucleoside of the modified oligonucleotide is Formula V and R′ is S. In certain embodiments, the 5′ nucleoside of the modified oligonucleotide is Formula VI and R′ is O. In certain embodiments, the 5′ nucleoside of the modified oligonucleotide is Formula VI and R′ is S. In certain embodiments, the 5′ nucleoside of the modified oligonucleotide is Formula VII and R′ is O. In certain embodiments, the 5′ nucleoside of the modified oligonucleotide is Formula VII and R′ is S. In certain embodiments, the 5′ nucleoside of the modified oligonucleotide is Formula VIII and R′ is O. In certain embodiments, the 5′ nucleoside of the modified oligonucleotide is Formula VIII and R′ is S.

### Target Nucleic Acids and Target Regions

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain embodiments, the target nucleic acid is non-coding. In certain such embodiments, the target nucleic acid is selected from an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an exon. In certain such embodiments, the target region is entirely within an intron. In certain embodiments, the target region spans an intron/exon junction. In certain embodiments, the target region is at least 50% within an intron.

In certain embodiments, compounds disclosed herein hybridize with a PKK nucleic acid. The most common mechanism of hybridization involves hydrogen bonding between complementary nucleobases of the nucleic acid molecules. Hybridization can occur under varying conditions. Hybridization conditions are sequence-dependent and are determined by the nature and composition of the nucleic acid molecules to be hybridized. Methods of determining whether a sequence hybridizes specifically to a target nucleic acid are well known in the art. In certain embodiments, the compounds provided herein specifically hybridize with a PKK nucleic acid.

Nucleotide sequences that encode PKK include, without limitation, the following: GENBANK Accession Nos. NM_000892.5 (incorporated herein as SEQ ID NO: 1), NG_012095.2 truncated from 23529 . . . 54493 (incorporated herein as SEQ ID NO: 2), XM_017008181.1 (incorporated herein as SEQ ID NO: 3), NC_000004.12 truncated from 186215714 to 186258477 (incorporated herein as SEQ ID NO: 4), NM_001318394.2 (incorporated herein as SEQ ID NO: 5) and NM_001318396.2 (incorporated herein as SEQ ID NO: 6).

### Complementarity

Oligonucleotides provided herein may have a defined percent complementarity to a particular nucleic acid, target region, oligonucleotide, or portion thereof. Non-complementary nucleobases may be tolerated provided that the oligonucleotide remains able to specifically hybridize to the nucleic acid, oligonucleotide, or portion thereof. In certain embodiments, the oligonucleotides provided herein, or a specified portion thereof are at least, or are up to 70%, 80%, 85%, 86%, 87%, 88%, 89%, 90%, 91%, 92%, 93%, 94%, 95%, 96%, 97%, 98%, 99%, or 100% complementary to a target nucleic acid, a target region, an oligonucleotide or specified portion thereof. In certain embodiments, the oligonucleotides provided herein, or a specified portion thereof, are 70% to 75%, 75% to 80%, 80% to 85%, 85% to 90%, 90% to 95%, 95% to 100%, or any number in between these ranges, complementary to a target nucleic acid, a target region, an oligonucleotide or specified portion thereof. Percent complementarity of an oligonucleotide with a target nucleic acid, a target region, an oligonucleotide or specified portion thereof can be determined using routine methods. For example, an oligonucleotide in which 18 of 20 nucleobases of the oligonucleotide are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an oligonucleotide which is 18 nucleobases in length having four non-complementary nucleobases which are flanked by two regions of complete complementarity with the target nucleic acid would have 77.8% overall complementarity with the target nucleic acid. Percent complementarity of an oligonucleotide with a region of a target nucleic acid, a target region, an oligonucleotide or specified portion thereof can be determined routinely using BLAST programs (basic local alignment search tools) known in the art. In certain embodiments, oligonucleotides described herein, or specified portions thereof, are fully complementary (i.e. 100% complementary) to a target nucleic acid, a target region, an oligonucleotide or specified portion thereof. For example, an oligonucleotide may be fully complementary to a target nucleic acid, a target region, an oligonucleotide, or specified portion thereof. As used herein, “fully complementary” means each nucleobase of an oligonucleotide is complementary to the corresponding nucleobase of a target nucleic acid, a target region, an oligonucleotide, or a specified portion thereof. For example, a 20 nucleobase oligonucleotide is fully complementary to a target sequence that is 400 nucleobases long, so long as there is a corresponding 20 nucleobase portion of the target nucleic acid that is fully complementary to the compound. “Fully complementary” can also be used in reference to a specified portion of the first and/or the second nucleic acid. For example, a 20 nucleobase portion of a 30 nucleobase oligonucleotide can be “fully complementary” to a 20 nucleobase region of a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, oligonucleotides described herein comprise one or more mismatched nucleobases relative to a target nucleic acid, a target region, an oligonucleotide or a specified portion thereof. In certain embodiments, oligonucleotides described herein that are, or are up to 12, 13, 14, 15, 16, 17, 18, 19, 20, 21, 22, or 23 nucleobases in length comprise no more than 4, no more than 3, no more than 2, or no more than 1 non-complementary nucleobase(s) relative to a target nucleic acid, or specified portion thereof. In certain embodiments, oligonucleotides described herein that are, or are up to 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, a target region, an oligonucleotide, or specified portion thereof. In certain embodiments, the mismatch is at position 1, 2, 3, 4, 5, 6, 7, 8, 9, 10, 11, 12, 13 or 14 from the 5′-end of the oligonucleotide. In certain embodiments, the mismatch is at position 1, 2, 3, 4, 5, 6, 7, 8, 9, 10, 11, or 12, 13 or 14 from the 3′-end of the oligonucleotide. In certain embodiments, the mismatch forms a wobble base pair with a corresponding nucleobase on the target nucleic acid. For example, in certain embodiments, the mismatch forms a wobble base pair selected from hypoxanthine (nucleobase of inosine) and uracil (I:U base pair); guanine and uracil (G:U base pair); hypoxanthine and adenine (I:A base pair); and hypoxanthine and cytosine (I:C base pair). Accordingly, in certain embodiments, a mismatched nucleobase on an oligonucleotide comprises hypoxanthine, guanine, or uracil.

In certain embodiments, oligonucleotides described herein may be complementary to a portion of a nucleic acid. As used herein, “portion” refers to a defined number of contiguous nucleobases within a region of a nucleic acid. A “portion” can also refer to a defined number of contiguous nucleobases of an oligonucleotide. In certain embodiments, the oligonucleotides are complementary to at least an 8 nucleobase portion of a nucleic acid. In certain embodiments, the oligonucleotides are complementary to at least a 9 nucleobase portion of a nucleic acid. In certain embodiments, the oligonucleotides are complementary to at least a 10 nucleobase portion of a nucleic acid. In certain embodiments, the oligonucleotides are complementary to at least an 11 nucleobase portion of a nucleic acid. In certain embodiments, the oligonucleotides are complementary to at least a 12 nucleobase portion of a nucleic acid. In certain embodiments, the oligonucleotides are complementary to at least a 13 nucleobase portion of a nucleic acid. In certain embodiments, the oligonucleotides are complementary to at least a 14 nucleobase portion of a nucleic acid. In certain embodiments, the oligonucleotides are complementary to at least a 15 nucleobase portion of a nucleic acid. In certain embodiments, the oligonucleotides are complementary to at least a 16 nucleobase portion of a nucleic acid. Also contemplated are oligonucleotides that are complementary to at least a 9, 10, 17, 18, 19, 20, 21, 22, 23 or more nucleobase portion of a nucleic acid, or a range defined by any two of these values. In certain embodiments, the oligonucleotide is an antisense oligonucleotide. In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid. In certain embodiments, the oligonucleotide is a sense oligonucleotide. In certain embodiments, a portion of the sense oligonucleotide is compared to an equal length portion of an antisense oligonucleotide. In certain embodiments, an 8, 9, 10, 11, 12, 13, 14, 15, 16, 17, 18, 19, 20, 21, 22, 23, 24, or 25 nucleobase portion of a sense oligonucleotide is compared to an equal length portion of an antisense oligonucleotide.

### Identity

The oligonucleotides provided herein may also have a defined percent identity to a particular nucleic acid, target region, oligonucleotide, or specified portion thereof. As used herein, an oligonucleotide is identical to a sequence disclosed herein if it has the same nucleobase pairing ability. For example, a DNA which contains thymidine in place of uracil in a disclosed RNA sequence would be considered identical to the RNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an oligonucleotide is calculated according to the number of bases that have identical base pairing relative to the sequence to which it is being compared. In certain embodiments, oligonucleotides described herein, or portions thereof, are, or are at least, 70%, 75%, 80%, 85%, 90%, 91%, 92%, 93%, 94%, 95%, 96%, 97%, 98%, 99% or 100% identical to one or more of the nucleic acids, oligonucleotides, or a portion thereof, disclosed herein. In certain embodiments, oligonucleotides described herein are about 70%, 75%, 80%, 85%, 90%, 91%, 92%, 93%, 94%, 95%, 96%, 97%, 98%, or 99% identical, or any percentage between such values, to a particular nucleic acid or oligonucleotide, or portion thereof.

In certain embodiments, an oligonucleotide may have one or more mismatched nucleobases. In certain such embodiments, the mismatch is at position 1, 2, 3, 4, 5, 6, 7, 8, 9, 10, 11, 12, 13 or 14 from the 5′-end of the oligonucleotide. In certain such embodiments, the mismatch is at position 1, 2, 3, 4, 5, 6, 7, 8, 9, 10, 11, or 12, 13 or 14 from the 3′-end of the oligonucleotide. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid. In certain embodiments, the oligonucleotide is a sense oligonucleotides. In certain embodiments, a portion of the sense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Pharmaceutical Compositions and Formulations

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. Certain embodiments provide pharmaceutical compositions comprising one or more compounds or a salt thereof. In certain embodiments, the compounds are antisense oligonucleotides. In certain embodiments, the compounds are oligomeric compounds. In certain embodiments, the compounds comprise or consist of one or more modified oligonucleotides. In certain such embodiments, the pharmaceutical composition comprises one or more compound and a suitable pharmaceutically acceptable diluent or carrier. In certain embodiments, a pharmaceutical composition comprises one or more compound and a sterile saline solution. In certain embodiments, such pharmaceutical composition consists of one compound and a sterile saline solution. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compounds and phosphate-buffered saline (PBS). In certain embodiments, a pharmaceutical composition consists of one compound and sterile PBS. In certain embodiments, the sterile PBS is pharmaceutical grade PBS.

A compound described herein targeted to PKK can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to PKK and a pharmaceutically acceptable diluent. In certain embodiments, the pharmaceutically acceptable diluent is water. In certain embodiments, the compound comprises or consists of one or more modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense oligonucleotides. In certain embodiments, the compounds are oligomeric compounds. In certain embodiments, the compound comprises or consists of one or more modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts. A prodrug can include the incorporation of additional nucleosides at one or both ends of a compound which are cleaved by endogenous nucleases within the body, to form the active compound. In certain embodiments, the compounds or compositions further comprise a pharmaceutically acceptable carrier or diluent.

### Examples

The following examples describe the process to identify lead compounds targeted to PKK. Certain compounds are distinguished as having high potency and tolerability.

The following examples serve only to illustrate the compounds described herein and are not intended to limit the same. The following examples and related sequence listing accompanying this filing may identify sequence as either “RNA” or “DNA”; however, as disclosed herein, those sequences may be modified with any combination of chemical modifications. One of skill in the art will readily appreciate that the designation of a sequence as “RNA” or “DNA” is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (methylated uracil for natural uracil of RNA). Accordingly, nucleic acid sequences provided herein, including, but not limited to, those in the sequence listing, are intended to encompass nucleic acids containing any combination of natural or modified RNA and/or DNA, including, but not limited to, such nucleic acids having modified nucleobases.

Each of the references recited in the present application is incorporated herein by reference in its entirety.

Unless otherwise indicated in a separate compound chemistry table below, compounds are unmodified. Abbreviations for chemical modifications are provided in Table 1 below. IA and IS in a Ref ID NO: identifies an antisense strand and sense strand of a compound, respectively.

TABLE 1
Chemical Nomenclature

| Abbreviation | Structure |
|---|---|
| ‘m’ | 2′-O-methyl sugar modification (e.g., mA, mG, mC, mU) |
| ‘f’ | 2′-F sugar modification (e.g., fA, fG, fC, fU) |
| ‘*’ | Phosphorothioate intemucleoside linkage |
| ‘.’ | Phosphate internucleoside linkage |
| ‘dQ’ | Inverted abasic deoxyribose |
| ‘H1’ | Formula I |
| ‘H2’ | Formula II |
| ‘H4” | Formula III |
| ‘H6” | Formula IV |
| ‘H7’ | Formula V |
| ‘H9’ | Formula VI |
| ‘Hd’ | Formula VII |
| Hl | Formula VIII |

### Example 1—Inhibition of PKK in HEK-293T Cells

HEK-293T cells were seeded in antibiotic-free media at 20,000 cells/well in white-walled 96-well plates. The following day, the cells were co-transfected with 50 ng of PKK-siCHECK-2 and 10 nM PKK compound using Lipofectamine 2000 (each transfection was performed in triplicate). The cells were then incubated at 37° C./5% CO₂ for 48 hours.

Dual-Luciferase Reporter 1000 Assay (Promega Cat #E1980) was used to evaluate expression of firefly and renilla luciferase according to kit instructions. Firefly and renilla luciferase expression were measured using a luminimeter. Renilla luciferase was the readout for PKK gene expression while firefly served as the internal control. All renillla readings from each well were normalized to its corresponding firefly reading to obtain a renilla:firefly ratio. The ratios obtained for each well of transfected compound were then further normalized to the ratios obtained for cells that were not transfected with compound. These untransfected cells served as the 100% control. PKK inhibition was determined by comparing PKK expression to the untransfected cells and reported as % PKK inhibition (Tables 2-4).

TABLE 2
Inhibition of PKK mRNA by double-stranded compounds targeting SEQ ID NO: 1

| | Seq | SEQ | | | | | |
|---|---|---|---|---|---|---|---|
| | ID | ID | | | | | PKK |
| | NO: 1 | NO: 1 | | SEQ | | SEQ | % |
| Compound | Start | Stop | Antisense Sequence | ID | Sense Sequence | ID | inhi- |
| Number | Site | Site | (5′-3′) | NO: | (5′-3′) | NO: | bition |
| RD1839 | 171 | 191 | UUACAUCCCCACCUCUGAAGA | 10 | UCUUCAGAGGUGGGGAUGUAA | 314 | 70 |
| RD1840 | 172 | 192 | UCUACAUCCCCACCUCUGAAG | 11 | CUUCAGAGGUGGGGAUGUAGA | 315 | 56 |
| RD1841 | 173 | 193 | AGCUACAUCCCCACCUCUGAA | 12 | UUCAGAGGUGGGGAUGUAGCU | 316 | 34 |
| RD1842 | 174 | 194 | AAGCUACAUCCCCACCUCUGA | 13 | UCAGAGGUGGGGAUGUAGCUU | 317 | 53 |
| RD1843 | 175 | 195 | UAAGCUACAUCCCCACCUCUG | 14 | CAGAGGUGGGGAUGUAGCUUA | 318 | 64 |
| RD1844 | 176 | 196 | UGAAGCUACAUCCCCACCUCU | 15 | AGAGGUGGGGAUGUAGCUUCA | 319 | 71 |
| RD1845 | 177 | 197 | UGGAAGCUACAUCCCCACCUC | 16 | GAGGUGGGGAUGUAGCUUCCA | 320 | 70 |
| RD1846 | 178 | 198 | AUGGAAGCUACAUCCCCACCU | 17 | AGGUGGGGAUGUAGCUUCCAU | 321 | 81 |
| RD1847 | 179 | 199 | UAUGGAAGCUACAUCCCCACC | 18 | GGUGGGGAUGUAGCUUCCAUA | 322 | 74 |
| RD1848 | 180 | 200 | ACAUGGAAGCUACAUCCCCAC | 19 | GUGGGGAUGUAGCUUCCAUGU | 323 | 64 |
| RD1849 | 181 | 201 | UACAUGGAAGCUACAUCCCCA | 20 | UGGGGAUGUAGCUUCCAUGUA | 324 | 74 |
| RD1850 | 182 | 202 | UUACAUGGAAGCUACAUCCCC | 21 | GGGGAUGUAGCUUCCAUGUAA | 325 | 70 |
| RD1851 | 232 | 252 | UACCUUGGGUGGAAUGUGCAC | 22 | GUGCACAUUCCACCCAAGGUA | 326 | 77 |
| RD1852 | 233 | 253 | ACACCUUGGGUGGAAUGUGCA | 23 | UGCACAUUCCACCCAAGGUGU | 327 | 62 |
| RD1853 | 234 | 254 | AACACCUUGGGUGGAAUGUGC | 24 | GCACAUUCCACCCAAGGUGUU | 328 | 72 |
| RD1854 | 237 | 257 | UCAAACACCUUGGGUGGAAUG | 25 | CAUUCCACCCAAGGUGUUUGA | 329 | 61 |
| RD1855 | 238 | 258 | AGCAAACACCUUGGGUGGAAU | 26 | AUUCCACCCAAGGUGUUUGCU | 330 | 51 |
| RD1856 | 241 | 261 | AAUAGCAAACACCUUGGGUGG | 27 | CCACCCAAGGUGUUUGCUAUU | 331 | 82 |
| RD1857 | 242 | 262 | UAAUAGCAAACACCUUGGGUG | 28 | CACCCAAGGUGUUUGCUAUUA | 332 | 62 |
| RD1858 | 243 | 263 | UGAAUAGCAAACACCUUGGGU | 29 | ACCCAAGGUGUUUGCUAUUCA | 333 | 91 |
| RD1859 | 245 | 265 | ACUGAAUAGCAAACACCUUGG | 30 | CCAAGGUGUUUGCUAUUCAGU | 334 | 91 |
| RD1860 | 246 | 266 | AACUGAAUAGCAAACACCUUG | 31 | CAAGGUGUUUGCUAUUCAGUU | 335 | 94 |
| RD1861 | 247 | 267 | AAACUGAAUAGCAAACACCUU | 32 | AAGGUGUUUGCUAUUCAGUUU | 336 | 90 |
| RD1862 | 248 | 268 | AAAACUGAAUAGCAAACACCU | 33 | AGGUGUUUGCUAUUCAGUUUU | 337 | 91 |
| RD1863 | 249 | 269 | UAAAACUGAAUAGCAAACACC | 34 | GGUGUUUGCUAUUCAGUUUUA | 338 | 92 |
| RD1864 | 250 | 270 | AGAAAACUGAAUAGCAAACAC | 35 | GUGUUUGCUAUUCAGUUUUCU | 339 | 90 |
| RD1865 | 254 | 274 | UGGAAGAAAACUGAAUAGCAA | 36 | UUGCUAUUCAGUUUUCUUCCA | 340 | 91 |
| RD1866 | 262 | 282 | UAACUUGCUGGAAGAAAACUG | 37 | CAGUUUUCUUCCAGCAAGUUA | 341 | 89 |
| RD1867 | 284 | 304 | UCUUUUCUCCAUGUCAUUGAU | 38 | AUCAAUGACAUGGAGAAAAGA | 342 | 93 |

TABLE 3
Inhibition of PKK mRNA by double-stranded compounds targeting SEQ ID NO: 1

| | Seq | SEQ | | | | | |
|---|---|---|---|---|---|---|---|
| | ID | ID | | | | | PKK |
| | NO: 1 | NO: 1 | | SEQ | | SEQ | % |
| Compound | Start | Stop | Antisense Sequence | ID | Sense Sequence | ID | inhi- |
| Number | Site | Site | (5′-3′) | NO: | (5′-3′) | NO: | bition |
| RD1868 | 287 | 307 | AAACCUUUUCUCCAUGUCAUU | 39 | AAUGACAUGGAGAAAAGGUUU | 343 | 65 |
| RD1869 | 288 | 308 | UAAACCUUUUCUCCAUGUCAU | 40 | AUGACAUGGAGAAAAGGUUUA | 344 | 44 |
| RD1870 | 484 | 504 | UACCUUUUUUGGCAUUCUUCA | 41 | UGAAGAAUGCCAAAAAAGGUA | 345 | 66 |
| RD1871 | 489 | 509 | UGGUGCACCUUUUUUGGCAUU | 42 | AAUGCCAAAAAAGGUGCACCA | 346 | 45 |
| RD1872 | 662 | 682 | ACCAAUUUCUGAAAGGGCACA | 43 | UGUGCCCUUUCAGAAAUUGGU | 347 | 66 |
| RD1873 | 663 | 683 | AACCAAUUUCUGAAAGGGCAC | 44 | GUGCCCUUUCAGAAAUUGGUU | 348 | 50 |
| RD1874 | 691 | 711 | UCAAGAUGCUGGAAGAUGUUC | 45 | GAACAUCUUCCAGCAUCUUGA | 349 | 49 |
| RD1875 | 867 | 887 | AGGAACUUGGUGUGCCACUUU | 46 | AAAGUGGCACACCAAGUUCCU | 350 | 17 |
| RD1876 | 868 | 888 | UAGGAACUUGGUGUGCCACUU | 47 | AAGUGGCACACCAAGUUCCUA | 351 | 52 |
| RD1877 | 869 | 889 | AGAGGAACUUGGUGUGCCACU | 48 | AGUGGCACACCAAGUUCCUCU | 352 | 70 |
| RD1878 | 870 | 890 | UAGAGGAACUUGGUGUGCCAC | 49 | GUGGCACACCAAGUUCCUCUA | 353 | 68 |
| RD1879 | 871 | 891 | UUAGAGGAACUUGGUGUGCCA | 50 | UGGCACACCAAGUUCCUCUAA | 354 | 38 |
| RD1880 | 876 | 896 | UAGGAGUAGAGGAACUUGGUG | 51 | CACCAAGUUCCUCUACUCCUA | 355 | 75 |
| RD1881 | 877 | 897 | UGAGGAGUAGAGGAACUUGGU | 52 | ACCAAGUUCCUCUACUCCUCA | 356 | 69 |
| RD1882 | 878 | 898 | UUGAGGAGUAGAGGAACUUGG | 53 | CCAAGUUCCUCUACUCCUCAA | 357 | 85 |
| RD1883 | 879 | 899 | UUUGAGGAGUAGAGGAACUUG | 54 | CAAGUUCCUCUACUCCUCAAA | 358 | 82 |
| RD1884 | 880 | 900 | UCUUGAGGAGUAGAGGAACUU | 55 | AAGUUCCUCUACUCCUCAAGA | 359 | 69 |
| RD1885 | 881 | 901 | UUCUUGAGGAGUAGAGGAACU | 56 | AGUUCCUCUACUCCUCAAGAA | 360 | 68 |
| RD1886 | 882 | 902 | UUUCUUGAGGAGUAGAGGAAC | 57 | GUUCCUCUACUCCUCAAGAAA | 361 | 63 |
| RD1887 | 883 | 903 | UUUUCUUGAGGAGUAGAGGAA | 58 | UUCCUCUACUCCUCAAGAAAA | 362 | 71 |
| RD1888 | 886 | 906 | UUGUUUUCUUGAGGAGUAGAG | 59 | CUCUACUCCUCAAGAAAACAA | 363 | 64 |
| RD1889 | 887 | 907 | UGUGUUUUCUUGAGGAGUAGA | 60 | UCUACUCCUCAAGAAAACACA | 364 | 75 |
| RD1890 | 888 | 908 | UGGUGUUUUCUUGAGGAGUAG | 61 | CUACUCCUCAAGAAAACACCA | 365 | 88 |
| RD1891 | 920 | 940 | UCUUUUGCAGGUUAAAAGGCU | 62 | AGCCUUUUAACCUGCAAAAGA | 366 | 88 |
| RD1892 | 936 | 956 | AGGGUUCAGGUAAAGUUCUUU | 63 | AAAGAACUUUACCUGAACCCU | 367 | 58 |
| RD1893 | 937 | 957 | UAGGGUUCAGGUAAAGUUCUU | 64 | AAGAACUUUACCUGAACCCUA | 368 | 83 |
| RD1894 | 938 | 958 | UCAGGGUUCAGGUAAAGUUCU | 65 | AGAACUUUACCUGAACCCUGA | 369 | 78 |
| RD1895 | 939 | 959 | UGCAGGGUUCAGGUAAAGUUC | 66 | GAACUUUACCUGAACCCUGCA | 370 | 76 |
| RD1924 | 1060 | 1080 | UAAGUGAAAAACUGACAGCGA | 95 | UCGCUGUCAGUUUUUCACUUA | 399 | 77 |
| RD1925 | 1061 | 1081 | AUAAGUGAAAAACUGACAGCG | 96 | CGCUGUCAGUUUUUCACUUAU | 400 | 73 |
| RD1926 | 1062 | 1082 | AAUAAGUGAAAAACUGACAGC | 97 | GCUGUCAGUUUUUCACUUAUU | 401 | 81 |
| RD1927 | 1065 | 1085 | AAGAAUAAGUGAAAAACUGAC | 98 | GUCAGUUUUUCACUUAUUCUU | 402 | 58 |
| RD1928 | 1066 | 1086 | AAAGAAUAAGUGAAAAACUGA | 99 | UCAGUUUUUCACUUAUUCUUU | 403 | 43 |
| RD1929 | 1070 | 1090 | UAGUAAAGAAUAAGUGAAAAA | 100 | UUUUUCACUUAUUCUUUACUA | 404 | 0 |
| RD1930 | 1071 | 1091 | UGAGUAAAGAAUAAGUGAAAA | 101 | UUUUCACUUAUUCUUUACUCA | 405 | 4 |
| RD1931 | 1072 | 1092 | UGGAGUAAAGAAUAAGUGAAA | 102 | UUUCACUUAUUCUUUACUCCA | 406 | 8 |
| RD1932 | 1073 | 1093 | UGGGAGUAAAGAAUAAGUGAA | 103 | UUCACUUAUUCUUUACUCCCA | 407 | 65 |
| RD1933 | 1074 | 1094 | UUGGGAGUAAAGAAUAAGUGA | 104 | UCACUUAUUCUUUACUCCCAA | 408 | 75 |
| RD1934 | 1075 | 1095 | UCUGGGAGUAAAGAAUAAGUG | 105 | CACUUAUUCUUUACUCCCAGA | 409 | 85 |
| RD1935 | 1076 | 1096 | UUCUGGGAGUAAAGAAUAAGU | 106 | ACUUAUUCUUUACUCCCAGAA | 410 | 69 |
| RD1936 | 1077 | 1097 | UUUCUGGGAGUAAAGAAUAAG | 107 | CUUAUUCUUUACUCCCAGAAA | 411 | 75 |
| RD1937 | 1078 | 1098 | UCUUCUGGGAGUAAAGAAUAA | 108 | UUAUUCUUUACUCCCAGAAGA | 412 | 55 |
| RD1938 | 1080 | 1100 | AGUCUUCUGGGAGUAAAGAAU | 109 | AUUCUUUACUCCCAGAAGACU | 413 | 53 |
| RD1939 | 1081 | 1101 | UAGUCUUCUGGGAGUAAAGAA | 110 | UUCUUUACUCCCAGAAGACUA | 414 | 58 |
| RD1940 | 1085 | 1105 | UUUACAGUCUUCUGGGAGUAA | 111 | UUACUCCCAGAAGACUGUAAA | 415 | 75 |
| RD1941 | 1086 | 1106 | UCUUACAGUCUUCUGGGAGUA | 112 | UACUCCCAGAAGACUGUAAGA | 416 | 80 |
| RD1942 | 1087 | 1107 | UCCUUACAGUCUUCUGGGAGU | 113 | ACUCCCAGAAGACUGUAAGGA | 417 | 53 |
| RD1943 | 1109 | 1129 | UAAGAAACACUUACACUUCUC | 114 | GAGAAGUGUAAGUGUUUCUUA | 418 | 85 |
| RD1944 | 1110 | 1130 | UUAAGAAACACUUACACUUCU | 115 | AGAAGUGUAAGUGUUUCUUAA | 419 | 87 |
| RD1945 | 1111 | 1131 | UUUAAGAAACACUUACACUUC | 116 | GAAGUGUAAGUGUUUCUUAAA | 420 | 77 |
| RD1946 | 1117 | 1137 | UAUAAUCUUAAGAAACACUUA | 117 | UAAGUGUUUCUUAAGAUUAUA | 421 | 84 |
| RD1947 | 1141 | 1161 | AUCCUAGUUGGAGAACCAUCC | 118 | GGAUGGUUCUCCAACUAGGAU | 422 | 77 |
| RD1948 | 1142 | 1162 | AAUCCUAGUUGGAGAACCAUC | 119 | GAUGGUUCUCCAACUAGGAUU | 423 | 82 |
| RD1949 | 1167 | 1187 | UAGAGCUCCCUUGUGUCCCAU | 120 | AUGGGACACAAGGGAGCUCUA | 424 | 86 |
| RD1950 | 1168 | 1188 | UCAGAGCUCCCUUGUGUCCCA | 121 | UGGGACACAAGGGAGCUCUGA | 425 | 73 |
| RD1951 | 1170 | 1190 | AACCAGAGCUCCCUUGUGUCC | 122 | GGACACAAGGGAGCUCUGGUU | 426 | 74 |
| RD1952 | 1171 | 1191 | UAACCAGAGCUCCCUUGUGUC | 123 | GACACAAGGGAGCUCUGGUUA | 427 | 32 |
| RD1953 | 1172 | 1192 | UUAACCAGAGCUCCCUUGUGU | 124 | ACACAAGGGAGCUCUGGUUAA | 428 | 64 |
| RD1954 | 1174 | 1194 | UAGUAACCAGAGCUCCCUUGU | 125 | ACAAGGGAGCUCUGGUUACUA | 429 | 78 |
| RD1955 | 1175 | 1195 | AGAGUAACCAGAGCUCCCUUG | 126 | CAAGGGAGCUCUGGUUACUCU | 430 | 81 |
| RD1956 | 1179 | 1199 | UCAAAGAGUAACCAGAGCUCC | 127 | GGAGCUCUGGUUACUCUUUGA | 431 | 61 |
| RD1957 | 1196 | 1216 | UCCAGUGUUACACAAUCUCAA | 128 | UUGAGAUUGUGUAACACUGGA | 432 | 85 |
| RD1958 | 1197 | 1217 | UCCCAGUGUUACACAAUCUCA | 129 | UGAGAUUGUGUAACACUGGGA | 433 | 71 |
| RD1959 | 1200 | 1220 | UGUCCCCAGUGUUACACAAUC | 130 | GAUUGUGUAACACUGGGGACA | 434 | 70 |
| RD1960 | 1261 | 1281 | UCCCAAGAAGAGUUUGUUCCU | 131 | AGGAACAAACUCUUCUUGGGA | 435 | 37 |
| RD1961 | 1262 | 1282 | UCCCCAAGAAGAGUUUGUUCC | 132 | GGAACAAACUCUUCUUGGGGA | 436 | 40 |
| RD1962 | 1263 | 1283 | UUCCCCAAGAAGAGUUUGUUC | 133 | GAACAAACUCUUCUUGGGGAA | 437 | 25 |
| RD1963 | 1264 | 1284 | UCUCCCCAAGAAGAGUUUGUU | 134 | AACAAACUCUUCUUGGGGAGA | 438 | 2 |
| RD1964 | 1265 | 1285 | UUCUCCCCAAGAAGAGUUUGU | 135 | ACAAACUCUUCUUGGGGAGAA | 439 | 0 |
| RD1965 | 1266 | 1286 | ACUCUCCCCAAGAAGAGUUUG | 136 | CAAACUCUUCUUGGGGAGAGU | 440 | 0 |
| RD1966 | 1268 | 1288 | UCACUCUCCCCAAGAAGAGUU | 137 | AACUCUUCUUGGGGAGAGUGA | 441 | 12 |
| RD1967 | 1269 | 1289 | UCCACUCUCCCCAAGAAGAGU | 138 | ACUCUUCUUGGGGAGAGUGGA | 442 | 8 |
| RD1968 | 1272 | 1292 | AGGGCCACUCUCCCCAAGAAG | 139 | CUUCUUGGGGAGAGUGGCCCU | 443 | 0 |
| RD1969 | 1273 | 1293 | UAGGGCCACUCUCCCCAAGAA | 140 | UUCUUGGGGAGAGUGGCCCUA | 444 | 11 |
| RD1970 | 1274 | 1294 | UCAGGGCCACUCUCCCCAAGA | 141 | UCUUGGGGAGAGUGGCCCUGA | 445 | 42 |
| RD1971 | 1343 | 1363 | UUGGUGUCCUAUGAGUGACCC | 142 | GGGUCACUCAUAGGACACCAA | 446 | 60 |
| RD1972 | 1344 | 1364 | ACUGGUGUCCUAUGAGUGACC | 143 | GGUCACUCAUAGGACACCAGU | 447 | 37 |
| RD1973 | 1347 | 1367 | UCCACUGGUGUCCUAUGAGUG | 144 | CACUCAUAGGACACCAGUGGA | 448 | 43 |
| RD1974 | 1348 | 1368 | ACCCACUGGUGUCCUAUGAGU | 145 | ACUCAUAGGACACCAGUGGGU | 449 | 8 |
| RD1975 | 1349 | 1369 | UACCCACUGGUGUCCUAUGAG | 146 | CUCAUAGGACACCAGUGGGUA | 450 | 59 |
| RD1976 | 1381 | 1401 | UGAAGCCCAUCAAAGCAGUGG | 147 | CCACUGCUUUGAUGGGCUUCA | 451 | 77 |
| RD1977 | 1425 | 1445 | ACAGAUUUAAAAUGCCACUAU | 148 | AUAGUGGCAUUUUAAAUCUGU | 452 | 78 |
| RD1978 | 1426 | 1446 | UACAGAUUUAAAAUGCCACUA | 149 | UAGUGGCAUUUUAAAUCUGUA | 453 | 91 |
| RD1979 | 1427 | 1447 | UGACAGAUUUAAAAUGCCACU | 150 | AGUGGCAUUUUAAAUCUGUCA | 454 | 90 |
| RD1980 | 1428 | 1448 | UUGACAGAUUUAAAAUGCCAC | 151 | GUGGCAUUUUAAAUCUGUCAA | 455 | 83 |
| RD1981 | 1429 | 1449 | UCUGACAGAUUUAAAAUGCCA | 152 | UGGCAUUUUAAAUCUGUCAGA | 456 | 90 |
| RD1982 | 1430 | 1450 | UUCUGACAGAUUUAAAAUGCC | 153 | GGCAUUUUAAAUCUGUCAGAA | 457 | 68 |
| RD1983 | 1431 | 1451 | UGUCUGACAGAUUUAAAAUGC | 154 | GCAUUUUAAAUCUGUCAGACA | 458 | 72 |
| RD1984 | 1436 | 1456 | UGUAAUGUCUGACAGAUUUAA | 155 | UUAAAUCUGUCAGACAUUACA | 459 | 92 |
| RD1985 | 1441 | 1461 | UCUUUUGUAAUGUCUGACAGA | 156 | UCUGUCAGACAUUACAAAAGA | 460 | 92 |
| RD1986 | 1513 | 1533 | AUAUCAUGAUUCCCUUCUGAG | 157 | CUCAGAAGGGAAUCAUGAUAU | 461 | 63 |
| RD1987 | 1516 | 1536 | UCGAUAUCAUGAUUCCCUUCU | 158 | AGAAGGGAAUCAUGAUAUCGA | 462 | 71 |
| RD1988 | 1517 | 1537 | UGCGAUAUCAUGAUUCCCUUC | 159 | GAAGGGAAUCAUGAUAUCGCA | 463 | 80 |
| RD1989 | 1518 | 1538 | AGGCGAUAUCAUGAUUCCCUU | 160 | AAGGGAAUCAUGAUAUCGCCU | 464 | 77 |
| RD1990 | 1545 | 1565 | AAUUCAAAGGAGCCUGGAGUU | 161 | AACUCCAGGCUCCUUUGAAUU | 465 | 61 |
| RD1991 | 1546 | 1566 | UAAUUCAAAGGAGCCUGGAGU | 162 | ACUCCAGGCUCCUUUGAAUUA | 466 | 55 |
| RD1992 | 1547 | 1567 | UUAAUUCAAAGGAGCCUGGAG | 163 | CUCCAGGCUCCUUUGAAUUAA | 467 | 65 |
| RD1993 | 1548 | 1568 | UGUAAUUCAAAGGAGCCUGGA | 164 | UCCAGGCUCCUUUGAAUUACA | 468 | 51 |
| RD1994 | 1549 | 1569 | UUGUAAUUCAAAGGAGCCUGG | 165 | CCAGGCUCCUUUGAAUUACAA | 469 | 82 |
| RD1995 | 1550 | 1570 | AGUGUAAUUCAAAGGAGCCUG | 166 | CAGGCUCCUUUGAAUUACACU | 470 | 74 |
| RD1996 | 1551 | 1571 | UAGUGUAAUUCAAAGGAGCCU | 167 | AGGCUCCUUUGAAUUACACUA | 471 | 80 |
| RD1997 | 1552 | 1572 | UCAGUGUAAUUCAAAGGAGCC | 168 | GGCUCCUUUGAAUUACACUGA | 472 | 71 |
| RD1998 | 1557 | 1577 | UGAAUUCAGUGUAAUUCAAAG | 169 | CUUUGAAUUACACUGAAUUCA | 473 | 0 |
| RD1999 | 1567 | 1587 | AUUGGUUUUUGGAAUUCAGUG | 170 | CACUGAAUUCCAAAAACCAAU | 474 | 88 |
| RD2000 | 1568 | 1588 | UAUUGGUUUUUGGAAUUCAGU | 171 | ACUGAAUUCCAAAAACCAAUA | 475 | 91 |
| RD2001 | 1570 | 1590 | UAUAUUGGUUUUUGGAAUUCA | 172 | UGAAUUCCAAAAACCAAUAUA | 476 | 77 |
| RD2002 | 1571 | 1591 | UCAUAUUGGUUUUUGGAAUUC | 173 | GAAUUCCAAAAACCAAUAUGA | 477 | 83 |
| RD2003 | 1575 | 1595 | UUAGGCAUAUUGGUUUUUGGA | 174 | UCCAAAAACCAAUAUGCCUAA | 478 | 70 |
| RD2004 | 1576 | 1596 | UGUAGGCAUAUUGGUUUUUGG | 175 | CCAAAAACCAAUAUGCCUACA | 479 | 63 |
| RD2005 | 1577 | 1597 | AGGUAGGCAUAUUGGUUUUUG | 176 | CAAAAACCAAUAUGCCUACCU | 480 | 52 |
| RD2006 | 1578 | 1598 | AAGGUAGGCAUAUUGGUUUUU | 177 | AAAAACCAAUAUGCCUACCUU | 481 | 71 |
| RD2007 | 1579 | 1599 | UAAGGUAGGCAUAUUGGUUUU | 178 | AAAACCAAUAUGCCUACCUUA | 482 | 77 |
| RD2008 | 1580 | 1600 | UGAAGGUAGGCAUAUUGGUUU | 179 | AAACCAAUAUGCCUACCUUCA | 483 | 0 |
| RD2009 | 1581 | 1601 | UGGAAGGUAGGCAUAUUGGUU | 180 | AACCAAUAUGCCUACCUUCCA | 484 | 66 |
| RD2010 | 1588 | 1608 | UCACCUUUGGAAGGUAGGCAU | 181 | AUGCCUACCUUCCAAAGGUGA | 485 | 65 |
| RD2011 | 1591 | 1611 | UUGUCACCUUUGGAAGGUAGG | 182 | CCUACCUUCCAAAGGUGACAA | 486 | 37 |
| RD2012 | 1592 | 1612 | UGUGUCACCUUUGGAAGGUAG | 183 | CUACCUUCCAAAGGUGACACA | 487 | 57 |
| RD2013 | 1593 | 1613 | UUGUGUCACCUUUGGAAGGUA | 184 | UACCUUCCAAAGGUGACACAA | 488 | 22 |
| RD2014 | 1849 | 1869 | UUGAUGCCCACCAAACGCCAC | 185 | GUGGCGUUUGGUGGGCAUCAA | 489 | 18 |
| RD2015 | 1880 | 1900 | UUCCCUGCGGGCACAGCCUUC | 186 | GAAGGCUGUGCCCGCAGGGAA | 490 | 36 |
| RD2016 | 1889 | 1909 | ACCAGGUUGCUCCCUGCGGGC | 187 | GCCCGCAGGGAGCAACCUGGU | 491 | −6 |
| RD2017 | 1890 | 1910 | UACCAGGUUGCUCCCUGCGGG | 188 | CCCGCAGGGAGCAACCUGGUA | 492 | 45 |
| RD2018 | 1891 | 1911 | ACACCAGGUUGCUCCCUGCGG | 189 | CCGCAGGGAGCAACCUGGUGU | 493 | 51 |
| RD2019 | 1892 | 1912 | UACACCAGGUUGCUCCCUGCG | 190 | CGCAGGGAGCAACCUGGUGUA | 494 | 51 |
| RD2020 | 1893 | 1913 | AGACACCAGGUUGCUCCCUGC | 191 | GCAGGGAGCAACCUGGUGUCU | 495 | 2 |
| RD2021 | 1894 | 1914 | UAGACACCAGGUUGCUCCCUG | 192 | CAGGGAGCAACCUGGUGUCUA | 496 | 67 |
| RD2022 | 1895 | 1915 | UUAGACACCAGGUUGCUCCCU | 193 | AGGGAGCAACCUGGUGUCUAA | 497 | 85 |
| RD2023 | 1896 | 1916 | UGUAGACACCAGGUUGCUCCC | 194 | GGGAGCAACCUGGUGUCUACA | 498 | 75 |
| RD2024 | 1897 | 1917 | UUGUAGACACCAGGUUGCUCC | 195 | GGAGCAACCUGGUGUCUACAA | 499 | 55 |
| RD2025 | 1907 | 1927 | AGCGACUUUGGUGUAGACACC | 196 | GGUGUCUACACCAAAGUCGCU | 500 | 66 |
| RD2026 | 1922 | 1942 | UCAGUCCAUGUACUCAGCGAC | 197 | GUCGCUGAGUACAUGGACUGA | 501 | 69 |
| RD2027 | 1923 | 1943 | UCCAGUCCAUGUACUCAGCGA | 198 | UCGCUGAGUACAUGGACUGGA | 502 | 71 |
| RD2028 | 1924 | 1944 | AUCCAGUCCAUGUACUCAGCG | 199 | CGCUGAGUACAUGGACUGGAU | 503 | 75 |
| RD2029 | 1941 | 1961 | UCUGUGUUUUCUCUAAAAUCC | 200 | GGAUUUUAGAGAAAACACAGA | 504 | 73 |
| RD2030 | 1942 | 1962 | UUCUGUGUUUUCUCUAAAAUC | 201 | GAUUUUAGAGAAAACACAGAA | 505 | 67 |
| RD2031 | 1943 | 1963 | UCUCUGUGUUUUCUCUAAAAU | 202 | AUUUUAGAGAAAACACAGAGA | 506 | 68 |
| RD2032 | 1944 | 1964 | UGCUCUGUGUUUUCUCUAAAA | 203 | UUUUAGAGAAAACACAGAGCA | 507 | 71 |
| RD2033 | 1945 | 1965 | UUGCUCUGUGUUUUCUCUAAA | 204 | UUUAGAGAAAACACAGAGCAA | 508 | 82 |
| RD2034 | 1950 | 1970 | UAUCACUGCUCUGUGUUUUCU | 205 | AGAAAACACAGAGCAGUGAUA | 509 | 72 |
| RD2035 | 1953 | 1973 | UUCCAUCACUGCUCUGUGUUU | 206 | AAACACAGAGCAGUGAUGGAA | 510 | 81 |

TABLE 4
Inhibition of PKK mRNA by double-stranded compounds targeting SEQ ID NO: 1

| | Seq | SEQ | | | | | |
|---|---|---|---|---|---|---|---|
| | ID | ID | | | | | PKK |
| | NO: 1 | NO: 1 | | SEQ | | SEQ | % |
| Compound | Start | Stop | Antisense Sequence | ID | Sense Sequence | ID | inhi- |
| Number | Site | Site | (5′-3′) | NO: | (5′-3′) | NO: | bition |
| RD1896 | 940 | 960 | UGGCAGGGUUCAGGUAAAGUU | 67 | AACUUUACCUGAACCCUGCCA | 371 | 44 |
| RD1897 | 941 | 961 | AUGGCAGGGUUCAGGUAAAGU | 68 | ACUUUACCUGAACCCUGCCAU | 372 | 41 |
| RD1898 | 943 | 963 | UAAUGGCAGGGUUCAGGUAAA | 69 | UUUACCUGAACCCUGCCAUUA | 373 | 75 |
| RD1899 | 944 | 964 | AGAAUGGCAGGGUUCAGGUAA | 70 | UUACCUGAACCCUGCCAUUCU | 374 | 67 |
| RD1900 | 945 | 965 | UAGAAUGGCAGGGUUCAGGUA | 71 | UACCUGAACCCUGCCAUUCUA | 375 | 73 |
| RD1901 | 946 | 966 | UUAGAAUGGCAGGGUUCAGGU | 72 | ACCUGAACCCUGCCAUUCUAA | 376 | 72 |
| RD1902 | 947 | 967 | UUUAGAAUGGCAGGGUUCAGG | 73 | CCUGAACCCUGCCAUUCUAAA | 377 | 79 |
| RD1903 | 948 | 968 | UUUUAGAAUGGCAGGGUUCAG | 74 | CUGAACCCUGCCAUUCUAAAA | 378 | 80 |
| RD1904 | 949 | 969 | AUUUUAGAAUGGCAGGGUUCA | 75 | UGAACCCUGCCAUUCUAAAAU | 379 | 79 |
| RD1905 | 950 | 970 | AAUUUUAGAAUGGCAGGGUUC | 76 | GAACCCUGCCAUUCUAAAAUU | 380 | 67 |
| RD1906 | 951 | 971 | AAAUUUUAGAAUGGCAGGGUU | 77 | AACCCUGCCAUUCUAAAAUUU | 381 | 57 |
| RD1907 | 952 | 972 | UAAAUUUUAGAAUGGCAGGGU | 78 | ACCCUGCCAUUCUAAAAUUUA | 382 | 74 |
| RD1908 | 953 | 973 | UUAAAUUUUAGAAUGGCAGGG | 79 | CCCUGCCAUUCUAAAAUUUAA | 383 | 84 |
| RD1909 | 954 | 974 | UGUAAAUUUUAGAAUGGCAGG | 80 | CCUGCCAUUCUAAAAUUUACA | 384 | 83 |
| RD1910 | 957 | 977 | UCGGGUAAAUUUUAGAAUGGC | 81 | GCCAUUCUAAAAUUUACCCGA | 385 | 81 |
| RD1911 | 958 | 978 | UCCGGGUAAAUUUUAGAAUGG | 82 | CCAUUCUAAAAUUUACCCGGA | 386 | 78 |
| RD1912 | 989 | 1009 | UACAUUCAAUUCUUCUCCUCC | 83 | GGAGGAGAAGAAUUGAAUGUA | 387 | 73 |
| RD1913 | 990 | 1010 | UCACAUUCAAUUCUUCUCCUC | 84 | GAGGAGAAGAAUUGAAUGUGA | 388 | 91 |
| RD1914 | 992 | 1012 | AGUCACAUUCAAUUCUUCUCC | 85 | GGAGAAGAAUUGAAUGUGACU | 389 | 77 |
| RD1915 | 1017 | 1037 | UGCAAACAUUCACUCCUUUAA | 86 | UUAAAGGAGUGAAUGUUUGCA | 390 | 85 |
| RD1916 | 1050 | 1070 | ACUGACAGCGAAUCAUCUUUG | 87 | CAAAGAUGAUUCGCUGUCAGU | 391 | 93 |
| RD1917 | 1051 | 1071 | AACUGACAGCGAAUCAUCUUU | 88 | AAAGAUGAUUCGCUGUCAGUU | 392 | 92 |
| RD1918 | 1052 | 1072 | AAACUGACAGCGAAUCAUCUU | 89 | AAGAUGAUUCGCUGUCAGUUU | 393 | 91 |
| RD1919 | 1053 | 1073 | AAAACUGACAGCGAAUCAUCU | 90 | AGAUGAUUCGCUGUCAGUUUU | 394 | 81 |
| RD1920 | 1054 | 1074 | AAAAACUGACAGCGAAUCAUC | 91 | GAUGAUUCGCUGUCAGUUUUU | 395 | 93 |
| RD1921 | 1057 | 1077 | UUGAAAAACUGACAGCGAAUC | 92 | GAUUCGCUGUCAGUUUUUCAA | 396 | 91 |
| RD1922 | 1058 | 1078 | AGUGAAAAACUGACAGCGAAU | 93 | AUUCGCUGUCAGUUUUUCACU | 397 | 85 |
| RD1923 | 1059 | 1079 | AAGUGAAAAACUGACAGCGAA | 94 | UUCGCUGUCAGUUUUUCACUU | 398 | 93 |
| RD2036 | 1954 | 1974 | UUUCCAUCACUGCUCUGUGUU | 207 | AACACAGAGCAGUGAUGGAAA | 511 | 69 |
| RD2037 | 2037 | 2057 | UGCUCAGAAUUUGACUUGAAC | 208 | GUUCAAGUCAAAUUCUGAGCA | 512 | 0 |
| RD2038 | 2038 | 2058 | AGGCUCAGAAUUUGACUUGAA | 209 | UUCAAGUCAAAUUCUGAGCCU | 513 | 0 |
| RD2039 | 2039 | 2059 | UAGGCUCAGAAUUUGACUUGA | 210 | UCAAGUCAAAUUCUGAGCCUA | 514 | 0 |
| RD2040 | 2040 | 2060 | UCAGGCUCAGAAUUUGACUUG | 211 | CAAGUCAAAUUCUGAGCCUGA | 515 | 0 |
| RD2041 | 2041 | 2061 | UCCAGGCUCAGAAUUUGACUU | 212 | AAGUCAAAUUCUGAGCCUGGA | 516 | 0 |
| RD2042 | 2043 | 2063 | UCCCCAGGCUCAGAAUUUGAC | 213 | GUCAAAUUCUGAGCCUGGGGA | 517 | 0 |
| RD2043 | 2067 | 2087 | UCUCCAUGCUUUGCAGAUGAG | 214 | CUCAUCUGCAAAGCAUGGAGA | 518 | 0 |
| RD2044 | 2070 | 2090 | UACUCUCCAUGCUUUGCAGAU | 215 | AUCUGCAAAGCAUGGAGAGUA | 519 | 0 |
| RD2045 | 2071 | 2091 | UCACUCUCCAUGCUUUGCAGA | 216 | UCUGCAAAGCAUGGAGAGUGA | 520 | 11 |
| RD2046 | 2072 | 2092 | UCCACUCUCCAUGCUUUGCAG | 217 | CUGCAAAGCAUGGAGAGUGGA | 521 | 16 |
| RD2047 | 2073 | 2093 | UGCCACUCUCCAUGCUUUGCA | 218 | UGCAAAGCAUGGAGAGUGGCA | 522 | 28 |
| RD2048 | 2074 | 2094 | AUGCCACUCUCCAUGCUUUGC | 219 | GCAAAGCAUGGAGAGUGGCAU | 523 | 28 |
| RD2049 | 2075 | 2095 | UAUGCCACUCUCCAUGCUUUG | 220 | CAAAGCAUGGAGAGUGGCAUA | 524 | 35 |
| RD2050 | 2076 | 2096 | AGAUGCCACUCUCCAUGCUUU | 221 | AAAGCAUGGAGAGUGGCAUCU | 525 | 0 |
| RD2051 | 2131 | 2151 | UAUUGUCCUCAGCAGCUCUGA | 222 | UCAGAGCUGCUGAGGACAAUA | 526 | 0 |
| RD2064 | 172 | 192 | UCUACAUCCCCACUUCUGAAG | 223 | CUUCAGAAGUGGGGAUGUAGA | 527 | 48 |
| RD2065 | 173 | 193 | AGCUACAUCCCCAUCUCUGAA | 224 | UUCAGAGAUGGGGAUGUAGCU | 528 | 45 |
| RD2066 | 175 | 195 | UAAGCUACAUCCCUACCUCUG | 225 | CAGAGGUAGGGAUGUAGCUUA | 529 | 47 |
| RD2067 | 176 | 196 | UGAAGCUACAUCCUCACCUCU | 226 | AGAGGUGAGGAUGUAGCUUCA | 530 | 65 |
| RD2068 | 177 | 197 | UGGAAGCUACAUCUCCACCUC | 227 | GAGGUGGAGAUGUAGCUUCCA | 531 | 66 |
| RD2069 | 178 | 198 | AUGGAAGCUACAUUCCCACCU | 228 | AGGUGGGAAUGUAGCUUCCAU | 532 | 83 |
| RD2070 | 181 | 201 | UACAUGGAAGCUAUAUCCCCA | 229 | UGGGGAUAUAGCUUCCAUGUA | 533 | 79 |
| RD2071 | 242 | 262 | UAAUAGCAAACACUUUGGGUG | 230 | CACCCAAAGUGUUUGCUAUUA | 534 | 10 |
| RD2072 | 243 | 263 | UGAAUAGCAAACAUCUUGGGU | 231 | ACCCAAGAUGUUUGCUAUUCA | 535 | 90 |
| RD2073 | 245 | 265 | ACUGAAUAGCAAAUACCUUGG | 232 | CCAAGGUAUUUGCUAUUCAGU | 536 | 82 |
| RD2074 | 249 | 269 | UAAAACUGAAUAGUAAACACC | 233 | GGUGUUUACUAUUCAGUUUUA | 537 | 74 |
| RD2075 | 288 | 308 | UAAACCUUUUCUCUAUGUCAU | 234 | AUGACAUAGAGAAAAGGUUUA | 538 | 48 |
| RD2076 | 312 | 332 | UAACACUAUCUUUUAAGAAGC | 235 | GCUUCUUAAAAGAUAGUGUUA | 539 | 0 |
| RD2077 | 316 | 336 | UCUGUAACACUAUUUUUCAAG | 236 | CUUGAAAAAUAGUGUUACAGA | 540 | 0 |
| RD2078 | 438 | 458 | AAUUGACUCCUCUUAUAUCAA | 237 | UUGAUAUAAGAGGAGUCAAUU | 541 | 85 |
| RD2079 | 440 | 460 | AAAAUUGACUCCUUUCAUAUC | 238 | GAUAUGAAAGGAGUCAAUUUU | 542 | 76 |
| RD2080 | 442 | 462 | UUAAAAUUGACUCUUCUCAUA | 239 | UAUGAGAAGAGUCAAUUUUAA | 543 | 63 |
| RD2081 | 443 | 463 | AUUAAAAUUGACUUCUCUCAU | 240 | AUGAGAGAAGUCAAUUUUAAU | 544 | 29 |
| RD2082 | 445 | 465 | ACAUUAAAAUUGAUUCCUCUC | 241 | GAGAGGAAUCAAUUUUAAUGU | 545 | 28 |
| RD2083 | 473 | 493 | UCAUUCUUCAACAUUGCUAAC | 242 | GUUAGCAAUGUUGAAGAAUGA | 546 | 82 |
| RD2084 | 475 | 495 | UGGCAUUCUUCAAUACUGCUA | 243 | UAGCAGUAUUGAAGAAUGCCA | 547 | 71 |
| RD2085 | 478 | 498 | UUUUGGCAUUCUUUAACACUG | 244 | CAGUGUUAAAGAAUGCCAAAA | 548 | 86 |
| RD2086 | 631 | 651 | UAGAAUCCAGAUUUCACGUUA | 245 | UAACGUGAAAUCUGGAUUCUA | 549 | 52 |
| RD2087 | 637 | 657 | UUCAGUGAGAAUCUAGAUUCC | 246 | GGAAUCUAGAUUCUCACUGAA | 550 | 65 |
| RD2088 | 926 | 946 | UAAAGUUCUUUUGUAGGUUAA | 247 | UUAACCUACAAAAGAACUUUA | 551 | 0 |
| RD2089 | 932 | 952 | UUCAGGUAAAGUUUUUUUGCA | 248 | UGCAAAAAAACUUUACCUGAA | 552 | 8 |
| RD2090 | 943 | 963 | UAAUGGCAGGGUUUAGGUAAA | 249 | UUUACCUAAACCCUGCCAUUA | 553 | 82 |
| RD2091 | 950 | 970 | AAUUUUAGAAUGGUAGGGUUC | 250 | GAACCCUACCAUUCUAAAAUU | 554 | 81 |
| RD2092 | 969 | 989 | UAAAGUCAACUCCUGGGUAAA | 251 | UUUACCCAGGAGUUGACUUUA | 555 | 50 |
| RD2093 | 970 | 990 | UCAAAGUCAACUCUCGGGUAA | 252 | UUACCCGAGAGUUGACUUUGA | 556 | 65 |
| RD2094 | 971 | 991 | UCCAAAGUCAACUUCCGGGUA | 253 | UACCCGGAAGUUGACUUUGGA | 557 | 84 |
| RD2095 | 985 | 1005 | UUCAAUUCUUCUCUUCCAAAG | 254 | CUUUGGAAGAGAAGAAUUGAA | 558 | 91 |
| RD2096 | 986 | 1006 | AUUCAAUUCUUCUUCUCCAAA | 255 | UUUGGAGAAGAAGAAUUGAAU | 559 | 80 |
| RD2097 | 1020 | 1040 | UUUGGCAAACAUUUACUCCUU | 256 | AAGGAGUAAAUGUUUGCCAAA | 560 | 92 |
| RD2098 | 1050 | 1070 | ACUGACAGCGAAUUAUCUUUG | 257 | CAAAGAUAAUUCGCUGUCAGU | 561 | 92 |
| RD2099 | 1058 | 1078 | AGUGAAAAACUGAUAGCGAAU | 258 | AUUCGCUAUCAGUUUUUCACU | 562 | 74 |
| RD2100 | 1062 | 1082 | AAUAAGUGAAAAAUUGACAGC | 259 | GCUGUCAAUUUUUCACUUAUU | 563 | 78 |
| RD2101 | 1087 | 1107 | UCCUUACAGUCUUUUGGGAGU | 260 | ACUCCCAAAAGACUGUAAGGA | 564 | 36 |
| RD2103 | 1171 | 1191 | UAACCAGAGCUCCUUUGUGUC | 261 | GACACAAAGGAGCUCUGGUUA | 565 | 67 |
| RD2104 | 1172 | 1192 | UUAACCAGAGCUCUCUUGUGU | 262 | ACACAAGAGAGCUCUGGUUAA | 566 | 89 |
| RD2105 | 1175 | 1195 | AGAGUAACCAGAGUUCCCUUG | 263 | CAAGGGAACUCUGGUUACUCU | 567 | 93 |
| RD2106 | 1181 | 1201 | UCUCAAAGAGUAAUCAGAGCU | 264 | AGCUCUGAUUACUCUUUGAGA | 568 | 77 |
| RD2107 | 1197 | 1217 | UCCCAGUGUUACAUAAUCUCA | 265 | UGAGAUUAUGUAACACUGGGA | 569 | 44 |
| RD2108 | 1249 | 1269 | UUUGUUCCUCCAAUAAUGCGU | 266 | ACGCAUUAUUGGAGGAACAAA | 570 | 70 |
| RD2109 | 1252 | 1272 | UAGUUUGUUCCUCUAACAAUG | 267 | CAUUGUUAGAGGAACAAACUA | 571 | 49 |
| RD2110 | 1253 | 1273 | AGAGUUUGUUCCUUCAACAAU | 268 | AUUGUUGAAGGAACAAACUCU | 572 | 69 |
| RD2111 | 1255 | 1275 | UAAGAGUUUGUUCUUCCAACA | 269 | UGUUGGAAGAACAAACUCUUA | 573 | 48 |
| RD2112 | 1256 | 1276 | AGAAGAGUUUGUUUCUCCAAC | 270 | GUUGGAGAAACAAACUCUUCU | 574 | 78 |
| RD2113 | 1272 | 1292 | AGGGCCACUCUCCUCAAGAAG | 271 | CUUCUUGAGGAGAGUGGCCCU | 575 | 33 |
| RD2114 | 1273 | 1293 | UAGGGCCACUCUCUCCAAGAA | 272 | UUCUUGGAGAGAGUGGCCCUA | 576 | 59 |
| RD2115 | 1274 | 1294 | UCAGGGCCACUCUUCCCAAGA | 273 | UCUUGGGAAGAGUGGCCCUGA | 577 | 61 |
| RD2116 | 1331 | 1351 | UAGUGACCCUCCAUACAGGUG | 274 | CACCUGUAUGGAGGGUCACUA | 578 | 58 |
| RD2117 | 1334 | 1354 | UAUGAGUGACCCUUCACACAG | 275 | CUGUGUGAAGGGUCACUCAUA | 579 | 90 |
| RD2118 | 1336 | 1356 | UCUAUGAGUGACCUUCCACAC | 276 | GUGUGGAAGGUCACUCAUAGA | 580 | 76 |
| RD2119 | 1337 | 1357 | UCCUAUGAGUGACUCUCCACA | 277 | UGUGGAGAGUCACUCAUAGGA | 581 | 76 |
| RD2120 | 1338 | 1358 | UUCCUAUGAGUGAUCCUCCAC | 278 | GUGGAGGAUCACUCAUAGGAA | 582 | 81 |
| RD2121 | 1348 | 1368 | ACCCACUGGUGUCUUAUGAGU | 279 | ACUCAUAAGACACCAGUGGGU | 583 | 42 |
| RD2122 | 1349 | 1369 | UACCCACUGGUGUUCUAUGAG | 280 | CUCAUAGAACACCAGUGGGUA | 584 | 54 |
| RD2123 | 1441 | 1461 | UCUUUUGUAAUGUUUGACAGA | 281 | UCUGUCAAACAUUACAAAAGA | 585 | 90 |
| RD2124 | 1513 | 1533 | AUAUCAUGAUUCCUUUCUGAG | 282 | CUCAGAAAGGAAUCAUGAUAU | 586 | 59 |
| RD2125 | 1545 | 1565 | AAUUCAAAGGAGCUUGGAGUU | 283 | AACUCCAAGCUCCUUUGAAUU | 587 | 78 |
| RD2126 | 1546 | 1566 | UAAUUCAAAGGAGUCUGGAGU | 284 | ACUCCAGACUCCUUUGAAUUA | 588 | 78 |
| RD2127 | 1844 | 1864 | UCCCACCAAACGCUACAUUCC | 285 | GGAAUGUAGCGUUUGGUGGGA | 589 | 40 |
| RD2128 | 1845 | 1865 | UGCCCACCAAACGUCACAUUC | 286 | GAAUGUGACGUUUGGUGGGCA | 590 | 64 |
| RD2129 | 1847 | 1867 | UAUGCCCACCAAAUGCCACAU | 287 | AUGUGGCAUUUGGUGGGCAUA | 591 | 76 |
| RD2130 | 1880 | 1900 | UUCCCUGCGGGCAUAGCCUUC | 288 | GAAGGCUAUGCCCGCAGGGAA | 592 | 71 |
| RD2131 | 1889 | 1909 | ACCAGGUUGCUCCUUGCGGGC | 289 | GCCCGCAAGGAGCAACCUGGU | 593 | 36 |
| RD2132 | 1890 | 1910 | UACCAGGUUGCUCUCUGCGGG | 290 | CCCGCAGAGAGCAACCUGGUA | 594 | 63 |
| RD2133 | 1891 | 1911 | ACACCAGGUUGCUUCCUGCGG | 291 | CCGCAGGAAGCAACCUGGUGU | 595 | 52 |
| RD2134 | 1893 | 1913 | AGACACCAGGUUGUUCCCUGC | 292 | GCAGGGAACAACCUGGUGUCU | 596 | 6 |
| RD2135 | 1915 | 1935 | AUGUACUCAGCGAUUUUGGUG | 293 | CACCAAAAUCGCUGAGUACAU | 597 | 82 |
| RD2136 | 1918 | 1938 | UCCAUGUACUCAGUGACUUUG | 294 | CAAAGUCACUGAGUACAUGGA | 598 | 83 |
| RD2137 | 1921 | 1941 | UAGUCCAUGUACUUAGCGACU | 295 | AGUCGCUAAGUACAUGGACUA | 599 | 71 |
| RD2138 | 1923 | 1943 | UCCAGUCCAUGUAUUCAGCGA | 296 | UCGCUGAAUACAUGGACUGGA | 600 | 68 |
| RD2139 | 1929 | 1949 | UUAAAAUCCAGUCUAUGUACU | 297 | AGUACAUAGACUGGAUUUUAA | 601 | 57 |
| RD2140 | 1930 | 1950 | UCUAAAAUCCAGUUCAUGUAC | 298 | GUACAUGAACUGGAUUUUAGA | 602 | 68 |
| RD2141 | 1942 | 1962 | UUCUGUGUUUUCUUUAAAAUC | 299 | GAUUUUAAAGAAAACACAGAA | 603 | 26 |
| RD2142 | 1944 | 1964 | UGCUCUGUGUUUUUUCUAAAA | 300 | UUUUAGAAAAAACACAGAGCA | 604 | 68 |
| RD2143 | 1953 | 1973 | UUCCAUCACUGCUUUGUGUUU | 301 | AAACACAAAGCAGUGAUGGAA | 605 | 85 |
| RD2144 | 2067 | 2087 | UCUCCAUGCUUUGUAGAUGAG | 302 | CUCAUCUACAAAGCAUGGAGA | 606 | 39 |
| RD2145 | 2072 | 2092 | UCCACUCUCCAUGUUUUGCAG | 303 | CUGCAAAACAUGGAGAGUGGA | 607 | 28 |
| RD2146 | 2076 | 2096 | AGAUGCCACUCUCUAUGCUUU | 304 | AAAGCAUAGAGAGUGGCAUCU | 608 | 0 |

### Example 2—Dose-Dependent Inhibition of Human PKK in HEK-293T Cells

Compounds from the studies described above exhibiting significant in vitro inhibition of PKK mRNA were selected and tested at various doses in HEK-293T Cells as described above. Compounds were tested at concentrations of 0.01, 0.1, 1 and 10 nM and IC50 values were calculated (Table 5).

TABLE 5

| Compound Number | IC50 (nM) |
|---|---|
| RD1872 | 0.33 |
| RD1874 | 0.41 |
| RD1913 | 0.033 |
| RD1923 | 0.044 |
| RD1948 | 0.16 |
| RD1956 | 0.11 |
| RD1978 | 0.14 |
| RD1993 | 0.038 |
| RD1996 | 0.086 |
| RD2022 | 0.16 |
| RD2028 | 0.10 |

### Example 3: Effect of Compounds Targeting Human PKK in Cynomolgus Monkeys

Compounds of interest, identified from in vitro gene expression screening, were evaluated in cynomolgus monkeys (Table 7). Prior to the study the monkeys were kept in quarantine during which the animals were observed daily for general health. Ten groups of 1 cynomolgus monkey each were injected with a single 6 mg/kg subcutaneous dose of oligonucleotide on Day 1 of the study. During the study period, the monkeys were observed daily for signs of illness or distress. Animals were bled on day −6 and on days 1 (prior to dosing), 4, 8, 15, 22, 29, 36, 43, 50, 57 and 64 for serum collection and analysis. The protocols described were approved by the Institutional Animal Care and Use Committee (IACUC).

Circulating PKK levels were quantified using an ELISA specific for human angiotensinogen (and cross-reactive with cynomolgus), according to manufacturer's protocol (IBL America #27412). PKK inhibition data were expressed as percent of baseline value (Day 1 prior to dosing) (Table 8).

TABLE 6
Compound Sequence

| | Seq | SEQ | | | | |
|---|---|---|---|---|---|---|
| | ID | ID | | | | |
| | NO: 1 | NO: 1 | | SEQ | | SEQ |
| Compound | Start | Stop | Antisense Sequence | ID | Sense Sequence | ID |
| Number | Site | Site | (5′-3′) | NO: | (5′-3′) | NO: |
| RD2305 | 660 | 682 | ACCAAUUUCUGAAAGGGCACAGG | 306 | UCCUGUGCCCUUUCAGAAAUUGGU | 610 |
| RD2306 | 988 | 1010 | UCACAUUCAAUUCUUCUCCUCCA | 307 | CGGAGGAGAAGAAUUGAAUGUGA | 611 |
| RD2307 | 1057 | 1079 | AAGUGAAAAACUGACAGCGAACC | 308 | UGGUUCGCUGUCAGUUUUUCACUU | 612 |
| RD2308 | 1140 | 1162 | AAUCCUAGUUGGAGAACCAUCCG | 309 | CGGAUGGUUCUCCAACUAGGAUU | 613 |
| RD2309 | 1177 | 1199 | UCAAAGAGUAACCAGAGCUCCCU | 310 | UGGAGCUCUGGUUACUCUUUGA | 614 |
| RD2310 | 1424 | 1446 | UACAGAUUUAAAAUGCCACUGCG | 311 | UCGCAGUGGCAUUUUAAAUCUGUA | 615 |
| RD2311 | 1546 | 1568 | UGUAAUUCAAAGGAGCCUGGAGU | 312 | UCUCCAGGCUCCUUUGAAUUACA | 616 |
| RD2312 | 1895 | 1915 | UUAGACACCAGGUUGCUCCCU | 193 | UAGGGAGCAACCUGGUGUCUAA | 617 |
| RD2313 | 1924 | 1944 | AUCCAGUCCAUGUACUCAGCG | 199 | UCGCUGAGUACAUGGACUGGAU | 618 |
| RD2192 | 1549 | 1571 | UAGUGUAAUUCAAAGGAGCCUGG | 305 | CCAGGCUCCUUUGAAUUACACUA | 609 |

TABLE 7
Compound Chemistry

| | | Ref | SEQ |
|---|---|---|---|
| Com- | | ID | ID |
| pound | Modified Strands (5′-3′) | NO: | NO: |
| RD2305 | mA*fC*mC.fA.mA.fU.mU. | IA0812 | 306 |
| | fU.mC.fU.mG.fA.mA.fA. | | |
| | mG.fG.mG.fC.mA.fC.mA* | | |
| | mG*mG | | |
| | H1.mC*mC*mU*mG*mU.mG. | IS1001 | 610 |
| | mC.mC.fC.mU.fU.fU.fC. | | |
| | fA.mG.mA.mA.mA.mU.mU. | | |
| | mG.mG*mU*dQ | | |
| RD2306 | mU*fC*mA.fC.mA.fU.mU. | IA0813 | 307 |
| | fC.mA.fA.mU.fU.mC.fU. | | |
| | mU.fC.mU.fC.mC.fU.mC* | | |
| | mC*mA | | |
| | H7.mG*mG*mA.mG.mG.mA. | IS1002 | 611 |
| | mG.fA.mA.fG.fA.fA.fU. | | |
| | mU.mG.mA.mA.mU.mG.mU. | | |
| | mG*mA*dQ | | |
| RD2307 | mA*fA*mG.fU.mG.fA.mA. | IA0814 | 308 |
| | fA.mA.fA.mC.fU.mG.fA. | | |
| | mC.fA.mG.fC.mG.fA.mA* | | |
| | mC*mC | | |
| | H1.mG*mG*mU.mU.mC.mG. | IS1003 | 612 |
| | mC.mU.fG.mU.fC.fA.fG. | | |
| | fU.mU.mU.mU.mU.mC.mA. | | |
| | mC.mU*mU*dO | | |
| RD2309 | mU*fC*mA.fA.mA.fG.mA. | IA0816 | 310 |
| | fG.mU.fA.mA.fC.mC.fA. | | |
| | mG.fA.mG.fC.mU.fC.mC* | | |
| | mC*mU | | |
| | Hl.mG*mG*mA.mG.mC.mU. | IS1005 | 614 |
| | fC.mU.fG.fG.fU.fU.mA. | | |
| | mC.mU.mC.mU.mU.mU.mG* | | |
| | mA*dQ | | |
| RD2308 | mA*fA*mU.fC.mC.fU.mA. | IA0815 | 309 |
| | fG.mU.fU.mG.fG.mA.fG. | | |
| | mA.fA.mC.fC.mA.fU.mC* | | |
| | mC*mG | | |
| | H7.mG.mG*mA*mU.mG.mG. | IS1004 | 613 |
| | mU.fU.mC.fU.fC.fC.fA. | | |
| | mA.mC.mU.mA.mG.mG.mA. | | |
| | mU*mU*dQ | | |
| RD2310 | mU*fA*mC.fA.mG.fA.mU. | IA0817 | 311 |
| | fU.mU.fA.mA.fA.mA.fU. | | |
| | mG.fC.mC.fA.mC.fU.mG* | | |
| | mC*mG | | |
| | H1.mC*mG*mC.mA.mG.mU. | IS1006 | 615 |
| | mG.mG.fC.mA.fU.fU.fU. | | |
| | fU.mA.mA.mA.mU.mC.mU. | | |
| | mG.mU*mA*dQ | | |
| RD2311 | mu*fG*mU.fA.mA.fU.mU. | IA0818 | 312 |
| | fC.mA.fA.mA.fG.mG.fA. | | |
| | mG.fC.mC.fU.mG.fG.mA* | | |
| | mG*mU | | |
| | H1.mC*mU*mC.mC.mA.mG. | IS1007 | 616 |
| | mG.fC.mU.fC.fC.fU.fU. | | |
| | mU.mG.mA.mA.mU.mU.mA. | | |
| | mC*mA*dQ | | |
| RD2192 | mU*fA*mG.fU.mG.fU.mA. | IA0819 | 305 |
| | fA.mU.fU.mC.fA.mA.fA. | | |
| | mG.fG.mA.fG.mC.fC.mU* | | |
| | mG*mG | | |
| | H6.mC*mA*mG.mG.mC.mU. | IS0925 | 609 |
| | mC.fC.mU.fU.fU.fG.fA. | | |
| | mA.mU.mU.mA.mC.mA.mC. | | |
| | mU*mA*dO | | |
| RD2312 | mU*fU*mA.fG.mA.fC.mA. | IA0820 | 193 |
| | fC.mC.fA.mG.fG.mU.fU. | | |
| | mG.fC.mU.fC.mC*fC*mU | | |
| | H1.mA*mG*mG.mG.mA.mG. | IS1008 | 617 |
| | fC.mA.fA.fC.fC.fU.mG. | | |
| | mG.mU.mG.mU.mC.mU.mA* | | |
| | mA*dQ | | |
| RD2313 | mA*fU*mC.fC.mA.fG.mU. | IA0821 | 199 |
| | fC.mC.fA.mU.fG.mU.fA. | | |
| | mC.fU.mC.fA.mG*fC*mG | | |
| | H1.mC*mG*mC.mU.mG.mA. | IS1009 | 618 |
| | fG.mU.fA.fC.fA.fU.mG. | | |
| | mG.mA.mC.mU.mG.mG.mA* | | |
| | mU*dQ | | |

TABLE 8
Average PKK Inhibition

| | Days | | | | | | | | | |
|---|---|---|---|---|---|---|---|---|---|---|
| Compound | 4 | 8 | 15 | 22 | 29 | 36 | 43 | 50 | 57 | 64 |
| RD2305 | 1 | 0 | 2 | 37 | 11 | 5 | 0 | 0 | 0 | 0 |
| RD2306 | 43 | 60 | 74 | 88 | 89 | 84 | 72 | 70 | 72 | 63 |
| RD2307 | 35 | 54 | 60 | 69 | 70 | 79 | 55 | 54 | 55 | 50 |
| RD2309 | 18 | 58 | 47 | 69 | 58 | 71 | 44 | 54 | 49 | 45 |
| RD2308 | 47 | 32 | 33 | 42 | 44 | 45 | 28 | 24 | 20 | 18 |
| RD2310 | 4 | 37 | 46 | 64 | 69 | 72 | 59 | 62 | 58 | 53 |
| RD2311 | 29 | 55 | 74 | 85 | 92 | 92 | 80 | 75 | 71 | 63 |
| RD2192 | 20 | −6 | −46 | 26 | 2 | 18 | −4 | 5 | −6 | 0 |
| RD2312 | 34 | 42 | 74 | 78 | 75 | 72 | 59 | 51 | 57 | 54 |
| RD2313 | 33 | 50 | 75 | 90 | 84 | 93 | 82 | 82 | 83 | 84 |

### Example 4: Effect of Compounds Targeting Human PKK in Cynomolgus Monkeys

Compounds of interest, identified from in vitro gene expression screening, were evaluated in cynomolgus monkeys (Table 10). Prior to the study the monkeys were kept in quarantine during which the animals were observed daily for general health. Eight groups of 2 cynomolgus monkey each were injected with a single 4 mg/kg subcutaneous dose of oligonucleotide on Day 1 of the study. During the study period, the monkeys were observed daily for signs of illness or distress. Animals were bled on day −6 and on days 1 (prior to dosing), 4, 8, and 15 for serum collection and analysis. Future collections will be on days 22, 29, 36, 43, 50, 57, 64, 71, 78 and 85. In a second set of experiments, animals were bled on day-6 and days 1 (prior to dosing), 4, 8, 15 and 22 for serum collection and analysis. Future collections for the second set of experiments will be on days 29, 36, 43, 50, 57, 64, 71, 78 and 85. In a third set of experiments, animals were bled on day −6 and days 1 (prior to dosing), 4, 8, 15, 22, 29 and 36 for serum collection and analysis. Future collections for the third set of experiments will be on days 43, 50, 57, 64, 71, 78 and 85. In a fourth set of experiments, animals were bled on day −6 and days 1 (prior to dosing), 4, 8, 15, 22, 29, 36, 43, 50, 57, 64, 71, 78 and 85 for serum collection and analysis. The protocols described were approved by the Institutional Animal Care and Use Committee (IACUC). Circulating PKK levels were quantified using an ELISA specific for human angiotensinogen (and cross-reactive with cynomolgus), according to manufacturer's protocol (IBL America #27412). PKK inhibition data were expressed as percent of baseline value (Day 1 prior to dosing) and as an average of the group for each compound. Results from the four sets of experiments are shown in Tables 11, 12, 13 and 14. Clinical chemistry was performed on Day −1 or Day −6 and Day 63 or 64 and Day 92. There were no test article-related effects on body weight (Table 15) and all serum chemistry values were within reference ranges (Table 16 and 17).

TABLE 9
Compound Sequence

| | Seq | SEQ | | | | |
|---|---|---|---|---|---|---|
| | ID | ID | | | | |
| | NO: 1 | NO: 1 | Antisense | SEQ | Sense | SEQ |
| Compound | Start | Stop | Sequence | ID | Sequence | ID |
| Number | Site | Site | (5′-3′) | NO: | (5′-3′) | NO: |
| RD2423 | 1546 | 1568 | UGUAAUU | 312 | CUCCAGG | 619 |
| | | | CAAAGGA | | CUCCUUU | |
| | | | GCCUGGA | | GAAUUAC | |
| | | | GU | | A | |
| RD2436 | 1546 | 1568 | UGUAAUU | 312 | UCUCCAG | 616 |
| | | | CAAAGGA | | GCUCCUU | |
| | | | GCCUGGA | | UGAAUUA | |
| | | | GU | | CA | |
| RD2437 | 1546 | 1568 | UGUAAUU | 312 | UCUCCAG | 620 |
| | | | CAAAGGA | | GCUCCUU | |
| | | | GCCUGGA | | UGAAUUA | |
| | | | GU | | CAU | |
| RD2438 | 1546 | 1568 | UGUAAUU | 312 | UCCAGGC | 468 |
| | | | CAAAGGA | | UCCUUUG | |
| | | | GCCUGGA | | AAUUACA | |
| | | | GU | | | |
| RD2439 | 988 | 1010 | UCACAUU | 307 | UGGAGGA | 622 |
| | | | CAAUUCU | | GAAGAAU | |
| | | | UCUCCUC | | UGAAUGU | |
| | | | CA | | GA | |
| RD2440 | 988 | 1010 | UCACAUU | 307 | UGGAGGA | 623 |
| | | | CAAUUCU | | GAAGAAU | |
| | | | UCUCCUC | | UGAAUGU | |
| | | | CA | | GAU | |
| RD2442 | 1569 | 1591 | UCAUAUU | 313 | CUGAAUU | 624 |
| | | | GGUUUUU | | CCAAAAA | |
| | | | GGAAUUC | | CCAAUAU | |
| | | | AG | | GA | |
| RD2492 | 1548 | 1568 | UGUAAUU | 164 | UUCCUUU | 625 |
| | | | CAAAGGA | | GAAUUAC | |
| | | | GCCUGGA | | A | |

TABLE 10
Compound Chemistry

| | | | SEQ |
|---|---|---|---|
| | Modified Strands | Ref ID | ID |
| Compound | (5′-3′) | NO: | NO: |
| RD2423 | mu*fG*mU.fA.mA.fU. | IA0818 | 312 |
| | mU.fC.mA.fA.mA.fG. | | |
| | mG.fA.mG.fC.mC.fU. | | |
| | mG.fG.mA*mG*mU | | |
| | H9*mU*mC.mC.mA.mG. | IS1058 | 619 |
| | mG.fC.mU.fC.fC.fU. | | |
| | fU.mU.mG.mA.mA.mU. | | |
| | mU.mA.mC*mA*dQ | | |
| RD2436 | mu*fG*mU.fA.mA.fU. | IA0818 | 312 |
| | mU.fC.mA.fA.mA.fG. | | |
| | mG.fA.mG.fC.mC.fU. | | |
| | mG.fG.mA*mG*mU | | |
| | H2*mC*mU.mC.mC.mA. | IS1066 | 616 |
| | mG.mG.fC.mU.fC.fC. | | |
| | fU.fU.mU.mG.mA.mA. | | |
| | mU.mU.mA.mC*mA*dQ | | |
| RD2437 | mu*fG*mU.fA.mA.fU. | IA0818 | 312 |
| | mU.fC.mA.fA.mA.fG. | | |
| | mG.fA.mG.fC.mC.fU. | | |
| | mG.fG.mA*mG*mU | | |
| | H2*mC*mU.mC.mC.mA. | IS1067 | 620 |
| | mG.mG.fC.mU.fC.fC. | | |
| | fU.fU.mU.mG.mA.mA. | | |
| | mU.mU.mA.mC.mA*mU* | | |
| | dQ | | |
| RD2438 | mu*fG*mU.fA.mA.fU. | IA0818 | 312 |
| | mU.fC.mA.fA.mA.fG. | | |
| | mG.fA.mG.fC.mC.fU. | | |
| | mG.fG.mA*mG*mU | | |
| | H2*mC*mC.mA.mG.mG. | IS1068 | 468 |
| | fC.mU.fC.fC.fU.fU. | | |
| | mU.mG.mA.mA.mU.mU. | | |
| | mA.mC*mA*dQ | | |
| RD2439 | mU*fC*mA.fC.mA.fU. | IA0813 | 307 |
| | mU.fC.mA.fA.mU.fU. | | |
| | mC.fU.mU.fC.mU.fC. | | |
| | mC.fU.mC*mC*mA | | |
| | H2*mG*mG.mA.mG.mG. | IS1069 | 622 |
| | mA.mG.fA.mA.fG.fA. | | |
| | fA.fU.mU.mG.mA.mA. | | |
| | mU.mG.mU.mG*mA*dQ | | |
| RD2440 | mU*fC*mA.fC.mA.fU. | IA0813 | 307 |
| | mU.fC.mA.fA.mU.fU. | | |
| | mC.fU.mU.fC.mU.fC. | | |
| | mC.fU.mC*mC*mA | | |
| | H2*mG*mG.mA.mG.mG. | IS1070 | 623 |
| | mA.mG.fA.mA.fG.fA. | | |
| | fA.fU.mU.mG.mA.mA. | | |
| | mU.mG.mU.mG.mA*mU* | | |
| | dQ | | |
| RD2442 | mU*fC*mA.fU.mA.fU. | IA0828 | 313 |
| | mU.fG.mG.fU.mU.fU. | | |
| | mU.fU.mG.fG.mA.fA. | | |
| | mU.fU.mC*mA*mG | | |
| | H9*mU*mG.mA.mA.mU. | IS1072 | 624 |
| | mU.mC.fC.mA.fA.fA. | | |
| | fA.fA.mC.mC.mA.mA. | | |
| | mU.mA.mU.mG*mA*dO | | |
| RD2492 | mU*fG*mU.fA.mA.fU. | IA0867 | 164 |
| | mU.fC.mA.fA.mA.fG. | | |
| | mG.fA*mG*fC*mC*fU* | | |
| | mG*fG*mA | | |
| | H2*mU.fC.fC.fU.fU. | IS1074 | 625 |
| | mU.mG.mA.mA.mU.mU. | | |
| | mA.mC.mA*dQ | | |

TABLE 11
Average PKK Inhibition

| | Days | | |
|---|---|---|---|
| Compound | 4 | 8 | 15 |
| RD2423 | 28 | 52 | 66 |
| RD2436 | 22 | 43 | 67 |
| RD2437 | 62 | 45 | 66 |
| RD2438 | 21 | 50 | 77 |
| RD2439 | 26 | 43 | 55 |
| RD2440 | 21 | 31 | 46 |
| RD2442 | 20 | 41 | 71 |
| RD2492 | 23 | 37 | 55 |

TABLE 12
Average PKK Inhibition

| | Days | | | |
|---|---|---|---|---|
| Compound | 4 | 8 | 15 | 22 |
| RD2423 | 26 | 51 | 79 | 85 |
| RD2436 | 20 | 40 | 69 | 76 |
| RD2437 | 5 | 33 | 64 | 75 |
| RD2438 | 30 | 49 | 74 | 81 |
| RD2439 | 4 | 36 | 58 | 64 |
| RD2440 | 4 | 27 | 46 | 39 |
| RD2442 | 9 | 39 | 63 | 70 |
| RD2492 | 17 | 33 | 52 | 54 |

TABLE 13
Average PKK Inhibition

Days

| Compound | 4 | 8 | 15 | 22 | 29 | 36 |
|---|---|---|---|---|---|---|
| RD2423 | 26 | 51 | 79 | 85 | 85 | 84 |
| RD2436 | 20 | 40 | 69 | 76 | 77 | 79 |
| RD2437 | 5 | 33 | 64 | 75 | 77 | 79 |
| RD2438 | 30 | 49 | 74 | 81 | 76 | 76 |
| RD2439 | 4 | 36 | 58 | 64 | 63 | — |
| RD2440 | 4 | 27 | 46 | 39 | 47 | — |
| RD2442 | 9 | 39 | 63 | 70 | 67 | — |
| RD2492 | 17 | 33 | 52 | 54 | 52 | — |

TABLE 14
Average PKK Inhibition

| | Day | Day | Day | Day | Day | Day | Day | Day | Day | Day | Day | Day | Day |
|---|---|---|---|---|---|---|---|---|---|---|---|---|---|
| Compound | 4 | 8 | 15 | 22 | 29 | 36 | 43 | 50 | 57 | 64 | 71 | 78 | 85 |
| RD2423 | 14 | 41 | 74 | 88 | 91 | 90 | 88 | 92 | 81 | 85 | 87 | 80 | 68 |
| RD2436 | 20 | 40 | 69 | 76 | 77 | 79 | 73 | 77 | 76 | 69 | 61 | 64 | 37 |
| RD2437 | 5 | 33 | 64 | 75 | 77 | 79 | 70 | 80 | 79 | 71 | 70 | 68 | 54 |
| RD2438 | 30 | 49 | 74 | 81 | 76 | 76 | 68 | 71 | 60 | 52 | 42 | 36 | |
| RD2439 | 4 | 36 | 58 | 64 | 63 | 58 | 65 | 66 | | | | | |
| RD2440 | 4 | 27 | 46 | 39 | 47 | | | | | | | | |
| RD2442 | 9 | 39 | 63 | 70 | 67 | 73 | 72 | 72 | 71 | 59 | 60 | 62 | 56 |
| RD2492 | 17 | 33 | 52 | 54 | 52 | | | | | | | | |

TABLE 15
Body Weight (kg)

| Compound | Day −1 | Day 63 | Day 91 |
|---|---|---|---|
| RD2423 Cyno#1 | 2.4 | 2.4 | 2.4 |
| RD2423 Cyno#2 | 2.6 | 2.6 | 2.7 |
| RD2436 Cyno#1 | 2.9 | 3.1 | 3.2 |
| RD2436 Cyno#2 | 2.5 | 2.6 | 2.7 |
| RD2437 Cyno#1 | 3.1 | 3.2 | 3.2 |
| RD2437 Cyno#2 | 2.6 | 2.7 | 2.9 |
| RD2438 Cyno#1 | 3.2 | 3.2 | 3.4 |
| RD2438 Cyno#2 | 2.5 | 2.6 | 2.6 |
| RD2439 Cyno#1 | 3.6 | 4.1 | N/A |
| RD2439 Cyno#2 | 2.7 | 2.8 | N/A |
| RD2440 Cyno#1 | 3.9 | 4.3 | N/A |
| RD2440 Cyno#2 | 2.3 | 2.4 | N/A |
| RD2442 Cyno#1 | 4.4 | 4.5 | 4.6 |
| RD2442 Cyno#2 | 2.3 | 2.4 | 2.5 |
| RD2492 Cyno#1 | 4 | 4 | N/A |
| RD2492 Cyno#2 | 2.3 | 2.3 | N/A |

TABLE 16
Liver Function Markers

| | ALT (U/L) | | | AST (U/L) | | | Bilirubin (mg/dL) | | | Albumin (g/dL) | | |
|---|---|---|---|---|---|---|---|---|---|---|---|---|
| | Day | Day | Day | Day | Day | Day | Day | Day | Day | Day | Day | Day |
| Compound | −6 | 64 | 92 | −6 | 64 | 92 | −6 | 64 | 92 | −6 | 64 | 92 |
| RD2423 Cyno#1 | 47 | 47 | 51 | 53 | 46 | 46 | 0.25 | 0.26 | 0.28 | 4.3 | 4.5 | 4.5 |
| RD2423 Cyno#2 | 76 | 75 | 58 | 69 | 65 | 46 | 0.15 | 0.36 | 0.27 | 4.0 | 4.3 | 3.9 |
| RD2436 Cyno#1 | 25 | 22 | 25 | 45 | 48 | 39 | 0.24 | 0.34 | 0.32 | 4.5 | 4.3 | 4.1 |
| RD2436 Cyno#2 | 65 | 73 | 78 | 64 | 41 | 42 | 0.19 | 0.23 | 0.25 | 4.4 | 4.5 | 4.5 |
| RD2437 Cyno#1 | 41 | 40 | 51 | 39 | 43 | 43 | 0.24 | 0.28 | 0.27 | 4.3 | 4.5 | 4.4 |
| RD2437 Cyno#2 | 55 | 51 | 50 | 55 | 57 | 48 | 0.14 | 0.24 | 0.26 | 4.5 | 4.6 | 4.4 |
| RD2438 Cyno#1 | 66 | 64 | 64 | 101 | 62 | 50 | 0.18 | 0.17 | 0.17 | 4.2 | 4.5 | 4.2 |
| RD2438 Cyno#2 | 25 | 21 | 25 | 38 | 33 | 33 | 0.33 | 0.44 | 0.53 | 4.8 | 4.6 | 4.5 |
| RD2439 Cyno#1 | 80 | 64 | N/A | 87 | 48 | N/A | 0.16 | 0.16 | N/A | 4.4 | 4.6 | N/A |
| RD2439 Cyno#2 | 105 | 88 | N/A | 49 | 54 | N/A | 0.15 | 0.23 | N/A | 4.5 | 4.4 | N/A |
| RD2440 Cyno#1 | 66 | 48 | N/A | 115 | 52 | N/A | 0.32 | 0.30 | N/A | 4.4 | 4.6 | N/A |
| RD2440 Cyno#2 | 50 | 58 | N/A | 45 | 48 | N/A | 0.25 | 0.39 | N/A | 4.8 | 5.0 | N/A |
| RD2442 Cyno#1 | 66 | 61 | 69 | 54 | 38 | 55 | 0.17 | 0.17 | 0.30 | 4.1 | 4.2 | 4.1 |
| RD2442 Cyno#2 | 63 | 70 | 70 | 51 | 54 | 56 | 0.21 | 0.23 | 0.17 | 4.3 | 4.4 | 4.3 |
| RD2492 Cyno#1 | 57 | 39 | N/A | 84 | 38 | N/A | 0.12 | 0.17 | N/A | 4.2 | 4.2 | N/A |
| RD2492 Cyno#2 | 92 | 78 | N/A | 72 | 85 | N/A | 0.18 | 0.27 | N/A | 4.7 | 4.5 | N/A |

TABLE 17
Kidney Function Markers

| | BUN (mg/dL) | | | Creatinine (mg/dL) | | |
|---|---|---|---|---|---|---|
| | Day | Day | Day | Day | Day | Day |
| Compound | −6 | 64 | 92 | −6 | 64 | 92 |
| RD2423 Cyno#1 | 24 | 20 | 18 | 0.51 | 0.62 | 0.58 |
| RD2423 Cyno#2 | 34 | 30 | 28 | 0.51 | 0.61 | 0.55 |
| RD2436 Cyno#1 | 27 | 26 | 23 | 0.48 | 0.49 | 0.47 |
| RD2436 Cyno#2 | 22 | 19 | 19 | 0.46 | 0.51 | 0.56 |
| RD2437 Cyno#1 | 31 | 24 | 21 | 0.61 | 0.71 | 0.75 |
| RD2437 Cyno#2 | 25 | 21 | 23 | 0.48 | 0.48 | 0.43 |
| RD2438 Cyno#1 | 23 | 27 | 20 | 0.46 | 0.64 | 0.61 |
| RD2438 Cyno#2 | 21 | 18 | 22 | 0.50 | 0.47 | 0.46 |
| RD2439 Cyno#1 | 21 | 21 | N/A | 0.53 | 0.63 | N/A |
| RD2439 Cyno#2 | 28 | 27 | N/A | 0.39 | 0.36 | N/A |
| RD2440 Cyno#1 | 23 | 24 | N/A | 0.53 | 0.74 | N/A |
| RD2440 Cyno#2 | 24 | 19 | N/A | 0.32 | 0.44 | N/A |
| RD2442 Cyno#1 | 30 | 26 | 25 | 0.57 | 0.75 | 0.62 |
| RD2442 Cyno#2 | 28 | 30 | 27 | 0.50 | 0.61 | 0.59 |
| RD2492 Cyno#1 | 23 | 22 | N/A | 0.52 | 0.59 | N/A |
| RD2492 Cyno#2 | 33 | 26 | N/A | 0.66 | 0.44 | N/A |

### Example 5: Effect of Compounds Targeting Human PKK in Cynomolgus Monkeys

Compounds of interest were evaluated in cynomolgus monkeys (Table 19). Prior to the study the monkeys were kept in quarantine during which the animals were observed daily for general health. Eight groups of 2 cynomolgus monkey each were injected with a single 4 mg/kg subcutaneous dose of oligonucleotide on Day 1 of the study. During the study period, the monkeys were observed daily for signs of illness or distress. Animals were bled on day −6 and on days 1 (prior to dosing), 8, 15 and 22 for serum collection and analysis. Future collections will be on days 29, 36, 43, 50, 57, 64 and 71. In a second set of experiments, animals were bled on day −6 and days 1 (prior to dosing), 8, 15, 22, 29, 36, 43, 50, 57, 64 and 71. The protocols described were approved by the Institutional Animal Care and Use Committee (IACUC). Circulating PKK levels were quantified using an ELISA specific for human angiotensinogen (and cross-reactive with cynomolgus), according to manufacturer's protocol (IBL America #27412). PKK inhibition data were expressed as percent of baseline value (Day 1 prior to dosing) and as an average of the group for each compound. Results from the two sets of experiments are shown in Tables 20 and 21. Clinical chemistry was performed on Day-1 or Day-6 and Day 64 and Day 92. There were no test article-related effects on body weight (Table 22) and all serum chemistry values were within reference ranges (Tables 23 and 24).

TABLE 18
Compound Sequence

| | Seq | SEQ | | | | |
|---|---|---|---|---|---|---|
| | ID | ID | Anti- | | | |
| | NO: | NO: | sense | | Sense | |
| Com- | 1 | 1 | Se- | SEQ | Se- | SEQ |
| pound | Start | Stop | quence | ID | quence | ID |
| Number | Site | Site | (5′-3′) | NO: | (5′-3′) | NO: |
| RD2424 | 630 | 652 | UGAGAA | 626 | UAACGU | 629 |
| | | | UCCAGA | | GGAAUC | |
| | | | UUCCAC | | UGGAUU | |
| | | | GUUAC | | CUCA | |
| RD2425 | 689 | 711 | UCAAGA | 627 | UGAACA | 630 |
| | | | UGCUGG | | CUCUUC | |
| | | | AAGAUG | | AGCAUC | |
| | | | UUCAU | | UUGA | |
| RD2426 | 1183 | 1205 | ACAAUC | 628 | CUCUGG | 631 |
| | | | UCAAAG | | UUACUC | |
| | | | AGUAAC | | UUUGAG | |
| | | | CAGAG | | AUUGU | |

TABLE 19
Compound Chemistry

| | Modified | | SEQ |
|---|---|---|---|
| | Strands | Ref ID | ID |
| Compound | (5′-3′) | NO: | NO: |
| RD2424 | mu*fG*mA.fG.mA.fA. | IA0864 | 626 |
| | mU.fC.mC.fA.mG.fA. | | |
| | mU.fU.mC.fC.mA.fC. | | |
| | mG.fU.mU*mA*mC | | |
| | H4*mA*mA.mC.mG.mU. | IS1059 | 629 |
| | mG.fG.mA.fA.fU.fC. | | |
| | fU.mG.mG.mA.mU.mU. | | |
| | mC.mU.mC*mA*dQ | | |
| RD2425 | mU*fC*mA.fA.mG.fA. | IA0865 | 627 |
| | mU.fG.mC.fU.mG.fG. | | |
| | mA.fA.mG.fA.mU.fG. | | |
| | mU.fU.mC*mA*mU | | |
| | H4*mG*mA.mA.mC.mA. | IS1060 | 630 |
| | mU.fC.mU.fU.fC.fC. | | |
| | fA.mG.mC.mA.mU.mC. | | |
| | mU.mU.mG*mA*dO | | |
| RD2426 | mA*fC*mA.fA.mU.fC. | IA0866 | 628 |
| | mU.fC.mA.fA.mA.fG. | | |
| | mA.fG.mU.fA.mA.fC. | | |
| | mC.fA.mG*mA*mG | | |
| | H9*mU*mC.mU.mG.mG. | IS1061 | 631 |
| | mU.mU.fA.mC.fU.fC. | | |
| | fU.fU.mU.mG.mA.mG. | | |
| | mA.mU.mU.mG*mU*dQ | | |

TABLE 20
Average PKK Inhibition

| | Days | | |
|---|---|---|---|
| Compound | 8 | 15 | 22 |
| RD2424 | 53 | 80 | 87 |
| RD2425 | 31 | 60 | 69 |
| RD2426 | 30 | 54 | 58 |

TABLE 21
Average PKK Inhibition

| | Day | Day | Day | Day | Day | Day | Day | Day | Day | Day |
|---|---|---|---|---|---|---|---|---|---|---|
| Compound | 8 | 15 | 22 | 29 | 36 | 43 | 50 | 57 | 64 | 71 |
| RD2424 | 71 | 87 | 94 | 91 | 91 | 84 | 88 | 80 | 79 | 74 |
| RD2425 | 42 | 60 | 76 | 73 | 80 | 75 | 72 | 72 | 68 | |
| RD2426 | 30 | 54 | 58 | 57 | | 57 | 65 | 58 | 58 | |

TABLE 22
Body Weight (kg)

| Compound | Day −1 | Day 64 | Day 92 |
|---|---|---|---|
| RD2424 Cyno#1 | 3.1 | 3.1 | 3.1 |
| RD2424 Cyno#2 | 3.2 | 3.6 | 3.7 |
| RD2425 Cyno#1 | 2.9 | 3.3 | N/A |
| RD2425 Cyno#2 | 3.1 | 3.5 | N/A |
| RD2426 Cyno#1 | 3.5 | 3.9 | N/A |
| RD2426 Cyno#2 | 2.8 | 3.2 | N/A |

TABLE 23
Liver Function Markers

| | ALT (U/L) | | | AST (U/L) | | | Bilirubin (mg/dL) | | | Albumin (g/dL) | | |
|---|---|---|---|---|---|---|---|---|---|---|---|---|
| | Day | Day | Day | Day | Day | Day | Day | Day | Day | Day | Day | Day |
| Compound | −6 | 64 | 92 | −6 | 64 | 92 | −6 | 64 | 92 | −6 | 64 | 92 |
| RD2424 Cyno#1 | 58 | N/A | 65 | 50 | N/A | 45 | <0.2 | N/A | <0.2 | 4.3 | N/A | 4.4 |
| RD2424 Cyno#2 | 50 | N/A | 42 | 34 | N/A | 32 | 0.3 | N/A | 0.2 | 4.5 | N/A | 4.5 |
| RD2425 Cyno#1 | 57 | 49 | N/A | 49 | 38 | N/A | 0.2 | <0.2 | N/A | 4.2 | 4.2 | N/A |
| RD2425 Cyno#2 | 37 | 42 | N/A | 39 | 39 | N/A | 0.4 | 0.2 | N/A | 4.5 | 4.5 | N/A |
| RD2426 Cyno#1 | 46 | 40 | N/A | 46 | 40 | N/A | 0.2 | 0.3 | N/A | 4.2 | 4.6 | N/A |
| RD2426 Cyno#2 | 68 | 52 | N/A | 68 | 52 | N/A | 0.3 | <0.2 | N/A | 4.4 | 4.4 | N/A |

TABLE 24
Kidney Function Markers

| | BUN (mg/dL) | | | Creatinine (mg/dL) | | |
|---|---|---|---|---|---|---|
| | Day | Day | Day | Day | Day | Day |
| Compound | −6 | 64 | 92 | −6 | 64 | 92 |
| RD2424 Cyno#1 | 22 | N/A | 14 | 0.5 | N/A | 0.5 |
| RD2424 Cyno#2 | 22 | N/A | 18 | 0.8 | N/A | 0.8 |
| RD2425 Cyno#1 | 21 | 15 | N/A | 0.4 | 0.4 | N/A |
| RD2425 Cyno#2 | 28 | 22 | N/A | 0.6 | 0.5 | N/A |
| RD2426 Cyno#1 | 23 | 20 | N/A | 0.6 | 0.6 | N/A |
| RD2426 Cyno#2 | 31 | 26 | N/A | 0.7 | 0.5 | N/A |

### Example 6: Effect of Compounds Targeting Human PKK in Lewis Rats

Female Lewis rats (5 females/group, 8 weeks of age) were given single subcutaneous SC injections of 0 (Vehicle Control (phosphate buffered saline (PBS)), 200 mg/kg RD2423 or 200 mg/kg RD2438 at a dose volume of mL/kg to groups 1-3, respectively, on Day 1. Observations included viability, clinical signs and body weight (Days 1 and 10). Blood samples were collected for clinical chemistry (Day 0 (predose), 3 and 10), hematology (Day 10) and coagulation (Day 10). On Day 10, heart, liver, and both kidneys were harvested from each rat and fixed in 10% neutral buffered formalin after weighing.

Results: There were no drug-related effects on viability, clinical observations, body weight or organ weight (heart, liver, and kidneys), clinical chemistry, hematology or coagulation (prothrombin clotting time) parameters. A single subcutaneous administration of 200 mg/kg of compounds RD2423 or RD2438 to female Lewis rats was well tolerated.

SEQ ID NO: 1
AGTGCCACATTAGAACAGCTTGAAGACCGTTCATTT
TTAAGTGACAAGAGACTCACCTCCAAGAAGCAATT
GTGTTTTCAGAATGATTTTATTCAAGCAAGCAACT
TATTTCATTTCCTTGTTTGCTACAGTTTCCTGTGG
ATGTCTGACTCAACTCTATGAAAACGCCTTCTTCA
GAGGTGGGGATGTAGCTTCCATGTACACCCCAAAT
GCCCAATACTGCCAGATGAGGTGCACATTCCACCC
AAGGTGTTTGCTATTCAGTTTTCTTCCAGCAAGTT
CAATCAATGACATGGAGAAAAGGTTTGGTTGCTTC
TTGAAAGATAGTGTTACAGGAACCCTGCCAAAAGT
ACATCGAACAGGTGCAGTTTCTGGACATTCCTTGA
AGCAATGTGGTCATCAAATAAGTGCTTGCCATCGA
GACATTTATAAAGGAGTTGATATGAGAGGAGTCAA
TTTTAATGTGTCTAAGGTTAGCAGTGTTGAAGAAT
GCCAAAAAAGGTGCACCAGTAACATTCGCTGCCAG
ITTTTTTCATATGCCACGCAAACATTTCACAAGGC
AGAGTACCGGAACAATTGCCTATTAAAGTACAGTC
CCGGAGGAACACCTACCGCTATAAAGGTGCTGAGT
AACGTGGAATCTGGATTCTCACTGAAGCCCTGTGC
CCTTTCAGAAATTGGTTGCCACATGAACATCTTCC
AGCATCTTGCGTTCTCAGATGTGGATGTTGCCAGG
GTTCTCACTCCAGATGCTTTTGTGTGTCGGACCAT
CTGCACCTATCACCCCAACTGCCTCTTCTTTACAT
TCTATACAAATGTATGGAAAATCGAGTCACAAAGA
AATGTTTGTCTTCTTAAAACATCTGAAAGTGGCAC
ACCAAGTTCCTCTACTCCTCAAGAAAACACCATAT
CTGGATATAGCCTTTTAACCTGCAAAAGAACTTTA
CCTGAACCCTGCCATTCTAAAATTTACCCGGGAGT
TGACTTTGGAGGAGAAGAATTGAATGTGACTTTTG
TTAAAGGAGTGAATGTTTGCCAAGAGACTTGCACA
AAGATGATTCGCTGTCAGTTTTTCACTTATTCTTT
ACTCCCAGAAGACTGTAAGGAAGAGAAGTGTAAGT
GTTTCTTAAGATTATCTATGGATGGTTCTCCAACT
AGGATTGCGTATGGGACACAAGGGAGCTCTGGTTA
CTCTTTGAGATTGTGTAACACTGGGGACAACTCTG
TCTGCACAACAAAAACAAGCACACGCATTGTTGGA
GGAACAAACTCTTCTTGGGGAGAGTGGCCCTGGCA
GGTGAGCCTGCAGGTGAAGCTGACAGCTCAGAGGC
ACCTGTGTGGAGGGTCACTCATAGGACACCAGTGG
GTCCTCACTGCTGCCCACTGCTTTGATGGGCTTCC
CCTGCAGGATGTTTGGCGCATCTATAGTGGCATTT
TAAATCTGTCAGACATTACAAAAGATACACCTTTC
TCACAAATAAAAGAGATTATTATTCACCAAAACTA
TAAAGTCTCAGAAGGGAATCATGATATCGCCTTGA
TAAAACTCCAGGCTCCTTTGAATTACACTGAATTC
CAAAAACCAATATGCCTACCTTCCAAAGGTGACAC
AAGCACAATTTATACCAACTGTTGGGTAACCGGAT
GGGGCTTCTCGAAGGAGAAAGGTGAAATCCAAAAT
ATTCTACAAAAGGTAAATATTCCTTTGGTAACAAA
TGAAGAATGCCAGAAAAGATATCAAGATTATAAAA
TAACCCAACGGATGGTCTGTGCTGGCTATAAAGAA
GGGGGAAAAGATGCTTGTAAGGGAGATTCAGGTGG
TCCCTTAGTTTGCAAACACAATGGAATGTGGCGTT
TGGTGGGCATCACCAGCTGGGGTGAAGGCTGTGCC
CGCAGGGAGCAACCTGGTGTCTACACCAAAGTCGC
TGAGTACATGGACTGGATTTTAGAGAAAACACAGA
GCAGTGATGGAAAAGCTCAGATGCAGTCACCAGCA
TGAGAAGCAGTCCAGAGTCTAGGCAATTTTTACAA
CCTGAGTTCAAGTCAAATTCTGAGCCTGGGGGGTC
CTCATCTGCAAAGCATGGAGAGTGGCATCTTCTTT
GCATCCTAAGGACGAAAAACACAGTGCACTCAGAG
CTGCTGAGGACAATGTCTGGCTGAAGCCCGCTTTC
AGCACGCCGTAACCAGGGGCTGACAATGCGAGGTC
GCAACTGAGATCTCCATGACTGTGTGTTGTGAAAT
AAAATGGTGAAAGATCA

1-88. (canceled)

89. A compound comprising a modified oligonucleotide 14 to 23 linked nucleosides in length having a nucleobase sequence comprising at least 14 contiguous nucleobases of the nucleobase sequence of SEQ ID NO: 619 or SEQ ID NO: 312.

90. A compound comprising a modified oligonucleotide 22 to 30 linked nucleosides in length having a nucleobase sequence comprising the nucleobase sequence of SEQ ID NO: 619 or SEQ ID NO: 312.

91. A compound comprising a first modified oligonucleotide 14 to 23 linked nucleosides in length having a nucleobase sequence comprising at least 14 contiguous nucleobases of the nucleobase sequence of SEQ ID NO: 619, and a second modified oligonucleotide 14 to 23 linked nucleosides in length having a region of complementarity to the first modified oligonucleotide.

92. The compound of claim 91, wherein the nucleobase sequence of the first modified oligonucleotide has at least 80% complementarity to the second modified oligonucleotide.

93. The compound of claim 91, wherein the nucleobase sequence of the first modified oligonucleotide has at least 1, at least 2, or at least 3 mismatches to a region of the second modified oligonucleotide that is the same length as the first modified oligonucleotide.

94. The compound of claim 91, wherein the region of complementarity between the first modified oligonucleotide and the second modified oligonucleotide is 14 to 23 linked nucleosides in length.

95. The compound of claim 91, wherein the first modified oligonucleotide is fully complementary to the second modified oligonucleotide.

96. The compound of claim 91, wherein the second modified oligonucleotide has a nucleobase sequence comprising at least 14 contiguous nucleobases of the nucleobase sequence of SEQ ID NO: 312.

97. The compound of claim 91, wherein the first and/or second modified oligonucleotide comprises at least one modification selected from a modified internucleoside linkage, a modified sugar, and a modified nucleobase.

98. The compound of claim 97, wherein the modified internucleoside linkage is a phosphorothioate internucleoside linkage or a methylphosphonate internucleoside linkage.

99. The compound of claim 98, wherein the phosphorothioate internucleoside linkage or methylphosphonate internucleoside linkage is at the 3′ terminus of the first or second modified oligonucleotide or at the 5′ terminus of the first modified oligonucleotide.

100. The compound of claim 97, wherein the modified sugar comprises a 2′-F modification or a 2′-OMe modification.

101. The compound of claim 91, wherein the first modified oligonucleotide comprises a conjugate group.

102. The compound of claim 101, wherein the conjugate group is attached to the 5′ end of the first modified oligonucleotide.

103. The compound of claim 101, wherein the conjugate group comprises a targeting moiety.

104. The compound of claim 103, wherein the targeting moiety comprises one or more N-Acetyl-galactosamine (GalNAc).

105. The compound of claim 104, wherein the one or more GalNAc are attached to the 2′ or 3′ position of the ribosyl ring of the 5′ nucleoside of the first modified oligonucleotide.

106. The compound of claim 105, wherein the 5′ nucleoside is of the following formula: 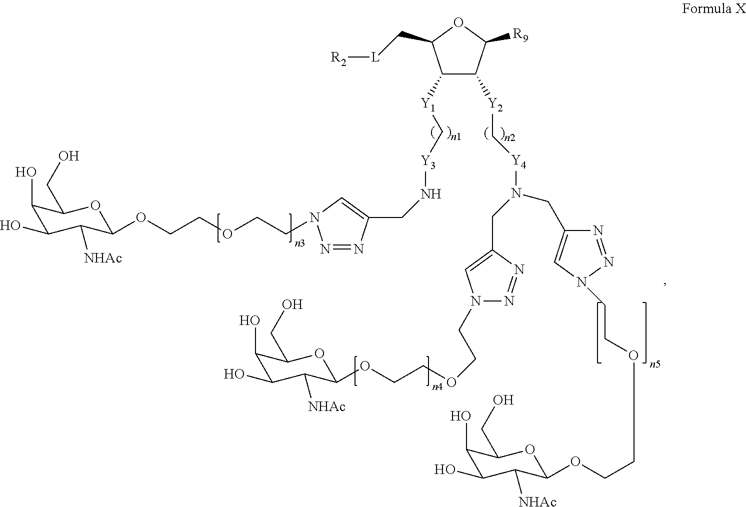
wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is the oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each n₁, n₃, n₄ and n₅ is independently 0, 1, 2, 3, 4, 5, 6, 7, 8, 9, or 10.

107. A compound comprising a first modified oligonucleotide consisting of Ref ID NO: IS1058 and a second modified oligonucleotide consisting of Ref ID NO: IA0818.

108. A composition comprising the compound of claim 91 and a pharmaceutically acceptable carrier.

109. A method comprising administering the compound of claim 91 to an individual.

110. The method of claim 109, wherein administering the compound inhibits or reduces or improves an inflammatory or thrombotic disease, disorder or condition or a symptom thereof, hereditary angioedema (HAE), edema, angioedema, swelling, angioedema of the lids, ocular edema, macular edema, cerebral edema, thrombosis, embolism, thromboembolism, deep vein thrombosis, pulmonary embolism, myocardial infarction, stroke, or infarct.

111. The method of claim 109, wherein the compound is administered parenterally.

112. A method of inhibiting expression of PKK in a cell comprising contacting the cell with a compound of claim 91, thereby inhibiting expression of PKK in the cell.

113. The method of claim 112, wherein the cell is in the liver of an individual.


SEQUENCE LISTING (630 sequences)

SEQ ID NO: 1 - DNA, 2258 bp, Homo sapiens
  source 1..2258: mol_type = genomic DNA
agtgccacattagaacagcttgaagaccgttcatttttaagtgacaagagactcacctccaagaagcaattgtgttttcagaatgattttattcaagcaagcaacttatttcatttccttgtttgctacagtttcctgtggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaactctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatca

SEQ ID NO: 2 - DNA, 30965 bp, Homo sapiens
  source 1..30965: mol_type = genomic DNA
agtgccacattagaacagcttgaagaccgttcatttttaagtgacaagagactcacctccaagaagcaattgtgttttcaggtagcaaatttttattattctgattgtttccaaataaactataatttttaagtataattttttactttatgagaaaattaatcatttatattctaatttcctgagtatgtagagagtatagataatgttcctttatgtagaaatatttaaatgtaagatgattttaaatcagaaagaatatttgattgatttaaaatttttaaatgggctttaatattttcagaggttttctttacttagggatttttggactgacattattgccattatttattaattttgtttttgcccaaatcaagaggtttcataattgtttactctctctctaccaattccctttccaacattactagccacagagttggccaatgaacaataaacacaacagtagtctggaggtctcaatttgtatcttgggaagcattataaattttccaactccctagacacaaatgtaccaaaaaaaaacccttgttttctataccagtaattgtgtgctttgtcttgcaattcagacatttacaagaaaatctaaatcaccttaaattaagatttatgttaaatgtggtctaaaaccagcagagttatgtattgttttcttttttagaatgattttattcaagcaagcaacttatttcatttccttgtttgctacagtttcctgtggtaagtgaattatctataaaacatggaattcaggctaagacaggagtagccaagcaagtggcaccacccctggagaaagctattgaacatacagcttcgggggtggagattgtccctgatgattcaggacacgtgtctatttaatgttccacaacaaggaccacttgtcaggtatattgctgtagacatatgttgcagaccagaggaaggagctcagaagtaggaatgtcttgggacttgtgttaacaaaaacttctgttcgcagatgacactctgcaaagcaaaacttgaaacaaaaaaaaattagtcctctatttttattatcaacagtaaaaaattaaactttatctgaaaattcaaaagagtgctaggcattttatagtgtctgggtccaatccaagtatctgttaggaacaccatacatagttttactctggaccgctagggaaccatttcaaaaatgaaagtaactggtttaaatttaacttagcaaaccatgcatttggatagttctaggtgaatagctttcaacaccagatttagatctcatttctctattaatttcattaatttttggagaataaaaatgattctggacatttcattaatcattacagagggagttttctctgtgtccccacaaggcatgattctgggtctatggtgacttaagagggccacacaacaatgagtatttaattttcctcagatgtatggctacaataaacatctataggtaaggtttacattcataaagagacctttttttttccaggaaaaaagacttttattccccctaaatcacaactcccctgtgtctgtccctcaaccctgatttctcttctaaaccgtaatttacaaacccatgtgcaaacccactgaaaggtgagaaggagcataagccagagacactgggaaccacagccaactacagggggtttttcatttttttgttttgttttgttttttggcaaaataaatcatgattatggctaggaaaatcagggatgtaagtaagcaaaaatttagaaactacatattgcatgtagtccccaaattcagaaacagtcatcgttaaacatcttttatttagtctttcagatatttttctacatatacctatttgcacatttcacaaaaaagagttattaactgtggacactctttgacttgcattttccacttatagctatcttttggtgcaaataagtaatatattttggagcttgctattcttctctttttatggtatttttgtgtgcacattcttatgctcttattcagttatttctgtagaatacaatttttgaaataaaaatactagattcaaaggtgtgaatatttttgaaggttttgtggtgtggtatcatgaaattaggtttcagaaagtttactccatagttgactttttctaccagctaataagagtgctcatttaccccacttaggccaactctaacaggcttctgttgctttgcatcctgacatatttatctttctggaaacagtgtcctaatttatttctgcagagctactgcctctcagttttggtccatgtggttctggtgaccctgattcttctgctgaaccagaaagtgcacacatcctcctggctgtggtgacccaatcagagccaacgtcttgcaatgaagcttttctttagacatctagaaataagactcttatgttttttttttccaatttgacttgaaacttaagacaatacacaggcctgagttgttggtactattttgctactacgtggagcttgagagtaatggtaacaccaagactggagcaagtggaaggagacatattgtccactgatgctatcagttgagcacaatgtgcagttactttttaagccagattcaccatgggctttttggctcctgaaccaatagatttcatttgttttcaagccactttgtactaggttttccatcatttgcaatcaaaaccagtatacaaacactggttatttaatttttcatttttgctaatctcagggataaaatggcttctagttgttttaacttggctatatttgtgattcttcccattttcatatacttactaatcacttctatttcttttgtaaattatcttttcatatttgttatctaattctttaaaatttgagtactttaattctcattattgtttgtggaattgttaaacaagactgaataatctgcccaaagtccatggacatgggggcccatgtaaaagctttgaaagccaccatcattatgagataaattatataatagtactttacataggctccaaaatacagcacagacacagctatcattgtcatggtcatcattatcatcatgatcaccaacttatgaggataagcaagaacacctactagaagtttctttccattcagcaacaaaattggtgtctttctagtcactcccttccctgactgtcacatagcagttcacagaggctcagttgcagaatgagaagctctgggccaggacctgccattgtatgcatccttgcatgggaactgggggctggaagaggagtgactgcttgataattatgagtcagtcaaaaccaccaactgtctgaaaaaaataggcctttttgtaactagtattgtcactaaaccaactcctccatgttttgtgcatacatgaaatctaggcaatacacttgtattcccaaaagcttccacttgaagagatctgtgctctttccaaatataaaccttacccgagaggtggtcatcttggccacacctcagagagggagagaggcagtcttgttgggttgggtggtcataatggggctcagggccaaatccccagggggtaggatagtgcagagaagatggcactctccagtgcttaataaaatgcacgtggtctaagctgcccactccctcaaaggcaataaaaaataggtactatttaaattgaagagtaactactgccccagggaatggacaggttgtcattggaatagccatggttaatggtcccagttgacaactgaaatgaatgtgctacctgaacaggaaagcttattaaccagatttcaaagacagtctttcccggtaaatccaaatttacaaattaaagccagtaaagacaccgaattctctaataatatgtggggtgcagtatattttagagctgggaataattccaaacagcaaatagttcaaaatttattttcaatttagcatatgcatgcatttgcttaaactgtcttaaaaatgagtaaaaaatactgtcagtttgtttcaatcatactgagatgaggaacaatgtttagcattgcatgctagaaaaggacacaggattgggagtcagggctgggtgaccgtcacaggactttcactaactgtgtggatcttgggcaagtctgtgtgccctgagactcagttatttaactttcttttaaaaaacatagtccagatgcagataacaaagcctgcctctaatttcccttacagaattgtgagaagctggtggagatgtttgttacaaaagtgttttgaaaatagagcaaatattattctttttaaggcatgatgttttcatagcatgtcaggcaacaggaaaaaactaagttaggattttattttattgtggggaatttatgtgcaaattattgtgcaatttaatgaaaataagccaatgttttatacagaagtacccagaaaattaaataacactatacattgttcaaatagttgccttaatatattttattttctcagcataattagagttgtattatacaggtctttgagtagtcagtcagtgggagaagttaagacaacagatatctttttattaaaattattatatgaattatcgcaaattaatttttatggttctgtcacaggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtaaaagttggtatttcattattggagaagctgtttttcaaaactgaatcagttttgtgcagaaaggtgtagtataactgagagttcttcctcacacggggttcaaggaccagcttcagcaaaatcccgtcaagtggttcttacaaatgcagattcctaggccacaacccagatctgctgaaccaaagtttcttgtgaccaggaatctgcattttaaacaatcactgtgtttctttaaagtagtagaagctagtcattttctattcaaagcctcaaaatgcttgaatatcattgggctaagggattgtctcaagaaagagtctaacaggtgcacatttcatctgaataaagaaacagatttaactgtgtgacccatgatcacattagcggatagcacagtccaaagaaaataacataagacaagcattttgctgagaatgtaattgagaaagatctagaacttgtgattttgggacagggcagttctaaatggggcctatagtgagccagtttgggcacctgtggcatgatgctatgtatggtgtgtgtgtgtatgtgtgcttgtgcttgtgtgaatatgtattattaactggaaatttgtaaaagtattggaaaaatagtacttacgattttgtgtgtgtgtgtgatggagtctcgctttgtagcccaggctggagtgcagtggcgtgatctcggctcactgcaacttccacctcccaggttcaagcgattctcctgcctcagcctcccaagtagctgggattacaggcacgcaccaccacgtccagctaatttttgtagttttagtagagacggggtttcaccacattggccaggctggccttgaactcctgacctcgtgatccacctgcctctacctcctaatgtgctgggattacaggcatgagccaccgcacccagcggtaattacaatttttattaggtcagagagatgcttattaatcacgagccacagtttcatcttaatgatttttccttttgattaatatccagagtaagcttttctttgttgttcccatttccatgttcataactctttactcatcttcactctatgtgagtttaccaactagaaattggatagtcattctctgatcccacatgttaaacttgtagagaaaactcagattgtatgtgaggatcatcatattaaaagtggaggaaggttctagaattcttataaataatgaaattaacatgaaggtggacatctaagacagagggaagtcttccattaagtgcagactacaaggagttaataagcaagatgaacacgatatacaaatccagctcttatcactaagttaactttttaagtaaatgaaagtatttgcaaaaataattaccaattgagaacatagttgcctgaaagtttaagaacacaggaaaaatcattaactctttaatatggttgatttcctgtacttaaaaaatgtgagtgtaaagaaaacgcagtgatggagttagatattatggggtgttataaaattagctctaagagtgttctttccagcaagtattggggaagctatattattttccttattcctggttttatttgttagtgtgtagaaaatgctagacatttcctcaatgtatgtttattattctacttcctaagtaaagctacttttaaaataggtttggttgcttcttgaaagatagtgttacaggaaccctgccaaaagtacatcgaacaggtgcagtttctggacattccttgaagcaatgtggtcatcaaataagtggtaagttgtgaatttcttagctacatttgagttaatattggatctcgcttagaacagcttttgctcaaagtttgtactgctacagctttttggaaggcatcactcataaagataggagatggggcagtattctggacacaaaagagggacccatattcatctggacacttctattgtctttataaatcaacacatacttaatgagcctctattatttatgaggttagcgctcaagtgtaagatttgcagaaaatgaatccaaataattgtgtctcgtttccagataagaatttttaagaaaacacaagggaacatctctctcaagttcacttgagggtaatttttacatcagtgattctcaaccagcagtgattttgtctcttccactggggacatttgacaatgtctggagacatttttggtggctacaactagggaggatgctattagcatttactaagtagaggccagaatgtttgaatgctgaccaacattctacaaggcacagggcagtcgtccacagcaaataattttctggcccaaaacgtcaacagtgctgacatcaagaaactctgtgatataccactaggcccaaattgaagaactgagttctgcaaatcttgctaagaataatacttcctaaaggaaacttgaggactaggatgctagagaactttgattctgacatctgaagctactgatgtcttgggaaacagtttccaatgctatcctaataaatttaagacaaatgaactatttctcaaacatgactgggactgataagaaagtgaaaagtgctgaaaagattcaactgatgggttgtcagaatcttaaaataactgctgttattctatgtatgactatatatcattactattttattttcattatgcacaattaattttgtaggttcaaatttcagatgtttttaaatttgtcatcctttcctccctcattgatatcacctcttcaatacgtacacactttgagcctgctgtttgcattttaaccagttatcaaaggatggcaatgccttcattataaatgtgggcctgacttagccagtataataggtgtagtctacgtgaggtggagtacatttcctattttaaaagatcaatttttatgttaatccaatttggtataaattattcgagtaagtgctatttctgattgttgtatcttgtagcaaaatttaaagaaaaagtaatttgtgcctttctcaatattcctgttattgttcatgtattctaaaactcactgttactcacttagcttgcttttaatgttttttaaagtgaaaaattgttcccaagtacataaaatctctacactcaagaacaattctagtcaaaagcatttagagcttccgtatgaacacttaaagagtttttatttgtaagagtcgcatcccaactcttagcctgttcttttctcacatgcagaaaaataggaaagagacttcgtttccacagtctgcaaattcctgtgtttaagaaccacagtgaataatccacctccctgcccaactcatcgtactgtcatatagttttcctgacagtttgtgtattttctgtctttcccacccttaaatttagttttatgacttcaaccatacttcttaggagtggaaaggtatctgtagtagattatggtttattccacataatcttggggaataaaactttaaaaaagtatacagtttatacttctggttacattacttccttaaccaaaagtctaaccaagaaatttgaatctttaaaaaaaaaagaggccgggcgcggtggctcacgcctgtaatcccagcactttgggaggccgagacgggcggatcacgaggtcaggagatcgagaccatcctggctgacacggtgaaaccccgtctctactaaaaatacaaaaattagccgggcgtggtggcgcgcgcctgtagtcccagctactcgggaggctgaggcaggagaatggcgtgaacccgggaggcggagcttgcagtgagtcgagatcgcgccactgcgctccagcctgggcgacagagcgagactccgtctcaaaaaaaaaaaaaaaaaaaagagcctaattttgcttcactgtctgtgaaaagaattatctgtatcttttgcatgtaagacaaatctcaatgaaaagggtgcttaaatagaagttaacactattttaaagcaagaatggaagtggtttcatcatgcgtaaacaacaactctccacattttgtaatgattgatctggatgcaatttgtcatcagacaggagaagtcgaaagcaaagaaataacactgggagatagagaagctctttcattcaatgcgaaaggtcaaaggcacatcagtttctttaataatgcaaacctcagcacacattatcagtgtcctcattattattgccttgtttatttcccactgctcattgataatttcaacgtgaaatttacctgtattgctgcatgcatcttgcagtttaagaagtgaagtaacccaatttcaaagctagtgctttagggaaaatattggattgtatttacttcaagcagagttcgataatttatgtacataataaaaattttaaatcccttagttaatatagcagttgccaaaactgggctattatcattctaaactaccaacccaaatggtagtgggtatctaatctacctctagaaagaaaatggactgtatttgctctatgtatttttcttgtacagcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggtgagtacaattcaaggtgtgtgttctttgtattggtgcctccaggatttcactgtattcttcttaacctcttttgttcccaaactaaaaaccaaacagggcttttattctaaccactttcctcatttacttactctattttatttttttatcttttatttatttatttatttattgagatggagtcgcgctctgtcgcccaggctagagtgcagtggcgtaatctcgactcactacaacctccgcttcccgggttcaagcgattctcctgcctcagcctcccgagtagctgggattacaggcgtccgccaccatgcctggctaatttttgtatttttagtagagtcggggttttactatgttggtcaggctggtctcgaactcctgatcttgtgatccacccacctcagcctcccaaagcgctgggattacaggcatgagccactgcgcctcgcccttcattttttaattaaataattcatttaatttcatttgtttctctactcttttcccctggcatgtaattgtaccgcatcttcaaagcctgacatcctcttcccctacttctccaaagctgattcttgcaggtctttcctcaaataccgtcccctccaaaagcccatttctgagcattctcttttaagtcacactcagctctgtttatttcattcatagagctaatcacaatttgatattaacttgtgatttttttccttatttttaaatcttatttttatttgcatagatgtatggggtacaagtgtaattttgttactttgatgtattttacagtggtaaagtctgggcttttggtatatccatcactggagtcatgtacattgtacccactaagctatttttcaacgcccgcccccttcccacctctctgtcaccttcccagtctctactgtctatcgctccatgctctactcaattttggtgtcattttttcctgaagcaaaattttggtgtcattttttctgaagtcattttctgaagtctgtgtcatttttcgctttcctgaagcgaattttggtgtcatttttcctgaagttccgtttgccccacacataggccttgcttatagaatgagggtttagtgtcacggagtctcctgcctcattctcaccctaacttttcctttacccctttgcgaggggaaggatgtccattaggttaataatgcagacccctaacccactcattatcagggtcattgtttttccactgtgcattttaatactaactgttacctgcactgctccctgcccctcaaagtgcagaaagcaaagtaacctcttttcttcccattcaggaacaattgcctattaaagtacagtcccggaggaacacctaccgctataaaggtgctgagtaacgtggaatctggattctcactgaagccctgtgccctttcagaaattggtaattgtaggactacttcactttgtgattgtggtaggtggaataggagcccccagagacgtccctgtgctgagccctgggacctgtgcgtgtgttcccatagctggcaaaagcgtttctgtcaatggcatgcagttacgggcttcgagatggggagtttactctggattttctgaatgggcccaatgtactcacagggttgagtgctcacaaggctcataagaaaaagagagaggcggaaggctcagagccagagagagaggtttgaaggtattacactgctggctttgaagatgaaggtccgtgagccaaggaatgcaggcggcctctagaagttgaaaagggcgaggaaagagtttccctgtggagcgtcctggaggaagaagccctgctgatgtcttgattttagcccagtaagacccaatctctagaacagtaagataattaatttgtgttgtttttaaccactaagtttgtggttatgcccctagagcagcagttataggaaactagtacagtgatactgttagagttataggacagtgatataggacagtgatactgttatagttataggaaactagtacagtgatactgttagagttataggacagtgatataggacagtgatattgttatagttataggaaactagtacagtgatactgttagagttataggtacagtgatattgttatagttataggaaactagtacagtgatactgttacaggtacagtgatataggacagtgatactgttataggaaactagtacagtgatactgttagagttataggtacagtgacataggacagtgatactgttatagttataggaaactagtacagtgatactgttagagttataggtagagtgatataggacagtgatactgttatagttatagaaaactagtacagtgatactgttatagttataggacagtgatataggacagtgatattgttatagttataggaaactagtacagtgatactgttagagttataggtacagtgatataggacagtgatattgttatagttataggaaactagtacagtgatactgttagagttataggtacagtgatattgttatagttataggaaactagtacagtgatactgttataggtacagtgatataggacagtgatactgttataggaaactagtacagtgatactgttagagttataggtacagtgacataggacagtgatactgttatagttataggaaactagtacagtgatactgttagagttataggtacagtgatataggacagtgatactgttatagttataggaaactagtacagtgatactgttatagttataggacagtgatattgttatagttataggaaactagtacagtgatactgttagagttataggtacagtgatataggacagtgatattgttatagttataggaaactagtacagtgatactgttatagttataggacagtgatataggacagtgatattgttatagttataggaaactagtacggtgatactgttatagttataggtacagtgatattgttatagttataggaaactagtacagtgatactgttagagttataggtacagtgatataggacagtgatattgttatagttataggaaactagtacagtgatactgttatagttgtaggacagtgatattgttatagttataggaaactagtacagtgatactgttagagttataggtacagtgatataggacagtgatactgttatagttataggacagtgatattcttatagttaccgtggtatagttatagttaaaggtacagtgatattgttatagttataggacagtgatattgttatagttataggacagtgatgtagttacagtgatataggtacagtgatattgttatagttataggacagtgatatagttataggacagtgatattgttacagttataggtacagtgacgttgtaatagttataggacagtgatattgttatagttataggtacagtgatgttgtagcaaaactgtaaggtcattccttggttgtgtccctatctagtgaaatgactctaccaggggtagggaaataaaactctgtcgtttcacacataaaggtaatttcaatggaattatccagaaaattgccatgacattccacctcatttagcatgtcaggatgttaatgacaagatgttactaaaagcaaatcccttacggccagttttccgcagtactgggtgctggctctgtgcctggccctgtattgggtgctgggctaggatttccctgtggaagattgggaaggttggttacaaggtggctattttcctgtctcctctttgcgacagcacaccttctccatggtgtgtgccaggttcacgtgtactggtgatttaattttaacgttcatattatttttttctgggagagtttttgaaggctgccaggaggcaggactcgatgcaaacatgctccattctgtacccagccctgttgctggaaggatttgctgcacttacccagggaacaggcaagctcgtgctgtggctctgggctgtcacagctgctgtccacacctgggagagcaccctggatggctcatctgtgtacttgctttcttgttaaattgcagtgagttcacatgtgatttaatcggatcaaatggcctttacagactgataaaaatatggctgtttcaggtggtgttttgagctgctaagggcgtggcttttcactgagtacgtggtccccgttcctcagggaacacccagtagccacatgcctcctaaacctagagtagggctgtctcctggcctaactgcccaaatgagattcataagttagggatgatctgtagttatcactacagatttgtccttggtttcaccaaggattttcctaattttacaaacaaaacccctaaggctcctggaaggagggtagaagtgaaggtgctccgggggcaacacagctgatgagctgaaccagaactcgacccttgggtcacacacatttcacagtgctcactccaccttttgtttttttaatggatttaatggtgttttaaagcctcctgcctctcaacacatatgaattcattatatttacagatttccttctcttgtggtccatcttcctgcatagatcttgagagatgtcaggctaaccacgtttcctcagttaatttaacaaaaccatttgcaactctgacatggaaaattcctaccatgtgacttattaatttatcaattgagatggtacacatattttcaagccaaaaggaggaaaacataaattggaaaaaaaaggtttttttatttttatcacctctggggaagaaagtctgataaacgaagctggttgataaaattgcaattaggggaagcaacatcatggtttctgttcggaggctaaccagatggcatacttgaaatagagaatgtcctagaaatcaactggttgcttggccaaaatatctataaatagtgcccaacatattagataggaaaagcaaagtaaaaacaattttaacaggttaggacattgggctgaagtattgcatatatttaatgtcatgtgcgtccgtgtgaagagaccactaaacaggctttgtgtgagcaagaaagctttttaatcacctgggtgcaggcaggctgagtccgaaaagagagtcagtgaagggagatggggtggggacgttttataggatttgggtaggtagtggaaaattacagtcaaagggggttgttctctggcgggcagcggtggggggtcacaaggtgctcagtgggggagcttctgagccaggagaaggaatttcacaaggtaacgtcatcagttaaggcaggaaccggccattttcacttcttttgtctttcttcagttacttcaggtcatctagatgtatacgtgcaggcctgggcccagaggcctgacattcctgtcttcttatattaataagaaaaagaaaacgaaatagtggtaaagtgttggggtggcgaaagtttttgggggtggtatggagagataatgggcgatgtttctcagggctgcttcgagcgggattaggggcggcgtgggaacctacagtgggagagatgaagctgaaggaatattttatggtaaggggtgatattgtggggttgttagaagcagcatttgtcatatagaatgattggtgatggcctggatatggttttgtgtgaaatgagaaactaaatggaagacacaaggtctgaataagagaaggagaaaaacaggtgttaaaggactaagaattgggaggacccaggacatctaattagagagtgcctaagggggttcagtgtaattacttgcttggttggtgagtttttgggctctatccttgacagagtcctcctttttaagttggaggctgagcttggtgaggtgtgtttttaaaagaccattagtctgttctacctttcctgaagattgaggatggtgaggggtatgaaggttttactgaataccaagagcctgagaaactgcttgggtgatttgactaataaaggccggtctgttatcggattgtatagagatggaaaggccaaactgaggaattatgtctgacagaagggaagaaatgaccacggtggccttctcagaccctgtgggaaaggcctctacccatccagtgaaagtgtctacccagaccaagaggtattttagtttcctgactccgggtatgtgagtaaagtcaatctgccagtcctgggcgggggcaaatccccgagcttgatgtgtagggaagggagggggcctgagcaatccctgaggaggagtggagtagcagatggaacactgagcagttattttttgaggatagatttttacgacggaaaggaaaagtgaggttttaagaggtgggttagtggcttgtaacttacatggaagagtttatgaaatgatgacagaatagaatgggcctgtgaggctggaggagatattttccttggtccaagaattatttgccttgtgtgggaagagattgataggtggaagtttcaatgggggagtagatgggagtgacagatgaggaagaaaaaaactggctgtgagggatagaagttggaatgctcgctgcttttttagctaccttatcagcataggcattgtcctgagcagtgggatctgatgccttttggtggcccttgcagtgaatggcttcagcttcctttggaagtaaagtggccttgagaggagtttttattaaagaggcattaagatggagaacccttgtgtagtgaggaaacctccttcagcccatataaccgcatggtggtgcagaatatggaaggcatatttagagtcagtataaatattgacacgtagtccctttgcaagagtgagggcctgagttaaggcaatgagttcagcttgctgagaggtagtggagcggggcagagcagtagcctcagtgatagatgtggaagatactacagcatagcctgcctttgctggtgagtggtgattaggcctggtggaactgccatcaataaaccaagtgtgatcagggtaaggaacaggaaagaaggaaatatggggaaatggagtggatgtcaggtggatcagagagatacagtcatgggggtgggggccagcctaaaacagtaaggtcaagttgtttgaacagaaaggctacagggcgtggtcctggctcttgtgtaagaattttgactgcgcagccctgcacttcggctgtgtgtaatgaaaagggttgggatgagttagggagagctagtgtgggagcagtttctagggctgtttttaaggaatggcaagaggagtggctaaaggatttaggatctttggggtcagctagctttgcttttgtgagtttatataatggtttagtcaggatggtaaaacttagtatccaaaggcggaagtacttaaccatacctaggaagaaaaggagttgttttgtagaaggggttggggtttgggagatgagccagacacaatcagcagggagagcacatgtgttttcatgaagaattatgccgagataggtaatggatgaggaagaaatttgggcttgactgaagtaatgggggctgtcctcgaagccttgtggcagtacagcccaagtaagttgctgaggctgacgggtgtcagggtcagtccaagtgaaagcgaagagaggctgggatgaagggtgcaaaggaatagtaaagaaagcatgtttgagatccagaacagaataatgggttgtggagggaggtattgaggataggagagtatatggctttggtaccatggggtgaataggcaagacaatttggttaatgaggcacagatcctgaactaacctgtaaggcttgtccggtttttggacaggtaaaatgggggaattgtaaggagagtttataggcttcaaaaggccacgctgtaacaggtgagtgataacaggctttaatccttttaaagcatgctgtgggatgggatattggcattgagcggggtaagtgtgattaggttttaatgggatggtaaggggtgcacgataggttgccaaggagggagcagaggtgtcctatacttgtggattaaggtggggacacacaaggggaggatgtgaaggaggctttgaactggggaaagggtggcattgaggtgtggctgtggcctaagaacagtcagggaagcggataattgagttaaaatgcctcgacctagtaagggagctgggcaggtggtgataactaaaaaggagtgcataaaagaatgttgtccaagttggcaccagagttggggagttttaagaggtttagaagcctggcggtcaatacctacaacagttatggaggcaagggaaacaggcccttgaaaagaacgtaatgtggagtgggtagcctctgtattaattaagaaggggatggatttaccctccactgtaagagttacctaaagcatctgtgatggtccaggaggcttctaaggtgatcgggcagcgtcagtcttcagccgctaagccaagaagatctgggaagcagtcagtcagagagccttgggccagagttccaggggctctgggagtggcagccaggccagttagacagtccgatttctagtggggtcccacacagatgagacacagcttaggaggaatcccaggctgcgggcattccttggcccattggccagatttctggcacttgaaacaagatcctgatggaggaggtcctgtaggaatgcttgaccactgcagtttaggcattttgaagtttttgtgtgtgctggagatgtggctgggttttgtctcacagcagaggcaaggaatcgcaactcagaaatacattgctacttggctgcctctattattgtacatcttgaaggcgaggttaattaagtcctcttgtggggtttgagggctggaatctaatttttggagtttttttttgtttgttttttggttttttttttttaatgtcaggagctgactgggtgataaaatgcatattgagaataagaggccttctgacccttctgggtctagggctgtaaagcgtctcagggttgctgccaaacgggccatgaactgggctgggtttttcatatttgatgaaaaagagcctaaacgctaactgatttgggagaggtcggataaataaaaaggaacattaatcttgactatgcctttagctccaaccacctctttaagaggaaattgttgggcaggtgggggagggctagtcgtggaatgaaactgtaagctggaccgggtgtgaggaggggaggtgatagaaggattatagggtggaggagcagaggctgaggaagaattgggatctggcttggcctggcaaggagcagcctggggaggaggggagaggtcagatgggtccatagaaaaggaggattggaaagactcagcaacacttggggttgggattgagaggacagatgggttgggattgaggggacagatgggagggaaagaaggaagatttgggacaagttgcattgggaacagagactagggagggaccaatgtgtaaaagaatgcctggacgtcaggcacctcagaccgtttgcccattttatgacaagaattatctagatcttgtaggatggaaaaatcgaaagtgccgttttctggctatttggaaccattgtcgagtttgtattggggttaagcagcattgcagaagaaaataaggcatttaggttttaggtcaggtgtgagttgaagaggttttaagttcttgagaacataggctaagggagaagaaggaggaatggagggtggaaagttgcctatagtgaaggaggcaagcccagagaaaagagagggtagagacatggagagaaggggtgggggggtgcttgcccccaggaaagtggttcttgccactaagggtgaaggatcaaggcaggcattcgcgcggtgatcagatacctctgaaacgtgggtgaataatcaagcaggtgtccctgcagtgattaaacagcaaggaaagactatcttcccaagtccatgaccagtgccagagttttgggttcatggataaaacgcgtctcctctgtctctaccagaaaatgaaaggaattgaaattaagagaagggagagattgaaggatggcgccaagattgaaaggaaaaagaggttgagggatagggagagaggttggataagagagtaaaaagaggctgcttacccaatttaaaatcggtgagatgttccttgggcttgttggtctgaggaccagaggtcatgggtggatctttctcatggagcaaagagcagggggacaggggattgatttcccaagggaggtcccctgatctgagtcacagcaccaaatatcacgtgtgtccatgcgaagagaccaccaaacaggctttgtgtgagcaagaaagctttttaatcacctgggtgcaggcgggctgagtccaaaaagagagtcagtgaagggagataggggtggggacgttttataggatttgggtaggtagtggaaaattacagtcaaagggggttgttctctggcgggcaggggtggggggtcacaaggtgctcagtgggggagcttctgagccaggagaaggaatttcacaaggtaacgtcatcagttaaggcaggaaccggccattttcacttcttttgtcattcttcagttacttcaggccatctggatgtatgcatgtaggcttgggcccagaggcctgacatttaacatggataaatgtaaagttcttagaatcatacatacactttggaaaagatggggcttaatcgcactttataagacttgaaggatgtttgagaatcactatgaaactgctgaaaataccaagaaaatttaattcttatgtatataaataatgtgtctgttttacatgaatcccttctacaagcttggtatttaatatggcatatattgttttttcatagtagatttaaaatttttgatatctaatttagataacataaaattaaccctttgaaagtgtacaactccgtggtttttagtatatccacctgattgcacaacgatcaccactgtctagttccagaacatttttatcaccacaaaagaaaggctgtatccaggccgggcacggtggctcacgcctgtaatcccagcactttgggaggccgaggcgggcggatcacggggtcaggagattgaaaccatcctgggtaacacggtgaagccctatctgtactaaagatacaaaaaaattagctgggcatgatggcagatgcctgtagtcccagctactcaggaggctgaggcaggagaatggcctgaacccaggaagcggagcttgcagtgagccaagattgcgccactgcactccagcctgggcgacagagcaagactccatctcacaaaaaaataataaaaataaaataaaaaaaaaaaagaaaggctgtctttctcctttcccattggccgtctttctccattccctactcctccaatcccctggcaaccactaaatctactttccatgtctgtggacttgactcttcgggacattttacataaatggaatcatgcaatgcagcacattttgcatctggcttttttcacctggcgtgttttcaaggctcattcgtattctagcatatatcaatactttgttcctatttaggactaaataagattctattgtatgaataaaacatattttgtttatatacttagtttgatgaacatttgagttgtttctggatttttttttttttttttttgcctcttatgaataatgctgctatggacaacagtttttgggtaggcatgcattttaaattctcttatgtatatacttaggattaaaattgctggatcacagagtaactccatgtttaactttttgatgaattgccaaactgtttttgagagcagctacacaatgttacattcttaccagcaacaattgagggcttcagtctctctacaaccttaacaacacttgttattgtctttgtaattattgcctttctaggcagtgtgaagtggtgtctcactgtggttttgatatgcatttccctaatgactaacaatgttgtgtatcttttcgtgtgctcattttcaatttgaatacattctttgggaaaatctctgtttaaatcttttggccattaaaaataattgggttattttcattgttgagttgtatgaactctttatatactctggatactacactcttatgacatatattattttcaaaaattttctgtgcatctgcaggtcatcttttcactttactgatggtgttctttgaagcaccaaagtttttaaacttgatgaaatccagtctggctttattcttgcacgtgctttacctaaaacgccaaaacctaattcatggttttgaagatttttgcttatgatttgttcttagaggtttatagttttagctcttacatttaggcatttgatgcattttaaattaaattttgtatatggtgtaaagtagcaatccaacttaattcttgcatgtgggtattcagttattccattgtcttgaaacccttttcaaaatcaattgtctataaatgtaagagtttatttttggaccatcagttctatcctgttgacctatatatgcctatcctaatgccagttacacacagtcttgattaccataactttgcagtaagttttgaactcagacagtctgagtgcttttattttgtcctttttcaagattaatttggttattccggatcttttgcatttccatatgaattttaggatggttgtcaatttctgcaatagaaaagcagcaggattttgatagagagtgcattgaatctgtagaccaatgagtatcaatggaattatgtgttatcaaatttagtaaactacataaacgatatgtacacaactaaaacaaaactaagatataagatcatattaatgtaatgataataaaagtggattgtctcctgttctaattttaataggcacaaggcatttttgttaatcacttcttattaaagaattttttataatattcaggaaaatacaacaaaaaaacccttacattcctaaggcctcagagtcagagtaatataaaggaaatgtaaacacatgctgagtaatgcaagcatttggcaatggtggtgactggagctgggagcacagcttttatttctctgaaataggaatttgccccttagagttagaccaattttgccttcctctaaatggcaaacagtttggagatactttaaaggacattttttcacttaggatgtttagtactatgaataataaatagtcacaatttccttaactatggtgacaaaatacaagcaaatttagcctcatgtcatttcctaaggaacatcttctctctgtgagttcacaggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaaggcgagtatgcatggaaaatcgcatcacaaaggcgagtatgcatggggagcacttgctgctgtactttcatcacttttatagtctgagttcttaaaagtttcgttcatttccctcaaaacacttgaacctgcagtttcagtaggtactgttctgccaggtgcagattagttaagagattagcagacttctctgcctatcttctcttactttaaaacaaatgttaccattgaatcaaggaagcaatagccatgagaaaaaagaaggatctgacgcctttgaatgaagattcaaaacatgatcttcatgttttgtattagcttggagtaaaatccacttgctggcaatatagcccttaagcttgttgcctcttctctttgtttcagaaactagagccctgtttattctgatcaaggctctggcccactgtctttatctcagataacccaccctcttctgcacacagcatggagctaagagaagggtgtctagttatgtaatatcatcggcagcataaattcccagaatttgttctttgatttttttgtttgtttttccagttagaaggtggaacttcatcattgtcctcttttcaggttgtctgtgcctattagttttctccagagggagaggtggcttgatttacatttaatctctgcaatttattagagtcctgtagttggatttactttgaagagagtttcccagaagaataaaatttgctgcgttgctttttgggtgtgagctgcttttgtatttgcctaatgcctttaatgcaaatttctttctttctctcttgcttttttttaaaaaaaatagaaatgtttgtcttcttaaaacatctgaaagtggcacaccaagttcctctactcctcaagaaaacaccatatctggatatagccttttaacctgcaaaagaactttacctggtaatgtgatttgataataatattacataaaatgtaacctatttcatgacttttaacagcaacagtgatgaaacaatcctcaaggtaacagaaacttgtgtaaatgtcgttcattgcttttcccatctgatatctttttgtgtttataattgacacagaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtaaaggaaattttatttttcaaagacagttgacatgaccatttcatattctctttccccctgtgaaggcttactctttctactgttcatttcatctaggtgtaagtgtttcttaagattatctatggatggttctccaactaggattgcgtatgggacacaagggagctctggttactctttgagattgtgtaacactggggacaactctggtgagtaacctcactttttcgtggacctgtcagggatgtctgtcatgttgatagtttgcttagtcttaaggaattatgtgtcttgttctccttggttagaagggactttgattcacttctaattccaaccattagcgtcaacgctctcttttcagtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggtaagtgttggatgcatctcatccagagtcttatcttggcttttcattttgaaggatctatgatcagctgcttcaccgccatgtgactttatgaatagagacgtgttaaagcggggatggtattcacaacatttaacttatagggtccaagcactgaccaacctgaccattagaacagagtgtggtctctgtacagggcagatggcgctgagtgggtattctccacagaaagagaaacgaagacagtaccccactcctccaacccaccacccaccaccaatcccaccaccaattccaccaccaatcctgccacccaccaccaatctcaccaccaatcccaccaccaatcctaccacccaccatcaatctcagcaccaatcccaccaccaatcccaccaccaatcccaccacctaccccaccaccaatcccgccacccacgaccaatcccaccaccgatcccgccaccaatcccaccaccaatcccaccacctaccccaccaccaatcccgccacccacgaccaatcccaccaccgatcccgccaccaaccaccaatcccaccaccaatcccaccaccaatccctgatgtgttcttcaaagacttatttgtcaggcccatagaaatgttacttcttgctctttgattcataaatatactaagtcataataatttttaaaagtgagagtttcgtactctgtatatttcaatgtatataatttgatctatttcaatttattggtcaaatagtagacatgttaggtaagtcttaaaatactgaggctttggagttagacagaacatggcttaagtgacagctttgctgcttattagaggtgtggccctagaagatttgtaaatccctctgagctttatttgatctaaaatatgaatagtaatagtcccgaatttgtaacgttgttgggaagattaagtgacacatttaaaatgcttagtactgtgtgtagaacataaacacttcaaaaaatgtaaactgtgatttctatattcaataagaaatgtagaaatggacaaagcatataaaaagcaaaagaaatactagaagacacttgatttttctcaaaaataaacacaccaagtatttttgttttagtgaaattcatgcttacatgctgtatactaggattgaacatactgccaccaaaatatagcagtcggtggtacatgtgggtggagcaagacccctccaccttgtcatcgtgtgaaggggctctgccatacatgaccttgcatgtgactttaaggtggttggcctggaagaaaagtcccaagatgggaaatagtaggtgtcttttttactaaatgcactccaatttgggaccaaaaattttcattcttgaaggctcagtattgtgagtttataagagataatagacataaaagtgtaatgatttcattgcaaataaaaaaaggcccctttgcacctgatatctccatcatttttctagaattttgtgcacacatgccttgcactacttggtgatgataaagatttccagatctttgcacagaataaggctttgctttagatcagaattttggatgtacttagtatacattcatcttttaaataatctatttacattttcatactttccaaaatacagatatattttattttatttatatatttatttaatttattttttgagatggagtctctctctgttgcccagagtagagtgcagtggcacaatcttggttcactgcagcctctgcctcccgggttcaagcgattctcctgcctcagcctcctgagtagctgggattacaggcgcgcgccacacctggctaatttttgtatttttagtagagacgaggtttcatcatgttggtcaggctggtctcgaactcctggcctcaagggatccacccacctcggcctcccgaagtgctgggattacaggtgtgggccactgtgcccagctgtatagagatattttaaacaacactaaagtcctcctactttgactaattagaagagcattagaagatcagcctgacttcttgacagttctgaatttagtggagcaatgaggttcagctttggtgaatgagcttaatttttccatgataaactgctagtttcttcccactacagtgtctctcaaaaatgggacagcaacattctttttgttttcacttgcagtaagcatgatgcaattacataaatgtacacttttcaatttgttaaatagaatcttcagagattcactactgccgctattggtgatgaaaaattaccagaaggaggaattaggtaggagaaaatgtgtcctatgtatttccttcccagttctttgaaagagagtgataggaaaaaggaacactattgaaggaaggactgcccagtttcaaacaggtatttatttttctctcctaggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactggtatgtagcatatgtaagaaggtggagagcagaattgcgctggttgatattttcatatcagtttgaacaagagggcagacctagagagactgtcgtcgttttctgactggtggagttgagggaaacgtgagggttgctgggaagtgaagaccccgcgacttgccgtgaaatctcttctacttaaagagcaagacatgtgaattaattctttcagggagggatacaactgcatgcaggtgatggaaataatgggcgtgggaaatgtctgtgccgtctgagaggcactgggcttgctttgacaagagtagcagaactgtcattgctttgggcttagggatattcgaatgtgtgagggcaagtgggatcagatatctacttccaggtataatttgggtaggaaagagactcatgcagaaagaagccctggaaggccagagcatcgtggtcagaggtgttgcctttggagggtcattgctgccaggagccgaatacccactgtatccaataacattcatggtcaggaatggtggctcacacctgtaatcccaacactttgggatgcccaggtgggaggattgcttgaggccaggagtttgagaccagcctgggcaacacagtgagaccccgtctctacataaaattagaaaaaaacaattaactgggtgtggtggtgtgcacctgtagtcccagctagtcaagaggctgagacaagaggatctcttgagcccaggagctcaaggctgtagtgagccaagatcgtgccactgcacacaaacaattatgtgacctcgggcaagttgctttacctctttacacctcttaatttccttatctgtaaaatgaggatgataatttcttcctgggtctgttgtaataattaatacatcaaagcacttcatgtctggaacagtgaagataccctgctatgactattaaggatagtatacatggaataagacacaggaacttctaaatgcttttgaccatagatttaggttctgagttttaagaatttaactcaggaaattgtaacaccaaaaatgtcatgtgaaaaatggtggtgacaaattttcttgaatcattagccttagaggttgggcagaaagcaaaaaattattcttgatgctactctatagaaagagaagacagaaaaagagaaagatgtatttttaaagtctatatccataactttatttgaccaaactctaatttaaaaattatgtttcagaattccaaaaaccaatatgcctaccttccaaaggtgacacaagcacaatttataccaactgttgggtaaccggatggggcttctcgaaggagaaaggtaagcatgacgctttaaatattgcttctagagtaagtctcacatgttgaaatacatggagtgggtcgttttaatcggtttctgtctgaaattatatctaaactctttatctttcctatctatttattcccaaatatttattcagttattcttaaaaaatgtatttttgctttggcttgaaaaaaaattttagggagacttttaagcatcttacttcattataaagatcagttgcttgactttgcatgaagcagattgggcccttctagggctgacaagcccgtgcaagaccacccgctcctcagtgttagtagcgttcccgtctcccaaaaccatgttctcccttgatgctaatggccgggagcacaggcaggtgtgtcgtctcactatggagaataatatttgtgtcattctttacagaagaaggtagcttgccaaactgtctccatctttcccgattcagtcttttgttcaagtaattcacatttttagattttttattggtaatctgagacaagaagaaatttaaagtaatcttcactaagccatgaaagctcccaacattgttctccatgagagatgctggcctgcatttattcaaaaacaaaagacccctctgttgccaaagctcggagggcttttcagaaacgatatagttgtaaattataattttgaatatataaagcaaaaaaatgaaaagtgagaacttccaggctttggattgttgtaggtgataaatataaaatgggatttctggggggctgctactgagatgaggggatggcagaaaacatggaagcaaggtctctggtcagcccagggtgctgggcttgtcccaacaccacgtaggcaataagaggacagtacagggtgccgtctctctccctcttcctctctctgtctctctctctctgtgtgtgtgtgtgtgtgtgtgtgtaacactaccttcccaatttttactgtctatttgtattcaaagataaggtccttatgaaaaatacactgctctgattcactttaaaacttatttccatatttattatttattgtgggaataataatattcccaatattattatttattatttaagttattattattaacttccctctgaggttatatattggttactcacaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaaggtaactcatgagattatgaaaaacacaataggctgcttgagaaaattcatttcaaaatatattttccaatagcataattcaatcatagtttttaaaaaaattcagagacaaatgatctgataaattgataagcaacttttaacaaattgaatatacataatatatatttatattatttatgatatatgtcacaatctatgcatgtgctatttaagaggggcaaatatacatgcaataattgtgctagaatataaaaacattagacttcatcattgggatgatgatatcaagatttctttgttagatttatttcagatagaaaaggggatacgaaaaatgcaggcacatgagatacttggagaactttaagaaagagtgagtgtgtgtgtgtgtgtgtgtgtgtgtgtgtgtgtgtctggcaagcaaggtcttgcacacacacagcactttgggaggccaatgcaggtggatcacttgagcctaggaatttgagaccagtctgggcaatgtgatgaaacccatctctacaaaaaaatatgaaagtatctgtgtgtgtgtgttgctgtgtagtggactacagaactttagaggcagtcacttatttgaatcccattgtcgtaactttctactattttatttttccactgtgactcagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatca

SEQ ID NO: 3 - DNA, 3050 bp, Homo sapiens
  source 1..3050: mol_type = genomic DNA
caggtgcatgccaccgtgcctggctaatttttgtaatttttcttgtacagttggggtttggccatgtttcccaggctaaattaagacatttaaatcactcaggcacaactaattttttcatattgccacatcttcctaggagtgggattgtgcttcagttttttcagctgtctatgccttaattttgactcccatcaataatgcttgatgttgtagccagtgttggtttattattaccaaaagcaatgctgttctggaaaggcaaggactgcagtgtctcatatgatgcaattggacggaacctcaagacgacatttgctcccagcaaagggcattcagcagaacaaggaccaatcagaattcatccaacaatcattttcagtgtcatctaagactggcaggtaatagcttacatgtgcgattgtgactttcatgtgaggatgagatggtccacaactgcggggaaagcaaccttcctgtcctcaatctcttgtttttaaaagatatttcattccttattcatgtgcttatttatttatcgttttactctttcattcataaaagtctgccaagttctattgagggagctggggaaagacagcaagcaagccccagtcctaccagcaaggtgttacagacagtgggcgtctgaaaaacaacagagaaatggacaaggaggtaacagaatggcagggagtgagaagcgttacgcagaaagacaaagaatggtgaagagacaggaggtggaagagatggaaggtgccttttcagatgagatgcttgctggaggatggtatcctgaggagatgatgtttgagcagtggtctgattgaaaatgattttattcaagcaagcaacttatttcatttccttgtttgctacagtttcctgtggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggtaattgtaggactacttcactttgtgattgtggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaactctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatcacgatta

SEQ ID NO: 4 - DNA, 42764 bp, Homo sapiens
  source 1..42764: mol_type = genomic DNA
caggtgcatgccaccgtgcctggctaatttttgtaatttttcttgtacagttggggtttggccatgtttcccaggctaaattaagacatttaaatcactcaggcacaactaattttttcatattgccacatcttcctaggagtgggattgtgcttcagttttttcagctgtctatgccttaattttgactcccatcaataatgcttgatgttgtagccagtgttggtttattattaccaaaagcaatgctgttctggaaaggcaaggactgcagtgtctcatatgatgcaattggacggaacctcaagacgacatttgctcccagcaaagggcattcagcagaacaaggaccaatcagaattcatccaacaatcattttcagtgtcatctaagactggcaggtaatagcttacatgtgcgattgtgactttcatgtgaggatgagatggtccacaactgcggggaaagcaaccttcctgtcctcaatctcttgtttttaaaagatatttcattccttattcatgtgcttatttatttatcgttttactctttcattcataaaagtctgccaagttctattgagggagctggggaaagacagcaagcaagccccagtcctaccagcaaggtgttacagacagtgggcgtctgaaaaacaacagagaaatggacaaggaggtaacagaatggcagggagtgagaagcgttacgcagaaagacaaagaatggtgaagagacaggaggtggaagagatggaaggtgccttttcagatgagatgcttgctggaggatggtatcctgaggagatgatgtttgagcagtggtctgattgaagtgagtcacgtggggatctgggacaaggggaggtggagagggctccatcctggcacgtgtctaaggggaggttctggagaaaaatcctctttcaagcacgtgcagtttgttggaatattcagtgctttgggatggtaggggtgaaatccccatgttctttttggctcttgtccagagatgactctcaggtccttccccgtgacccatctcaaagccagcgctggagcacttccctgctgtcaatccctcttaacgctctgaatctctctgacatctccttatgctgaccgcagaggaaaactctgcttttaatgggctcatgtaataagatttggcccactctgattgactccattttgattggcacaaagccaagtgtttagtaatcttaaccccatctgcagaattgcttttctcatgtaacataatcatgggcagatgtgatatctcgttttattcactgtccccaggatttgagtgggagatttgggagaatgttttagaattctacttaccacgcctcttttcaatttagttctgatttggttcacctgaatctacagaggtttttttagttttattgtgctactagaatgtaacatgtttcactgtaagaaaactccaactaaacctagaagcatatttaaataggttaaaagtctgtaatctcaccttttagaaaaacaaaccctgagggtttctgctgtgatgtgaaggcagggtgcttttgtcccagttgttttgttgttgttgttgttgttcttgttttctgctgagcctggtagctgatgaaatctaaaatataaactcttttctactgcacaaagtggagagatttgttgactactgctgcaccacttgatatcctaaaagacagcacaacatgcatatttaaaatatgctacttccttctcaccagaaaatattttattggcaacattatcctacatttctaataaatgtagttctgcattccagtaatatacataaaatatttatttaagaatcgactccaaaaactctaatgtactaatcaagatgatatactgtaatttcaaagaatagaaaacaaaggagatttagtgggcaggtcattgtggtctttaaatattttaaaagcagaaaaaggagaatcagttgttttgtgtgggtctagtacgtaaatctaggactgaaggatagaagtttgcaaggaggtgtatttcagtatacaggaagtttttttcttttcttaaaaataaaattaaaatagagctccctgagctctgaaataaaataaaattaaaatagagctccctgagttggagcaggctgcttaggaaaccagtttctcctcagtcgaagtattttgacaggaaggtctaaagtctatgtcagaagtgttatccagcagatttcagccaaccttatctgtagagtcagatagtaaatcttcttggcttttgaggccatgctgcctctgtagcaacttcttaactctgccattgtaaggtgaaagcaggcatagacagcaagtgaatgaatggccatggctgtgtttcaataaaactttatttacaaaaacaggtgcaaagccaaatgtggtctgtgaactgtggtttgtcaagcctgatatattgcatttcctgaattgggtgggaataggacctaaacatcaagccagtcccttccaaatctaagatttcagtgttatatgattctttggtagggtgtaggaaggcatagatatcatttgctaaaatatccctctaggaggagctatccatacccatccattattcactgtgcctgcaagcaactctaataaaaaaaagttctgttagatgaggtttcaactacatgccactaaatactgggaatgttataagcattttttaaaatggaaacctgtgcataaactgtgtgaccctaaaatgaaattagggtccttttgagtgcagcatttttctacctttttttatgtatcatttacaaataaaaattatatatatctactgtgtacaagatagtgttttgatatacatgtacattgtgaaatgattaccatagtcaagctaattaacatgtccattattttacatagtgtgtgtgtgtgtgtgtgtgtgcgcatgtgtgtgtgctgagaacccatcagatctattctctagtgaagttcatgtatacgatacagtattatgaactatagtcaccatgtttgcattagtcagggttctctagaaggacagaactaataggatagatgtacatataaaggggagtttattaaagagtattgactcacacgatcacaaggtgaagtcccacaatagtctgcctgcaggctgaggagcaaggaagccagtctaagtcccaaaacctcaaaagcaagaaagtcaacagtgcagccttcagtctgtggtcaaaggtccaagagcccttggcaaaccactggtgtaactccaagagtccaaaagctgaagaacttggagtacaatgttcaagggcaggaaacatccagcacaggagaaagatgagggccagaagacttagccagtctagtccttccacgttcctctgcctgcttttatcctagcccggctggcagctgagatggtgcccacccagattgagggtgggtctgcctctcccagtcaactgactcaaatattaatctcctttggcaacaccctcacagacacatccaggaacaatacttggcatccttcaatccagtcaagccgacactcaatattaaccatcacaagtccaccccttgtctacttgaacccatacgcatctcctgaaatcatatataatcttcaaataaagataataataaggtcataattatgttaggtaatacataatacagctatccttcgtacaaccagaagtgtaccaatccccaacccaaatgctgttacataaagttaacaacatttaaatgctgatttgaagtaaataaatcttatgtcacatgataaaggaaaaagaaaggaaataaaatgaagatattttcttagtacaagtgtatacatacacaaacatgttcctaacaaaataaggaggaaatatgacaattacagtcctcatttctgcagctggtcacgtgggcatagccggtattgatgactaccttcttctactacccattctgtattccttttgccttcagcaagcatctcagcaggttgtggttttttacctggtggagtgacccaaacattcattcctaaggggtctgggccttatgtagcactgcctggattgggctgttgtagtttcccattgaccttaatcacagggcatggtaatactaagagttgcctgaagggatctcctgtattccttgcatactcttccttacctccactgtggagtaatagactaacttcatcttgatagtccaggtcaatcaccacagccaacactgcaactcccttctgagcctgttgacttagaggtaaaaggatctcaaagtgtccaggtggcaagcttaacttccagtttaatggaatccttgttgtgtcttctggtggcagcatttttccctctgaaactaacacaactaagcaagcagaaggtaatgtattgggaacaggaagcaaaaattttgctagtggatagctaggggtgatggtgagtggtgccacttccacttctgccccttgattcctagacctgtgaatcctggctatgggagaaacggtgccatatattggatgttgattcagagcatacatagccttctggagaattttgccccagccctgcaaagtattgtcccctagttggcattgtaattgtgacttcaaaaggccattccaccgttctatcaatacagctgcttcaggatgacgggaaacaatgttgtacattagatcctccaacttattcacactgcataattggaactttggaccttttgaccaaaaccaccccatagctggcactccccatactctggctaccaccattctactctctgttagcatgactttgacttttttagattctgcatgtagttgtaagtgcctctcttcagatgatcatatgatttttatccttcattttgtgaatgtggtatatagcatttattgagttgcatatgttgaaccatccttgcatcctaggctcagttgatcatgatccttttaatgtgttgttgaatttagtctggctaatattttcttcaggatttctggatctatgttcatcaggaatattgatctataattttaattttattttattgtgatgtccttgtttggctctgctatcaaagtaatgctggcctcattaaatgagattggaagtgtttcctccttttcaattatttgaaagagtttgagaaggattggtgttaattctttaaatgtttatagacttcaccaatgaagacatcaggtgctgagcttttcttagttgagagtttttatttctgattcaacctccttactcattattggtttgttcagattttctatttcttcctgattcagtcttggtaggttgtatgttcctaggaatttatctatttcttctacattatttaattagttgatgtactgtttttcatagtagtctcatgattttctgtatttctgtggtatccattgtaatctttcctccttcatttataattttatttgagttttttttctttttctcctaattaggctaaagatttcttgatttatctttttaaaaacaataacagtgtcattgaccttttctgttgtttttctagtctctatttcatttatctctgctctgatctttattattttctttcttcctctgtctttgggcttagttttttttttttactttcttattgcttcaggtgtaaagttaggatatttcagatctttccattttcttaaggtaggtatttatcgctataaacttccttcttagaactgcttttgctgcatcccataaattttcaatgccacaaattttgtttctcttttcatttttctcaagacattttttgattttccttttgatttcttctttgactcattggttgttcaggagtatgttaattttcatgtatttgtgaattttccatttttccttctgttactgagttctacttttatgccattatggttggaaaagatacttgatatgatttcaatccttaaatttgttaagatgttttccatggcctaacatataacctatcctggagagtgttctgtgtgcacttaaaaagaatgtgcattctgctgcttttgaaaggaatgttctgcacatgtccgttaggtctattggtttatagcattttgaaatccacagtttccttgttgattttctgtccaggtgatctattcattgttgaaagcatgttgctatttatttctcctttagttatgttaatatttgctttatagatttagatgcactgattttgggtacatgtgtatttacaattgttatatcctcttaatgaattcacccttttaccattatataatgaccttctctgtctcttgtgacagtatttgacttaaactctatctcatctgttataaggatagccacctctgctttcgtttggttatcatgtgaatgcaatctctttttcatcccttcacttccagcttatgtctgtccttaaagctaaagttagtctcttacaggcagcatatagttagatcttgttttttattcactcatccactctctcttttgattggataatttaatacatttacatctaaagtaattattggtaggtaaaggcttactactatcatgttgttaattgttttctgattgttttgtagttcctttgttgcttctttttgtctcactgtcttcctttgtgacttaataacttttttggagcaatttgctttggttcctttctctttatgttttgtatatctactacagactttttctttgtggttatcatgaggtttacataaaatatttcatagttataaccatccattttaatctggtaacaactttgactgcatttaaaaatactgtattttaacttctcctcctcccacattttattattcatgccatagtttatatcttttgtatgttgtgtattcattagcaaattgttgtagttatagttatttgttaatacttttgttttttaacttttatactagagttaaaagtgactgttgcacaataacagtattggagtattctgtatttgactgtataagaatacctttaatagagttttattctttcatttgttttcatgttagttagtgtccttttgtttaaacttgagaattccctttagcattttttgtaaggcaggtttaatggtgttgaaatcccacagcttttatttgtctggtgatatgatttgttcctgaacttccacccaaatctcatctcgaattgcaatccccaggtgtcaaaggagggacctgttgggagatgattggatcacgaggtgttttcccccattatgttctcatgatagtgagggtgttctcatgagatctgatggtttaaaagtggtggttacccgtgcgcacatgctctctctctctcctgctgccttgtgaagaaggtacctgcttccccttcaccttccaccataattgtaagtttcctgaggcctccccagccatgaggaactgtgagtcatttagacctcttttgtttataaattacccagtctcaggtgatatcttttagcagtgtgaaaataaacaatacagagaattggtactggcagagtgaggtactgctataaagacaacctgaaaatgtgaaggcaactttgaaactgggtaacaggcagaggttggaacagtttggagggcttagaagataggaagatgtgggaaagtttgaaacttcctagagacttgttgaatggttttgaccaaaatgctgatagtcgtattgacaatgaagtccaggctgagatggtctcagatggagacgagaaactattgggaactggagcaaaggccattcttgctatgctttagcagagactggcagcattttgcccctgccctagagatctgtggagctttgaacttgagagagatgatttagggtatctgtcagaataaatttctaagcagaaaagcattcaaatataacctggctttttctgaaagcatacagtcaagtcatatgcgttcacaaagagatggtatgaaattggaacttatgtttaaaagggaagcaaagcataaaagtttggaaattttgcagcctgaccatgtggtagaaaagaaaaaccaattttctgaggaggaattcaagccggctgcagaaatttacataagtaatcaggagctgaatgttaattgccaaacaatggggaaatgtctccagggcatttcagaggtcttcaaggcagcccctcccattacaagcccagaggcctaggagggaaaaatggtttcatgggccctgctgctctgtgcagccttgggacttggtgccctgtgccccagccactccagctccagctgtgactaaaagggtccatagctcaggctgtcgcttcaagggtgcaagccccaagccttggtggcttccacgtgctcttgggtctgcgagttcacagaaaacaagagctgagctttgagagcctctgcctagatttcacaggatgtgtggaaacacctggctgtccaggcagaagcctgctgcagggacccagccctcatggagaacctctattagggcagtgcagaggtgaactgtggggttggagcccacacacagagaccccactggagcactgcccagtggagctgtgaaaagagttcccccatcttccagatgccagaatggtagatccactgactggaaagttgcaggcactcagtgccggcccatgaaggcagccgtaggggctgtaccctgcaaagccacagggacagagctgcccaaggccttaacagcccacaccttgcatcagcatgccctgggtgtgagacaggggctccaaggagattattttggagctttaaaatttaatgacttccctgctaggttttgcatatgcttgggacctgtggcccctttattttggccaatttctcccatttggaatgggaacatttacccaattcctgtacccacattgtgttttggaagtaactaacttgttttttattttaaaggctcattgagggaagggacttgccttgtctcaaatgagactttggactcggacttttgggttactgtggaatgagttaagactttgggggactgttggaaagacatcattggttttgaaatctgaaaaggacatgagatttgggaaaggccaggggaagaatgatatggtttggctctgtgtcctcatccaaatctcgtcttgaattgtaatccccacatgggattacatgggaggggcctggtgagaggtgattggatcatggaggtggtttcccctgtgctgtgatctcatgatagtgagggatttaaaagtgacagtttcccctgcacatacacactctctctctcgtatcaccttgtgaagaaggtgtctgcttcccctctgccttccatcgtgattataagtttcctgaggcctccccagccatgggtaactgtgagtcagttaaacctcttttgtttataaattacccagtctcaggtagtatctttacagcagtgtgaaactggactaatacatctgggaaattctttttgtctccttcctttctgaaggacagctttgcctgatattatattcttggttgccaggttttgtactttcaatactttgaatatatcatcccactctctcttggcctacatgatttctgctgagaaatccaccaataatcttatgaagcttcccttgtatgtgaagaattgcttttctcttgcttctttgaaaattctctctttgtcatggtgaggatttcttttttttttttttttttttagatggagtctagctctgttgtccaggctggagtgcagtgtgcagtggcgcaatctcggctcactgcaagctccgtctcctgggttcacaccattttcctgtctcagcctctggagtagctgagactacaggtgcccaccaccacgcctggctaaattttttttgtatttttagtagagatggggtttcactgtgttagccaggatggtctcgatctcctgacctcatgatccacccatcttggcctcccaaagtgctgggattacaggcatgagccactgcacctggccgaggatctctttatatttaatctatttggagttcttttgggcttcataaatctggatgtttatttcatttatgtctccaaattttgagagttttctattcttatttttttaaataagtttctgcaactttctctttctctacttcttctggaactcttctaatgcatattaattttcttaacagtgtcctataagtcttgtcagctttctgcaatttttatcattccttttattactctgactgggtaatttcaaatgacctgtatctgagcatgctgattctttctcctgcttgatctagtctgctgctgaagctgtttgtgaattttttcaattcagtcattttgttctttagctccagaatttctgtttcattcttttttatggttcctatctatctttttgttgaacttctaattttgctcatgtattgttttcctgattttgtttactgtctatctatattgttttgtagcttattgagctttcttaaggcaattatttttagctcttgtcatgcagtaaagatctccatttctttagggtcacctcctgctgctttatttccttcctttggtggtttcttgtttccccgattatttgtgatcagtgtggccttgcactgaagtaggcacctatttcagtctttacatactagcttcagcagagaaagccattcactagtcagattgaccagagattgttggtgggctgtctgttggggtctatgcacaggatttctgctggagttctaaggtgggaagggctggattctgggttcattcactgtggttgtctgtattctgtgcacaaggactggcttgaagcatggatccttgggggctgacttggcactgaaatgagccttaagcctgcgtctgcaggggccagcctaacatggggatcacctggcacctgagttcatggggataggcctgttcctgagtttattcaggctgtcctgggaacaaggtccactggggtgagcccagcatctgggtccacatggcccagcatggagccaagatctctggaggctgacctggtgctggatctgcaggggatggcctggattctaggcccatgggtgccaacttggagcctggggttgctggggctaacgtggaggctagatagagtcttgggggccaggctagagctggagcaggcctgaagtctaggttttgtgtggccatcttggagcctgaagccccaggggctgacctggtctggggtgagcatggggctgaggccacagaggctggtctggcctgtggcaggcctgaatcctggtgctggggtttactggagtgggcttggtgcttgggatctgtggtgaagttaggttctatcttaactgtccctcctccatgcaagagggcatctctctccatactgtgctgcccaggcttgaaggtgagatgacaccggtaatgtgaaattgtccttcctatacacttctatgtgtcttttcttatttctgtgctgcaaccaggtggcataacctctcacctgattccttagctctagtgaagttattttcgtgcatggatacttgttcgaattgatgtttctgcaagggatgagcgctagaaactcctgttctgacaaactcctattcctattcttgctgacatcactccctgaaatagttaatatacttaacagctgaacacggataggatgttcatggaatatgttgacaggacaaaaagttgaaactgttggcagaaacccaaagtcaatattgaagccaagcaaaatattgcctgcagtgccacattagaacagcttgaagaccgttcatttttaagtgacaagagactcacctccaagaagcaattgtgttttcaggtagcaaatttttattattctgattgtttccaaataaactataatttttaagtataattttttactttatgagaaaattaatcatttatattctaatttcctgagtatgtagagagtatagataatgttcctttatgtagaaatatttaaatgtaagatgattttaaatcagaaagaatatttgattgatttaaaatttttaaatgggctttaatattttcagaggttttctttacttagggatttttggactgacattattgccattatttattaattttgtttttgcccaaatcaagaggtttcataattgtttactctctctctaccaattccctttccaacattactagccacagagttggccaatgaacaataaacacaacagtagtctggaggtctcaatttgtatcttgggaagcattataaattttccaactccctagacacaaatgtaccaaaaaaaaacccttgttttctataccagtaattgtgtgctttgtcttgcaattcagacatttacaagaaaatctaaatcaccttaaattaagatttatgttaaatgtggtctaaaaccagcagagttatgtattgttttcttttttagaatgattttattcaagcaagcaacttatttcatttccttgtttgctacagtttcctgtggtaagtgaattatctataaaacatggaattcaggctaagacaggagtagccaagcaagtggcaccacccctggagaaagctattgaacatacagcttcgggggtggagattgtccctgatgattcaggacacgtgtctatttaatgttccacaacaaggaccacttgtcaggtatattgctgtagacatatgttgcagaccagaggaaggagctcagaagtaggaatgtcttgggacttgtgttaacaaaaacttctgttcgcagatgacactctgcaaagcaaaacttgaaacaaaaaaaaattagtcctctatttttattatcaacagtaaaaaattaaactttatctgaaaattcaaaagagtgctaggcattttatagtgtctgggtccaatccaagtatctgttaggaacaccatacatagttttactctggaccgctagggaaccatttcaaaaatgaaagtaactggtttaaatttaacttagcaaaccatgcatttggatagttctaggtgaatagctttcaacaccagatttagatctcatttctctattaatttcattaatttttggagaataaaaatgattctggacatttcattaatcattacagagggagttttctctgtgtccccacaaggcatgattctgggtctatggtgacttaagagggccacacaacaatgagtatttaattttcctcagatgtatggctacaataaacatctataggtaaggtttacattcataaagagacctttttttttccaggaaaaaagacttttattccccctaaatcacaactcccctgtgtctgtccctcaaccctgatttctcttctaaaccgtaatttacaaacccatgtgcaaacccactgaaaggtgagaaggagcataagccagagacactgggaaccacagccaactacagggggtttttcatttttttgttttgttttgttttttggcaaaataaatcatgattatggctaggaaaatcagggatgtaagtaagcaaaaatttagaaactacatattgcatgtagtccccaaattcagaaacagtcatcgttaaacatcttttatttagtctttcagatatttttctacatatacctatttgcacatttcacaaaaaagagttattaactgtggacactctttgacttgcattttccacttatagctatcttttggtgcaaataagtaatatattttggagcttgctattcttctctttttatggtatttttgtgtgcacattcttatgctcttattcagttatttctgtagaatacaatttttgaaataaaaatactagattcaaaggtgtgaatatttttgaaggttttgtggtgtggtatcatgaaattaggtttcagaaagtttactccatagttgactttttctaccagctaataagagtgctcatttaccccacttaggccaactctaacaggcttctgttgctttgcatcctgacatatttatctttctggaaacagtgtcctaatttatttctgcagagctactgcctctcagttttggtccatgtggttctggtgaccctgattcttctgctgaaccagaaagtgcacacatcctcctggctgtggtgacccaatcagagccaacgtcttgcaatgaagcttttctttagacatctagaaataagactcttatgttttttttttccaatttgacttgaaacttaagacaatacacaggcctgagttgttggtactattttgctactacgtggagcttgagagtaatggtaacaccaagactggagcaagtggaaggagacatattgtccactgatgctatcagttgagcacaatgtgcagttactttttaagccagattcaccatgggctttttggctcctgaaccaatagatttcatttgttttcaagccactttgtactaggttttccatcatttgcaatcaaaaccagtatacaaacactggttatttaatttttcatttttgctaatctcagggataaaatggcttctagttgttttaacttggctatatttgtgattcttcccattttcatatacttactaatcacttctatttcttttgtaaattatcttttcatatttgttatctaattctttaaaatttgagtactttaattctcattattgtttgtggaattgttaaacaagactgaataatctgcccaaagtccatggacatgggggcccatgtaaaagctttgaaagccaccatcattatgagataaattatataatagtactttacataggctccaaaatacagcacagacacagctatcattgtcatggtcatcattatcatcatgatcaccaacttatgaggataagcaagaacacctactagaagtttctttccattcagcaacaaaattggtgtctttctagtcactcccttccctgactgtcacatagcagttcacagaggctcagttgcagaatgagaagctctgggccaggacctgccattgtatgcatccttgcatgggaactgggggctggaagaggagtgactgcttgataattatgagtcagtcaaaaccaccaactgtctgaaaaaaataggcctttttgtaactagtattgtcactaaaccaactcctccatgttttgtgcatacatgaaatctaggcaatacacttgtattcccaaaagcttccacttgaagagatctgtgctctttccaaatataaaccttacccgagaggtggtcatcttggccacacctcagagagggagagaggcagtcttgttgggttgggtggtcataatggggctcagggccaaatccccagggggtaggatagtgcagagaagatggcactctccagtgcttaataaaatgcacgtggtctaagctgcccactccctcaaaggcaataaaaaataggtactatttaaattgaagagtaactactgccccagggaatggacaggttgtcattggaatagccatggttaatggtcccagttgacaactgaaatgaatgtgctacctgaacaggaaagcttattaaccagatttcaaagacagtctttcccggtaaatccaaatttacaaattaaagccagtaaagacaccgaattctctaataatatgtggggtgcagtatattttagagctgggaataattccaaacagcaaatagttcaaaatttattttcaatttagcatatgcatgcatttgcttaaactgtcttaaaaatgagtaaaaaatactgtcagtttgtttcaatcatactgagatgaggaacaatgtttagcattgcatgctagaaaaggacacaggattgggagtcagggctgggtgaccgtcacaggactttcactaactgtgtggatcttgggcaagtctgtgtgccctgagactcagttatttaactttcttttaaaaaacatagtccagatgcagataacaaagcctgcctctaatttcccttacagaattgtgagaagctggtggagatgtttgttacaaaagtgttttgaaaatagagcaaatattattctttttaaggcatgatgttttcatagcatgtcaggcaacaggaaaaaactaagttaggattttattttattgtggggaatttatgtgcaaattattgtgcaatttaatgaaaataagccaatgttttatacagaagtacccagaaaattaaataacactatacattgttcaaatagttgccttaatatattttattttctcagcataattagagttgtattatacaggtctttgagtagtcagtcagtgggagaagttaagacaacagatatctttttattaaaattattatatgaattatcgcaaattaatttttatggttctgtcacaggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtaaaagttggtatttcattattggagaagctgtttttcaaaactgaatcagttttgtgcagaaaggtgtagtataactgagagttcttcctcacacggggttcaaggaccagcttcagcaaaatcccgtcaagtggttcttacaaatgcagattcctaggccacaacccagatctgctgaaccaaagtttcttgtgaccaggaatctgcattttaaacaatcactgtgtttctttaaagtagtagaagctagtcattttctattcaaagcctcaaaatgcttgaatatcattgggctaagggattgtctcaagaaagagtctaacaggtgcacatttcatctgaataaagaaacagatttaactgtgtgacccatgatcacattagcggatagcacagtccaaagaaaataacataagacaagcattttgctgagaatgtaattgagaaagatctagaacttgtgattttgggacagggcagttctaaatggggcctatagtgagccagtttgggcacctgtggcatgatgctatgtatggtgtgtgtgtgtatgtgtgcttgtgcttgtgtgaatatgtattattaactggaaatttgtaaaagtattggaaaaatagtacttacgattttgtgtgtgtgtgtgatggagtctcgctttgtagcccaggctggagtgcagtggcgtgatctcggctcactgcaacttccacctcccaggttcaagcgattctcctgcctcagcctcccaagtagctgggattacaggcacgcaccaccacgtccagctaatttttgtagttttagtagagacggggtttcaccacattggccaggctggccttgaactcctgacctcgtgatccacctgcctctacctcctaatgtgctgggattacaggcatgagccaccgcacccagcggtaattacaatttttattaggtcagagagatgcttattaatcacgagccacagtttcatcttaatgatttttccttttgattaatatccagagtaagcttttctttgttgttcccatttccatgttcataactctttactcatcttcactctatgtgagtttaccaactagaaattggatagtcattctctgatcccacatgttaaacttgtagagaaaactcagattgtatgtgaggatcatcatattaaaagtggaggaaggttctagaattcttataaataatgaaattaacatgaaggtggacatctaagacagagggaagtcttccattaagtgcagactacaaggagttaataagcaagatgaacacgatatacaaatccagctcttatcactaagttaactttttaagtaaatgaaagtatttgcaaaaataattaccaattgagaacatagttgcctgaaagtttaagaacacaggaaaaatcattaactctttaatatggttgatttcctgtacttaaaaaatgtgagtgtaaagaaaacgcagtgatggagttagatattatggggtgttataaaattagctctaagagtgttctttccagcaagtattggggaagctatattattttccttattcctggttttatttgttagtgtgtagaaaatgctagacatttcctcaatgtatgtttattattctacttcctaagtaaagctacttttaaaataggtttggttgcttcttgaaagatagtgttacaggaaccctgccaaaagtacatcgaacaggtgcagtttctggacattccttgaagcaatgtggtcatcaaataagtggtaagttgtgaatttcttagctacatttgagttaatattggatctcgcttagaacagcttttgctcaaagtttgtactgctacagctttttggaaggcatcactcataaagataggagatggggcagtattctggacacaaaagagggacccatattcatctggacacttctattgtctttataaatcaacacatacttaatgagcctctattatttatgaggttagcgctcaagtgtaagatttgcagaaaatgaatccaaataattgtgtctcgtttccagataagaatttttaagaaaacacaagggaacatctctctcaagttcacttgagggtaatttttacatcagtgattctcaaccagcagtgattttgtctcttccactggggacatttgacaatgtctggagacatttttggtggctacaactagggaggatgctattagcatttactaagtagaggccagaatgtttgaatgctgaccaacattctacaaggcacagggcagtcgtccacagcaaataattttctggcccaaaacgtcaacagtgctgacatcaagaaactctgtgatataccactaggcccaaattgaagaactgagttctgcaaatcttgctaagaataatacttcctaaaggaaacttgaggactaggatgctagagaactttgattctgacatctgaagctactgatgtcttgggaaacagtttccaatgctatcctaataaatttaagacaaatgaactatttctcaaacatgactgggactgataagaaagtgaaaagtgctgaaaagattcaactgatgggttgtcagaatcttaaaataactgctgttattctatgtatgactatatatcattactattttattttcattatgcacaattaattttgtaggttcaaatttcagatgtttttaaatttgtcatcctttcctccctcattgatatcacctcttcaatacgtacacactttgagcctgctgtttgcattttaaccagttatcaaaggatggcaatgccttcattataaatgtgggcctgacttagccagtataataggtgtagtctacgtgaggtggagtacatttcctattttaaaagatcaatttttatgttaatccaatttggtataaattattcgagtaagtgctatttctgattgttgtatcttgtagcaaaatttaaagaaaaagtaatttgtgcctttctcaatattcctgttattgttcatgtattctaaaactcactgttactcacttagcttgcttttaatgttttttaaagtgaaaaattgttcccaagtacataaaatctctacactcaagaacaattctagtcaaaagcatttagagcttccgtatgaacacttaaagagtttttatttgtaagagtcgcatcccaactcttagcctgttcttttctcacatgcagaaaaataggaaagagacttcgtttccacagtctgcaaattcctgtgtttaagaaccacagtgaataatccacctccctgcccaactcatcgtactgtcatatagttttcctgacagtttgtgtattttctgtctttcccacccttaaatttagttttatgacttcaaccatacttcttaggagtggaaaggtatctgtagtagattatggtttattccacataatcttggggaataaaactttaaaaaagtatacagtttatacttctggttacattacttccttaaccaaaagtctaaccaagaaatttgaatctttaaaaaaaaaagaggccgggcgcggtggctcacgcctgtaatcccagcactttgggaggccgagacgggcggatcacgaggtcaggagatcgagaccatcctggctgacacggtgaaaccccgtctctactaaaaatacaaaaattagccgggcgtggtggcgcgcgcctgtagtcccagctactcgggaggctgaggcaggagaatggcgtgaacccgggaggcggagcttgcagtgagtcgagatcgcgccactgcgctccagcctgggcgacagagcgagactccgtctcaaaaaaaaaaaaaaaaaaaagagcctaattttgcttcactgtctgtgaaaagaattatctgtatcttttgcatgtaagacaaatctcaatgaaaagggtgcttaaatagaagttaacactattttaaagcaagaatggaagtggtttcatcatgcgtaaacaacaactctccacattttgtaatgattgatctggatgcaatttgtcatcagacaggagaagtcgaaagcaaagaaataacactgggagatagagaagctctttcattcaatgcgaaaggtcaaaggcacatcagtttctttaataatgcaaacctcagcacacattatcagtgtcctcattattattgccttgtttatttcccactgctcattgataatttcaacgtgaaatttacctgtattgctgcatgcatcttgcagtttaagaagtgaagtaacccaatttcaaagctagtgctttagggaaaatattggattgtatttacttcaagcagagttcgataatttatgtacataataaaaattttaaatcccttagttaatatagcagttgccaaaactgggctattatcattctaaactaccaacccaaatggtagtgggtatctaatctacctctagaaagaaaatggactgtatttgctctatgtatttttcttgtacagcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggtgagtacaattcaaggtgtgtgttctttgtattggtgcctccaggatttcactgtattcttcttaacctcttttgttcccaaactaaaaaccaaacagggcttttattctaaccactttcctcatttacttactctattttatttttttatcttttatttatttatttatttattgagatggagtcgcgctctgtcgcccaggctagagtgcagtggcgtaatctcgactcactacaacctccgcttcccgggttcaagcgattctcctgcctcagcctcccgagtagctgggattacaggcgtccgccaccatgcctggctaatttttgtatttttagtagagtcggggttttactatgttggtcaggctggtctcgaactcctgatcttgtgatccacccacctcagcctcccaaagcgctgggattacaggcatgagccactgcgcctcgcccttcattttttaattaaataattcatttaatttcatttgtttctctactcttttcccctggcatgtaattgtaccgcatcttcaaagcctgacatcctcttcccctacttctccaaagctgattcttgcaggtctttcctcaaataccgtcccctccaaaagcccatttctgagcattctcttttaagtcacactcagctctgtttatttcattcatagagctaatcacaatttgatattaacttgtgatttttttccttatttttaaatcttatttttatttgcatagatgtatggggtacaagtgtaattttgttactttgatgtattttacagtggtaaagtctgggcttttggtatatccatcactggagtcatgtacattgtacccactaagctatttttcaacgcccgcccccttcccacctctctgtcaccttcccagtctctactgtctatcgctccatgctctactcaattttggtgtcattttttcctgaagcaaaattttggtgtcattttttctgaagtcattttctgaagtctgtgtcatttttcgctttcctgaagcgaattttggtgtcatttttcctgaagttccgtttgccccacacataggccttgcttatagaatgagggtttagtgtcacggagtctcctgcctcattctcaccctaacttttcctttacccctttgcgaggggaaggatgtccattaggttaataatgcagacccctaacccactcattatcagggtcattgtttttccactgtgcattttaatactaactgttacctgcactgctccctgcccctcaaagtgcagaaagcaaagtaacctcttttcttcccattcaggaacaattgcctattaaagtacagtcccggaggaacacctaccgctataaaggtgctgagtaacgtggaatctggattctcactgaagccctgtgccctttcagaaattggtaattgtaggactacttcactttgtgattgtggtaggtggaataggagcccccagagacgtccctgtgctgagccctgggacctgtgcgtgtgttcccatagctggcaaaagcgtttctgtcaatggcatgcagttacgggcttcgagatggggagtttactctggattttctgaatgggcccaatgtactcacagggttgagtgctcacaaggctcataagaaaaagagagaggcggaaggctcagagccagagagagaggtttgaaggtattacactgctggctttgaagatgaaggtccgtgagccaaggaatgcaggcggcctctagaagttgaaaagggcgaggaaagagtttccctgtggagcgtcctggaggaagaagccctgctgatgtcttgattttagcccagtaagacccaatctctagaacagtaagataattaatttgtgttgtttttaaccactaagtttgtggttatgcccctagagcagcagttataggaaactagtacagtgatactgttagagttataggacagtgatataggacagtgatactgttatagttataggaaactagtacagtgatactgttagagttataggacagtgatataggacagtgatattgttatagttataggaaactagtacagtgatactgttagagttataggtacagtgatattgttatagttataggaaactagtacagtgatactgttacaggtacagtgatataggacagtgatactgttataggaaactagtacagtgatactgttagagttataggtacagtgacataggacagtgatactgttatagttataggaaactagtacagtgatactgttagagttataggtagagtgatataggacagtgatactgttatagttatagaaaactagtacagtgatactgttatagttataggacagtgatataggacagtgatattgttatagttataggaaactagtacagtgatactgttagagttataggtacagtgatataggacagtgatattgttatagttataggaaactagtacagtgatactgttagagttataggtacagtgatattgttatagttataggaaactagtacagtgatactgttataggtacagtgatataggacagtgatactgttataggaaactagtacagtgatactgttagagttataggtacagtgacataggacagtgatactgttatagttataggaaactagtacagtgatactgttagagttataggtacagtgatataggacagtgatactgttatagttataggaaactagtacagtgatactgttatagttataggacagtgatattgttatagttataggaaactagtacagtgatactgttagagttataggtacagtgatataggacagtgatattgttatagttataggaaactagtacagtgatactgttatagttataggacagtgatataggacagtgatattgttatagttataggaaactagtacggtgatactgttatagttataggtacagtgatattgttatagttataggaaactagtacagtgatactgttagagttataggtacagtgatataggacagtgatattgttatagttataggaaactagtacagtgatactgttatagttgtaggacagtgatattgttatagttataggaaactagtacagtgatactgttagagttataggtacagtgatataggacagtgatactgttatagttataggacagtgatattcttatagttaccgtggtatagttatagttaaaggtacagtgatattgttatagttataggacagtgatattgttatagttataggacagtgatgtagttacagtgatataggtacagtgatattgttatagttataggacagtgatatagttataggacagtgatattgttacagttataggtacagtgacgttgtaatagttataggacagtgatattgttatagttataggtacagtgatgttgtagcaaaactgtaaggtcattccttggttgtgtccctatctagtgaaatgactctaccaggggtagggaaataaaactctgtcgtttcacacataaaggtaatttcaatggaattatccagaaaattgccatgacattccacctcatttagcatgtcaggatgttaatgacaagatgttactaaaagcaaatcccttacggccagttttccgcagtactgggtgctggctctgtgcctggccctgtattgggtgctgggctaggatttccctgtggaagattgggaaggttggttacaaggtggctattttcctgtctcctctttgcgacagcacaccttctccatggtgtgtgccaggttcacgtgtactggtgatttaattttaacgttcatattatttttttctgggagagtttttgaaggctgccaggaggcaggactcgatgcaaacatgctccattctgtacccagccctgttgctggaaggatttgctgcacttacccagggaacaggcaagctcgtgctgtggctctgggctgtcacagctgctgtccacacctgggagagcaccctggatggctcatctgtgtacttgctttcttgttaaattgcagtgagttcacatgtgatttaatcggatcaaatggcctttacagactgataaaaatatggctgtttcaggtggtgttttgagctgctaagggcgtggcttttcactgagtacgtggtccccgttcctcagggaacacccagtagccacatgcctcctaaacctagagtagggctgtctcctggcctaactgcccaaatgagattcataagttagggatgatctgtagttatcactacagatttgtccttggtttcaccaaggattttcctaattttacaaacaaaacccctaaggctcctggaaggagggtagaagtgaaggtgctccgggggcaacacagctgatgagctgaaccagaactcgacccttgggtcacacacatttcacagtgctcactccaccttttgtttttttaatggatttaatggtgttttaaagcctcctgcctctcaacacatatgaattcattatatttacagatttccttctcttgtggtccatcttcctgcatagatcttgagagatgtcaggctaaccacgtttcctcagttaatttaacaaaaccatttgcaactctgacatggaaaattcctaccatgtgacttattaatttatcaattgagatggtacacatattttcaagccaaaaggaggaaaacataaattggaaaaaaaaggtttttttatttttatcacctctggggaagaaagtctgataaacgaagctggttgataaaattgcaattaggggaagcaacatcatggtttctgttcggaggctaaccagatggcatacttgaaatagagaatgtcctagaaatcaactggttgcttggccaaaatatctataaatagtgcccaacatattagataggaaaagcaaagtaaaaacaattttaacaggttaggacattgggctgaagtattgcatatatttaatgtcatgtgcgtccgtgtgaagagaccactaaacaggctttgtgtgagcaagaaagctttttaatcacctgggtgcaggcaggctgagtccgaaaagagagtcagtgaagggagatggggtggggacgttttataggatttgggtaggtagtggaaaattacagtcaaagggggttgttctctggcgggcagcggtggggggtcacaaggtgctcagtgggggagcttctgagccaggagaaggaatttcacaaggtaacgtcatcagttaaggcaggaaccggccattttcacttcttttgtctttcttcagttacttcaggtcatctagatgtatacgtgcaggcctgggcccagaggcctgacattcctgtcttcttatattaataagaaaaagaaaacgaaatagtggtaaagtgttggggtggcgaaagtttttgggggtggtatggagagataatgggcgatgtttctcagggctgcttcgagcgggattaggggcggcgtgggaacctacagtgggagagatgaagctgaaggaatattttatggtaaggggtgatattgtggggttgttagaagcagcatttgtcatatagaatgattggtgatggcctggatatggttttgtgtgaaatgagaaactaaatggaagacacaaggtctgaataagagaaggagaaaaacaggtgttaaaggactaagaattgggaggacccaggacatctaattagagagtgcctaagggggttcagtgtaattacttgcttggttggtgagtttttgggctctatccttgacagagtcctcctttttaagttggaggctgagcttggtgaggtgtgtttttaaaagaccattagtctgttctacctttcctgaagattgaggatggtgaggggtatgaaggttttactgaataccaagagcctgagaaactgcttgggtgatttgactaataaaggccggtctgttatcggattgtatagagatggaaaggccaaactgaggaattatgtctgacagaagggaagaaatgaccacggtggccttctcagaccctgtgggaaaggcctctacccatccagtgaaagtgtctacccagaccaagaggtattttagtttcctgactccgggtatgtgagtaaagtcaatctgccagtcctgggcgggggcaaatccccgagcttgatgtgtagggaagggagggggcctgagcaatccctgaggaggagtggagtagcagatggaacactgagcagttattttttgaggatagatttttacgacggaaaggaaaagtgaggttttaagaggtgggttagtggcttgtaacttacatggaagagtttatgaaatgatgacagaatagaatgggcctgtgaggctggaggagatattttccttggtccaagaattatttgccttgtgtgggaagagattgataggtggaagtttcaatgggggagtagatgggagtgacagatgaggaagaaaaaaactggctgtgagggatagaagttggaatgctcgctgcttttttagctaccttatcagcataggcattgtcctgagcagtgggatctgatgccttttggtggcccttgcagtgaatggcttcagcttcctttggaagtaaagtggccttgagaggagtttttattaaagaggcattaagatggagaacccttgtgtagtgaggaaacctccttcagcccatataaccgcatggtggtgcagaatatggaaggcatatttagagtcagtataaatattgacacgtagtccctttgcaagagtgagggcctgagttaaggcaatgagttcagcttgctgagaggtagtggagcggggcagagcagtagcctcagtgatagatgtggaagatactacagcatagcctgcctttgctggtgagtggtgattaggcctggtggaactgccatcaataaaccaagtgtgatcagggtaaggaacaggaaagaaggaaatatggggaaatggagtggatgtcaggtggatcagagagatacagtcatgggggtgggggccagcctaaaacagtaaggtcaagttgtttgaacagaaaggctacagggcgtggtcctggctcttgtgtaagaattttgactgcgcagccctgcacttcggctgtgtgtaatgaaaagggttgggatgagttagggagagctagtgtgggagcagtttctagggctgtttttaaggaatggcaagaggagtggctaaaggatttaggatctttggggtcagctagctttgcttttgtgagtttatataatggtttagtcaggatggtaaaacttagtatccaaaggcggaagtacttaaccatacctaggaagaaaaggagttgttttgtagaaggggttggggtttgggagatgagccagacacaatcagcagggagagcacatgtgttttcatgaagaattatgccgagataggtaatggatgaggaagaaatttgggcttgactgaagtaatgggggctgtcctcgaagccttgtggcagtacagcccaagtaagttgctgaggctgacgggtgtcagggtcagtccaagtgaaagcgaagagaggctgggatgaagggtgcaaaggaatagtaaagaaagcatgtttgagatccagaacagaataatgggttgtggagggaggtattgaggataggagagtatatggctttggtaccatggggtgaataggcaagacaatttggttaatgaggcacagatcctgaactaacctgtaaggcttgtccggtttttggacaggtaaaatgggggaattgtaaggagagtttataggcttcaaaaggccacgctgtaacaggtgagtgataacaggctttaatccttttaaagcatgctgtgggatgggatattggcattgagcggggtaagtgtgattaggttttaatgggatggtaaggggtgcacgataggttgccaaggagggagcagaggtgtcctatacttgtggattaaggtggggacacacaaggggaggatgtgaaggaggctttgaactggggaaagggtggcattgaggtgtggctgtggcctaagaacagtcagggaagcggataattgagttaaaatgcctcgacctagtaagggagctgggcaggtggtgataactaaaaaggagtgcataaaagaatgttgtccaagttggcaccagagttggggagttttaagaggtttagaagcctggcggtcaatacctacaacagttatggaggcaagggaaacaggcccttgaaaagaacgtaatgtggagtgggtagcctctgtattaattaagaaggggatggatttaccctccactgtaagagttacctaaagcatctgtgatggtccaggaggcttctaaggtgatcgggcagcgtcagtcttcagccgctaagccaagaagatctgggaagcagtcagtcagagagccttgggccagagttccaggggctctgggagtggcagccaggccagttagacagtccgatttctagtggggtcccacacagatgagacacagcttaggaggaatcccaggctgcgggcattccttggcccattggccagatttctggcacttgaaacaagatcctgatggaggaggtcctgtaggaatgcttgaccactgcagtttaggcattttgaagtttttgtgtgtgctggagatgtggctgggttttgtctcacagcagaggcaaggaatcgcaactcagaaatacattgctacttggctgcctctattattgtacatcttgaaggcgaggttaattaagtcctcttgtggggtttgagggctggaatctaatttttggagtttttttttgtttgttttttggttttttttttttaatgtcaggagctgactgggtgataaaatgcatattgagaataagaggccttctgacccttctgggtctagggctgtaaagcgtctcagggttgctgccaaacgggccatgaactgggctgggtttttcatatttgatgaaaaagagcctaaacgctaactgatttgggagaggtcggataaataaaaaggaacattaatcttgactatgcctttagctccaaccacctctttaagaggaaattgttgggcaggtgggggagggctagtcgtggaatgaaactgtaagctggaccgggtgtgaggaggggaggtgatagaaggattatagggtggaggagcagaggctgaggaagaattgggatctggcttggcctggcaaggagcagcctggggaggaggggagaggtcagatgggtccatagaaaaggaggattggaaagactcagcaacacttggggttgggattgagaggacagatgggttgggattgaggggacagatgggagggaaagaaggaagatttgggacaagttgcattgggaacagagactagggagggaccaatgtgtaaaagaatgcctggacgtcaggcacctcagaccgtttgcccattttatgacaagaattatctagatcttgtaggatggaaaaatcgaaagtgccgttttctggctatttggaaccattgtcgagtttgtattggggttaagcagcattgcagaagaaaataaggcatttaggttttaggtcaggtgtgagttgaagaggttttaagttcttgagaacataggctaagggagaagaaggaggaatggagggtggaaagttgcctatagtgaaggaggcaagcccagagaaaagagagggtagagacatggagagaaggggtgggggggtgcttgcccccaggaaagtggttcttgccactaagggtgaaggatcaaggcaggcattcgcgcggtgatcagatacctctgaaacgtgggtgaataatcaagcaggtgtccctgcagtgattaaacagcaaggaaagactatcttcccaagtccatgaccagtgccagagttttgggttcatggataaaacgcgtctcctctgtctctaccagaaaatgaaaggaattgaaattaagagaagggagagattgaaggatggcgccaagattgaaaggaaaaagaggttgagggatagggagagaggttggataagagagtaaaaagaggctgcttacccaatttaaaatcggtgagatgttccttgggcttgttggtctgaggaccagaggtcatgggtggatctttctcatggagcaaagagcagggggacaggggattgatttcccaagggaggtcccctgatctgagtcacagcaccaaatatcacgtgtgtccatgcgaagagaccaccaaacaggctttgtgtgagcaagaaagctttttaatcacctgggtgcaggcgggctgagtccaaaaagagagtcagtgaagggagataggggtggggacgttttataggatttgggtaggtagtggaaaattacagtcaaagggggttgttctctggcgggcaggggtggggggtcacaaggtgctcagtgggggagcttctgagccaggagaaggaatttcacaaggtaacgtcatcagttaaggcaggaaccggccattttcacttcttttgtcattcttcagttacttcaggccatctggatgtatgcatgtaggcttgggcccagaggcctgacatttaacatggataaatgtaaagttcttagaatcatacatacactttggaaaagatggggcttaatcgcactttataagacttgaaggatgtttgagaatcactatgaaactgctgaaaataccaagaaaatttaattcttatgtatataaataatgtgtctgttttacatgaatcccttctacaagcttggtatttaatatggcatatattgttttttcatagtagatttaaaatttttgatatctaatttagataacataaaattaaccctttgaaagtgtacaactccgtggtttttagtatatccacctgattgcacaacgatcaccactgtctagttccagaacatttttatcaccacaaaagaaaggctgtatccaggccgggcacggtggctcacgcctgtaatcccagcactttgggaggccgaggcgggcggatcacggggtcaggagattgaaaccatcctgggtaacacggtgaagccctatctgtactaaagatacaaaaaaattagctgggcatgatggcagatgcctgtagtcccagctactcaggaggctgaggcaggagaatggcctgaacccaggaagcggagcttgcagtgagccaagattgcgccactgcactccagcctgggcgacagagcaagactccatctcacaaaaaaataataaaaataaaataaaaaaaaaaaagaaaggctgtctttctcctttcccattggccgtctttctccattccctactcctccaatcccctggcaaccactaaatctactttccatgtctgtggacttgactcttcgggacattttacataaatggaatcatgcaatgcagcacattttgcatctggcttttttcacctggcgtgttttcaaggctcattcgtattctagcatatatcaatactttgttcctatttaggactaaataagattctattgtatgaataaaacatattttgtttatatacttagtttgatgaacatttgagttgtttctggatttttttttttttttttttgcctcttatgaataatgctgctatggacaacagtttttgggtaggcatgcattttaaattctcttatgtatatacttaggattaaaattgctggatcacagagtaactccatgtttaactttttgatgaattgccaaactgtttttgagagcagctacacaatgttacattcttaccagcaacaattgagggcttcagtctctctacaaccttaacaacacttgttattgtctttgtaattattgcctttctaggcagtgtgaagtggtgtctcactgtggttttgatatgcatttccctaatgactaacaatgttgtgtatcttttcgtgtgctcattttcaatttgaatacattctttgggaaaatctctgtttaaatcttttggccattaaaaataattgggttattttcattgttgagttgtatgaactctttatatactctggatactacactcttatgacatatattattttcaaaaattttctgtgcatctgcaggtcatcttttcactttactgatggtgttctttgaagcaccaaagtttttaaacttgatgaaatccagtctggctttattcttgcacgtgctttacctaaaacgccaaaacctaattcatggttttgaagatttttgcttatgatttgttcttagaggtttatagttttagctcttacatttaggcatttgatgcattttaaattaaattttgtatatggtgtaaagtagcaatccaacttaattcttgcatgtgggtattcagttattccattgtcttgaaacccttttcaaaatcaattgtctataaatgtaagagtttatttttggaccatcagttctatcctgttgacctatatatgcctatcctaatgccagttacacacagtcttgattaccataactttgcagtaagttttgaactcagacagtctgagtgcttttattttgtcctttttcaagattaatttggttattccggatcttttgcatttccatatgaattttaggatggttgtcaatttctgcaatagaaaagcagcaggattttgatagagagtgcattgaatctgtagaccaatgagtatcaatggaattatgtgttatcaaatttagtaaactacataaacgatatgtacacaactaaaacaaaactaagatataagatcatattaatgtaatgataataaaagtggattgtctcctgttctaattttaataggcacaaggcatttttgttaatcacttcttattaaagaattttttataatattcaggaaaatacaacaaaaaaacccttacattcctaaggcctcagagtcagagtaatataaaggaaatgtaaacacatgctgagtaatgcaagcatttggcaatggtggtgactggagctgggagcacagcttttatttctctgaaataggaatttgccccttagagttagaccaattttgccttcctctaaatggcaaacagtttggagatactttaaaggacattttttcacttaggatgtttagtactatgaataataaatagtcacaatttccttaactatggtgacaaaatacaagcaaatttagcctcatgtcatttcctaaggaacatcttctctctgtgagttcacaggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaaggcgagtatgcatggaaaatcgcatcacaaaggcgagtatgcatggggagcacttgctgctgtactttcatcacttttatagtctgagttcttaaaagtttcgttcatttccctcaaaacacttgaacctgcagtttcagtaggtactgttctgccaggtgcagattagttaagagattagcagacttctctgcctatcttctcttactttaaaacaaatgttaccattgaatcaaggaagcaatagccatgagaaaaaagaaggatctgacgcctttgaatgaagattcaaaacatgatcttcatgttttgtattagcttggagtaaaatccacttgctggcaatatagcccttaagcttgttgcctcttctctttgtttcagaaactagagccctgtttattctgatcaaggctctggcccactgtctttatctcagataacccaccctcttctgcacacagcatggagctaagagaagggtgtctagttatgtaatatcatcggcagcataaattcccagaatttgttctttgatttttttgtttgtttttccagttagaaggtggaacttcatcattgtcctcttttcaggttgtctgtgcctattagttttctccagagggagaggtggcttgatttacatttaatctctgcaatttattagagtcctgtagttggatttactttgaagagagtttcccagaagaataaaatttgctgcgttgctttttgggtgtgagctgcttttgtatttgcctaatgcctttaatgcaaatttctttctttctctcttgcttttttttaaaaaaaatagaaatgtttgtcttcttaaaacatctgaaagtggcacaccaagttcctctactcctcaagaaaacaccatatctggatatagccttttaacctgcaaaagaactttacctggtaatgtgatttgataataatattacataaaatgtaacctatttcatgacttttaacagcaacagtgatgaaacaatcctcaaggtaacagaaacttgtgtaaatgtcgttcattgcttttcccatctgatatctttttgtgtttataattgacacagaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtaaaggaaattttatttttcaaagacagttgacatgaccatttcatattctctttccccctgtgaaggcttactctttctactgttcatttcatctaggtgtaagtgtttcttaagattatctatggatggttctccaactaggattgcgtatgggacacaagggagctctggttactctttgagattgtgtaacactggggacaactctggtgagtaacctcactttttcgtggacctgtcagggatgtctgtcatgttgatagtttgcttagtcttaaggaattatgtgtcttgttctccttggttagaagggactttgattcacttctaattccaaccattagcgtcaacgctctcttttcagtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggtaagtgttggatgcatctcatccagagtcttatcttggcttttcattttgaaggatctatgatcagctgcttcaccgccatgtgactttatgaatagagacgtgttaaagcggggatggtattcacaacatttaacttatagggtccaagcactgaccaacctgaccattagaacagagtgtggtctctgtacagggcagatggcgctgagtgggtattctccacagaaagagaaacgaagacagtaccccactcctccaacccaccacccaccaccaatcccaccaccaattccaccaccaatcctgccacccaccaccaatctcaccaccaatcccaccaccaatcctaccacccaccatcaatctcagcaccaatcccaccaccaatcccaccaccaatcccaccacctaccccaccaccaatcccgccacccacgaccaatcccaccaccgatcccgccaccaatcccaccaccaatcccaccacctaccccaccaccaatcccgccacccacgaccaatcccaccaccgatcccgccaccaaccaccaatcccaccaccaatcccaccaccaatccctgatgtgttcttcaaagacttatttgtcaggcccatagaaatgttacttcttgctctttgattcataaatatactaagtcataataatttttaaaagtgagagtttcgtactctgtatatttcaatgtatataatttgatctatttcaatttattggtcaaatagtagacatgttaggtaagtcttaaaatactgaggctttggagttagacagaacatggcttaagtgacagctttgctgcttattagaggtgtggccctagaagatttgtaaatccctctgagctttatttgatctaaaatatgaatagtaatagtcccgaatttgtaacgttgttgggaagattaagtgacacatttaaaatgcttagtactgtgtgtagaacataaacacttcaaaaaatgtaaactgtgatttctatattcaataagaaatgtagaaatggacaaagcatataaaaagcaaaagaaatactagaagacacttgatttttctcaaaaataaacacaccaagtatttttgttttagtgaaattcatgcttacatgctgtatactaggattgaacatactgccaccaaaatatagcagtcggtggtacatgtgggtggagcaagacccctccaccttgtcatcgtgtgaaggggctctgccatacatgaccttgcatgtgactttaaggtggttggcctggaagaaaagtcccaagatgggaaatagtaggtgtcttttttactaaatgcactccaatttgggaccaaaaattttcattcttgaaggctcagtattgtgagtttataagagataatagacataaaagtgtaatgatttcattgcaaataaaaaaaggcccctttgcacctgatatctccatcatttttctagaattttgtgcacacatgccttgcactacttggtgatgataaagatttccagatctttgcacagaataaggctttgctttagatcagaattttggatgtacttagtatacattcatcttttaaataatctatttacattttcatactttccaaaatacagatatattttattttatttatatatttatttaatttattttttgagatggagtctctctctgttgcccagagtagagtgcagtggcacaatcttggttcactgcagcctctgcctcccgggttcaagcgattctcctgcctcagcctcctgagtagctgggattacaggcgcgcgccacacctggctaatttttgtatttttagtagagacgaggtttcatcatgttggtcaggctggtctcgaactcctggcctcaagggatccacccacctcggcctcccgaagtgctgggattacaggtgtgggccactgtgcccagctgtatagagatattttaaacaacactaaagtcctcctactttgactaattagaagagcattagaagatcagcctgacttcttgacagttctgaatttagtggagcaatgaggttcagctttggtgaatgagcttaatttttccatgataaactgctagtttcttcccactacagtgtctctcaaaaatgggacagcaacattctttttgttttcacttgcagtaagcatgatgcaattacataaatgtacacttttcaatttgttaaatagaatcttcagagattcactactgccgctattggtgatgaaaaattaccagaaggaggaattaggtaggagaaaatgtgtcctatgtatttccttcccagttctttgaaagagagtgataggaaaaaggaacactattgaaggaaggactgcccagtttcaaacaggtatttatttttctctcctaggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactggtatgtagcatatgtaagaaggtggagagcagaattgcgctggttgatattttcatatcagtttgaacaagagggcagacctagagagactgtcgtcgttttctgactggtggagttgagggaaacgtgagggttgctgggaagtgaagaccccgcgacttgccgtgaaatctcttctacttaaagagcaagacatgtgaattaattctttcagggagggatacaactgcatgcaggtgatggaaataatgggcgtgggaaatgtctgtgccgtctgagaggcactgggcttgctttgacaagagtagcagaactgtcattgctttgggcttagggatattcgaatgtgtgagggcaagtgggatcagatatctacttccaggtataatttgggtaggaaagagactcatgcagaaagaagccctggaaggccagagcatcgtggtcagaggtgttgcctttggagggtcattgctgccaggagccgaatacccactgtatccaataacattcatggtcaggaatggtggctcacacctgtaatcccaacactttgggatgcccaggtgggaggattgcttgaggccaggagtttgagaccagcctgggcaacacagtgagaccccgtctctacataaaattagaaaaaaacaattaactgggtgtggtggtgtgcacctgtagtcccagctagtcaagaggctgagacaagaggatctcttgagcccaggagctcaaggctgtagtgagccaagatcgtgccactgcacacaaacaattatgtgacctcgggcaagttgctttacctctttacacctcttaatttccttatctgtaaaatgaggatgataatttcttcctgggtctgttgtaataattaatacatcaaagcacttcatgtctggaacagtgaagataccctgctatgactattaaggatagtatacatggaataagacacaggaacttctaaatgcttttgaccatagatttaggttctgagttttaagaatttaactcaggaaattgtaacaccaaaaatgtcatgtgaaaaatggtggtgacaaattttcttgaatcattagccttagaggttgggcagaaagcaaaaaattattcttgatgctactctatagaaagagaagacagaaaaagagaaagatgtatttttaaagtctatatccataactttatttgaccaaactctaatttaaaaattatgtttcagaattccaaaaaccaatatgcctaccttccaaaggtgacacaagcacaatttataccaactgttgggtaaccggatggggcttctcgaaggagaaaggtaagcatgacgctttaaatattgcttctagagtaagtctcacatgttgaaatacatggagtgggtcgttttaatcggtttctgtctgaaattatatctaaactctttatctttcctatctatttattcccaaatatttattcagttattcttaaaaaatgtatttttgctttggcttgaaaaaaaattttagggagacttttaagcatcttacttcattataaagatcagttgcttgactttgcatgaagcagattgggcccttctagggctgacaagcccgtgcaagaccacccgctcctcagtgttagtagcgttcccgtctcccaaaaccatgttctcccttgatgctaatggccgggagcacaggcaggtgtgtcgtctcactatggagaataatatttgtgtcattctttacagaagaaggtagcttgccaaactgtctccatctttcccgattcagtcttttgttcaagtaattcacatttttagattttttattggtaatctgagacaagaagaaatttaaagtaatcttcactaagccatgaaagctcccaacattgttctccatgagagatgctggcctgcatttattcaaaaacaaaagacccctctgttgccaaagctcggagggcttttcagaaacgatatagttgtaaattataattttgaatatataaagcaaaaaaatgaaaagtgagaacttccaggctttggattgttgtaggtgataaatataaaatgggatttctggggggctgctactgagatgaggggatggcagaaaacatggaagcaaggtctctggtcagcccagggtgctgggcttgtcccaacaccacgtaggcaataagaggacagtacagggtgccgtctctctccctcttcctctctctgtctctctctctctgtgtgtgtgtgtgtgtgtgtgtgtaacactaccttcccaatttttactgtctatttgtattcaaagataaggtccttatgaaaaatacactgctctgattcactttaaaacttatttccatatttattatttattgtgggaataataatattcccaatattattatttattatttaagttattattattaacttccctctgaggttatatattggttactcacaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaaggtaactcatgagattatgaaaaacacaataggctgcttgagaaaattcatttcaaaatatattttccaatagcataattcaatcatagtttttaaaaaaattcagagacaaatgatctgataaattgataagcaacttttaacaaattgaatatacataatatatatttatattatttatgatatatgtcacaatctatgcatgtgctatttaagaggggcaaatatacatgcaataattgtgctagaatataaaaacattagacttcatcattgggatgatgatatcaagatttctttgttagatttatttcagatagaaaaggggatacgaaaaatgcaggcacatgagatacttggagaactttaagaaagagtgagtgtgtgtgtgtgtgtgtgtgtgtgtgtgtgtgtgtctggcaagcaaggtcttgcacacacacagcactttgggaggccaatgcaggtggatcacttgagcctaggaatttgagaccagtctgggcaatgtgatgaaacccatctctacaaaaaaatatgaaagtatctgtgtgtgtgtgttgctgtgtagtggactacagaactttagaggcagtcacttatttgaatcccattgtcgtaactttctactattttatttttccactgtgactcagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatcacgatta

SEQ ID NO: 5 - DNA, 2187 bp, Homo sapiens
  source 1..2187: mol_type = genomic DNA
agtgccacattagaacagcttgaagaccgttcatttttaagtgacaagagactcacctccaagaagcaattgtgttttcagtttcctgtgagggagttttctctgtgtccccacaaggcatgattctgggtctatggtgacttaagagggccacacaacaatgagtatttaattttcctcagatgtatggctacaataaacatctataggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaactctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatca

SEQ ID NO: 6 - DNA, 2289 bp, Homo sapiens
  source 1..2289: mol_type = genomic DNA
agtgccacattagaacagcttgaagaccgttcatttttaagtgacaagagactcacctccaagaagcaattgtgttttcagaatgattttattcaagcaagcaacttatttcatttccttgtttgctacagtttcctgtggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaaggcgagtatgcatggaaaatcgcatcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaactctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaaaacacagtgcactcagagctgctgaggacaatgtctggctgaagcccgctttcagcacgccgtaaccaggggctgacaatgcgaggtcgcaactgagatctccatgactgtgtgttgtgaaataaaatggtgaaagatca

SEQ ID NO: 7-9 - skipped (3 entries)

SEQ ID NO: 10 - RNA, 21 nt, synthetic construct
  source 1..21: mol_type = other RNA
ttacatccccacctctgaaga

SEQ ID NO: 11 - RNA, 21 nt, synthetic construct
  source 1..21: mol_type = other RNA
tctacatccccacctctgaag

SEQ ID NO: 12 - RNA, 21 nt, synthetic construct
  source 1..21: mol_type = other RNA
agctacatccccacctctgaa

SEQ ID NO: 13 - RNA, 21 nt, synthetic construct
  source 1..21: mol_type = other RNA
aagctacatccccacctctga

SEQ ID NO: 14 - RNA, 21 nt, synthetic construct
  source 1..21: mol_type = other RNA
taagctacatccccacctctg

SEQ ID NO: 15 - RNA, 21 nt, synthetic construct
  source 1..21: mol_type = other RNA
tgaagctacatccccacctct

SEQ ID NO: 16 - RNA, 21 nt, synthetic construct
  source 1..21: mol_type = other RNA
tggaagctacatccccacctc

SEQ ID NO: 17 - RNA, 21 nt, synthetic construct
  source 1..21: mol_type = other RNA
atggaagctacatccccacct

SEQ ID NO: 18 - RNA, 21 nt, synthetic construct
  source 1..21: mol_type = other RNA
tatggaagctacatccccacc

SEQ ID NO: 19 - RNA, 21 nt, synthetic construct
  source 1..21: mol_type = other RNA
acatggaagctacatccccac

SEQ ID NO: 20 - RNA, 21 nt, synthetic construct
  source 1..21: mol_type = other RNA
tacatggaagctacatcccca

SEQ ID NO: 21 - RNA, 21 nt, synthetic construct
  source 1..21: mol_type = other RNA
ttacatggaagctacatcccc

SEQ ID NO: 22 - RNA, 21 nt, synthetic construct
  source 1..21: mol_type = other RNA
taccttgggtggaatgtgcac

SEQ ID NO: 23 - RNA, 21 nt, synthetic construct
  source 1..21: mol_type = other RNA
acaccttgggtggaatgtgca

SEQ ID NO: 24 - RNA, 21 nt, synthetic construct
  source 1..21: mol_type = other RNA
aacaccttgggtggaatgtgc

SEQ ID NO: 25 - RNA, 21 nt, synthetic construct
  source 1..21: mol_type = other RNA
tcaaacaccttgggtggaatg

SEQ ID NO: 26 - RNA, 21 nt, synthetic construct
  source 1..21: mol_type = other RNA
agcaaacaccttgggtggaat

SEQ ID NO: 27 - RNA, 21 nt, synthetic construct
  source 1..21: mol_type = other RNA
aatagcaaacaccttgggtgg

SEQ ID NO: 28 - RNA, 21 nt, synthetic construct
  source 1..21: mol_type = other RNA
taatagcaaacaccttgggtg

SEQ ID NO: 29 - RNA, 21 nt, synthetic construct
  source 1..21: mol_type = other RNA
tgaatagcaaacaccttgggt

SEQ ID NO: 30 - RNA, 21 nt, synthetic construct
  source 1..21: mol_type = other RNA
actgaatagcaaacaccttgg

SEQ ID NO: 31 - RNA, 21 nt, synthetic construct
  source 1..21: mol_type = other RNA
aactgaatagcaaacaccttg

SEQ ID NO: 32 - RNA, 21 nt, synthetic construct
  source 1..21: mol_type = other RNA
aaactgaatagcaaacacctt

SEQ ID NO: 33 - RNA, 21 nt, synthetic construct
  source 1..21: mol_type = other RNA
aaaactgaatagcaaacacct

SEQ ID NO: 34 - RNA, 21 nt, synthetic construct
  source 1..21: mol_type = other RNA
taaaactgaatagcaaacacc

SEQ ID NO: 35 - RNA, 21 nt, synthetic construct
  source 1..21: mol_type = other RNA
agaaaactgaatagcaaacac

SEQ ID NO: 36 - RNA, 21 nt, synthetic construct
  source 1..21: mol_type = other RNA
tggaagaaaactgaatagcaa

SEQ ID NO: 37 - RNA, 21 nt, synthetic construct
  source 1..21: mol_type = other RNA
taacttgctggaagaaaactg

SEQ ID NO: 38 - RNA, 21 nt, synthetic construct
  source 1..21: mol_type = other RNA
tcttttctccatgtcattgat

SEQ ID NO: 39 - RNA, 21 nt, synthetic construct
  source 1..21: mol_type = other RNA
aaaccttttctccatgtcatt

SEQ ID NO: 40 - RNA, 21 nt, synthetic construct
  source 1..21: mol_type = other RNA
taaaccttttctccatgtcat

SEQ ID NO: 41 - RNA, 21 nt, synthetic construct
  source 1..21: mol_type = other RNA
taccttttttggcattcttca

SEQ ID NO: 42 - RNA, 21 nt, synthetic construct
  source 1..21: mol_type = other RNA
tggtgcaccttttttggcatt

SEQ ID NO: 43 - RNA, 21 nt, synthetic construct
  source 1..21: mol_type = other RNA
accaatttctgaaagggcaca

SEQ ID NO: 44 - RNA, 21 nt, synthetic construct
  source 1..21: mol_type = other RNA
aaccaatttctgaaagggcac

SEQ ID NO: 45 - RNA, 21 nt, synthetic construct
  source 1..21: mol_type = other RNA
tcaagatgctggaagatgttc

SEQ ID NO: 46 - RNA, 21 nt, synthetic construct
  source 1..21: mol_type = other RNA
aggaacttggtgtgccacttt

SEQ ID NO: 47 - RNA, 21 nt, synthetic construct
  source 1..21: mol_type = other RNA
taggaacttggtgtgccactt

SEQ ID NO: 48 - RNA, 21 nt, synthetic construct
  source 1..21: mol_type = other RNA
agaggaacttggtgtgccact

SEQ ID NO: 49 - RNA, 21 nt, synthetic construct
  source 1..21: mol_type = other RNA
tagaggaacttggtgtgccac

SEQ ID NO: 50 - RNA, 21 nt, synthetic construct
  source 1..21: mol_type = other RNA
ttagaggaacttggtgtgcca

SEQ ID NO: 51 - RNA, 21 nt, synthetic construct
  source 1..21: mol_type = other RNA
taggagtagaggaacttggtg

SEQ ID NO: 52 - RNA, 21 nt, synthetic construct
  source 1..21: mol_type = other RNA
tgaggagtagaggaacttggt

SEQ ID NO: 53 - RNA, 21 nt, synthetic construct
  source 1..21: mol_type = other RNA
ttgaggagtagaggaacttgg

SEQ ID NO: 54 - RNA, 21 nt, synthetic construct
  source 1..21: mol_type = other RNA
tttgaggagtagaggaacttg

SEQ ID NO: 55 - RNA, 21 nt, synthetic construct
  source 1..21: mol_type = other RNA
tcttgaggagtagaggaactt

SEQ ID NO: 56 - RNA, 21 nt, synthetic construct
  source 1..21: mol_type = other RNA
ttcttgaggagtagaggaact

SEQ ID NO: 57 - RNA, 21 nt, synthetic construct
  source 1..21: mol_type = other RNA
tttcttgaggagtagaggaac

SEQ ID NO: 58 - RNA, 21 nt, synthetic construct
  source 1..21: mol_type = other RNA
ttttcttgaggagtagaggaa

SEQ ID NO: 59 - RNA, 21 nt, synthetic construct
  source 1..21: mol_type = other RNA
ttgttttcttgaggagtagag

SEQ ID NO: 60 - RNA, 21 nt, synthetic construct
  source 1..21: mol_type = other RNA
tgtgttttcttgaggagtaga

SEQ ID NO: 61 - RNA, 21 nt, synthetic construct
  source 1..21: mol_type = other RNA
tggtgttttcttgaggagtag

SEQ ID NO: 62 - RNA, 21 nt, synthetic construct
  source 1..21: mol_type = other RNA
tcttttgcaggttaaaaggct

SEQ ID NO: 63 - RNA, 21 nt, synthetic construct
  source 1..21: mol_type = other RNA
agggttcaggtaaagttcttt

SEQ ID NO: 64 - RNA, 21 nt, synthetic construct
  source 1..21: mol_type = other RNA
tagggttcaggtaaagttctt

SEQ ID NO: 65 - RNA, 21 nt, synthetic construct
  source 1..21: mol_type = other RNA
tcagggttcaggtaaagttct

SEQ ID NO: 66 - RNA, 21 nt, synthetic construct
  source 1..21: mol_type = other RNA
tgcagggttcaggtaaagttc

SEQ ID NO: 67 - RNA, 21 nt, synthetic construct
  source 1..21: mol_type = other RNA
tggcagggttcaggtaaagtt

SEQ ID NO: 68 - RNA, 21 nt, synthetic construct
  source 1..21: mol_type = other RNA
atggcagggttcaggtaaagt

SEQ ID NO: 69 - RNA, 21 nt, synthetic construct
  source 1..21: mol_type = other RNA
taatggcagggttcaggtaaa

SEQ ID NO: 70 - RNA, 21 nt, synthetic construct
  source 1..21: mol_type = other RNA
agaatggcagggttcaggtaa

SEQ ID NO: 71 - RNA, 21 nt, synthetic construct
  source 1..21: mol_type = other RNA
tagaatggcagggttcaggta

SEQ ID NO: 72 - RNA, 21 nt, synthetic construct
  source 1..21: mol_type = other RNA
ttagaatggcagggttcaggt

SEQ ID NO: 73 - RNA, 21 nt, synthetic construct
  source 1..21: mol_type = other RNA
tttagaatggcagggttcagg

SEQ ID NO: 74 - RNA, 21 nt, synthetic construct
  source 1..21: mol_type = other RNA
ttttagaatggcagggttcag

SEQ ID NO: 75 - RNA, 21 nt, synthetic construct
  source 1..21: mol_type = other RNA
attttagaatggcagggttca

SEQ ID NO: 76 - RNA, 21 nt, synthetic construct
  source 1..21: mol_type = other RNA
aattttagaatggcagggttc

SEQ ID NO: 77 - RNA, 21 nt, synthetic construct
  source 1..21: mol_type = other RNA
aaattttagaatggcagggtt

SEQ ID NO: 78 - RNA, 21 nt, synthetic construct
  source 1..21: mol_type = other RNA
taaattttagaatggcagggt

SEQ ID NO: 79 - RNA, 21 nt, synthetic construct
  source 1..21: mol_type = other RNA
ttaaattttagaatggcaggg

SEQ ID NO: 80 - RNA, 21 nt, synthetic construct
  source 1..21: mol_type = other RNA
tgtaaattttagaatggcagg

SEQ ID NO: 81 - RNA, 21 nt, synthetic construct
  source 1..21: mol_type = other RNA
tcgggtaaattttagaatggc

SEQ ID NO: 82 - RNA, 21 nt, synthetic construct
  source 1..21: mol_type = other RNA
tccgggtaaattttagaatgg

SEQ ID NO: 83 - RNA, 21 nt, synthetic construct
  source 1..21: mol_type = other RNA
tacattcaattcttctcctcc

SEQ ID NO: 84 - RNA, 21 nt, synthetic construct
  source 1..21: mol_type = other RNA
tcacattcaattcttctcctc

SEQ ID NO: 85 - RNA, 21 nt, synthetic construct
  source 1..21: mol_type = other RNA
agtcacattcaattcttctcc

SEQ ID NO: 86 - RNA, 21 nt, synthetic construct
  source 1..21: mol_type = other RNA
tgcaaacattcactcctttaa

SEQ ID NO: 87 - RNA, 21 nt, synthetic construct
  source 1..21: mol_type = other RNA
actgacagcgaatcatctttg

SEQ ID NO: 88 - RNA, 21 nt, synthetic construct
  source 1..21: mol_type = other RNA
aactgacagcgaatcatcttt

SEQ ID NO: 89 - RNA, 21 nt, synthetic construct
  source 1..21: mol_type = other RNA
aaactgacagcgaatcatctt

SEQ ID NO: 90 - RNA, 21 nt, synthetic construct
  source 1..21: mol_type = other RNA
aaaactgacagcgaatcatct

SEQ ID NO: 91 - RNA, 21 nt, synthetic construct
  source 1..21: mol_type = other RNA
aaaaactgacagcgaatcatc

SEQ ID NO: 92 - RNA, 21 nt, synthetic construct
  source 1..21: mol_type = other RNA
ttgaaaaactgacagcgaatc

SEQ ID NO: 93 - RNA, 21 nt, synthetic construct
  source 1..21: mol_type = other RNA
agtgaaaaactgacagcgaat

SEQ ID NO: 94 - RNA, 21 nt, synthetic construct
  source 1..21: mol_type = other RNA
aagtgaaaaactgacagcgaa

SEQ ID NO: 95 - RNA, 21 nt, synthetic construct
  source 1..21: mol_type = other RNA
taagtgaaaaactgacagcga

SEQ ID NO: 96 - RNA, 21 nt, synthetic construct
  source 1..21: mol_type = other RNA
ataagtgaaaaactgacagcg

SEQ ID NO: 97 - RNA, 21 nt, synthetic construct
  source 1..21: mol_type = other RNA
aataagtgaaaaactgacagc

SEQ ID NO: 98 - RNA, 21 nt, synthetic construct
  source 1..21: mol_type = other RNA
aagaataagtgaaaaactgac

SEQ ID NO: 99 - RNA, 21 nt, synthetic construct
  source 1..21: mol_type = other RNA
aaagaataagtgaaaaactga

SEQ ID NO: 100 - RNA, 21 nt, synthetic construct
  source 1..21: mol_type = other RNA
tagtaaagaataagtgaaaaa

SEQ ID NO: 101 - RNA, 21 nt, synthetic construct
  source 1..21: mol_type = other RNA
tgagtaaagaataagtgaaaa

SEQ ID NO: 102 - RNA, 21 nt, synthetic construct
  source 1..21: mol_type = other RNA
tggagtaaagaataagtgaaa

SEQ ID NO: 103 - RNA, 21 nt, synthetic construct
  source 1..21: mol_type = other RNA
tgggagtaaagaataagtgaa

SEQ ID NO: 104 - RNA, 21 nt, synthetic construct
  source 1..21: mol_type = other RNA
ttgggagtaaagaataagtga

SEQ ID NO: 105 - RNA, 21 nt, synthetic construct
  source 1..21: mol_type = other RNA
tctgggagtaaagaataagtg

SEQ ID NO: 106 - RNA, 21 nt, synthetic construct
  source 1..21: mol_type = other RNA
ttctgggagtaaagaataagt

SEQ ID NO: 107 - RNA, 21 nt, synthetic construct
  source 1..21: mol_type = other RNA
tttctgggagtaaagaataag

SEQ ID NO: 108 - RNA, 21 nt, synthetic construct
  source 1..21: mol_type = other RNA
tcttctgggagtaaagaataa

SEQ ID NO: 109 - RNA, 21 nt, synthetic construct
  source 1..21: mol_type = other RNA
agtcttctgggagtaaagaat

SEQ ID NO: 110 - RNA, 21 nt, synthetic construct
  source 1..21: mol_type = other RNA
tagtcttctgggagtaaagaa

SEQ ID NO: 111 - RNA, 21 nt, synthetic construct
  source 1..21: mol_type = other RNA
tttacagtcttctgggagtaa

SEQ ID NO: 112 - RNA, 21 nt, synthetic construct
  source 1..21: mol_type = other RNA
tcttacagtcttctgggagta

SEQ ID NO: 113 - RNA, 21 nt, synthetic construct
  source 1..21: mol_type = other RNA
tccttacagtcttctgggagt

SEQ ID NO: 114 - RNA, 21 nt, synthetic construct
  source 1..21: mol_type = other RNA
taagaaacacttacacttctc

SEQ ID NO: 115 - RNA, 21 nt, synthetic construct
  source 1..21: mol_type = other RNA
ttaagaaacacttacacttct

SEQ ID NO: 116 - RNA, 21 nt, synthetic construct
  source 1..21: mol_type = other RNA
tttaagaaacacttacacttc

SEQ ID NO: 117 - RNA, 21 nt, synthetic construct
  source 1..21: mol_type = other RNA
tataatcttaagaaacactta

SEQ ID NO: 118 - RNA, 21 nt, synthetic construct
  source 1..21: mol_type = other RNA
atcctagttggagaaccatcc

SEQ ID NO: 119 - RNA, 21 nt, synthetic construct
  source 1..21: mol_type = other RNA
aatcctagttggagaaccatc

SEQ ID NO: 120 - RNA, 21 nt, synthetic construct
  source 1..21: mol_type = other RNA
tagagctcccttgtgtcccat

SEQ ID NO: 121 - RNA, 21 nt, synthetic construct
  source 1..21: mol_type = other RNA
tcagagctcccttgtgtccca

SEQ ID NO: 122 - RNA, 21 nt, synthetic construct
  source 1..21: mol_type = other RNA
aaccagagctcccttgtgtcc

SEQ ID NO: 123 - RNA, 21 nt, synthetic construct
  source 1..21: mol_type = other RNA
taaccagagctcccttgtgtc

SEQ ID NO: 124 - RNA, 21 nt, synthetic construct
  source 1..21: mol_type = other RNA
ttaaccagagctcccttgtgt

SEQ ID NO: 125 - RNA, 21 nt, synthetic construct
  source 1..21: mol_type = other RNA
tagtaaccagagctcccttgt

SEQ ID NO: 126 - RNA, 21 nt, synthetic construct
  source 1..21: mol_type = other RNA
agagtaaccagagctcccttg

SEQ ID NO: 127 - RNA, 21 nt, synthetic construct
  source 1..21: mol_type = other RNA
tcaaagagtaaccagagctcc

SEQ ID NO: 128 - RNA, 21 nt, synthetic construct
  source 1..21: mol_type = other RNA
tccagtgttacacaatctcaa

SEQ ID NO: 129 - RNA, 21 nt, synthetic construct
  source 1..21: mol_type = other RNA
tcccagtgttacacaatctca

SEQ ID NO: 130 - RNA, 21 nt, synthetic construct
  source 1..21: mol_type = other RNA
tgtccccagtgttacacaatc

SEQ ID NO: 131 - RNA, 21 nt, synthetic construct
  source 1..21: mol_type = other RNA
tcccaagaagagtttgttcct

SEQ ID NO: 132 - RNA, 21 nt, synthetic construct
  source 1..21: mol_type = other RNA
tccccaagaagagtttgttcc

SEQ ID NO: 133 - RNA, 21 nt, synthetic construct
  source 1..21: mol_type = other RNA
ttccccaagaagagtttgttc

SEQ ID NO: 134 - RNA, 21 nt, synthetic construct
  source 1..21: mol_type = other RNA
tctccccaagaagagtttgtt

SEQ ID NO: 135 - RNA, 21 nt, synthetic construct
  source 1..21: mol_type = other RNA
ttctccccaagaagagtttgt

SEQ ID NO: 136 - RNA, 21 nt, synthetic construct
  source 1..21: mol_type = other RNA
actctccccaagaagagtttg

SEQ ID NO: 137 - RNA, 21 nt, synthetic construct
  source 1..21: mol_type = other RNA
tcactctccccaagaagagtt

SEQ ID NO: 138 - RNA, 21 nt, synthetic construct
  source 1..21: mol_type = other RNA
tccactctccccaagaagagt

SEQ ID NO: 139 - RNA, 21 nt, synthetic construct
  source 1..21: mol_type = other RNA
agggccactctccccaagaag

SEQ ID NO: 140 - RNA, 21 nt, synthetic construct
  source 1..21: mol_type = other RNA
tagggccactctccccaagaa

SEQ ID NO: 141 - RNA, 21 nt, synthetic construct
  source 1..21: mol_type = other RNA
tcagggccactctccccaaga

SEQ ID NO: 142 - RNA, 21 nt, synthetic construct
  source 1..21: mol_type = other RNA
ttggtgtcctatgagtgaccc

SEQ ID NO: 143 - RNA, 21 nt, synthetic construct
  source 1..21: mol_type = other RNA
actggtgtcctatgagtgacc

SEQ ID NO: 144 - RNA, 21 nt, synthetic construct
  source 1..21: mol_type = other RNA
tccactggtgtcctatgagtg

SEQ ID NO: 145 - RNA, 21 nt, synthetic construct
  source 1..21: mol_type = other RNA
acccactggtgtcctatgagt

SEQ ID NO: 146 - RNA, 21 nt, synthetic construct
  source 1..21: mol_type = other RNA
tacccactggtgtcctatgag

SEQ ID NO: 147 - RNA, 21 nt, synthetic construct
  source 1..21: mol_type = other RNA
tgaagcccatcaaagcagtgg

SEQ ID NO: 148 - RNA, 21 nt, synthetic construct
  source 1..21: mol_type = other RNA
acagatttaaaatgccactat

SEQ ID NO: 149 - RNA, 21 nt, synthetic construct
  source 1..21: mol_type = other RNA
tacagatttaaaatgccacta

SEQ ID NO: 150 - RNA, 21 nt, synthetic construct
  source 1..21: mol_type = other RNA
tgacagatttaaaatgccact

SEQ ID NO: 151 - RNA, 21 nt, synthetic construct
  source 1..21: mol_type = other RNA
ttgacagatttaaaatgccac

SEQ ID NO: 152 - RNA, 21 nt, synthetic construct
  source 1..21: mol_type = other RNA
tctgacagatttaaaatgcca

SEQ ID NO: 153 - RNA, 21 nt, synthetic construct
  source 1..21: mol_type = other RNA
ttctgacagatttaaaatgcc

SEQ ID NO: 154 - RNA, 21 nt, synthetic construct
  source 1..21: mol_type = other RNA
tgtctgacagatttaaaatgc

SEQ ID NO: 155 - RNA, 21 nt, synthetic construct
  source 1..21: mol_type = other RNA
tgtaatgtctgacagatttaa

SEQ ID NO: 156 - RNA, 21 nt, synthetic construct
  source 1..21: mol_type = other RNA
tcttttgtaatgtctgacaga

SEQ ID NO: 157 - RNA, 21 nt, synthetic construct
  source 1..21: mol_type = other RNA
atatcatgattcccttctgag

SEQ ID NO: 158 - RNA, 21 nt, synthetic construct
  source 1..21: mol_type = other RNA
tcgatatcatgattcccttct

SEQ ID NO: 159 - RNA, 21 nt, synthetic construct
  source 1..21: mol_type = other RNA
tgcgatatcatgattcccttc

SEQ ID NO: 160 - RNA, 21 nt, synthetic construct
  source 1..21: mol_type = other RNA
aggcgatatcatgattccctt

SEQ ID NO: 161 - RNA, 21 nt, synthetic construct
  source 1..21: mol_type = other RNA
aattcaaaggagcctggagtt

SEQ ID NO: 162 - RNA, 21 nt, synthetic construct
  source 1..21: mol_type = other RNA
taattcaaaggagcctggagt

SEQ ID NO: 163 - RNA, 21 nt, synthetic construct
  source 1..21: mol_type = other RNA
ttaattcaaaggagcctggag

SEQ ID NO: 164 - RNA, 21 nt, synthetic construct
  source 1..21: mol_type = other RNA
tgtaattcaaaggagcctgga

SEQ ID NO: 165 - RNA, 21 nt, synthetic construct
  source 1..21: mol_type = other RNA
ttgtaattcaaaggagcctgg

SEQ ID NO: 166 - RNA, 21 nt, synthetic construct
  source 1..21: mol_type = other RNA
agtgtaattcaaaggagcctg

SEQ ID NO: 167 - RNA, 21 nt, synthetic construct
  source 1..21: mol_type = other RNA
tagtgtaattcaaaggagcct

SEQ ID NO: 168 - RNA, 21 nt, synthetic construct
  source 1..21: mol_type = other RNA
tcagtgtaattcaaaggagcc

SEQ ID NO: 169 - RNA, 21 nt, synthetic construct
  source 1..21: mol_type = other RNA
tgaattcagtgtaattcaaag

SEQ ID NO: 170 - RNA, 21 nt, synthetic construct
  source 1..21: mol_type = other RNA
attggtttttggaattcagtg

SEQ ID NO: 171 - RNA, 21 nt, synthetic construct
  source 1..21: mol_type = other RNA
tattggtttttggaattcagt

SEQ ID NO: 172 - RNA, 21 nt, synthetic construct
  source 1..21: mol_type = other RNA
tatattggtttttggaattca

SEQ ID NO: 173 - RNA, 21 nt, synthetic construct
  source 1..21: mol_type = other RNA
tcatattggtttttggaattc

SEQ ID NO: 174 - RNA, 21 nt, synthetic construct
  source 1..21: mol_type = other RNA
ttaggcatattggtttttgga

SEQ ID NO: 175 - RNA, 21 nt, synthetic construct
  source 1..21: mol_type = other RNA
tgtaggcatattggtttttgg

SEQ ID NO: 176 - RNA, 21 nt, synthetic construct
  source 1..21: mol_type = other RNA
aggtaggcatattggtttttg

SEQ ID NO: 177 - RNA, 21 nt, synthetic construct
  source 1..21: mol_type = other RNA
aaggtaggcatattggttttt

SEQ ID NO: 178 - RNA, 21 nt, synthetic construct
  source 1..21: mol_type = other RNA
taaggtaggcatattggtttt

SEQ ID NO: 179 - RNA, 21 nt, synthetic construct
  source 1..21: mol_type = other RNA
tgaaggtaggcatattggttt

SEQ ID NO: 180 - RNA, 21 nt, synthetic construct
  source 1..21: mol_type = other RNA
tggaaggtaggcatattggtt

SEQ ID NO: 181 - RNA, 21 nt, synthetic construct
  source 1..21: mol_type = other RNA
tcacctttggaaggtaggcat

SEQ ID NO: 182 - RNA, 21 nt, synthetic construct
  source 1..21: mol_type = other RNA
ttgtcacctttggaaggtagg

SEQ ID NO: 183 - RNA, 21 nt, synthetic construct
  source 1..21: mol_type = other RNA
tgtgtcacctttggaaggtag

SEQ ID NO: 184 - RNA, 21 nt, synthetic construct
  source 1..21: mol_type = other RNA
ttgtgtcacctttggaaggta

SEQ ID NO: 185 - RNA, 21 nt, synthetic construct
  source 1..21: mol_type = other RNA
ttgatgcccaccaaacgccac

SEQ ID NO: 186 - RNA, 21 nt, synthetic construct
  source 1..21: mol_type = other RNA
ttccctgcgggcacagccttc

SEQ ID NO: 187 - RNA, 21 nt, synthetic construct
  source 1..21: mol_type = other RNA
accaggttgctccctgcgggc

SEQ ID NO: 188 - RNA, 21 nt, synthetic construct
  source 1..21: mol_type = other RNA
taccaggttgctccctgcggg

SEQ ID NO: 189 - RNA, 21 nt, synthetic construct
  source 1..21: mol_type = other RNA
acaccaggttgctccctgcgg

SEQ ID NO: 190 - RNA, 21 nt, synthetic construct
  source 1..21: mol_type = other RNA
tacaccaggttgctccctgcg

SEQ ID NO: 191 - RNA, 21 nt, synthetic construct
  source 1..21: mol_type = other RNA
agacaccaggttgctccctgc

SEQ ID NO: 192 - RNA, 21 nt, synthetic construct
  source 1..21: mol_type = other RNA
tagacaccaggttgctccctg

SEQ ID NO: 193 - RNA, 21 nt, synthetic construct
  source 1..21: mol_type = other RNA
ttagacaccaggttgctccct

SEQ ID NO: 194 - RNA, 21 nt, synthetic construct
  source 1..21: mol_type = other RNA
tgtagacaccaggttgctccc

SEQ ID NO: 195 - RNA, 21 nt, synthetic construct
  source 1..21: mol_type = other RNA
ttgtagacaccaggttgctcc

SEQ ID NO: 196 - RNA, 21 nt, synthetic construct
  source 1..21: mol_type = other RNA
agcgactttggtgtagacacc

SEQ ID NO: 197 - RNA, 21 nt, synthetic construct
  source 1..21: mol_type = other RNA
tcagtccatgtactcagcgac

SEQ ID NO: 198 - RNA, 21 nt, synthetic construct
  source 1..21: mol_type = other RNA
tccagtccatgtactcagcga

SEQ ID NO: 199 - RNA, 21 nt, synthetic construct
  source 1..21: mol_type = other RNA
atccagtccatgtactcagcg

SEQ ID NO: 200 - RNA, 21 nt, synthetic construct
  source 1..21: mol_type = other RNA
tctgtgttttctctaaaatcc

SEQ ID NO: 201 - RNA, 21 nt, synthetic construct
  source 1..21: mol_type = other RNA
ttctgtgttttctctaaaatc

SEQ ID NO: 202 - RNA, 21 nt, synthetic construct
  source 1..21: mol_type = other RNA
tctctgtgttttctctaaaat

SEQ ID NO: 203 - RNA, 21 nt, synthetic construct
  source 1..21: mol_type = other RNA
tgctctgtgttttctctaaaa

SEQ ID NO: 204 - RNA, 21 nt, synthetic construct
  source 1..21: mol_type = other RNA
ttgctctgtgttttctctaaa

SEQ ID NO: 205 - RNA, 21 nt, synthetic construct
  source 1..21: mol_type = other RNA
tatcactgctctgtgttttct

SEQ ID NO: 206 - RNA, 21 nt, synthetic construct
  source 1..21: mol_type = other RNA
ttccatcactgctctgtgttt

SEQ ID NO: 207 - RNA, 21 nt, synthetic construct
  source 1..21: mol_type = other RNA
tttccatcactgctctgtgtt

SEQ ID NO: 208 - RNA, 21 nt, synthetic construct
  source 1..21: mol_type = other RNA
tgctcagaatttgacttgaac

SEQ ID NO: 209 - RNA, 21 nt, synthetic construct
  source 1..21: mol_type = other RNA
aggctcagaatttgacttgaa

SEQ ID NO: 210 - RNA, 21 nt, synthetic construct
  source 1..21: mol_type = other RNA
taggctcagaatttgacttga

SEQ ID NO: 211 - RNA, 21 nt, synthetic construct
  source 1..21: mol_type = other RNA
tcaggctcagaatttgacttg

SEQ ID NO: 212 - RNA, 21 nt, synthetic construct
  source 1..21: mol_type = other RNA
tccaggctcagaatttgactt

SEQ ID NO: 213 - RNA, 21 nt, synthetic construct
  source 1..21: mol_type = other RNA
tccccaggctcagaatttgac

SEQ ID NO: 214 - RNA, 21 nt, synthetic construct
  source 1..21: mol_type = other RNA
tctccatgctttgcagatgag

SEQ ID NO: 215 - RNA, 21 nt, synthetic construct
  source 1..21: mol_type = other RNA
tactctccatgctttgcagat

SEQ ID NO: 216 - RNA, 21 nt, synthetic construct
  source 1..21: mol_type = other RNA
tcactctccatgctttgcaga

SEQ ID NO: 217 - RNA, 21 nt, synthetic construct
  source 1..21: mol_type = other RNA
tccactctccatgctttgcag

SEQ ID NO: 218 - RNA, 21 nt, synthetic construct
  source 1..21: mol_type = other RNA
tgccactctccatgctttgca

SEQ ID NO: 219 - RNA, 21 nt, synthetic construct
  source 1..21: mol_type = other RNA
atgccactctccatgctttgc

SEQ ID NO: 220 - RNA, 21 nt, synthetic construct
  source 1..21: mol_type = other RNA
tatgccactctccatgctttg

SEQ ID NO: 221 - RNA, 21 nt, synthetic construct
  source 1..21: mol_type = other RNA
agatgccactctccatgcttt

SEQ ID NO: 222 - RNA, 21 nt, synthetic construct
  source 1..21: mol_type = other RNA
tattgtcctcagcagctctga

SEQ ID NO: 223 - RNA, 21 nt, synthetic construct
  source 1..21: mol_type = other RNA
tctacatccccacttctgaag

SEQ ID NO: 224 - RNA, 21 nt, synthetic construct
  source 1..21: mol_type = other RNA
agctacatccccatctctgaa

SEQ ID NO: 225 - RNA, 21 nt, synthetic construct
  source 1..21: mol_type = other RNA
taagctacatccctacctctg

SEQ ID NO: 226 - RNA, 21 nt, synthetic construct
  source 1..21: mol_type = other RNA
tgaagctacatcctcacctct

SEQ ID NO: 227 - RNA, 21 nt, synthetic construct
  source 1..21: mol_type = other RNA
tggaagctacatctccacctc

SEQ ID NO: 228 - RNA, 21 nt, synthetic construct
  source 1..21: mol_type = other RNA
atggaagctacattcccacct

SEQ ID NO: 229 - RNA, 21 nt, synthetic construct
  source 1..21: mol_type = other RNA
tacatggaagctatatcccca

SEQ ID NO: 230 - RNA, 21 nt, synthetic construct
  source 1..21: mol_type = other RNA
taatagcaaacactttgggtg

SEQ ID NO: 231 - RNA, 21 nt, synthetic construct
  source 1..21: mol_type = other RNA
tgaatagcaaacatcttgggt

SEQ ID NO: 232 - RNA, 21 nt, synthetic construct
  source 1..21: mol_type = other RNA
actgaatagcaaataccttgg

SEQ ID NO: 233 - RNA, 21 nt, synthetic construct
  source 1..21: mol_type = other RNA
taaaactgaatagtaaacacc

SEQ ID NO: 234 - RNA, 21 nt, synthetic construct
  source 1..21: mol_type = other RNA
taaaccttttctctatgtcat

SEQ ID NO: 235 - RNA, 21 nt, synthetic construct
  source 1..21: mol_type = other RNA
taacactatcttttaagaagc

SEQ ID NO: 236 - RNA, 21 nt, synthetic construct
  source 1..21: mol_type = other RNA
tctgtaacactatttttcaag

SEQ ID NO: 237 - RNA, 21 nt, synthetic construct
  source 1..21: mol_type = other RNA
aattgactcctcttatatcaa

SEQ ID NO: 238 - RNA, 21 nt, synthetic construct
  source 1..21: mol_type = other RNA
aaaattgactcctttcatatc

SEQ ID NO: 239 - RNA, 21 nt, synthetic construct
  source 1..21: mol_type = other RNA
ttaaaattgactcttctcata

SEQ ID NO: 240 - RNA, 21 nt, synthetic construct
  source 1..21: mol_type = other RNA
attaaaattgacttctctcat

SEQ ID NO: 241 - RNA, 21 nt, synthetic construct
  source 1..21: mol_type = other RNA
acattaaaattgattcctctc

SEQ ID NO: 242 - RNA, 21 nt, synthetic construct
  source 1..21: mol_type = other RNA
tcattcttcaacattgctaac

SEQ ID NO: 243 - RNA, 21 nt, synthetic construct
  source 1..21: mol_type = other RNA
tggcattcttcaatactgcta

SEQ ID NO: 244 - RNA, 21 nt, synthetic construct
  source 1..21: mol_type = other RNA
ttttggcattctttaacactg

SEQ ID NO: 245 - RNA, 21 nt, synthetic construct
  source 1..21: mol_type = other RNA
tagaatccagatttcacgtta

SEQ ID NO: 246 - RNA, 21 nt, synthetic construct
  source 1..21: mol_type = other RNA
ttcagtgagaatctagattcc

SEQ ID NO: 247 - RNA, 21 nt, synthetic construct
  source 1..21: mol_type = other RNA
taaagttcttttgtaggttaa

SEQ ID NO: 248 - RNA, 21 nt, synthetic construct
  source 1..21: mol_type = other RNA
ttcaggtaaagtttttttgca

SEQ ID NO: 249 - RNA, 21 nt, synthetic construct
  source 1..21: mol_type = other RNA
taatggcagggtttaggtaaa

SEQ ID NO: 250 - RNA, 21 nt, synthetic construct
  source 1..21: mol_type = other RNA
aattttagaatggtagggttc

SEQ ID NO: 251 - RNA, 21 nt, synthetic construct
  source 1..21: mol_type = other RNA
taaagtcaactcctgggtaaa

SEQ ID NO: 252 - RNA, 21 nt, synthetic construct
  source 1..21: mol_type = other RNA
tcaaagtcaactctcgggtaa

SEQ ID NO: 253 - RNA, 21 nt, synthetic construct
  source 1..21: mol_type = other RNA
tccaaagtcaacttccgggta

SEQ ID NO: 254 - RNA, 21 nt, synthetic construct
  source 1..21: mol_type = other RNA
ttcaattcttctcttccaaag

SEQ ID NO: 255 - RNA, 21 nt, synthetic construct
  source 1..21: mol_type = other RNA
attcaattcttcttctccaaa

SEQ ID NO: 256 - RNA, 21 nt, synthetic construct
  source 1..21: mol_type = other RNA
tttggcaaacatttactcctt

SEQ ID NO: 257 - RNA, 21 nt, synthetic construct
  source 1..21: mol_type = other RNA
actgacagcgaattatctttg

SEQ ID NO: 258 - RNA, 21 nt, synthetic construct
  source 1..21: mol_type = other RNA
agtgaaaaactgatagcgaat

SEQ ID NO: 259 - RNA, 21 nt, synthetic construct
  source 1..21: mol_type = other RNA
aataagtgaaaaattgacagc

SEQ ID NO: 260 - RNA, 21 nt, synthetic construct
  source 1..21: mol_type = other RNA
tccttacagtcttttgggagt

SEQ ID NO: 261 - RNA, 21 nt, synthetic construct
  source 1..21: mol_type = other RNA
taaccagagctcctttgtgtc

SEQ ID NO: 262 - RNA, 21 nt, synthetic construct
  source 1..21: mol_type = other RNA
ttaaccagagctctcttgtgt

SEQ ID NO: 263 - RNA, 21 nt, synthetic construct
  source 1..21: mol_type = other RNA
agagtaaccagagttcccttg

SEQ ID NO: 264 - RNA, 21 nt, synthetic construct
  source 1..21: mol_type = other RNA
tctcaaagagtaatcagagct

SEQ ID NO: 265 - RNA, 21 nt, synthetic construct
  source 1..21: mol_type = other RNA
tcccagtgttacataatctca

SEQ ID NO: 266 - RNA, 21 nt, synthetic construct
  source 1..21: mol_type = other RNA
tttgttcctccaataatgcgt

SEQ ID NO: 267 - RNA, 21 nt, synthetic construct
  source 1..21: mol_type = other RNA
tagtttgttcctctaacaatg

SEQ ID NO: 268 - RNA, 21 nt, synthetic construct
  source 1..21: mol_type = other RNA
agagtttgttccttcaacaat

SEQ ID NO: 269 - RNA, 21 nt, synthetic construct
  source 1..21: mol_type = other RNA
taagagtttgttcttccaaca

SEQ ID NO: 270 - RNA, 21 nt, synthetic construct
  source 1..21: mol_type = other RNA
agaagagtttgtttctccaac

SEQ ID NO: 271 - RNA, 21 nt, synthetic construct
  source 1..21: mol_type = other RNA
agggccactctcctcaagaag

SEQ ID NO: 272 - RNA, 21 nt, synthetic construct
  source 1..21: mol_type = other RNA
tagggccactctctccaagaa

SEQ ID NO: 273 - RNA, 21 nt, synthetic construct
  source 1..21: mol_type = other RNA
tcagggccactcttcccaaga

SEQ ID NO: 274 - RNA, 21 nt, synthetic construct
  source 1..21: mol_type = other RNA
tagtgaccctccatacaggtg

SEQ ID NO: 275 - RNA, 21 nt, synthetic construct
  source 1..21: mol_type = other RNA
tatgagtgacccttcacacag

SEQ ID NO: 276 - RNA, 21 nt, synthetic construct
  source 1..21: mol_type = other RNA
tctatgagtgaccttccacac

SEQ ID NO: 277 - RNA, 21 nt, synthetic construct
  source 1..21: mol_type = other RNA
tcctatgagtgactctccaca

SEQ ID NO: 278 - RNA, 21 nt, synthetic construct
  source 1..21: mol_type = other RNA
ttcctatgagtgatcctccac

SEQ ID NO: 279 - RNA, 21 nt, synthetic construct
  source 1..21: mol_type = other RNA
acccactggtgtcttatgagt

SEQ ID NO: 280 - RNA, 21 nt, synthetic construct
  source 1..21: mol_type = other RNA
tacccactggtgttctatgag

SEQ ID NO: 281 - RNA, 21 nt, synthetic construct
  source 1..21: mol_type = other RNA
tcttttgtaatgtttgacaga

SEQ ID NO: 282 - RNA, 21 nt, synthetic construct
  source 1..21: mol_type = other RNA
atatcatgattcctttctgag

SEQ ID NO: 283 - RNA, 21 nt, synthetic construct
  source 1..21: mol_type = other RNA
aattcaaaggagcttggagtt

SEQ ID NO: 284 - RNA, 21 nt, synthetic construct
  source 1..21: mol_type = other RNA
taattcaaaggagtctggagt

SEQ ID NO: 285 - RNA, 21 nt, synthetic construct
  source 1..21: mol_type = other RNA
tcccaccaaacgctacattcc

SEQ ID NO: 286 - RNA, 21 nt, synthetic construct
  source 1..21: mol_type = other RNA
tgcccaccaaacgtcacattc

SEQ ID NO: 287 - RNA, 21 nt, synthetic construct
  source 1..21: mol_type = other RNA
tatgcccaccaaatgccacat

SEQ ID NO: 288 - RNA, 21 nt, synthetic construct
  source 1..21: mol_type = other RNA
ttccctgcgggcatagccttc

SEQ ID NO: 289 - RNA, 21 nt, synthetic construct
  source 1..21: mol_type = other RNA
accaggttgctccttgcgggc

SEQ ID NO: 290 - RNA, 21 nt, synthetic construct
  source 1..21: mol_type = other RNA
taccaggttgctctctgcggg

SEQ ID NO: 291 - RNA, 21 nt, synthetic construct
  source 1..21: mol_type = other RNA
acaccaggttgcttcctgcgg

SEQ ID NO: 292 - RNA, 21 nt, synthetic construct
  source 1..21: mol_type = other RNA
agacaccaggttgttccctgc

SEQ ID NO: 293 - RNA, 21 nt, synthetic construct
  source 1..21: mol_type = other RNA
atgtactcagcgattttggtg

SEQ ID NO: 294 - RNA, 21 nt, synthetic construct
  source 1..21: mol_type = other RNA
tccatgtactcagtgactttg

SEQ ID NO: 295 - RNA, 21 nt, synthetic construct
  source 1..21: mol_type = other RNA
tagtccatgtacttagcgact

SEQ ID NO: 296 - RNA, 21 nt, synthetic construct
  source 1..21: mol_type = other RNA
tccagtccatgtattcagcga

SEQ ID NO: 297 - RNA, 21 nt, synthetic construct
  source 1..21: mol_type = other RNA
ttaaaatccagtctatgtact

SEQ ID NO: 298 - RNA, 21 nt, synthetic construct
  source 1..21: mol_type = other RNA
tctaaaatccagttcatgtac

SEQ ID NO: 299 - RNA, 21 nt, synthetic construct
  source 1..21: mol_type = other RNA
ttctgtgttttctttaaaatc

SEQ ID NO: 300 - RNA, 21 nt, synthetic construct
  source 1..21: mol_type = other RNA
tgctctgtgttttttctaaaa

SEQ ID NO: 301 - RNA, 21 nt, synthetic construct
  source 1..21: mol_type = other RNA
ttccatcactgctttgtgttt

SEQ ID NO: 302 - RNA, 21 nt, synthetic construct
  source 1..21: mol_type = other RNA
tctccatgctttgtagatgag

SEQ ID NO: 303 - RNA, 21 nt, synthetic construct
  source 1..21: mol_type = other RNA
tccactctccatgttttgcag

SEQ ID NO: 304 - RNA, 21 nt, synthetic construct
  source 1..21: mol_type = other RNA
agatgccactctctatgcttt

SEQ ID NO: 305 - RNA, 23 nt, synthetic construct
  source 1..23: mol_type = other RNA
tagtgtaattcaaaggagcctgg

SEQ ID NO: 306 - RNA, 23 nt, synthetic construct
  source 1..23: mol_type = other RNA
accaatttctgaaagggcacagg

SEQ ID NO: 307 - RNA, 23 nt, synthetic construct
  source 1..23: mol_type = other RNA
tcacattcaattcttctcctcca

SEQ ID NO: 308 - RNA, 23 nt, synthetic construct
  source 1..23: mol_type = other RNA
aagtgaaaaactgacagcgaacc

SEQ ID NO: 309 - RNA, 23 nt, synthetic construct
  source 1..23: mol_type = other RNA
aatcctagttggagaaccatccg

SEQ ID NO: 310 - RNA, 23 nt, synthetic construct
  source 1..23: mol_type = other RNA
tcaaagagtaaccagagctccct

SEQ ID NO: 311 - RNA, 23 nt, synthetic construct
  source 1..23: mol_type = other RNA
tacagatttaaaatgccactgcg

SEQ ID NO: 312 - RNA, 23 nt, synthetic construct
  source 1..23: mol_type = other RNA
tgtaattcaaaggagcctggagt

SEQ ID NO: 313 - RNA, 23 nt, synthetic construct
  source 1..23: mol_type = other RNA
tcatattggtttttggaattcag

SEQ ID NO: 314 - RNA, 21 nt, synthetic construct
  source 1..21: mol_type = other RNA
tcttcagaggtggggatgtaa

SEQ ID NO: 315 - RNA, 21 nt, synthetic construct
  source 1..21: mol_type = other RNA
cttcagaggtggggatgtaga

SEQ ID NO: 316 - RNA, 21 nt, synthetic construct
  source 1..21: mol_type = other RNA
ttcagaggtggggatgtagct

SEQ ID NO: 317 - RNA, 21 nt, synthetic construct
  source 1..21: mol_type = other RNA
tcagaggtggggatgtagctt

SEQ ID NO: 318 - RNA, 21 nt, synthetic construct
  source 1..21: mol_type = other RNA
cagaggtggggatgtagctta

SEQ ID NO: 319 - RNA, 21 nt, synthetic construct
  source 1..21: mol_type = other RNA
agaggtggggatgtagcttca

SEQ ID NO: 320 - RNA, 21 nt, synthetic construct
  source 1..21: mol_type = other RNA
gaggtggggatgtagcttcca

SEQ ID NO: 321 - RNA, 21 nt, synthetic construct
  source 1..21: mol_type = other RNA
aggtggggatgtagcttccat

SEQ ID NO: 322 - RNA, 21 nt, synthetic construct
  source 1..21: mol_type = other RNA
ggtggggatgtagcttccata

SEQ ID NO: 323 - RNA, 21 nt, synthetic construct
  source 1..21: mol_type = other RNA
gtggggatgtagcttccatgt

SEQ ID NO: 324 - RNA, 21 nt, synthetic construct
  source 1..21: mol_type = other RNA
tggggatgtagcttccatgta

SEQ ID NO: 325 - RNA, 21 nt, synthetic construct
  source 1..21: mol_type = other RNA
ggggatgtagcttccatgtaa

SEQ ID NO: 326 - RNA, 21 nt, synthetic construct
  source 1..21: mol_type = other RNA
gtgcacattccacccaaggta

SEQ ID NO: 327 - RNA, 21 nt, synthetic construct
  source 1..21: mol_type = other RNA
tgcacattccacccaaggtgt

SEQ ID NO: 328 - RNA, 21 nt, synthetic construct
  source 1..21: mol_type = other RNA
gcacattccacccaaggtgtt

SEQ ID NO: 329 - RNA, 21 nt, synthetic construct
  source 1..21: mol_type = other RNA
cattccacccaaggtgtttga

SEQ ID NO: 330 - RNA, 21 nt, synthetic construct
  source 1..21: mol_type = other RNA
attccacccaaggtgtttgct

SEQ ID NO: 331 - RNA, 21 nt, synthetic construct
  source 1..21: mol_type = other RNA
ccacccaaggtgtttgctatt

SEQ ID NO: 332 - RNA, 21 nt, synthetic construct
  source 1..21: mol_type = other RNA
cacccaaggtgtttgctatta

SEQ ID NO: 333 - RNA, 21 nt, synthetic construct
  source 1..21: mol_type = other RNA
acccaaggtgtttgctattca

SEQ ID NO: 334 - RNA, 21 nt, synthetic construct
  source 1..21: mol_type = other RNA
ccaaggtgtttgctattcagt

SEQ ID NO: 335 - RNA, 21 nt, synthetic construct
  source 1..21: mol_type = other RNA
caaggtgtttgctattcagtt

SEQ ID NO: 336 - RNA, 21 nt, synthetic construct
  source 1..21: mol_type = other RNA
aaggtgtttgctattcagttt

SEQ ID NO: 337 - RNA, 21 nt, synthetic construct
  source 1..21: mol_type = other RNA
aggtgtttgctattcagtttt

SEQ ID NO: 338 - RNA, 21 nt, synthetic construct
  source 1..21: mol_type = other RNA
ggtgtttgctattcagtttta

SEQ ID NO: 339 - RNA, 21 nt, synthetic construct
  source 1..21: mol_type = other RNA
gtgtttgctattcagttttct

SEQ ID NO: 340 - RNA, 21 nt, synthetic construct
  source 1..21: mol_type = other RNA
ttgctattcagttttcttcca

SEQ ID NO: 341 - RNA, 21 nt, synthetic construct
  source 1..21: mol_type = other RNA
cagttttcttccagcaagtta

SEQ ID NO: 342 - RNA, 21 nt, synthetic construct
  source 1..21: mol_type = other RNA
atcaatgacatggagaaaaga

SEQ ID NO: 343 - RNA, 21 nt, synthetic construct
  source 1..21: mol_type = other RNA
aatgacatggagaaaaggttt

SEQ ID NO: 344 - RNA, 21 nt, synthetic construct
  source 1..21: mol_type = other RNA
atgacatggagaaaaggttta

SEQ ID NO: 345 - RNA, 21 nt, synthetic construct
  source 1..21: mol_type = other RNA
tgaagaatgccaaaaaaggta

SEQ ID NO: 346 - RNA, 21 nt, synthetic construct
  source 1..21: mol_type = other RNA
aatgccaaaaaaggtgcacca

SEQ ID NO: 347 - RNA, 21 nt, synthetic construct
  source 1..21: mol_type = other RNA
tgtgccctttcagaaattggt

SEQ ID NO: 348 - RNA, 21 nt, synthetic construct
  source 1..21: mol_type = other RNA
gtgccctttcagaaattggtt

SEQ ID NO: 349 - RNA, 21 nt, synthetic construct
  source 1..21: mol_type = other RNA
gaacatcttccagcatcttga

SEQ ID NO: 350 - RNA, 21 nt, synthetic construct
  source 1..21: mol_type = other RNA
aaagtggcacaccaagttcct

SEQ ID NO: 351 - RNA, 21 nt, synthetic construct
  source 1..21: mol_type = other RNA
aagtggcacaccaagttccta

SEQ ID NO: 352 - RNA, 21 nt, synthetic construct
  source 1..21: mol_type = other RNA
agtggcacaccaagttcctct

SEQ ID NO: 353 - RNA, 21 nt, synthetic construct
  source 1..21: mol_type = other RNA
gtggcacaccaagttcctcta

SEQ ID NO: 354 - RNA, 21 nt, synthetic construct
  source 1..21: mol_type = other RNA
tggcacaccaagttcctctaa

SEQ ID NO: 355 - RNA, 21 nt, synthetic construct
  source 1..21: mol_type = other RNA
caccaagttcctctactccta

SEQ ID NO: 356 - RNA, 21 nt, synthetic construct
  source 1..21: mol_type = other RNA
accaagttcctctactcctca

SEQ ID NO: 357 - RNA, 21 nt, synthetic construct
  source 1..21: mol_type = other RNA
ccaagttcctctactcctcaa

SEQ ID NO: 358 - RNA, 21 nt, synthetic construct
  source 1..21: mol_type = other RNA
caagttcctctactcctcaaa

SEQ ID NO: 359 - RNA, 21 nt, synthetic construct
  source 1..21: mol_type = other RNA
aagttcctctactcctcaaga

SEQ ID NO: 360 - RNA, 21 nt, synthetic construct
  source 1..21: mol_type = other RNA
agttcctctactcctcaagaa

SEQ ID NO: 361 - RNA, 21 nt, synthetic construct
  source 1..21: mol_type = other RNA
gttcctctactcctcaagaaa

SEQ ID NO: 362 - RNA, 21 nt, synthetic construct
  source 1..21: mol_type = other RNA
ttcctctactcctcaagaaaa

SEQ ID NO: 363 - RNA, 21 nt, synthetic construct
  source 1..21: mol_type = other RNA
ctctactcctcaagaaaacaa

SEQ ID NO: 364 - RNA, 21 nt, synthetic construct
  source 1..21: mol_type = other RNA
tctactcctcaagaaaacaca

SEQ ID NO: 365 - RNA, 21 nt, synthetic construct
  source 1..21: mol_type = other RNA
ctactcctcaagaaaacacca

SEQ ID NO: 366 - RNA, 21 nt, synthetic construct
  source 1..21: mol_type = other RNA
agccttttaacctgcaaaaga

SEQ ID NO: 367 - RNA, 21 nt, synthetic construct
  source 1..21: mol_type = other RNA
aaagaactttacctgaaccct

SEQ ID NO: 368 - RNA, 21 nt, synthetic construct
  source 1..21: mol_type = other RNA
aagaactttacctgaacccta

SEQ ID NO: 369 - RNA, 21 nt, synthetic construct
  source 1..21: mol_type = other RNA
agaactttacctgaaccctga

SEQ ID NO: 370 - RNA, 21 nt, synthetic construct
  source 1..21: mol_type = other RNA
gaactttacctgaaccctgca

SEQ ID NO: 371 - RNA, 21 nt, synthetic construct
  source 1..21: mol_type = other RNA
aactttacctgaaccctgcca

SEQ ID NO: 372 - RNA, 21 nt, synthetic construct
  source 1..21: mol_type = other RNA
actttacctgaaccctgccat

SEQ ID NO: 373 - RNA, 21 nt, synthetic construct
  source 1..21: mol_type = other RNA
tttacctgaaccctgccatta

SEQ ID NO: 374 - RNA, 21 nt, synthetic construct
  source 1..21: mol_type = other RNA
ttacctgaaccctgccattct

SEQ ID NO: 375 - RNA, 21 nt, synthetic construct
  source 1..21: mol_type = other RNA
tacctgaaccctgccattcta

SEQ ID NO: 376 - RNA, 21 nt, synthetic construct
  source 1..21: mol_type = other RNA
acctgaaccctgccattctaa

SEQ ID NO: 377 - RNA, 21 nt, synthetic construct
  source 1..21: mol_type = other RNA
cctgaaccctgccattctaaa

SEQ ID NO: 378 - RNA, 21 nt, synthetic construct
  source 1..21: mol_type = other RNA
ctgaaccctgccattctaaaa

SEQ ID NO: 379 - RNA, 21 nt, synthetic construct
  source 1..21: mol_type = other RNA
tgaaccctgccattctaaaat

SEQ ID NO: 380 - RNA, 21 nt, synthetic construct
  source 1..21: mol_type = other RNA
gaaccctgccattctaaaatt

SEQ ID NO: 381 - RNA, 21 nt, synthetic construct
  source 1..21: mol_type = other RNA
aaccctgccattctaaaattt

SEQ ID NO: 382 - RNA, 21 nt, synthetic construct
  source 1..21: mol_type = other RNA
accctgccattctaaaattta

SEQ ID NO: 383 - RNA, 21 nt, synthetic construct
  source 1..21: mol_type = other RNA
ccctgccattctaaaatttaa

SEQ ID NO: 384 - RNA, 21 nt, synthetic construct
  source 1..21: mol_type = other RNA
cctgccattctaaaatttaca

SEQ ID NO: 385 - RNA, 21 nt, synthetic construct
  source 1..21: mol_type = other RNA
gccattctaaaatttacccga

SEQ ID NO: 386 - RNA, 21 nt, synthetic construct
  source 1..21: mol_type = other RNA
ccattctaaaatttacccgga

SEQ ID NO: 387 - RNA, 21 nt, synthetic construct
  source 1..21: mol_type = other RNA
ggaggagaagaattgaatgta

SEQ ID NO: 388 - RNA, 21 nt, synthetic construct
  source 1..21: mol_type = other RNA
gaggagaagaattgaatgtga

SEQ ID NO: 389 - RNA, 21 nt, synthetic construct
  source 1..21: mol_type = other RNA
ggagaagaattgaatgtgact

SEQ ID NO: 390 - RNA, 21 nt, synthetic construct
  source 1..21: mol_type = other RNA
ttaaaggagtgaatgtttgca

SEQ ID NO: 391 - RNA, 21 nt, synthetic construct
  source 1..21: mol_type = other RNA
caaagatgattcgctgtcagt

SEQ ID NO: 392 - RNA, 21 nt, synthetic construct
  source 1..21: mol_type = other RNA
aaagatgattcgctgtcagtt

SEQ ID NO: 393 - RNA, 21 nt, synthetic construct
  source 1..21: mol_type = other RNA
aagatgattcgctgtcagttt

SEQ ID NO: 394 - RNA, 21 nt, synthetic construct
  source 1..21: mol_type = other RNA
agatgattcgctgtcagtttt

SEQ ID NO: 395 - RNA, 21 nt, synthetic construct
  source 1..21: mol_type = other RNA
gatgattcgctgtcagttttt

SEQ ID NO: 396 - RNA, 21 nt, synthetic construct
  source 1..21: mol_type = other RNA
gattcgctgtcagtttttcaa

SEQ ID NO: 397 - RNA, 21 nt, synthetic construct
  source 1..21: mol_type = other RNA
attcgctgtcagtttttcact

SEQ ID NO: 398 - RNA, 21 nt, synthetic construct
  source 1..21: mol_type = other RNA
ttcgctgtcagtttttcactt

SEQ ID NO: 399 - RNA, 21 nt, synthetic construct
  source 1..21: mol_type = other RNA
tcgctgtcagtttttcactta

SEQ ID NO: 400 - RNA, 21 nt, synthetic construct
  source 1..21: mol_type = other RNA
cgctgtcagtttttcacttat

SEQ ID NO: 401 - RNA, 21 nt, synthetic construct
  source 1..21: mol_type = other RNA
gctgtcagtttttcacttatt

SEQ ID NO: 402 - RNA, 21 nt, synthetic construct
  source 1..21: mol_type = other RNA
gtcagtttttcacttattctt

SEQ ID NO: 403 - RNA, 21 nt, synthetic construct
  source 1..21: mol_type = other RNA
tcagtttttcacttattcttt

SEQ ID NO: 404 - RNA, 21 nt, synthetic construct
  source 1..21: mol_type = other RNA
tttttcacttattctttacta

SEQ ID NO: 405 - RNA, 21 nt, synthetic construct
  source 1..21: mol_type = other RNA
ttttcacttattctttactca

SEQ ID NO: 406 - RNA, 21 nt, synthetic construct
  source 1..21: mol_type = other RNA
tttcacttattctttactcca

SEQ ID NO: 407 - RNA, 21 nt, synthetic construct
  source 1..21: mol_type = other RNA
ttcacttattctttactccca

SEQ ID NO: 408 - RNA, 21 nt, synthetic construct
  source 1..21: mol_type = other RNA
tcacttattctttactcccaa

SEQ ID NO: 409 - RNA, 21 nt, synthetic construct
  source 1..21: mol_type = other RNA
cacttattctttactcccaga

SEQ ID NO: 410 - RNA, 21 nt, synthetic construct
  source 1..21: mol_type = other RNA
acttattctttactcccagaa

SEQ ID NO: 411 - RNA, 21 nt, synthetic construct
  source 1..21: mol_type = other RNA
cttattctttactcccagaaa

SEQ ID NO: 412 - RNA, 21 nt, synthetic construct
  source 1..21: mol_type = other RNA
ttattctttactcccagaaga

SEQ ID NO: 413 - RNA, 21 nt, synthetic construct
  source 1..21: mol_type = other RNA
attctttactcccagaagact

SEQ ID NO: 414 - RNA, 21 nt, synthetic construct
  source 1..21: mol_type = other RNA
ttctttactcccagaagacta

SEQ ID NO: 415 - RNA, 21 nt, synthetic construct
  source 1..21: mol_type = other RNA
ttactcccagaagactgtaaa

SEQ ID NO: 416 - RNA, 21 nt, synthetic construct
  source 1..21: mol_type = other RNA
tactcccagaagactgtaaga

SEQ ID NO: 417 - RNA, 21 nt, synthetic construct
  source 1..21: mol_type = other RNA
actcccagaagactgtaagga

SEQ ID NO: 418 - RNA, 21 nt, synthetic construct
  source 1..21: mol_type = other RNA
gagaagtgtaagtgtttctta

SEQ ID NO: 419 - RNA, 21 nt, synthetic construct
  source 1..21: mol_type = other RNA
agaagtgtaagtgtttcttaa

SEQ ID NO: 420 - RNA, 21 nt, synthetic construct
  source 1..21: mol_type = other RNA
gaagtgtaagtgtttcttaaa

SEQ ID NO: 421 - RNA, 21 nt, synthetic construct
  source 1..21: mol_type = other RNA
taagtgtttcttaagattata

SEQ ID NO: 422 - RNA, 21 nt, synthetic construct
  source 1..21: mol_type = other RNA
ggatggttctccaactaggat

SEQ ID NO: 423 - RNA, 21 nt, synthetic construct
  source 1..21: mol_type = other RNA
gatggttctccaactaggatt

SEQ ID NO: 424 - RNA, 21 nt, synthetic construct
  source 1..21: mol_type = other RNA
atgggacacaagggagctcta

SEQ ID NO: 425 - RNA, 21 nt, synthetic construct
  source 1..21: mol_type = other RNA
tgggacacaagggagctctga

SEQ ID NO: 426 - RNA, 21 nt, synthetic construct
  source 1..21: mol_type = other RNA
ggacacaagggagctctggtt

SEQ ID NO: 427 - RNA, 21 nt, synthetic construct
  source 1..21: mol_type = other RNA
gacacaagggagctctggtta

SEQ ID NO: 428 - RNA, 21 nt, synthetic construct
  source 1..21: mol_type = other RNA
acacaagggagctctggttaa

SEQ ID NO: 429 - RNA, 21 nt, synthetic construct
  source 1..21: mol_type = other RNA
acaagggagctctggttacta

SEQ ID NO: 430 - RNA, 21 nt, synthetic construct
  source 1..21: mol_type = other RNA
caagggagctctggttactct

SEQ ID NO: 431 - RNA, 21 nt, synthetic construct
  source 1..21: mol_type = other RNA
ggagctctggttactctttga

SEQ ID NO: 432 - RNA, 21 nt, synthetic construct
  source 1..21: mol_type = other RNA
ttgagattgtgtaacactgga

SEQ ID NO: 433 - RNA, 21 nt, synthetic construct
  source 1..21: mol_type = other RNA
tgagattgtgtaacactggga

SEQ ID NO: 434 - RNA, 21 nt, synthetic construct
  source 1..21: mol_type = other RNA
gattgtgtaacactggggaca

SEQ ID NO: 435 - RNA, 21 nt, synthetic construct
  source 1..21: mol_type = other RNA
aggaacaaactcttcttggga

SEQ ID NO: 436 - RNA, 21 nt, synthetic construct
  source 1..21: mol_type = other RNA
ggaacaaactcttcttgggga

SEQ ID NO: 437 - RNA, 21 nt, synthetic construct
  source 1..21: mol_type = other RNA
gaacaaactcttcttggggaa

SEQ ID NO: 438 - RNA, 21 nt, synthetic construct
  source 1..21: mol_type = other RNA
aacaaactcttcttggggaga

SEQ ID NO: 439 - RNA, 21 nt, synthetic construct
  source 1..21: mol_type = other RNA
acaaactcttcttggggagaa

SEQ ID NO: 440 - RNA, 21 nt, synthetic construct
  source 1..21: mol_type = other RNA
caaactcttcttggggagagt

SEQ ID NO: 441 - RNA, 21 nt, synthetic construct
  source 1..21: mol_type = other RNA
aactcttcttggggagagtga

SEQ ID NO: 442 - RNA, 21 nt, synthetic construct
  source 1..21: mol_type = other RNA
actcttcttggggagagtgga

SEQ ID NO: 443 - RNA, 21 nt, synthetic construct
  source 1..21: mol_type = other RNA
cttcttggggagagtggccct

SEQ ID NO: 444 - RNA, 21 nt, synthetic construct
  source 1..21: mol_type = other RNA
ttcttggggagagtggcccta

SEQ ID NO: 445 - RNA, 21 nt, synthetic construct
  source 1..21: mol_type = other RNA
tcttggggagagtggccctga

SEQ ID NO: 446 - RNA, 21 nt, synthetic construct
  source 1..21: mol_type = other RNA
gggtcactcataggacaccaa

SEQ ID NO: 447 - RNA, 21 nt, synthetic construct
  source 1..21: mol_type = other RNA
ggtcactcataggacaccagt

SEQ ID NO: 448 - RNA, 21 nt, synthetic construct
  source 1..21: mol_type = other RNA
cactcataggacaccagtgga

SEQ ID NO: 449 - RNA, 21 nt, synthetic construct
  source 1..21: mol_type = other RNA
actcataggacaccagtgggt

SEQ ID NO: 450 - RNA, 21 nt, synthetic construct
  source 1..21: mol_type = other RNA
ctcataggacaccagtgggta

SEQ ID NO: 451 - RNA, 21 nt, synthetic construct
  source 1..21: mol_type = other RNA
ccactgctttgatgggcttca

SEQ ID NO: 452 - RNA, 21 nt, synthetic construct
  source 1..21: mol_type = other RNA
atagtggcattttaaatctgt

SEQ ID NO: 453 - RNA, 21 nt, synthetic construct
  source 1..21: mol_type = other RNA
tagtggcattttaaatctgta

SEQ ID NO: 454 - RNA, 21 nt, synthetic construct
  source 1..21: mol_type = other RNA
agtggcattttaaatctgtca

SEQ ID NO: 455 - RNA, 21 nt, synthetic construct
  source 1..21: mol_type = other RNA
gtggcattttaaatctgtcaa

SEQ ID NO: 456 - RNA, 21 nt, synthetic construct
  source 1..21: mol_type = other RNA
tggcattttaaatctgtcaga

SEQ ID NO: 457 - RNA, 21 nt, synthetic construct
  source 1..21: mol_type = other RNA
ggcattttaaatctgtcagaa

SEQ ID NO: 458 - RNA, 21 nt, synthetic construct
  source 1..21: mol_type = other RNA
gcattttaaatctgtcagaca

SEQ ID NO: 459 - RNA, 21 nt, synthetic construct
  source 1..21: mol_type = other RNA
ttaaatctgtcagacattaca

SEQ ID NO: 460 - RNA, 21 nt, synthetic construct
  source 1..21: mol_type = other RNA
tctgtcagacattacaaaaga

SEQ ID NO: 461 - RNA, 21 nt, synthetic construct
  source 1..21: mol_type = other RNA
ctcagaagggaatcatgatat

SEQ ID NO: 462 - RNA, 21 nt, synthetic construct
  source 1..21: mol_type = other RNA
agaagggaatcatgatatcga

SEQ ID NO: 463 - RNA, 21 nt, synthetic construct
  source 1..21: mol_type = other RNA
gaagggaatcatgatatcgca

SEQ ID NO: 464 - RNA, 21 nt, synthetic construct
  source 1..21: mol_type = other RNA
aagggaatcatgatatcgcct

SEQ ID NO: 465 - RNA, 21 nt, synthetic construct
  source 1..21: mol_type = other RNA
aactccaggctcctttgaatt

SEQ ID NO: 466 - RNA, 21 nt, synthetic construct
  source 1..21: mol_type = other RNA
actccaggctcctttgaatta

SEQ ID NO: 467 - RNA, 21 nt, synthetic construct
  source 1..21: mol_type = other RNA
ctccaggctcctttgaattaa

SEQ ID NO: 468 - RNA, 21 nt, synthetic construct
  source 1..21: mol_type = other RNA
tccaggctcctttgaattaca

SEQ ID NO: 469 - RNA, 21 nt, synthetic construct
  source 1..21: mol_type = other RNA
ccaggctcctttgaattacaa

SEQ ID NO: 470 - RNA, 21 nt, synthetic construct
  source 1..21: mol_type = other RNA
caggctcctttgaattacact

SEQ ID NO: 471 - RNA, 21 nt, synthetic construct
  source 1..21: mol_type = other RNA
aggctcctttgaattacacta

SEQ ID NO: 472 - RNA, 21 nt, synthetic construct
  source 1..21: mol_type = other RNA
ggctcctttgaattacactga

SEQ ID NO: 473 - RNA, 21 nt, synthetic construct
  source 1..21: mol_type = other RNA
ctttgaattacactgaattca

SEQ ID NO: 474 - RNA, 21 nt, synthetic construct
  source 1..21: mol_type = other RNA
cactgaattccaaaaaccaat

SEQ ID NO: 475 - RNA, 21 nt, synthetic construct
  source 1..21: mol_type = other RNA
actgaattccaaaaaccaata

SEQ ID NO: 476 - RNA, 21 nt, synthetic construct
  source 1..21: mol_type = other RNA
tgaattccaaaaaccaatata

SEQ ID NO: 477 - RNA, 21 nt, synthetic construct
  source 1..21: mol_type = other RNA
gaattccaaaaaccaatatga

SEQ ID NO: 478 - RNA, 21 nt, synthetic construct
  source 1..21: mol_type = other RNA
tccaaaaaccaatatgcctaa

SEQ ID NO: 479 - RNA, 21 nt, synthetic construct
  source 1..21: mol_type = other RNA
ccaaaaaccaatatgcctaca

SEQ ID NO: 480 - RNA, 21 nt, synthetic construct
  source 1..21: mol_type = other RNA
caaaaaccaatatgcctacct

SEQ ID NO: 481 - RNA, 21 nt, synthetic construct
  source 1..21: mol_type = other RNA
aaaaaccaatatgcctacctt

SEQ ID NO: 482 - RNA, 21 nt, synthetic construct
  source 1..21: mol_type = other RNA
aaaaccaatatgcctacctta

SEQ ID NO: 483 - RNA, 21 nt, synthetic construct
  source 1..21: mol_type = other RNA
aaaccaatatgcctaccttca

SEQ ID NO: 484 - RNA, 21 nt, synthetic construct
  source 1..21: mol_type = other RNA
aaccaatatgcctaccttcca

SEQ ID NO: 485 - RNA, 21 nt, synthetic construct
  source 1..21: mol_type = other RNA
atgcctaccttccaaaggtga

SEQ ID NO: 486 - RNA, 21 nt, synthetic construct
  source 1..21: mol_type = other RNA
cctaccttccaaaggtgacaa

SEQ ID NO: 487 - RNA, 21 nt, synthetic construct
  source 1..21: mol_type = other RNA
ctaccttccaaaggtgacaca

SEQ ID NO: 488 - RNA, 21 nt, synthetic construct
  source 1..21: mol_type = other RNA
taccttccaaaggtgacacaa

SEQ ID NO: 489 - RNA, 21 nt, synthetic construct
  source 1..21: mol_type = other RNA
gtggcgtttggtgggcatcaa

SEQ ID NO: 490 - RNA, 21 nt, synthetic construct
  source 1..21: mol_type = other RNA
gaaggctgtgcccgcagggaa

SEQ ID NO: 491 - RNA, 21 nt, synthetic construct
  source 1..21: mol_type = other RNA
gcccgcagggagcaacctggt

SEQ ID NO: 492 - RNA, 21 nt, synthetic construct
  source 1..21: mol_type = other RNA
cccgcagggagcaacctggta

SEQ ID NO: 493 - RNA, 21 nt, synthetic construct
  source 1..21: mol_type = other RNA
ccgcagggagcaacctggtgt

SEQ ID NO: 494 - RNA, 21 nt, synthetic construct
  source 1..21: mol_type = other RNA
cgcagggagcaacctggtgta

SEQ ID NO: 495 - RNA, 21 nt, synthetic construct
  source 1..21: mol_type = other RNA
gcagggagcaacctggtgtct

SEQ ID NO: 496 - RNA, 21 nt, synthetic construct
  source 1..21: mol_type = other RNA
cagggagcaacctggtgtcta

SEQ ID NO: 497 - RNA, 21 nt, synthetic construct
  source 1..21: mol_type = other RNA
agggagcaacctggtgtctaa

SEQ ID NO: 498 - RNA, 21 nt, synthetic construct
  source 1..21: mol_type = other RNA
gggagcaacctggtgtctaca

SEQ ID NO: 499 - RNA, 21 nt, synthetic construct
  source 1..21: mol_type = other RNA
ggagcaacctggtgtctacaa

SEQ ID NO: 500 - RNA, 21 nt, synthetic construct
  source 1..21: mol_type = other RNA
ggtgtctacaccaaagtcgct

SEQ ID NO: 501 - RNA, 21 nt, synthetic construct
  source 1..21: mol_type = other RNA
gtcgctgagtacatggactga

SEQ ID NO: 502 - RNA, 21 nt, synthetic construct
  source 1..21: mol_type = other RNA
tcgctgagtacatggactgga

SEQ ID NO: 503 - RNA, 21 nt, synthetic construct
  source 1..21: mol_type = other RNA
cgctgagtacatggactggat

SEQ ID NO: 504 - RNA, 21 nt, synthetic construct
  source 1..21: mol_type = other RNA
ggattttagagaaaacacaga

SEQ ID NO: 505 - RNA, 21 nt, synthetic construct
  source 1..21: mol_type = other RNA
gattttagagaaaacacagaa

SEQ ID NO: 506 - RNA, 21 nt, synthetic construct
  source 1..21: mol_type = other RNA
attttagagaaaacacagaga

SEQ ID NO: 507 - RNA, 21 nt, synthetic construct
  source 1..21: mol_type = other RNA
ttttagagaaaacacagagca

SEQ ID NO: 508 - RNA, 21 nt, synthetic construct
  source 1..21: mol_type = other RNA
tttagagaaaacacagagcaa

SEQ ID NO: 509 - RNA, 21 nt, synthetic construct
  source 1..21: mol_type = other RNA
agaaaacacagagcagtgata

SEQ ID NO: 510 - RNA, 21 nt, synthetic construct
  source 1..21: mol_type = other RNA
aaacacagagcagtgatggaa

SEQ ID NO: 511 - RNA, 21 nt, synthetic construct
  source 1..21: mol_type = other RNA
aacacagagcagtgatggaaa

SEQ ID NO: 512 - RNA, 21 nt, synthetic construct
  source 1..21: mol_type = other RNA
gttcaagtcaaattctgagca

SEQ ID NO: 513 - RNA, 21 nt, synthetic construct
  source 1..21: mol_type = other RNA
ttcaagtcaaattctgagcct

SEQ ID NO: 514 - RNA, 21 nt, synthetic construct
  source 1..21: mol_type = other RNA
tcaagtcaaattctgagccta

SEQ ID NO: 515 - RNA, 21 nt, synthetic construct
  source 1..21: mol_type = other RNA
caagtcaaattctgagcctga

SEQ ID NO: 516 - RNA, 21 nt, synthetic construct
  source 1..21: mol_type = other RNA
aagtcaaattctgagcctgga

SEQ ID NO: 517 - RNA, 21 nt, synthetic construct
  source 1..21: mol_type = other RNA
gtcaaattctgagcctgggga

SEQ ID NO: 518 - RNA, 21 nt, synthetic construct
  source 1..21: mol_type = other RNA
ctcatctgcaaagcatggaga

SEQ ID NO: 519 - RNA, 21 nt, synthetic construct
  source 1..21: mol_type = other RNA
atctgcaaagcatggagagta

SEQ ID NO: 520 - RNA, 21 nt, synthetic construct
  source 1..21: mol_type = other RNA
tctgcaaagcatggagagtga

SEQ ID NO: 521 - RNA, 21 nt, synthetic construct
  source 1..21: mol_type = other RNA
ctgcaaagcatggagagtgga

SEQ ID NO: 522 - RNA, 21 nt, synthetic construct
  source 1..21: mol_type = other RNA
tgcaaagcatggagagtggca

SEQ ID NO: 523 - RNA, 21 nt, synthetic construct
  source 1..21: mol_type = other RNA
gcaaagcatggagagtggcat

SEQ ID NO: 524 - RNA, 21 nt, synthetic construct
  source 1..21: mol_type = other RNA
caaagcatggagagtggcata

SEQ ID NO: 525 - RNA, 21 nt, synthetic construct
  source 1..21: mol_type = other RNA
aaagcatggagagtggcatct

SEQ ID NO: 526 - RNA, 21 nt, synthetic construct
  source 1..21: mol_type = other RNA
tcagagctgctgaggacaata

SEQ ID NO: 527 - RNA, 21 nt, synthetic construct
  source 1..21: mol_type = other RNA
cttcagaagtggggatgtaga

SEQ ID NO: 528 - RNA, 21 nt, synthetic construct
  source 1..21: mol_type = other RNA
ttcagagatggggatgtagct

SEQ ID NO: 529 - RNA, 21 nt, synthetic construct
  source 1..21: mol_type = other RNA
cagaggtagggatgtagctta

SEQ ID NO: 530 - RNA, 21 nt, synthetic construct
  source 1..21: mol_type = other RNA
agaggtgaggatgtagcttca

SEQ ID NO: 531 - RNA, 21 nt, synthetic construct
  source 1..21: mol_type = other RNA
gaggtggagatgtagcttcca

SEQ ID NO: 532 - RNA, 21 nt, synthetic construct
  source 1..21: mol_type = other RNA
aggtgggaatgtagcttccat

SEQ ID NO: 533 - RNA, 21 nt, synthetic construct
  source 1..21: mol_type = other RNA
tggggatatagcttccatgta

SEQ ID NO: 534 - RNA, 21 nt, synthetic construct
  source 1..21: mol_type = other RNA
cacccaaagtgtttgctatta

SEQ ID NO: 535 - RNA, 21 nt, synthetic construct
  source 1..21: mol_type = other RNA
acccaagatgtttgctattca

SEQ ID NO: 536 - RNA, 21 nt, synthetic construct
  source 1..21: mol_type = other RNA
ccaaggtatttgctattcagt

SEQ ID NO: 537 - RNA, 21 nt, synthetic construct
  source 1..21: mol_type = other RNA
ggtgtttactattcagtttta

SEQ ID NO: 538 - RNA, 21 nt, synthetic construct
  source 1..21: mol_type = other RNA
atgacatagagaaaaggttta

SEQ ID NO: 539 - RNA, 21 nt, synthetic construct
  source 1..21: mol_type = other RNA
gcttcttaaaagatagtgtta

SEQ ID NO: 540 - RNA, 21 nt, synthetic construct
  source 1..21: mol_type = other RNA
cttgaaaaatagtgttacaga

SEQ ID NO: 541 - RNA, 21 nt, synthetic construct
  source 1..21: mol_type = other RNA
ttgatataagaggagtcaatt

SEQ ID NO: 542 - RNA, 21 nt, synthetic construct
  source 1..21: mol_type = other RNA
gatatgaaaggagtcaatttt

SEQ ID NO: 543 - RNA, 21 nt, synthetic construct
  source 1..21: mol_type = other RNA
tatgagaagagtcaattttaa

SEQ ID NO: 544 - RNA, 21 nt, synthetic construct
  source 1..21: mol_type = other RNA
atgagagaagtcaattttaat

SEQ ID NO: 545 - RNA, 21 nt, synthetic construct
  source 1..21: mol_type = other RNA
gagaggaatcaattttaatgt

SEQ ID NO: 546 - RNA, 21 nt, synthetic construct
  source 1..21: mol_type = other RNA
gttagcaatgttgaagaatga

SEQ ID NO: 547 - RNA, 21 nt, synthetic construct
  source 1..21: mol_type = other RNA
tagcagtattgaagaatgcca

SEQ ID NO: 548 - RNA, 21 nt, synthetic construct
  source 1..21: mol_type = other RNA
cagtgttaaagaatgccaaaa

SEQ ID NO: 549 - RNA, 21 nt, synthetic construct
  source 1..21: mol_type = other RNA
taacgtgaaatctggattcta

SEQ ID NO: 550 - RNA, 21 nt, synthetic construct
  source 1..21: mol_type = other RNA
ggaatctagattctcactgaa

SEQ ID NO: 551 - RNA, 21 nt, synthetic construct
  source 1..21: mol_type = other RNA
ttaacctacaaaagaacttta

SEQ ID NO: 552 - RNA, 21 nt, synthetic construct
  source 1..21: mol_type = other RNA
tgcaaaaaaactttacctgaa

SEQ ID NO: 553 - RNA, 21 nt, synthetic construct
  source 1..21: mol_type = other RNA
tttacctaaaccctgccatta

SEQ ID NO: 554 - RNA, 21 nt, synthetic construct
  source 1..21: mol_type = other RNA
gaaccctaccattctaaaatt

SEQ ID NO: 555 - RNA, 21 nt, synthetic construct
  source 1..21: mol_type = other RNA
tttacccaggagttgacttta

SEQ ID NO: 556 - RNA, 21 nt, synthetic construct
  source 1..21: mol_type = other RNA
ttacccgagagttgactttga

SEQ ID NO: 557 - RNA, 21 nt, synthetic construct
  source 1..21: mol_type = other RNA
tacccggaagttgactttgga

SEQ ID NO: 558 - RNA, 21 nt, synthetic construct
  source 1..21: mol_type = other RNA
ctttggaagagaagaattgaa

SEQ ID NO: 559 - RNA, 21 nt, synthetic construct
  source 1..21: mol_type = other RNA
tttggagaagaagaattgaat

SEQ ID NO: 560 - RNA, 21 nt, synthetic construct
  source 1..21: mol_type = other RNA
aaggagtaaatgtttgccaaa

SEQ ID NO: 561 - RNA, 21 nt, synthetic construct
  source 1..21: mol_type = other RNA
caaagataattcgctgtcagt

SEQ ID NO: 562 - RNA, 21 nt, synthetic construct
  source 1..21: mol_type = other RNA
attcgctatcagtttttcact

SEQ ID NO: 563 - RNA, 21 nt, synthetic construct
  source 1..21: mol_type = other RNA
gctgtcaatttttcacttatt

SEQ ID NO: 564 - RNA, 21 nt, synthetic construct
  source 1..21: mol_type = other RNA
actcccaaaagactgtaagga

SEQ ID NO: 565 - RNA, 21 nt, synthetic construct
  source 1..21: mol_type = other RNA
gacacaaaggagctctggtta

SEQ ID NO: 566 - RNA, 21 nt, synthetic construct
  source 1..21: mol_type = other RNA
acacaagagagctctggttaa

SEQ ID NO: 567 - RNA, 21 nt, synthetic construct
  source 1..21: mol_type = other RNA
caagggaactctggttactct

SEQ ID NO: 568 - RNA, 21 nt, synthetic construct
  source 1..21: mol_type = other RNA
agctctgattactctttgaga

SEQ ID NO: 569 - RNA, 21 nt, synthetic construct
  source 1..21: mol_type = other RNA
tgagattatgtaacactggga

SEQ ID NO: 570 - RNA, 21 nt, synthetic construct
  source 1..21: mol_type = other RNA
acgcattattggaggaacaaa

SEQ ID NO: 571 - RNA, 21 nt, synthetic construct
  source 1..21: mol_type = other RNA
cattgttagaggaacaaacta

SEQ ID NO: 572 - RNA, 21 nt, synthetic construct
  source 1..21: mol_type = other RNA
attgttgaaggaacaaactct

SEQ ID NO: 573 - RNA, 21 nt, synthetic construct
  source 1..21: mol_type = other RNA
tgttggaagaacaaactctta

SEQ ID NO: 574 - RNA, 21 nt, synthetic construct
  source 1..21: mol_type = other RNA
gttggagaaacaaactcttct

SEQ ID NO: 575 - RNA, 21 nt, synthetic construct
  source 1..21: mol_type = other RNA
cttcttgaggagagtggccct

SEQ ID NO: 576 - RNA, 21 nt, synthetic construct
  source 1..21: mol_type = other RNA
ttcttggagagagtggcccta

SEQ ID NO: 577 - RNA, 21 nt, synthetic construct
  source 1..21: mol_type = other RNA
tcttgggaagagtggccctga

SEQ ID NO: 578 - RNA, 21 nt, synthetic construct
  source 1..21: mol_type = other RNA
cacctgtatggagggtcacta

SEQ ID NO: 579 - RNA, 21 nt, synthetic construct
  source 1..21: mol_type = other RNA
ctgtgtgaagggtcactcata

SEQ ID NO: 580 - RNA, 21 nt, synthetic construct
  source 1..21: mol_type = other RNA
gtgtggaaggtcactcataga

SEQ ID NO: 581 - RNA, 21 nt, synthetic construct
  source 1..21: mol_type = other RNA
tgtggagagtcactcatagga

SEQ ID NO: 582 - RNA, 21 nt, synthetic construct
  source 1..21: mol_type = other RNA
gtggaggatcactcataggaa

SEQ ID NO: 583 - RNA, 21 nt, synthetic construct
  source 1..21: mol_type = other RNA
actcataagacaccagtgggt

SEQ ID NO: 584 - RNA, 21 nt, synthetic construct
  source 1..21: mol_type = other RNA
ctcatagaacaccagtgggta

SEQ ID NO: 585 - RNA, 21 nt, synthetic construct
  source 1..21: mol_type = other RNA
tctgtcaaacattacaaaaga

SEQ ID NO: 586 - RNA, 21 nt, synthetic construct
  source 1..21: mol_type = other RNA
ctcagaaaggaatcatgatat

SEQ ID NO: 587 - RNA, 21 nt, synthetic construct
  source 1..21: mol_type = other RNA
aactccaagctcctttgaatt

SEQ ID NO: 588 - RNA, 21 nt, synthetic construct
  source 1..21: mol_type = other RNA
actccagactcctttgaatta

SEQ ID NO: 589 - RNA, 21 nt, synthetic construct
  source 1..21: mol_type = other RNA
ggaatgtagcgtttggtggga

SEQ ID NO: 590 - RNA, 21 nt, synthetic construct
  source 1..21: mol_type = other RNA
gaatgtgacgtttggtgggca

SEQ ID NO: 591 - RNA, 21 nt, synthetic construct
  source 1..21: mol_type = other RNA
atgtggcatttggtgggcata

SEQ ID NO: 592 - RNA, 21 nt, synthetic construct
  source 1..21: mol_type = other RNA
gaaggctatgcccgcagggaa

SEQ ID NO: 593 - RNA, 21 nt, synthetic construct
  source 1..21: mol_type = other RNA
gcccgcaaggagcaacctggt

SEQ ID NO: 594 - RNA, 21 nt, synthetic construct
  source 1..21: mol_type = other RNA
cccgcagagagcaacctggta

SEQ ID NO: 595 - RNA, 21 nt, synthetic construct
  source 1..21: mol_type = other RNA
ccgcaggaagcaacctggtgt

SEQ ID NO: 596 - RNA, 21 nt, synthetic construct
  source 1..21: mol_type = other RNA
gcagggaacaacctggtgtct

SEQ ID NO: 597 - RNA, 21 nt, synthetic construct
  source 1..21: mol_type = other RNA
caccaaaatcgctgagtacat

SEQ ID NO: 598 - RNA, 21 nt, synthetic construct
  source 1..21: mol_type = other RNA
caaagtcactgagtacatgga

SEQ ID NO: 599 - RNA, 21 nt, synthetic construct
  source 1..21: mol_type = other RNA
agtcgctaagtacatggacta

SEQ ID NO: 600 - RNA, 21 nt, synthetic construct
  source 1..21: mol_type = other RNA
tcgctgaatacatggactgga

SEQ ID NO: 601 - RNA, 21 nt, synthetic construct
  source 1..21: mol_type = other RNA
agtacatagactggattttaa

SEQ ID NO: 602 - RNA, 21 nt, synthetic construct
  source 1..21: mol_type = other RNA
gtacatgaactggattttaga

SEQ ID NO: 603 - RNA, 21 nt, synthetic construct
  source 1..21: mol_type = other RNA
gattttaaagaaaacacagaa

SEQ ID NO: 604 - RNA, 21 nt, synthetic construct
  source 1..21: mol_type = other RNA
ttttagaaaaaacacagagca

SEQ ID NO: 605 - RNA, 21 nt, synthetic construct
  source 1..21: mol_type = other RNA
aaacacaaagcagtgatggaa

SEQ ID NO: 606 - RNA, 21 nt, synthetic construct
  source 1..21: mol_type = other RNA
ctcatctacaaagcatggaga

SEQ ID NO: 607 - RNA, 21 nt, synthetic construct
  source 1..21: mol_type = other RNA
ctgcaaaacatggagagtgga

SEQ ID NO: 608 - RNA, 21 nt, synthetic construct
  source 1..21: mol_type = other RNA
aaagcatagagagtggcatct

SEQ ID NO: 609 - RNA, 22 nt, synthetic construct
  source 1..22: mol_type = other RNA
caggctcctttgaattacacta

SEQ ID NO: 610 - RNA, 24 nt, synthetic construct
  source 1..24: mol_type = other RNA
tcctgtgccctttcagaaattggt

SEQ ID NO: 611 - RNA, 23 nt, synthetic construct
  source 1..23: mol_type = other RNA
cggaggagaagaattgaatgtga

SEQ ID NO: 612 - RNA, 24 nt, synthetic construct
  source 1..24: mol_type = other RNA
tggttcgctgtcagtttttcactt

SEQ ID NO: 613 - RNA, 23 nt, synthetic construct
  source 1..23: mol_type = other RNA
cggatggttctccaactaggatt

SEQ ID NO: 614 - RNA, 22 nt, synthetic construct
  source 1..22: mol_type = other RNA
tggagctctggttactctttga

SEQ ID NO: 615 - RNA, 24 nt, synthetic construct
  source 1..24: mol_type = other RNA
tcgcagtggcattttaaatctgta

SEQ ID NO: 616 - RNA, 23 nt, synthetic construct
  source 1..23: mol_type = other RNA
tctccaggctcctttgaattaca

SEQ ID NO: 617 - RNA, 21 nt, synthetic construct
  source 1..21: mol_type = other RNA
agggagcaacctggtgtctaa

SEQ ID NO: 618 - RNA, 21 nt, synthetic construct
  source 1..21: mol_type = other RNA
cgctgagtacatggactggat

SEQ ID NO: 619 - RNA, 22 nt, synthetic construct
  source 1..22: mol_type = other RNA
ctccaggctcctttgaattaca

SEQ ID NO: 620 - RNA, 24 nt, synthetic construct
  source 1..24: mol_type = other RNA
tctccaggctcctttgaattacat

SEQ ID NO: 621 - RNA, 23 nt, synthetic construct
  source 1..23: mol_type = other RNA
tggaggagaagaattgaatgtga

SEQ ID NO: 622 - RNA, 24 nt, synthetic construct
  source 1..24: mol_type = other RNA
tggaggagaagaattgaatgtgat

SEQ ID NO: 623 - RNA, 23 nt, synthetic construct
  source 1..23: mol_type = other RNA
ctgaattccaaaaaccaatatga

SEQ ID NO: 624 - RNA, 15 nt, synthetic construct
  source 1..15: mol_type = other RNA
ttcctttgaattaca

SEQ ID NO: 625 - RNA, 23 nt, synthetic construct
  source 1..23: mol_type = other RNA
tgagaatccagattccacgttac

SEQ ID NO: 626 - RNA, 23 nt, synthetic construct
  source 1..23: mol_type = other RNA
tcaagatgctggaagatgttcat

SEQ ID NO: 627 - RNA, 23 nt, synthetic construct
  source 1..23: mol_type = other RNA
acaatctcaaagagtaaccagag

SEQ ID NO: 628 - RNA, 22 nt, synthetic construct
  source 1..22: mol_type = other RNA
taacgtggaatctggattctca

SEQ ID NO: 629 - RNA, 22 nt, synthetic construct
  source 1..22: mol_type = other RNA
tgaacatcttccagcatcttga

SEQ ID NO: 630 - RNA, 23 nt, synthetic construct
  source 1..23: mol_type = other RNA
ctctggttactctttgagattgt